(19) United States
(12) Patent Application Publication
(10) Pub. No.: US 2022/0033431 A1
(43) Pub. Date: Feb. 3, 2022

(54) Cyclic Di-Nucleotide Compounds as STING Agonists

(71) Applicant: Merck Sharp & Dohme Corp., Rahway, NJ (US)

(72) Inventors: Michael D. Altman, Needham, MA (US); Wonsuk Chang, Princeton, NJ (US); Jared N. Cumming, Winchester, MA (US); Hong Liu, Hillsborough, NJ (US); Benjamin Wesley Trotter, Medfield, MA (US)

(73) Assignee: Merck Sharp & Dohme Corp., Rahway, NJ (US)

(21) Appl. No.: 17/299,359

(22) PCT Filed: Dec. 2, 2019
(86) PCT No.: PCT/US19/63908

§ 371 Date: Jun. 3, 2021

Related U.S. Application Data
(60) Provisional application No. 62/776,717, filed on Dec. 7, 2018.

(51) Int. Cl. C07H 19/14; C07H 19/16; C07H 19/23

(52) U.S. Cl. CPC C07H 19/14; C07H 19/23; C07H 19/16

(57) ABSTRACT

A class of polycyclic compounds of general formula (I), wherein Base1, Base2, Y, Ya, Xa, Xa1, Xb, Xb1, Xc, Xc1, Xd, Xd1, R¹, R1a, R2a, R3, R3a, R4, R4a, R5, R6, R6a, R7, R7a, R8, R8a, and R9 are defined herein, that may be useful as inductors of type I interferon production, specifically as STING active agents, are provided. Also provided are processes for the synthesis and use of compounds. (I)

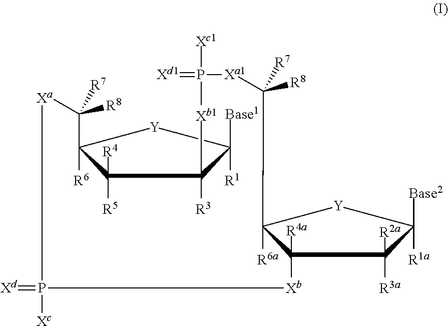

### FIELD OF THE INVENTION

The present disclosure relates to cyclic di-nucleotide compounds and derivatives thereof that may be useful as STING (Stimulator of Interferon Genes) agonists that activate the STING pathway. The present disclosure also relates to processes for the synthesis and to uses of such cyclic di-nucleotide compounds.

### REFERENCE TO SEQUENCE LISTING SUBMITTED ELECTRONICALLY

The sequence listing of the present application is submitted electronically via EFS-Web as an ASCII-formatted sequence listing, with a file name of “24678WOPCT-SEQLIST 22OCT2019”, a creation date of Oct. 22, 2019, and a size of 24.6 KB. This sequence listing submitted via EFS-Web is part of the specification and is herein incorporated by reference in its entirety.

### BACKGROUND OF THE INVENTION

The immune system has evolved to recognize and neutralize different types of threats in order to maintain the homeostasis of the host, and it is generally broken down into two arms: adaptive and innate. The adaptive immune system is specialized to recognize as foreign those antigens not naturally expressed in the host and to mount an anti-antigen response through the coordinated actions of many leukocyte subsets. The hallmark of adaptive immune responses is the ability to provide “memory” or long-lasting immunity against the encountered antigen. While this specific and long-lasting effect is critical to host health and survival, the adaptive immune response requires time to generate a full-blown response.

The innate immune system compensates for this time delay and is specialized to act quickly against different insults or danger signals. It provides the first line of defense against bacteria, viruses, parasites and other infectious threats, but it also responds strongly to certain danger signals associated with cellular or tissue damage. The innate immune system has no antigen specificity but does respond to a variety of effector mechanisms. Opsonization, phagocytosis, activation of the complement system, and production of soluble bioactive molecules such as cytokines or chemokines are all mechanisms by which the innate immune system mediates its response. By responding to these damage-associated molecular patterns (DAMPs) or pathogen-associated molecular patterns (PAMPs) described above, the innate immune system is able to provide broad protection against a wide range of threats to the host.

Free cytosolic DNA and RNA are among these PAMPs and DAMPs. It has recently been demonstrated that the main sensor for cytosolic DNA is cGAS (cyclic GMP-AMP synthase). Upon recognition of cytosolic DNA, cGAS catalyzes the generation of the cyclic-dinucleotide 2′3′-cGAMP, an atypical second messenger that strongly binds to the ER-transmembrane adaptor protein STING. A conformational change is undergone by cGAMP-bound STING, which translocates to a perinuclear compartment and induces the activation of critical transcription factors IRF-3 and NF-κB. This leads to a strong induction of type I interferons and production of pro-inflammatory cytokines such as IL-6, TNF-α and IFN-γ.

The importance of type I interferons and pro-inflammatory cytokines on various cells of the immune system has been very well established. In particular, these molecules strongly potentiate T-cell activation by enhancing the ability of dendritic cells and macrophages to uptake, process, present and cross-present antigens to T-cells. The T-cell stimulatory capacity of these antigen-presenting cells is augmented by the up-regulation of critical co-stimulatory molecules, such as CD80 or CD86. Finally, type I interferons can rapidly engage their cognate receptors and trigger the activation of interferon-responsive genes that can significantly contribute to adaptive immune cell activation.

From a therapeutic perspective, type I interferons are shown to have antiviral activities by directly inhibiting human hepatitis B virus and hepatitis C virus replication, and by stimulating immune responses to virally infected cells. Compounds that can induce type I interferon production are used in vaccines, where they act as adjuvants, enhancing specific immune responses to antigens and minimizing side effects by reducing dosage and broadening the immune response.

In addition, interferons, and compounds that can induce interferon production, have potential use in the treatment of human cancers. Such molecules are potentially useful as anti cancer agents with multiple pathways of activity. Interferons can inhibit human tumor cell proliferation directly and may be synergistic with various approved chemotherapeutic agents. Type I interferons can significantly enhance anti-tumor immune responses by inducing activation of both the adaptive and innate immune cells. Finally, tumor invasiveness may be inhibited by interferons by modulating enzyme expression related to tissue remodeling.

In view of the potential of type I interferons and type I interferon-inducing compounds as anti-viral and anti-cancer agents, there remains a need for new agents that can induce potent type I interferon production. With the growing body of data demonstrating that the cGAS-STING cytosolic DNA sensory pathway has a significant capacity to induce type I interferons, the development of STING activating agents is rapidly taking an important place in to day's anti-tumor therapy landscape.

### SUMMARY OF THE INVENTION

The present disclosure relates to novel compounds of general formula (I). In particular, the present disclosure relates to compounds having the general structural formula (I):

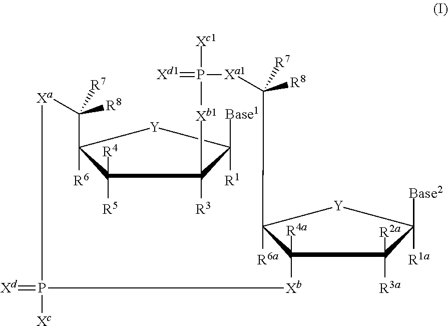

or pharmaceutically acceptable salts, hydrates, solvates, or prodrugs thereof, as described herein. Embodiments of the disclosure include compounds of general formula (I), and pharmaceutically acceptable salts, hydrates, solvates, or prodrugs thereof, as well as synthesis and isolation of compounds of general formula (I), and pharmaceutically acceptable salts, hydrates, solvates, or prodrugs thereof. The compounds of general formula (I), and their pharmaceutically acceptable salts, hydrates, solvates, and/or prodrugs, may be useful as agents to induce immune responses, to induce STING-dependent type I interferon production, and/or to treat a cell proliferation disorders, such as cancers, in a subject. The compounds of general formula (I) could further be used in combination with other therapeutically effective agents, including but not limited to, other drugs useful for the treatment of cell proliferation disorders, such as cancers. The invention further relates to processes for preparing compounds of general formula (I), and pharmaceutical compositions that comprise compounds of general formula (I) and pharmaceutically acceptable salts thereof.

Other embodiments, aspects and features of the present invention are either further described in or will be apparent from the ensuing description, examples and appended claims.

### DETAILED DESCRIPTION OF THE INVENTION

The present disclosure includes compounds of general formula (I), and pharmaceutically acceptable salts, hydrates, solvates, or prodrugs thereof. These compounds and their pharmaceutically acceptable salts, hydrates, solvates, and/or prodrugs may be useful as agents to induce immune responses, to induce STING-dependent type I interferon production, and/or to treat a cell proliferation disorder.

Embodiments disclosed herein relate to compounds of general formula (I):

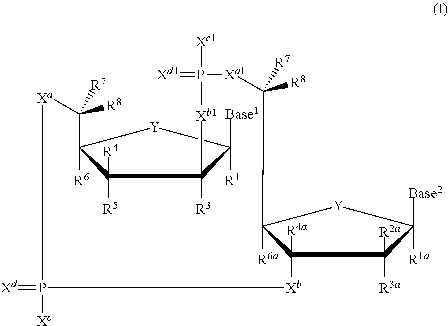

The present disclosure includes compounds of general formula (I), and pharmaceutically acceptable salts, hydrates, solvates, or prodrugs thereof. These compounds and their pharmaceutically acceptable salts, hydrates, solvates, and/or prodrugs may be useful as agents to induce immune responses, to induce STING-dependent type I interferon production, and/or to treat a cell proliferation disorder.

Embodiments disclosed herein relate to compounds of general formula (I) or pharmaceutically acceptable salts, hydrates, solvates, or prodrugs thereof, wherein Base¹ and Base² are each independently selected from the group consisting of

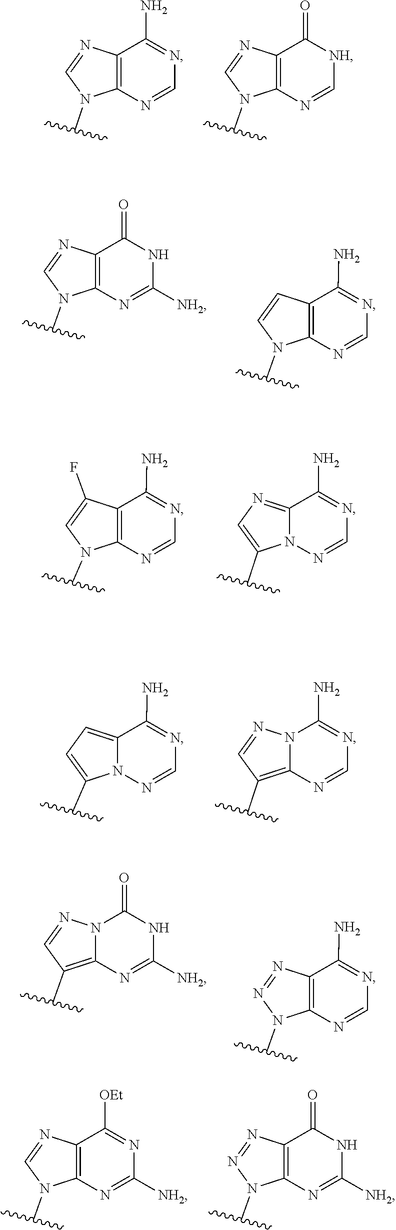

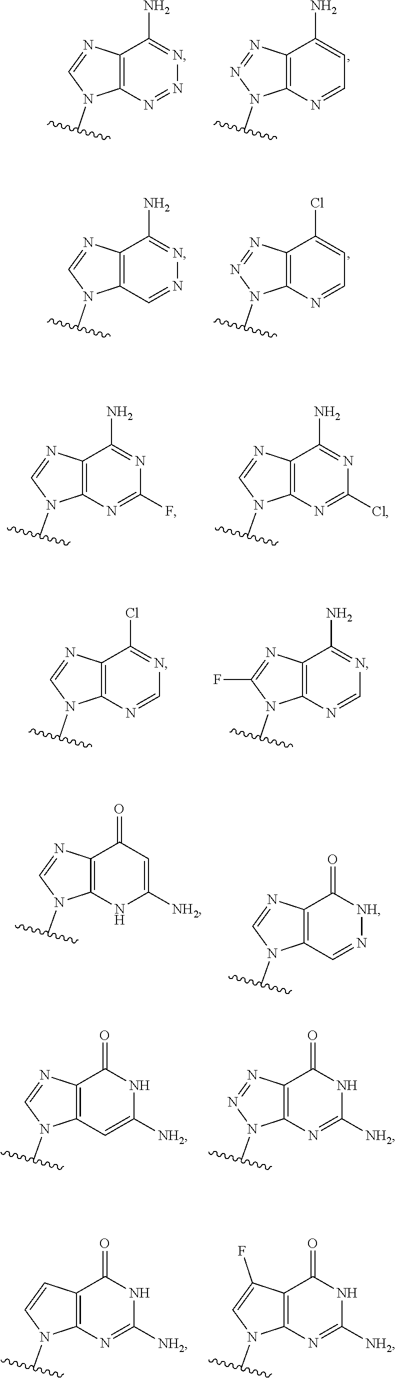

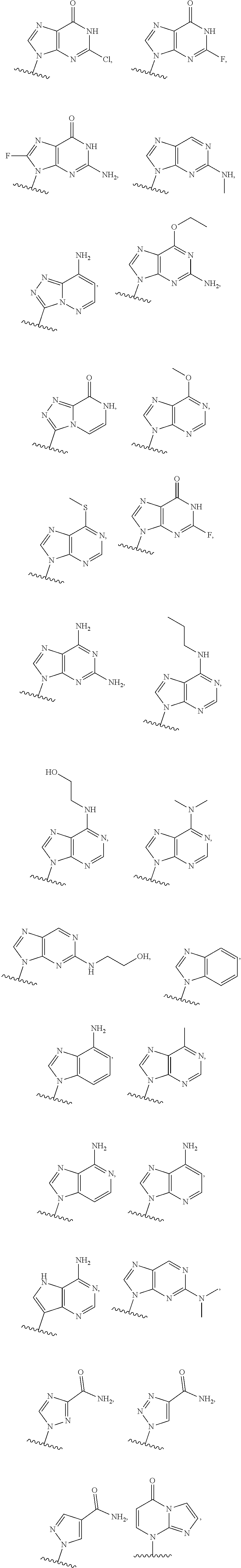

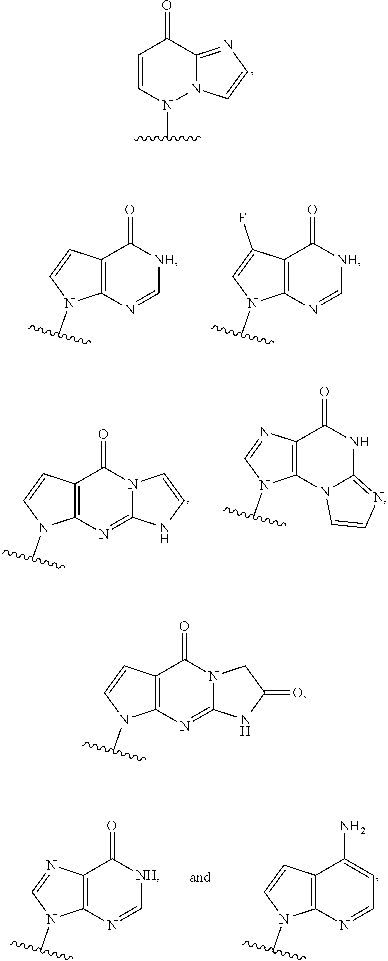

where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O—, —S—, —SO₂—, —CH₂—, and —CF₂—; X^{a} and X^{a1} are each independently selected from the group consisting of —O—, —S—, and —CH₂—; X^{b} and X^{b1} are each independently selected from the group consisting of —O—, —S—, and —CH₂—; X^{c} and X^{c1} are each independently selected from the group consisting of —SR⁹, —OR⁹, and —NR⁹R⁹; X^{d} and X^{d1} are each independently selected from the group consisting of O and S; R¹ and R^{1a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R¹ and R^{1a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R^{2a} is selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R^{2a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R³ and R^{3a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R³ and R^{3a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁴ and R^{4a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁴ and R^{4a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁵ is selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁵ C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁶ and R^{6a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁶ and R^{6a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁷ and R^{7a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁷ and R^{7a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; R⁸ and R^{8a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁸ and R^{8a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃; each R⁹ is independently selected from the group consisting of H, C₁-C₂₀ alkyl,

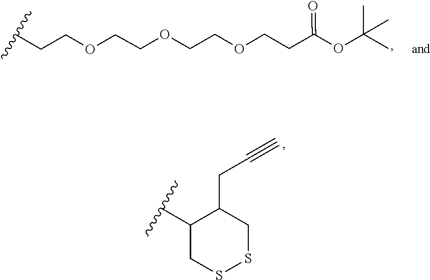

where each R⁹ C₁-C₂₀ alkyl is optionally substituted by 0 to 3 substituents independently selected from the group consisting of OH, —O—C₁-C₂₀ alkyl, —S—C(O)C₁-C₆ alkyl, and C(O)OC₁-C₆ alkyl; optionally R^{1a} and R^{3a} are connected to form C₁-C₆ alkylene, C₂-C₆ alkenylene, —O—C₁-C₆ alkylene, or —O—C₂-C₆ alkenylene, such that where R^{1a} and R^{3a} are connected to form —O—C₁-C₆ alkylene, or —O—C₂-C₆ alkenylene, said O is bound at the R^{3a} position; optionally R^{2a} and R^{3a} are connected to form C₁-C₆ alkylene, C₂-C₆ alkenylene, —O—C₁-C₆ alkylene, or —O—C₂-C₆ alkenylene, such that where R^{2a} and R^{3a} are connected to form —O—C₁-C₆ alkylene, or —O—C₂-C₆ alkenylene, said O is bound at the R^{3a} position; optionally R^{3a} and R^{6a} are connected to form C₁-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene, or —O—C₂-C₆ alkenylene, said O is bound at the R^{3a} position; optionally R⁴ and R⁵ are connected to form C₁-C₆ alkylene, C₂-C₆ alkenylene, —O—C₁-C₆ alkylene, or —O—C₂-C₆ alkenylene, such that where R⁴ and R⁵ are connected to form —O—C₁-C₆ alkylene, or —O—C₂-C₆ alkenylene, said O is bound at the R⁵ position; optionally R⁵ and R⁶ are connected to form —O—C₁-C₆ alkylene, —O—C₂-C₆ alkenylene, or —O—C₂-C₆ alkynylene, such that where R⁵ and R⁶ are connected to form —O—C₁-C₆ alkylene, —O—C₂-C₆ alkenylene, or —O—C₂-C₆ alkynylene, said O is bound at the R⁵ position; optionally R⁷ and R⁸ are connected to form C₁-C₆ alkylene or C₂-C₆ alkenylene; and optionally R^{7a} and R^{8a} are connected to form C₁-C₆ alkylene or C₂-C₆ alkenylene.

In each embodiment described herein, variables Base¹, Base¹, Y, Y^{a}, X^{a}, X^{a1}, X^{b}, X^{b1}, X^{c}, X^{c1}, X^{d}, X^{d1}, R¹, R^{1a}, R^{2a}, R³, R^{3a}, R⁴, R^{4a}, R⁵, R⁶, R^{6a}, R⁷, R^{7a}, R⁸, R^{8a}, and R⁹ of general formula (I), and the various aspects thereof, are each selected independently from each other.

In a first embodiment, Base¹ and Base² are each independently selected from the group consisting of

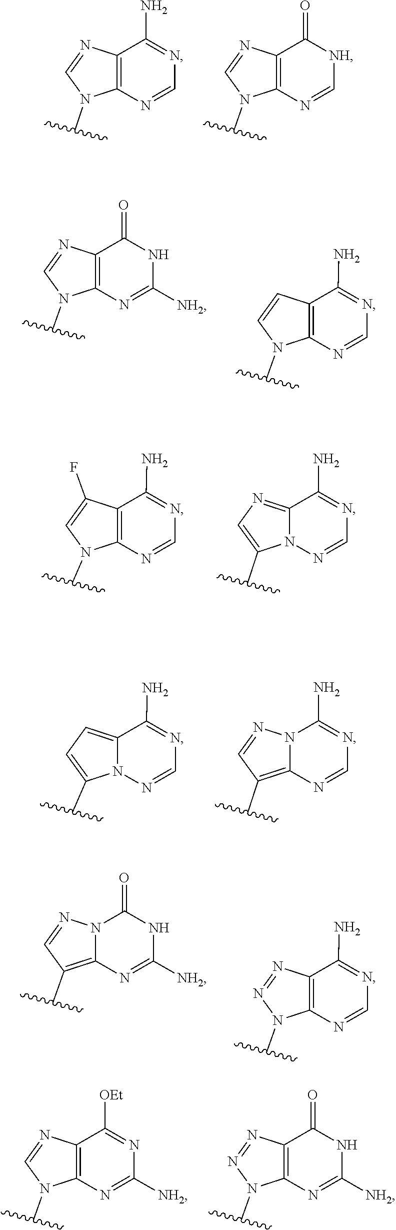

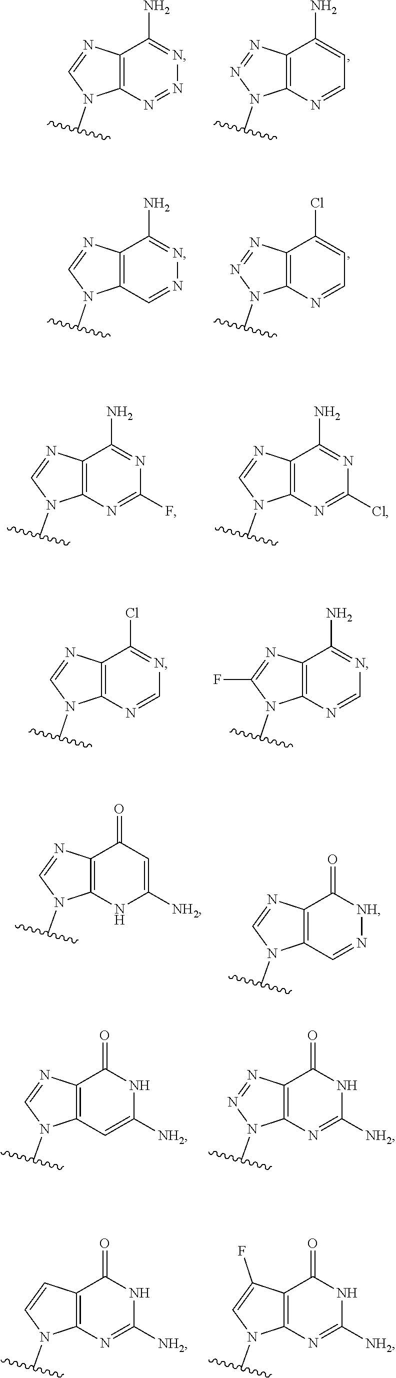

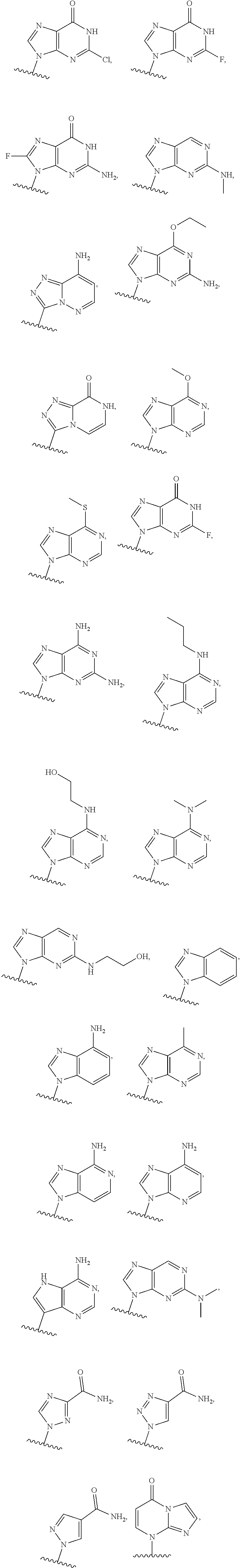

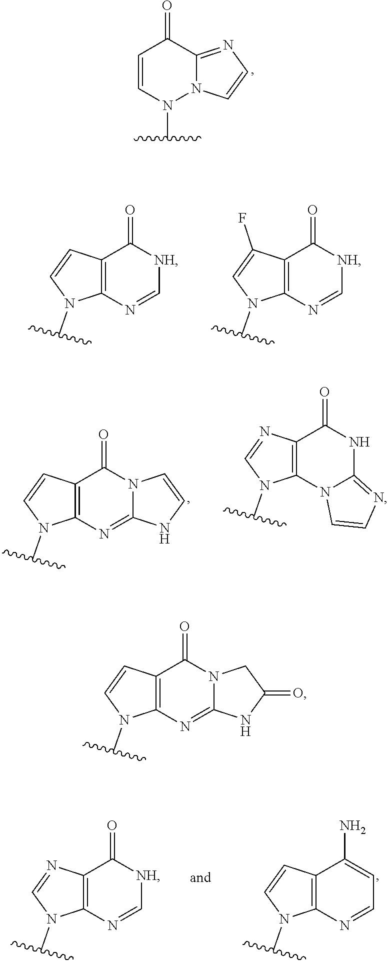

and, and where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂. In particular aspects, Base¹ and Base² are each independently selected from the group consisting of

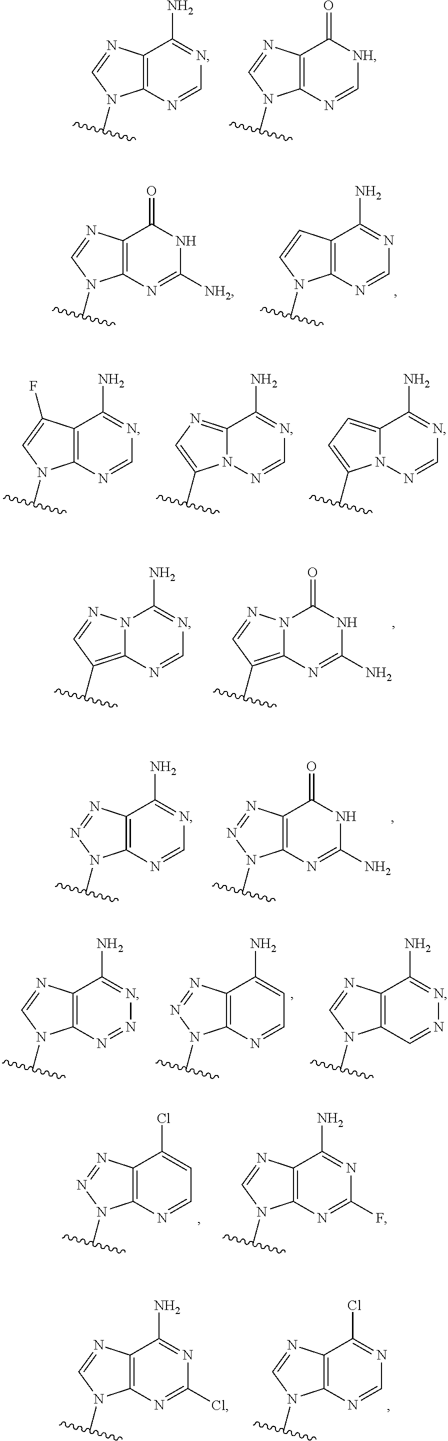

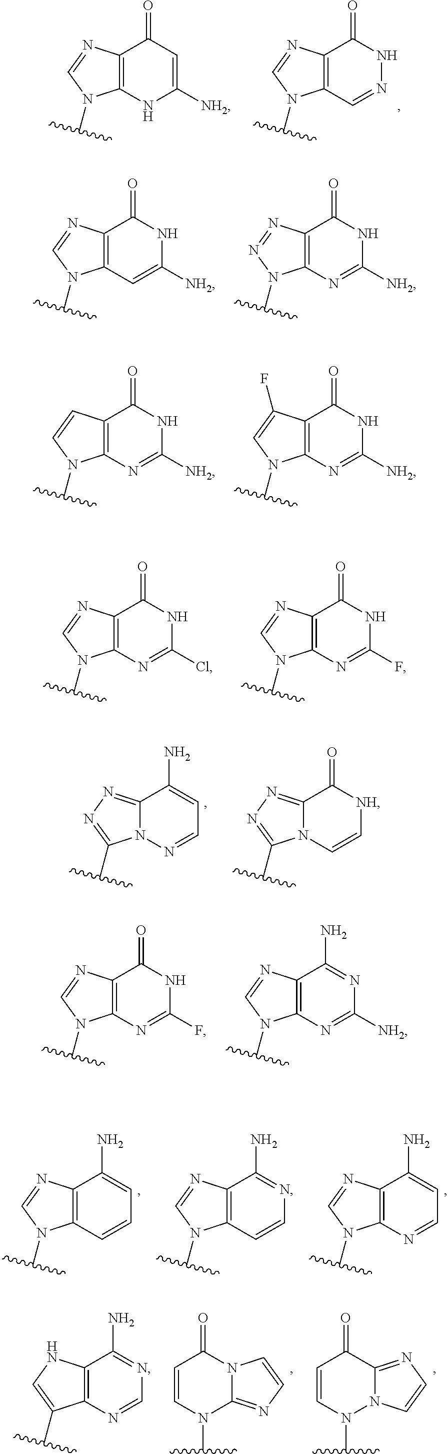

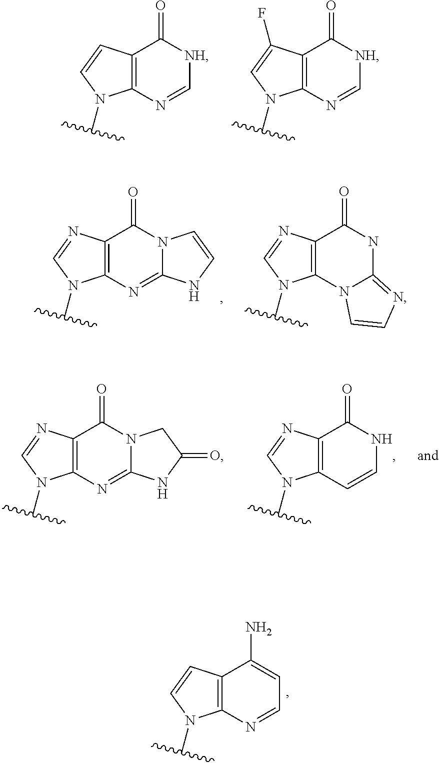

and where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂. In even more particular aspects, Base¹ and Base² are each independently selected from the group consisting of

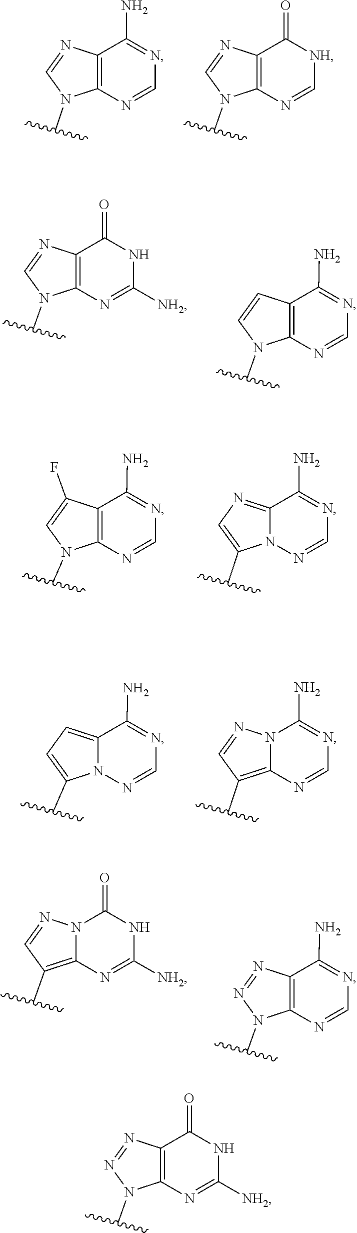

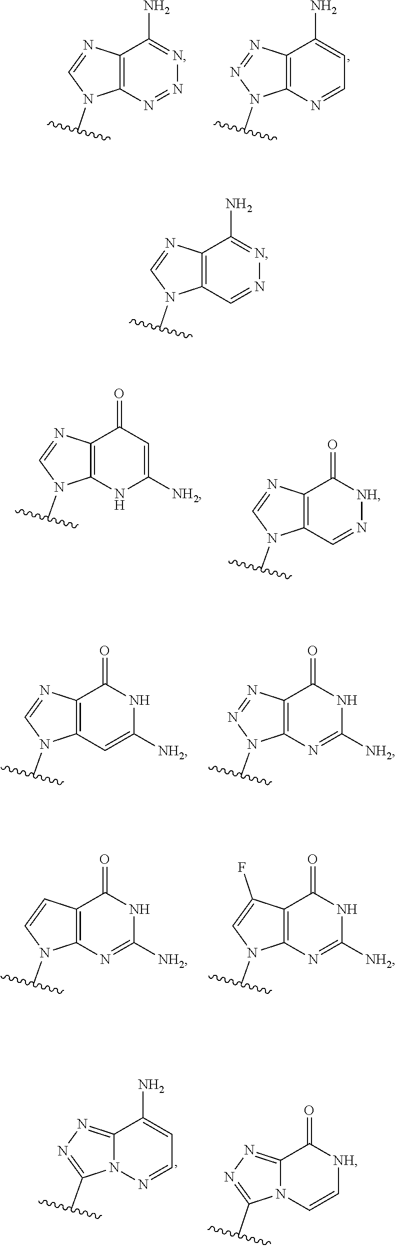

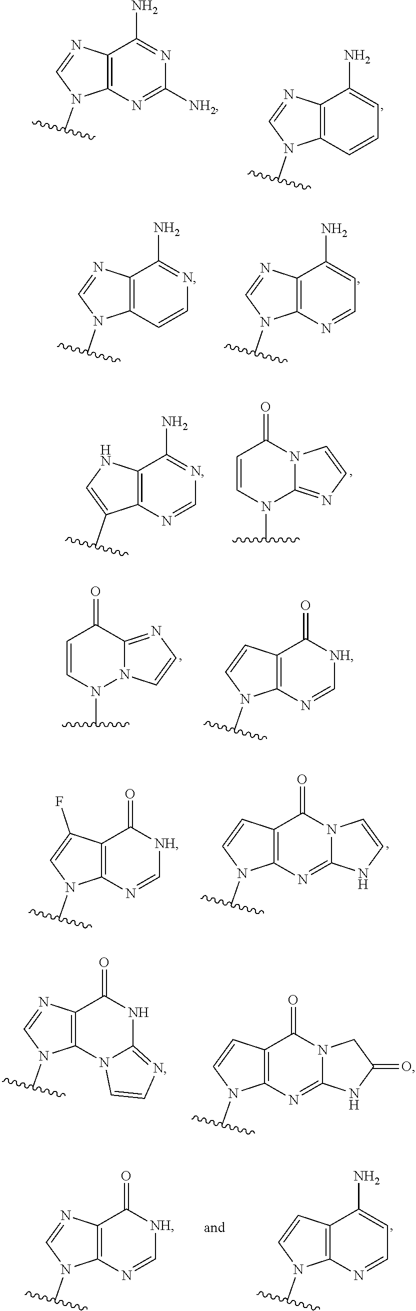

and where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂. In even more particular aspects, Base¹ and Base² are each independently selected from the group consisting of

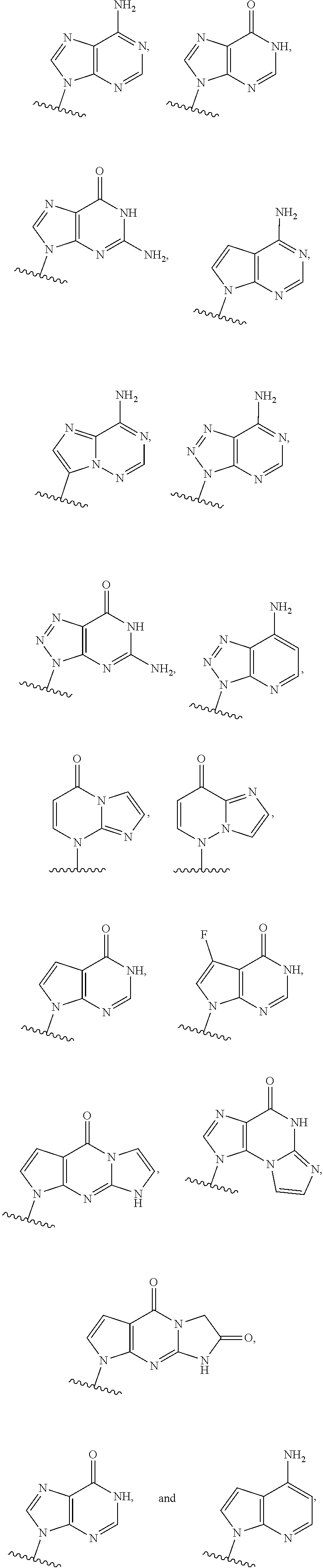

and where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂. In still further instances of this embodiment, Base¹ and Base² are each independently selected from the group consisting of

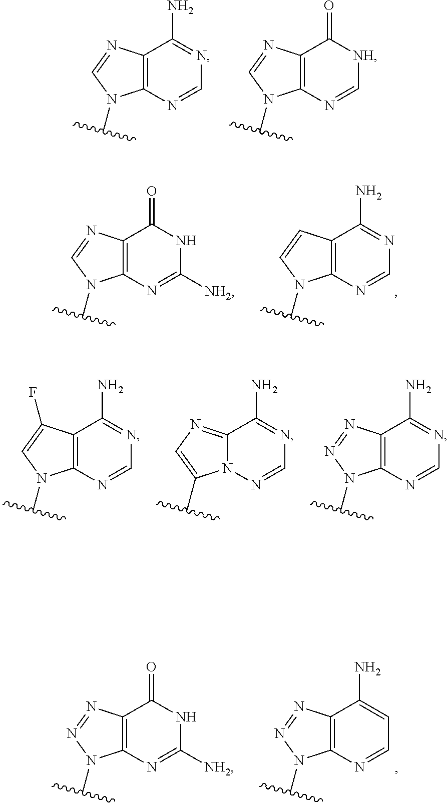

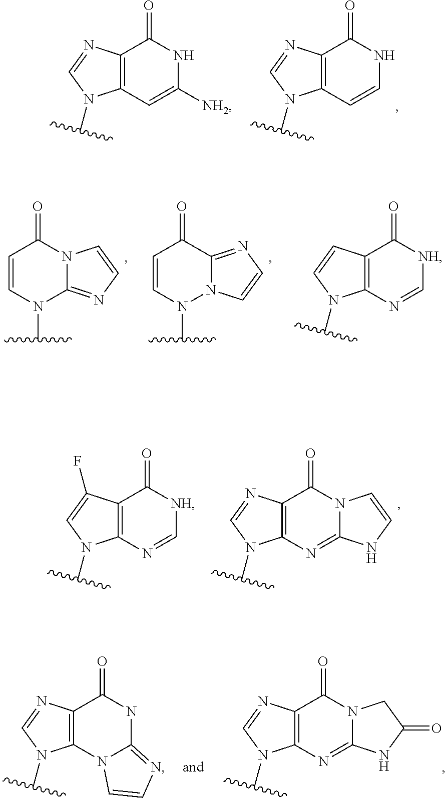

and where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂. In even more particular instances of this embodiment, Base¹ is selected from the group consisting of

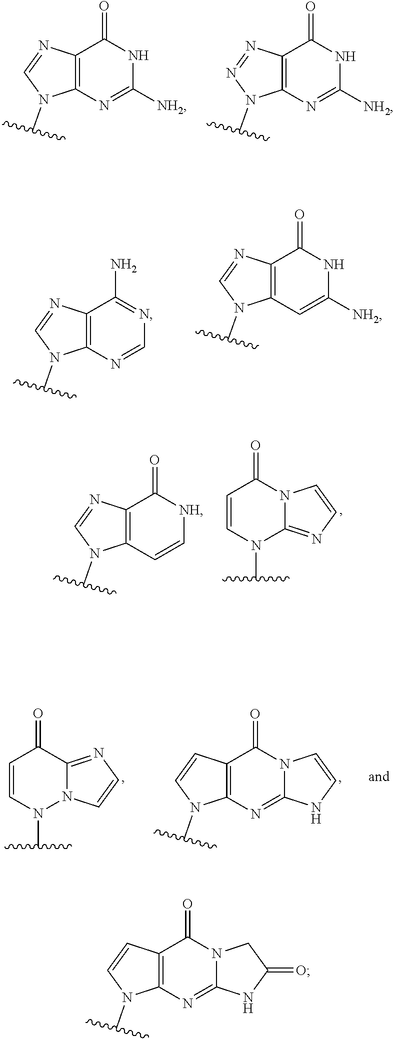

Base² is selected from the group consisting of

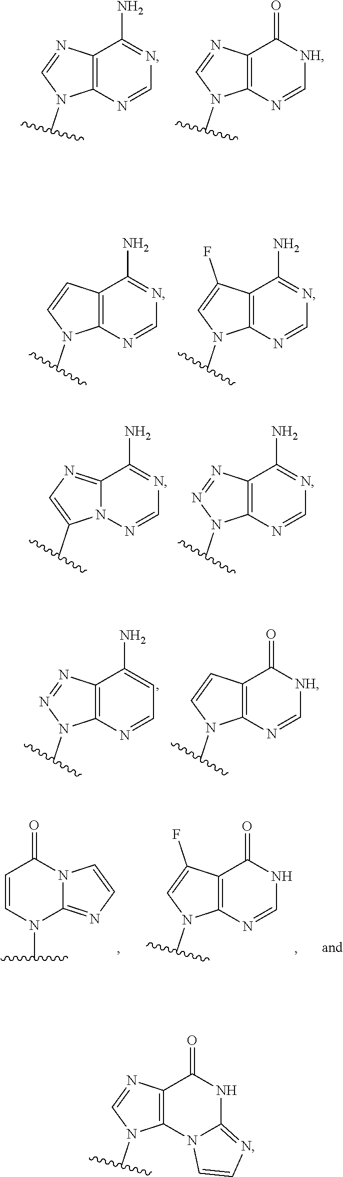

and where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂. In this embodiment, all other groups are as provided in the general formula (I) above.

In a second embodiment, Y and Y^{a} are each independently selected from the group consisting of —O— and —S—. In this embodiment, all other groups are as provided in the general formula (I) above or in the first embodiment described above.

In a third embodiment, X^{a} and X^{a1} are each independently selected from the group consisting of —O— and —S—. In this embodiment, all other groups are as provided in the general formula (I) above or in the first through second embodiments described above.

In a fourth embodiment, X^{b} and X^{b1} are each independently selected from the group consisting of —O— and —S—. In this embodiment, all other groups are as provided in the general formula (I) above or in the first through third embodiments described above.

In a fifth embodiment, X^{c} and X^{c1} are each independently selected from the group consisting of —OR⁹, —SR⁹, and —NR⁹R⁹, where each R⁹ is independently selected from the group consisting of H, C₁-C₂₀ alkyl,

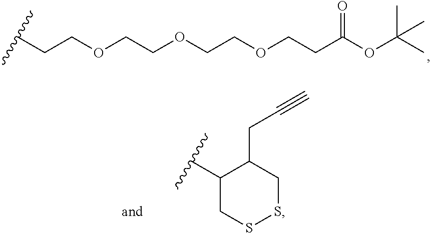

where each R⁹ C₁-C₂₀ alkyl is optionally substituted by 0 to 3 substituents independently selected from the group consisting of —OH, —O—C₁-C₂₀ alkyl, —S—C(O)C₁-C₆ alkyl, and —C(O)OC₁-C₆ alkyl. In particular aspects, X^{c} and X^{c1} are each independently selected from the group consisting of —OH, —SH,

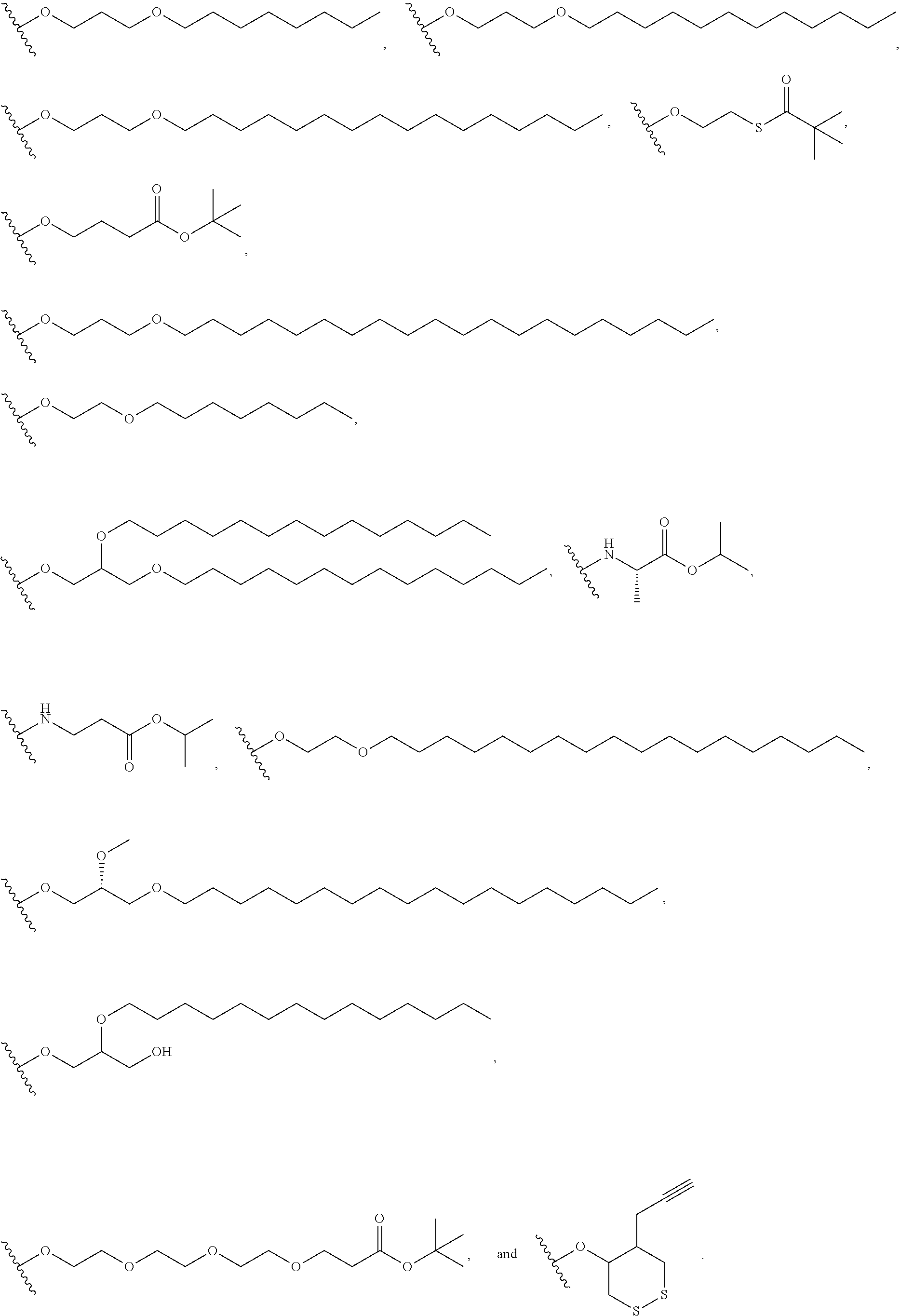

In more particular aspects, X^{c} and X^{c1} are each independently selected from the group consisting of —OH and —SH. In all aspects of this embodiment, all other groups are as provided in the general formula (I) above or in the first through fourth embodiment described above.

In a sixth embodiment, X^{d} and X^{d1} are each independently selected from the group consisting of O and S. In this embodiment, all other groups are as provided in the general formula (I) above or in the first through fifth embodiments described above.

In a seventh embodiment, R¹ and R^{1a} are each H. In this embodiment, all other groups are as provided in the general formula (I) above or in the first through sixth embodiments described above.

In an eighth embodiment, R^{2a} is selected from the group consisting of H, F, Cl, I, Br, OH, CN, N₃, C₁-C₆ alkyl, and C₁-C₆ haloalkyl, where said R^{2a} C₁-C₆ alkyl or C₁-C₆ haloalkyl are substituted by 0 to 3 substituents selected from the group consisting of OH, CN, and N₃. In particular aspects, R^{2a} is selected from the group consisting of H, F, Cl, I, Br, OH, CN, N₃, —CF₃, —CH₃, —CH₂OH, and —CH₂CH₃. In more particular aspects, R^{2a} is selected from the group consisting of H, F, Cl, OH, CN, N₃, and —CH₃. In even more particular aspects, R^{2a} is selected from the group consisting of H, F, and OH. In all aspects of this embodiment, all other groups are as provided in the general formula (I) above or in the first through seventh embodiments described above.

In a ninth embodiment, R³ and R^{3a} are each independently selected from the group consisting H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R³ and R^{3a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃. In particular aspects, R³ and R^{3a} are each independently selected from the group consisting of H, F, Cl, Br, I, OH, CN, N₃, C₁-C₆ alkyl, C₁-C₆ haloalkyl, —O—C₁-C₆ alkyl, —O—C₂-C₆ alkenyl, and —O—C₂-C₆ alkynyl, where said R³ and R^{3a} C₁-C₆ alkyl or C₁-C₆ haloalkyl are substituted by 0 to 3 substituents selected from the group consisting of OH, CN, and N₃. In more particular aspects, R³ and R^{3a} are each independently selected from the group consisting of H, F, Cl, I, Br, OH, CN, N₃, —CF₃, —CH₃, —CH₂OH, —CH₂CH₃, —OCH₃, —OCH₂, and —OCH₂CH₃. In all aspects of this embodiment, all other groups are as provided in the general formula (I) above or in the first through eighth embodiments described above.

In a tenth embodiment, R⁴ and R^{4a} are each independently selected from the group consisting of H, F, Cl, I, Br, CN, OH, N₃, C₁-C₆ alkyl, and C₁-C₆ haloalkyl, where said R⁴ and R^{4a} C₁-C₆ alkyl or C₁-C₆ haloalkyl are substituted by 0 to 3 substituents selected from the group consisting of OH, CN, and N₃. In particular aspects, R⁴ and R^{4a} are each independently selected from the group consisting of H, F, Cl, I, Br, OH, CN, N₃, —CF₃, —CH₃, —CH₂OH, and —CH₂CH₃. In more particular aspects, R⁴ and R^{4a} are each independently selected from the group consisting of H, F, Cl, OH, CN, N₃, and —CH₃. In even more particular aspects, R⁴ and R^{4a} are each independently selected from the group consisting of H, F, and OH. In all aspects of this embodiment, all other groups are as provided in the general formula (I) above or in the first through ninth embodiments described above.

In an eleventh embodiment, R⁵ is selected from the group consisting of H, F, Cl, I, Br, OH, CN, N₃, C₁-C₆ alkyl, and C₁-C₆ haloalkyl, where said R⁵ C₁-C₆ alkyl or C₁-C₆ haloalkyl are substituted by 0 to 3 substituents selected from the group consisting of OH, CN, and N₃. In particular aspects, R⁵ is selected from the group consisting of H, F, Cl, I, Br, OH, CN, N₃, —CF₃, —CH₃, —CH₂OH, and —CH₂CH₃. In more particular aspects, R⁵ is selected from the group consisting of H, F, Cl, OH, CN, N₃, and CH₃. In even more particular aspects, R⁵ is selected from the group consisting of H, F, and OH. In all aspects, all other groups are as provided in the general formula (I) above or in the first through tenth embodiments described above.

In a twelfth embodiment, R⁶ and R^{6a} are each independently selected from the group consisting of H, F, Cl, I, Br, OH, CN, N₃, C₁-C₆ alkyl, C₂-C₆ alkenyl, and C₂-C₆ alkynyl, where said R⁶ and R^{6a} C₁-C₆ alkyl, C₂-C₆ alkenyl, and C₂-C₆ alkynyl are substituted by 0 to 3 substituents selected from the group consisting of F, Cl, Br, I, OH, CN, and N₃. In particular aspects, R⁶ and R^{6a} are each independently selected from the group consisting of H, F, Cl, I, Br, OH, CN, N₃, —CF₃, —CH₃, —CH₂OH, CH₂CH₃, —CH═H₂, —C≡CH, and —C≡C—CH. In more particular aspects, R⁶ and R^{6a} are each independently selected from the group consisting of H, F, CN, N₃, —CH₃, —CH═H₂, and —C≡CH. In even more particular aspects, R⁶ and R^{6a} are each H. In all aspects of this embodiment, all other groups are as provided in the general formula (I) above or in the first through eleventh embodiments described above.

In a thirteenth embodiment, R⁷ and R^{7a} are each independently selected from the group consisting of H, C₁-C₆ alkyl, and C₁-C₆ haloalkyl, where said R⁷ and R^{7a} C₁-C₆ alkyl or C₁-C₆ haloalkyl are substituted by 0 to 3 substituents selected from the group consisting of OH, CN, and N₃. In particular aspects, R⁷ and R^{7a} are each independently selected from the group consisting of H, —CF₃, —CH₃, and —CH₂CH₃. In more particular aspects, R⁷ and R^{7a} are each H. In all aspects of this embodiment, all other groups are as provided in the general formula (I) above or in the first through twelfth embodiments described above.

In a fourteenth embodiment, R⁸ and R^{8a} are each independently selected from the group consisting of H, C₁-C₆ alkyl, and C₁-C₆ haloalkyl, where said R⁸ and R^{8a} C₁-C₆ alkyl or C₁-C₆ haloalkyl are substituted by 0 to 3 substituents selected from the group consisting of OH, CN, and N₃. In particular aspects, R⁸ and R^{8a} are each independently selected from the group consisting of H, —CF₃, —CH₃, and —CH₂CH₃. In more particular aspects, R⁸ and R^{8a} are each H. In all aspects of this embodiment, all other groups are as provided in the general formula (I) above or in the first through thirteenth embodiments described above.

In a fifteenth embodiment, R^{1a} and R^{3a} are connected to form C₁-C₆ alkylene, C₂-C₆ alkenylene, —O—C₁-C₆ alkylene, or —O—C₂-C₆ alkenylene, such that where R^{1a} and R^{3a} are connected to form —O—C₁-C₆ alkylene or —O—C₂-C₆ alkenylene, said O is bound at the R^{3a} position. In this embodiment, all other groups are as provided in the general formula (I) above or in the first through fourteenth embodiments described above.

In a sixteenth embodiment, R^{2a} and R^{3a} are connected to form C₁-C₆ alkylene, C₂-C₆ alkenylene, —O—C₁-C₆ alkylene, or —O—C₂-C₆ alkenylene, such that where R^{2a} and R^{3a} are connected to form —O—C₁-C₆ alkylene or —O—C₂-C₆ alkenylene, said O is bound at the R^{3a} position. In this embodiment, all other groups are as provided in the general formula (I) above or in the first through fourteenth embodiments described above.

In a seventeenth embodiment, R^{3a} and R^{6a} are connected to form C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position. In this embodiment, all other groups are as provided in the general formula (I) above or in the first through fourteenth embodiments described above.

In an eighteenth embodiment, R⁴ and R⁵ are connected to form C₁-C₆ alkylene, C₂-C₆ alkenylene, —O—C₁-C₆ alkylene, or —O—C₂-C₆ alkenylene, such that where R⁴ and R⁵ are connected to form —O—C₁-C₆ alkylene or —O—C₂-C₆ alkenylene, said O is bound at the R⁵ position. In this embodiment, all other groups are as provided in the general formula (I) above or in the first through fourteenth embodiments described above.

In a nineteenth embodiment, R⁵ and R⁶ are connected to form C₁-C₆ alkylene, C₂-C₆ alkenylene, —O—C₁-C₆ alkylene, or —O—C₂-C₆ alkenylene, such that where R⁵ and R⁶ are connected to form —O—C₁-C₆ alkylene or —O—C₂-C₆ alkenylene, said O is bound at the R⁵ position. In this embodiment, all other groups are as provided in the general formula (I) above or in the first through fourteenth embodiments described above.

In a twentieth embodiment, R⁷ and R⁸ are connected to form C₁-C₆ alkylene or C₂-C₆ alkenylene. In this embodiment, all other groups are as provided in the general formula (I) above or in the first through fourteenth embodiments described above.

In a twenty-first embodiment, R^{7a} and R^{8a} are connected to form C₁-C₆ alkylene or C₂-C₆ alkenylene. In this embodiment, all other groups are as provided in the general formula (I) above or in the first through fourteenth embodiments described above.

In a twenty-second embodiment, Base¹ and Base² are each independently selected from the group consisting of

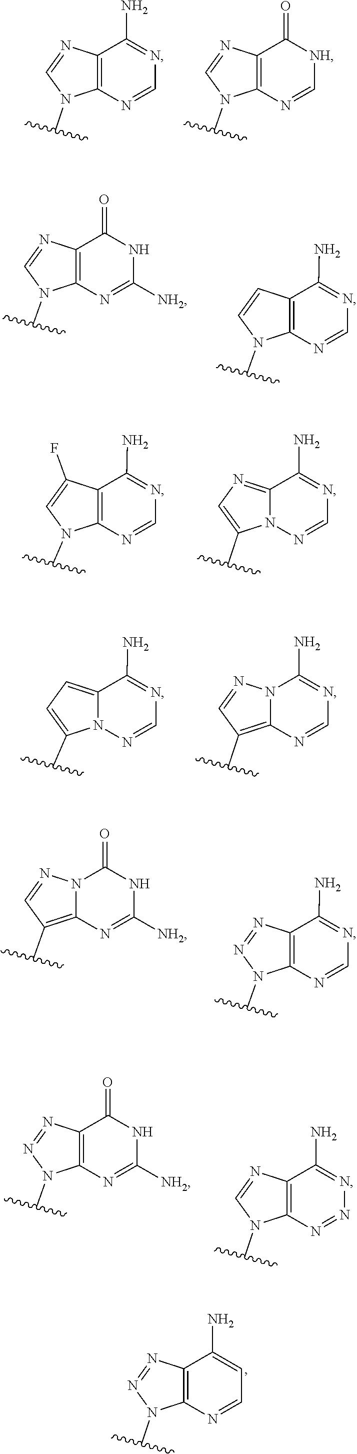

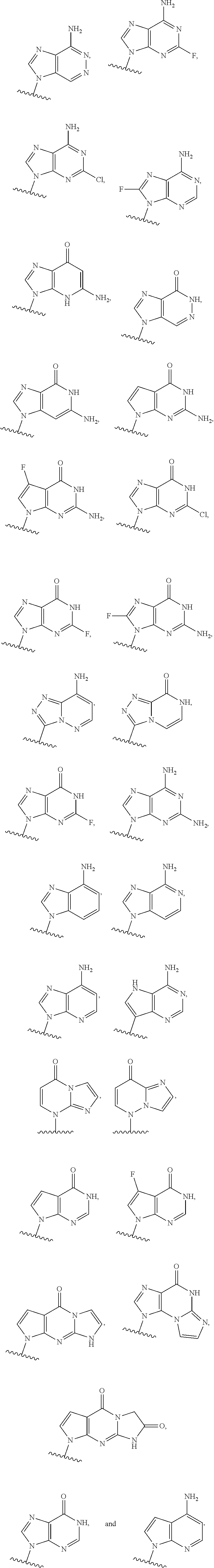

and where Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each independently selected from the group consisting of —O— and —S—; X^{b} and X^{b1} are each independently selected from the group consisting of —O and —S—; X^{c} and X^{c1} are each independently selected from the group consisting of —SR⁹, —OR⁹, and —NR⁹R⁹; X^{d} and X^{d1} are each independently selected from the group consisting of O and S; R¹ and R^{1a} are each H; R^{2a} is selected from the group consisting of H, F, Cl, Br, I, OH, CN, N₃, C₁-C₆ alkyl, and C₁-C₆ haloalkyl, where said R^{2a} C₁-C₆ alkyl or C₁-C₆ haloalkyl are substituted by 0 to 3 substituents selected from the group consisting of OH, CN, and N₃; R³ and R^{3a} are each independently selected from the group consisting of H, F, Cl, Br, I, OH, CN, N₃, C₁-C₆ alkyl, —C₆ haloalkyl, —O—C₁-C₆ alkyl, —O—C₂-C₆ alkenyl, and —O—C₂-C₆ alkynyl, where said R³ and R^{3a} C₁-C₆ alkyl or C₁-C₆ haloalkyl are substituted by 0 to 3 substituents selected from the group consisting of OH, CN, and N₃; R⁴ and R^{4a} are each independently selected from the group consisting of H, F, Cl, I, Br, CN, OH, N₃, C₁-C₆ alkyl, and C₁-C₆ haloalkyl, where said R⁴ and R^{4a} C₁-C₆ alkyl or C₁-C₆ haloalkyl are substituted by 0 to 3 substituents selected from the group consisting of OH, CN, and N₃; R⁵ is selected from the group consisting of H, F, Cl, Br, I, OH, N₃, C₁-C₆ alkyl, and C₁-C₆ haloalkyl, where said R⁵ C₁-C₆ alkyl or C₁-C₆ haloalkyl are substituted by 0 to 3 substituents selected from the group consisting of F, Cl, Br, I, OH, CN, and N₃; R⁶ and R^{6a} are each independently selected from the group consisting of H, F, Cl, I, Br, OH, CN, N₃, C₁-C₆ alkyl, C₂-C₆ alkenyl, and C₂-C₆ alkynyl, where said R⁶ and R^{6a} C₁-C₆ alkyl, C₂-C₆ alkenyl, and C₂-C₆ alkynyl are substituted by 0 to 3 substituents selected from the group consisting of F, Cl, Br, I, OH, CN, and N₃; R⁷ and R^{7a} are each independently selected from the group consisting of H, C₁-C₆ alkyl, and C₁-C₆ haloalkyl, where said R⁷ and R^{7a} C₁-C₆ alkyl or C₁-C₆ haloalkyl are substituted by 0 to 3 substituents selected from the group consisting of OH, CN, and N₃; R⁸ and R^{8a} are each independently selected from the group consisting of H, C₁-C₆ alkyl, and C₁-C₆ haloalkyl, where said R⁸ and R^{8a} C₁-C₆ alkyl or C₁-C₆ haloalkyl are substituted by 0 to 3 substituents selected from the group consisting of OH, CN, and N₃; each R⁹ is independently selected from the group consisting of H, C₁-C₆ alkyl,

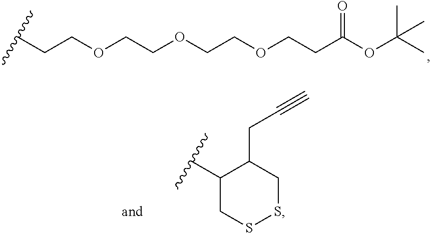

where each R⁹ C₁-C₆ alkyl is optionally substituted by 1 to 2 substituents independently selected from the group consisting of OH, —O—C₁-C₂₀ alkyl, —S—C(O)C₁-C₆ alkyl, and —C(O)OC₁-C₆ alkyl; optionally R^{3a} and R^{6a} are connected to form C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position; and optionally R⁵ and R⁶ are connected to form C₁-C₆ alkylene, C₂-C₆ alkenylene, —O—C₁-C₆ alkylene, or —O—C₂-C₆ alkenylene, such that where R⁵ and R⁶ are connected to form —O—C₁-C₆ alkylene or —O—C₂-C₆ alkenylene, said O is bound at the R⁵ position. In this embodiment, all other groups are as provided in the general formula (I) above or in the first through fourteenth embodiments described above.

In a twenty-third embodiment, Base¹ and Base² are each independently selected from the group consisting of

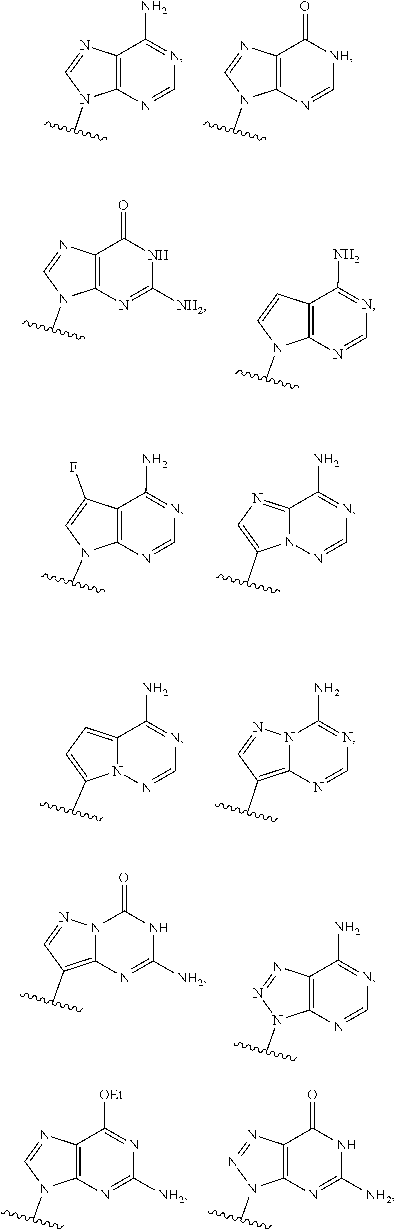

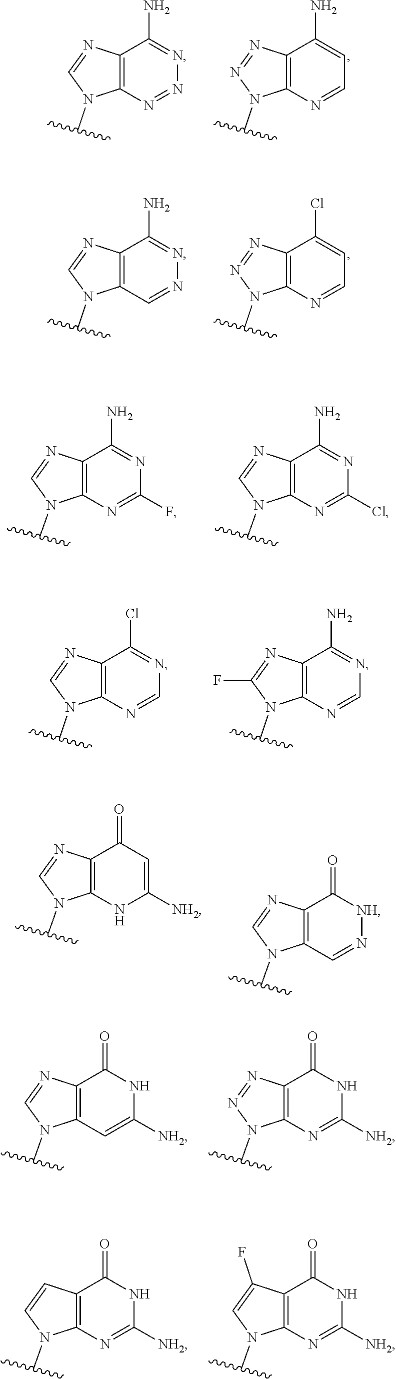

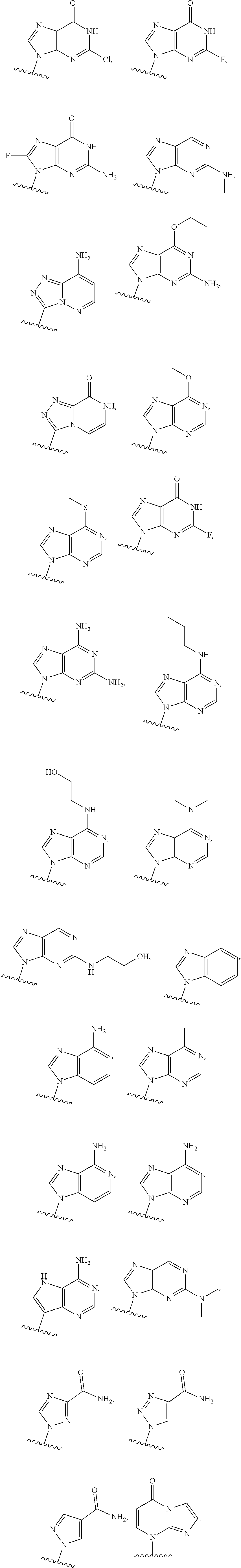

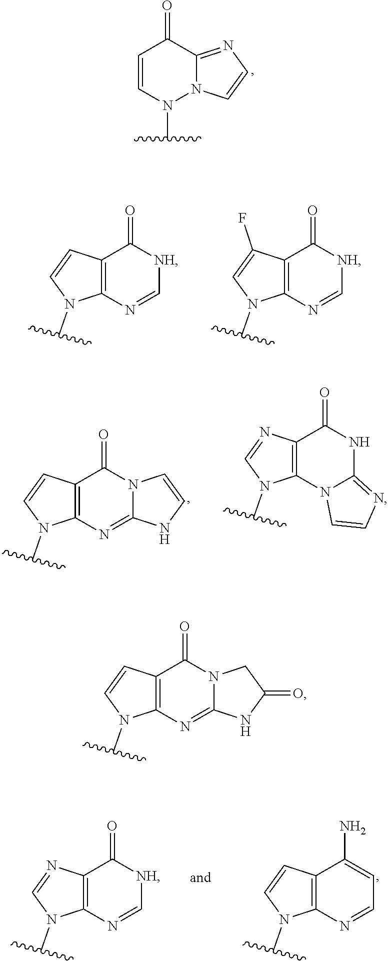

and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each independently selected from the group consisting of —O— and —S—; X^{b} and X^{b1} are each independently selected from the group consisting of —O— and —S—; X^{c} and X^{c1} are each independently selected from the group consisting of —OH, —SH,

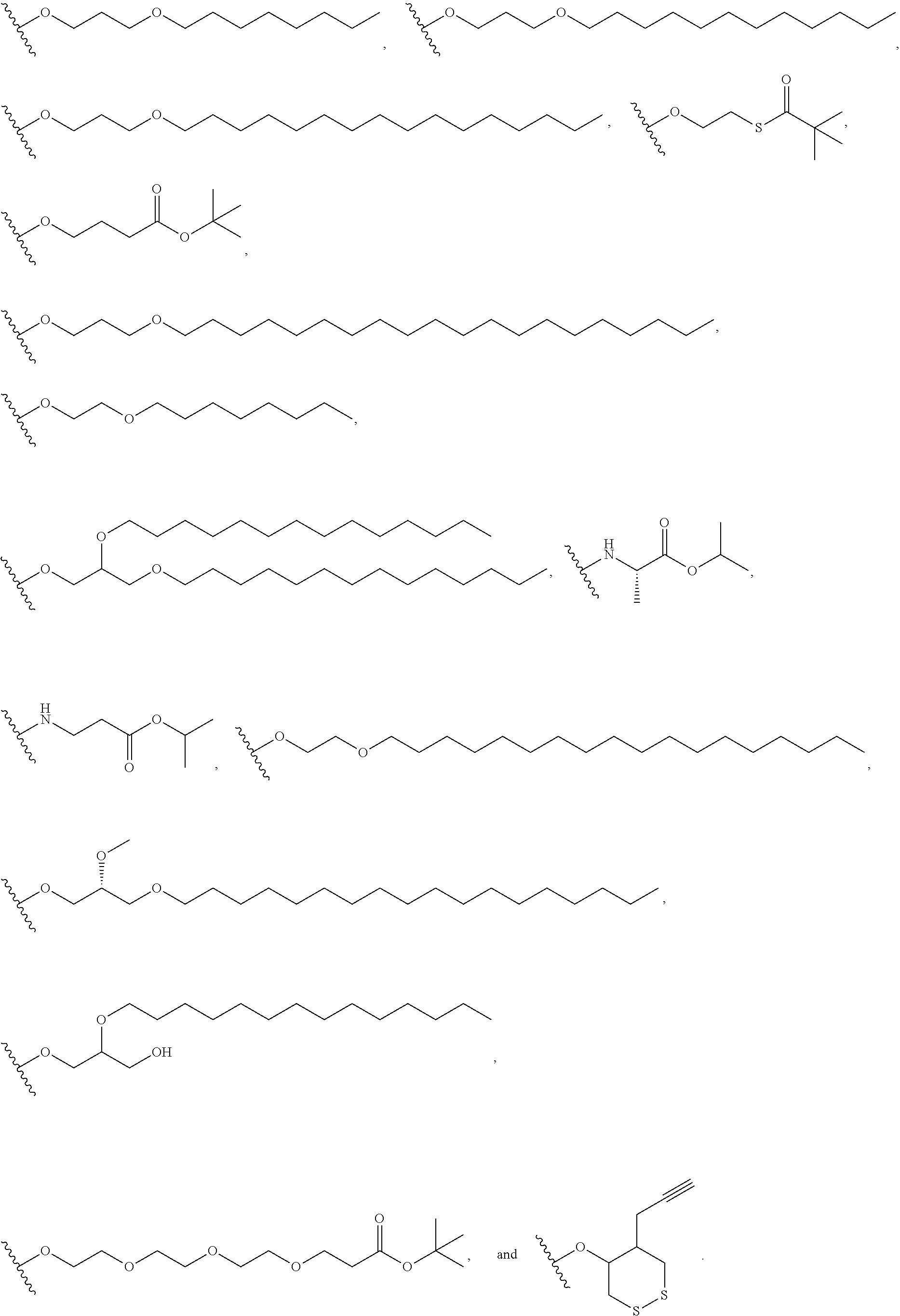

X^{d} and X^{d1} are each independently selected from the group consisting of O and S; R¹ and R^{1a} are each H; R^{2a} is selected from the group consisting of H, F, Cl, I, Br, OH, CN, N₃, —CF₃, —CH₃, —CH₂OH, and —CH₂CH₃; R³ and R^{3a} are each independently selected from the group consisting of H, F, Cl, I, Br, OH, CN, N₃, —CF₃, —CH₃, —CH₂OH, —CH₂CH₃, —OCH₃, —OCH₂, and —OCH₂CH₃; R⁴ and R^{4a} are each independently selected from the group consisting of H, F, Cl, I, Br, OH, CN, N₃, —CF₃, —CH₃, —CH₂OH, and —CH₂CH₃; R⁵ is selected from the group consisting of H, F, Cl, I, Br, OH, CN, N₃, —CF₃, —CH₃, —CH₂OH, and —CH₂CH₃; R⁶ and R^{6a} are each independently selected from the group consisting of H, F, Cl, I, Br, OH, CN, N₃, —CF₃, —CH₃, —CH₂OH, —CH₂CH₃, —C≡CH, and —C≡C—CH; R⁷ and R^{7a} are each independently selected from the group consisting of H, —CF₃, —CH₃, and —CH₂CH₃; R⁸ and R^{8a} are each independently selected from the group consisting of H, —CF₃, —CH₃, and —CH₂CH₃; optionally R^{3a} and R^{6a} are connected to form C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position; and optionally R⁵ and R⁶ are connected to form C₁-C₆ alkylene, C₂-C₆ alkenylene, —O—C₁-C₆ alkylene, or —O—C₂-C₆ alkenylene, such that where R⁵ and R⁶ are connected to form —O—C₁-C₆ alkylene or —O—C₂-C₆ alkenylene, said O is bound at the R⁵ position. In this embodiment, all other groups are as provided in the general formula (I) above or in the first through fourteenth embodiments described above.

In a twenty-fourth embodiment, Base¹ and Base² are each independently selected from the group consisting of

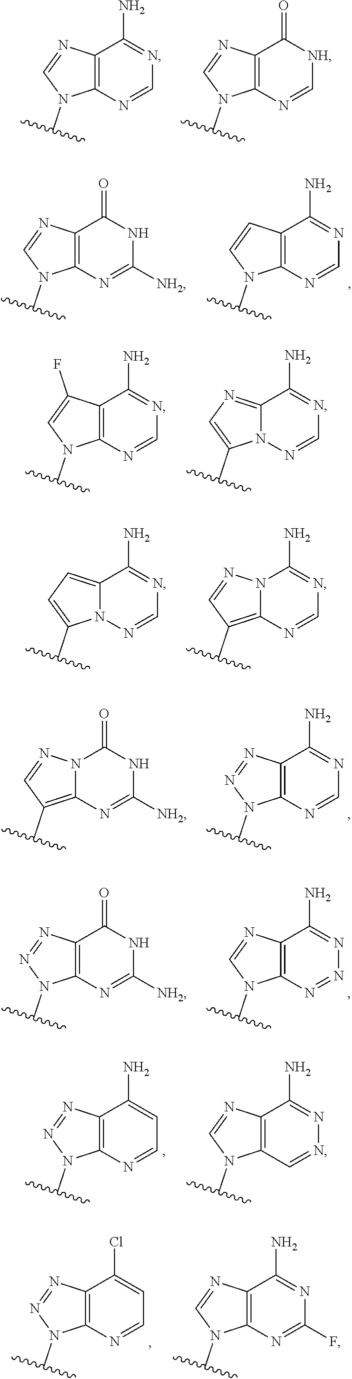

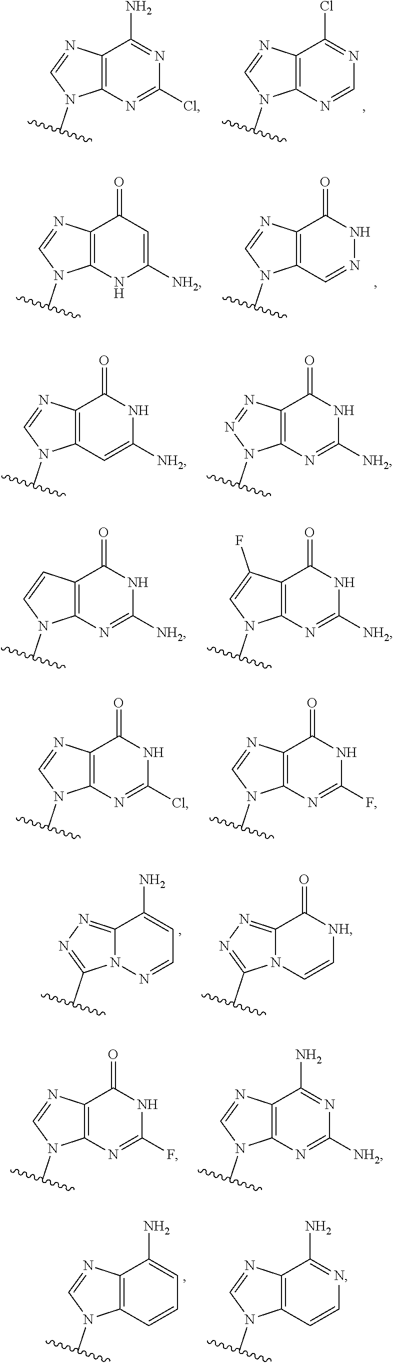

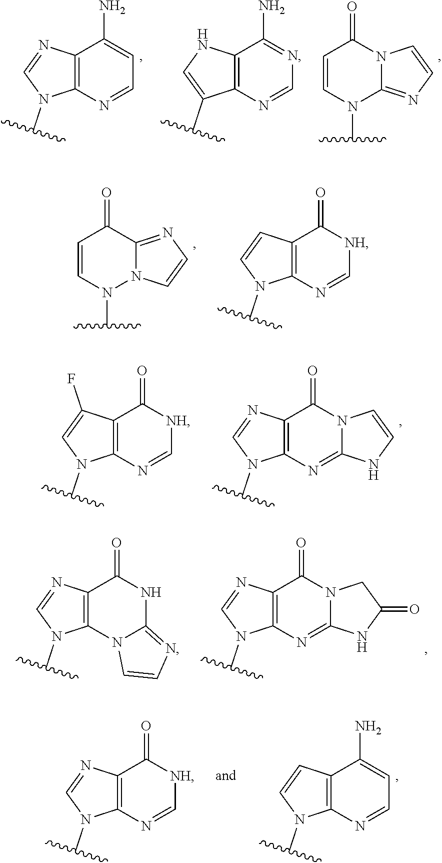

and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each —O—; X^{b} and X^{b1} are each —O—; X^{c} and X^{c1} are each independently selected from the group consisting of —OH and —SH; X^{d} and X^{d1} are each independently selected from the group consisting of O and S; R¹ and R^{1a} are each H; R^{2a} is selected from the group consisting of H, F, Cl, OH, CN, N₃, and —CH₃; R³ and R^{3a} are each independently selected from the group consisting of H, F, Cl, OH, CN, N₃, —CH₃, —CH₂OH, —CH₂CH₃, —OCH₃, —OCH₂, and —OCH₂CH₃; R⁴ and R^{4a} are each independently selected from the group consisting of H, F, Cl, OH, CN, N₃, and —CH₃; R⁵ is selected from the group consisting of H, F, Cl, OH, CN, N₃, and —CH₃; R⁶ and R^{6a} are each independently selected from the group consisting of H, F, CN, N₃, —CH₃, —CH═H₂, and —C≡CH; R⁷ and R^{7a} are each H; R⁸ and R^{8a} are each H; optionally R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position; and optionally R⁵ and R⁶ are connected to form C₁-C₆ alkylene, C₂-C₆ alkenylene, —O—C₁-C₆ alkylene, or —O—C₂-C₆ alkenylene, such that where R⁵ and R⁶ are connected to form —O—C₁-C₆ alkylene or —O—C₂-C₆ alkenylene, said O is bound at the R⁵ position. In this embodiment, all other groups are as provided in the general formula (I) above or in the first through fourteenth embodiments described above.

In a twenty-fifth embodiment, Base¹ and Base² are each independently selected from the group consisting of

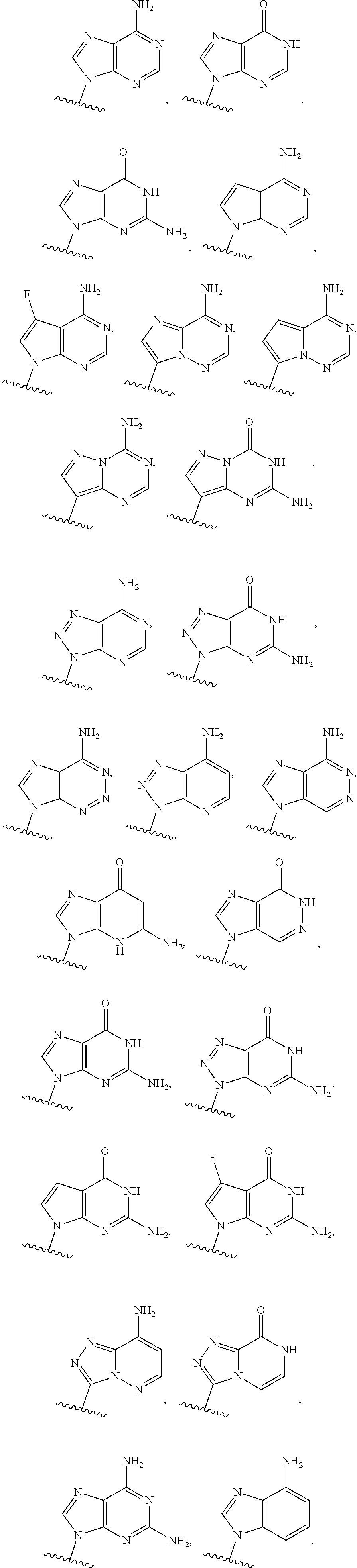

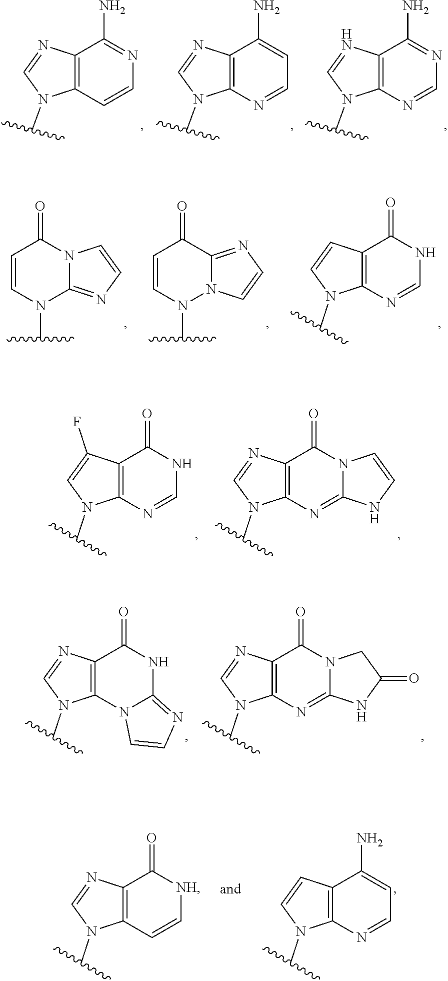

and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each O; X^{b} and X^{b1} are each O; X^{c} and X^{c1} are each independently selected from the group consisting of —OH and —SH; X^{d} and X^{d1} are each independently selected from the group consisting of O and S; R¹ and R^{1a} are each H; R^{2a} is selected from the group consisting of H, F, Cl, OH, CN, N₃, and —CH₃; R³ and R^{3′} are each independently selected from the group consisting of H, F, Cl, OH, CN, N₃, —CH₃, —CH₂OH, —CH₂CH₃, —OCH₃, —OCH₂, and —OCH₂CH₃; R⁴ and R^{4a} are each independently selected from the group consisting of H, F, Cl, OH, CN, N₃, and CH₃; R⁵ is selected from the group consisting of H, F, Cl, OH, CN, N₃, and —CH₃; R⁶ and R^{6a} are each independently selected from the group consisting of H, F, CN, N₃, —CH₃, —CH═H₂, and —C≡CH; R⁷ and R^{7a} are each H; R⁸ and R^{8a} are each H; optionally R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position; and optionally R⁵ and R⁶ are connected to form C₁-C₆ alkylene, C₂-C₆ alkenylene, —O—C₁-C₆ alkylene, or —O—C₂-C₆ alkenylene, such that where R⁵ and R⁶ are connected to form —O—C₁-C₆ alkylene or —O—C₂-C₆ alkenylene, said O is bound at the R⁵ position. In this embodiment, all other groups are as provided in the general formula (I) above or in the first through fourteenth embodiments described above.

In a twenty-sixth embodiment, Base¹ and Base² are each independently selected from the group consisting of

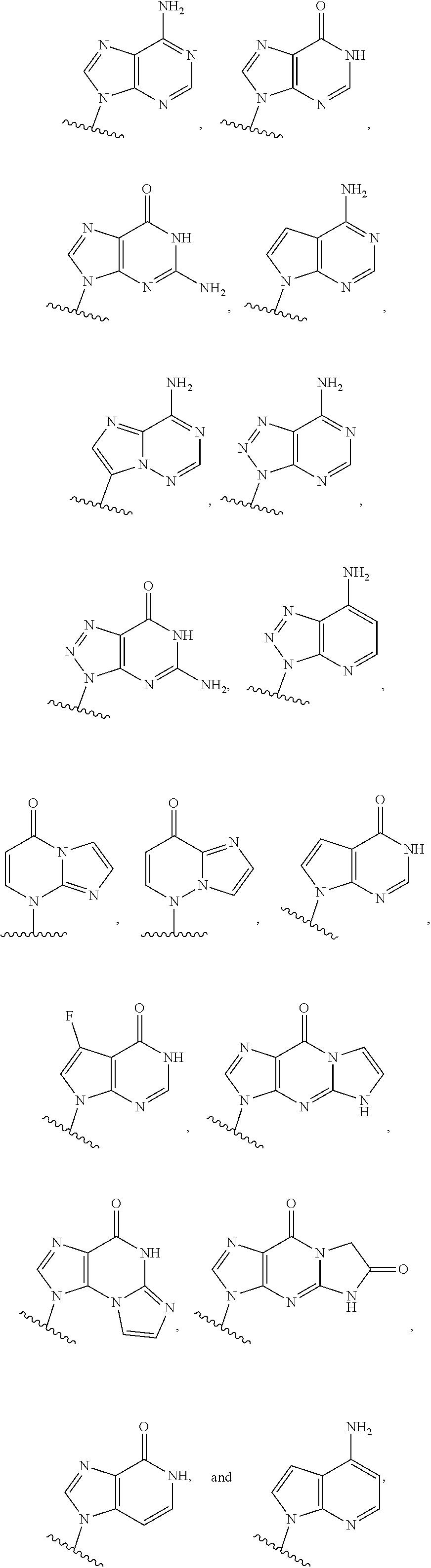

and Base¹ and Base¹ each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each —O—; X^{b} and X^{b1} are each —O—; X^{c} and X^{c1} are each independently selected from the group consisting of —SH and —OH; X^{d} and X^{d1} are each independently selected from the group consisting of O and S; R¹ and R^{1a} are each H; R^{2a} is selected from the group consisting of H, F, and OH; R³ and R^{3a} are each independently selected from the group consisting of H, F, OH, —CH₃, —CH₂OH, —CH₂CH₃, —OCH₃, —OCH₂, and —OCH₂CH₃; R⁴ and R^{4a} are each independently selected from the group consisting of H, F, and OH; R⁵ is selected from the group consisting of H, F, and OH; R⁶ and R^{6a} are each H; R⁷ and R^{7a} are each H; R⁸ and R^{8a} are each H; and optionally R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position. In this embodiment, all other groups are as provided in the general formula (I) above or in the first through fourteenth embodiments described above.

In a twenty-seventh embodiment, Base¹ and Base² are each independently selected from

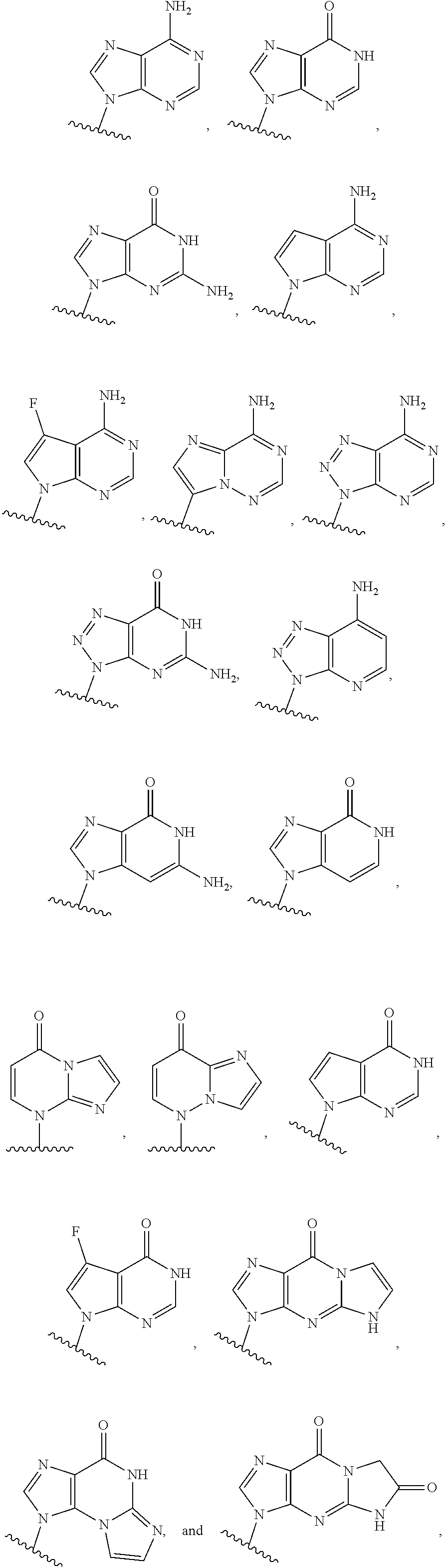

and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each —O—; X^{b} and X^{b1} are each —O—; X^{c} and X^{c1} are each independently selected from the group consisting of —SH and —OH; X^{d} and X^{d1} are each independently selected from the group consisting of O and S; R¹ and R^{1a} are each H; R^{2a} is selected from the group consisting of H, F, and OH; R³ and R^{3a} are each independently selected from the group consisting of H, F, OH, —CH₃, —CH₂OH, —CH₂CH₃, —OCH₃, —OCH₂, and —OCH₂CH₃; R⁴ is selected from the group consisting of H, F, and OH; R^{4a} is H; R⁵ is selected from the group consisting of H, F, and OH; R⁶ and R^{6a} are each H; R⁷ and R^{7a} are each H; R⁸ and R^{8a} are each H; and optionally R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position. In this embodiment, all other groups are as provided in the general formula (I) above or in the first through fourteenth embodiments described above.

In a twenty-eighth embodiment, Base¹ and Base² are each independently selected from the group consisting of

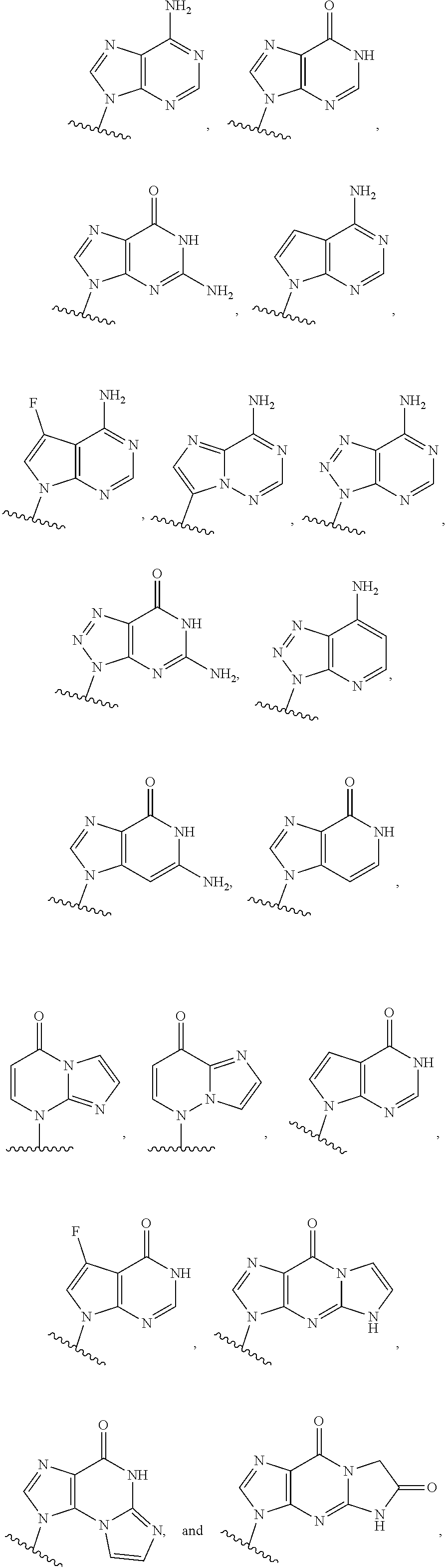

and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each —O—; X^{b} and X^{b1} are each —O—; X^{c} and X^{c1} are each independently selected from the group consisting of —SH and —OH; X^{d} and X^{d1} are each independently selected from the group consisting of O and S; R¹ and R^{1a} are each H; R^{2a} is selected from the group consisting of H, F, and OH; R³ and R^{3a} are each independently selected from the group consisting of H, F, OH, —CH₃, —CH₂OH, —CH₂CH₃, —OCH₃, —OCH₂, and —OCH₂CH₃; R⁴ is selected from the group consisting of H, F, and OH; R^{4a} is H; R⁵ is selected from the group consisting of H, F, and OH; R⁶ and R^{6a} are each H; R⁷ and R^{7a} are each H; and R⁸ and R^{8a} are each H. In this embodiment, all other groups are as provided in the general formula (I) above or in the first through fourteenth embodiments described above.

In a twenty-ninth embodiment, Base¹ is selected from the group consisting of

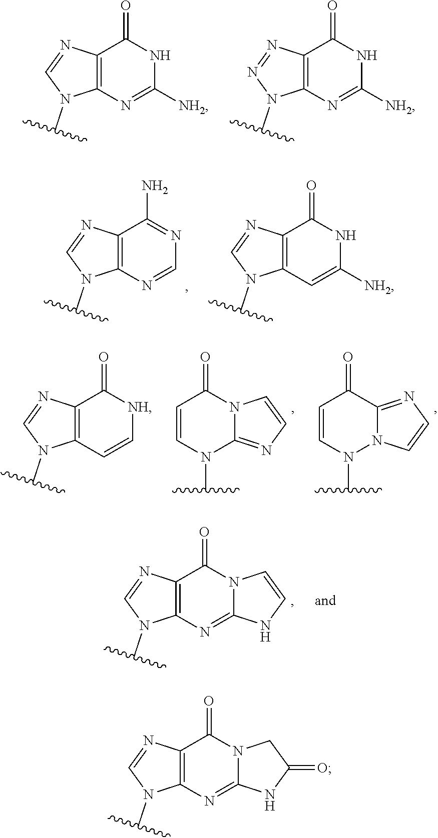

Base² is selected from the group consisting of

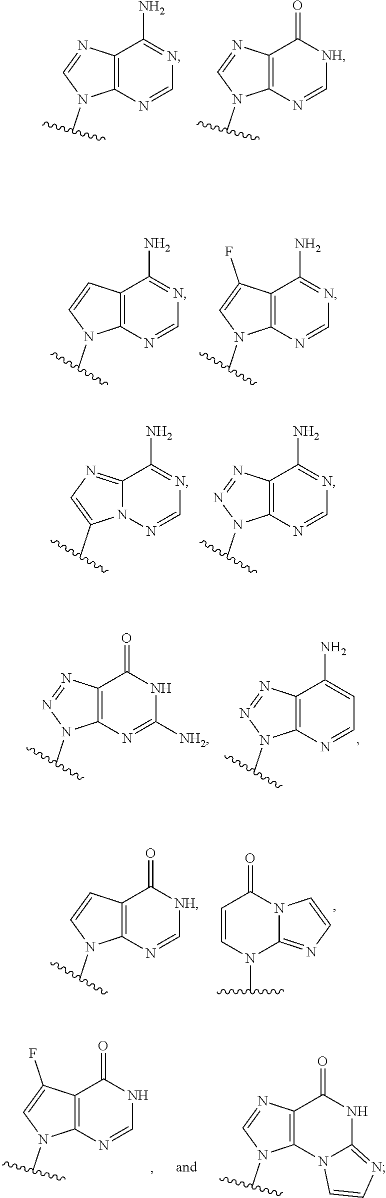

and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each —O—; X^{b} and X^{b1} are each —O—; X^{c} and X^{c1} are each independently selected from the group consisting of —SH and —OH; X^{d} and X^{d1} are each independently selected from the group consisting of O and S; R¹ and R^{1a} are each H; R^{2a} is selected from the group consisting of H, F, and OH; R³ and R^{3a} are each independently selected from the group consisting of H, F, OH, —CH₃, —CH₂OH, —CH₂CH₃, —OCH₃, —OCH₂, and —OCH₂CH₃; R⁴ is selected from the group consisting of H, F, and OH; R^{4a} is H; R⁵ is selected from the group consisting of H, F, and OH; R⁶ and R^{6a} are each H; R⁷ and R^{7a} are each H; R⁸ and R^{8a} are each H; and R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position. In this embodiment, all other groups are as provided in the general formula (I) above or in the first through fourteenth embodiments described above.

In a thirtieth embodiment, Base¹ is selected from the group consisting of

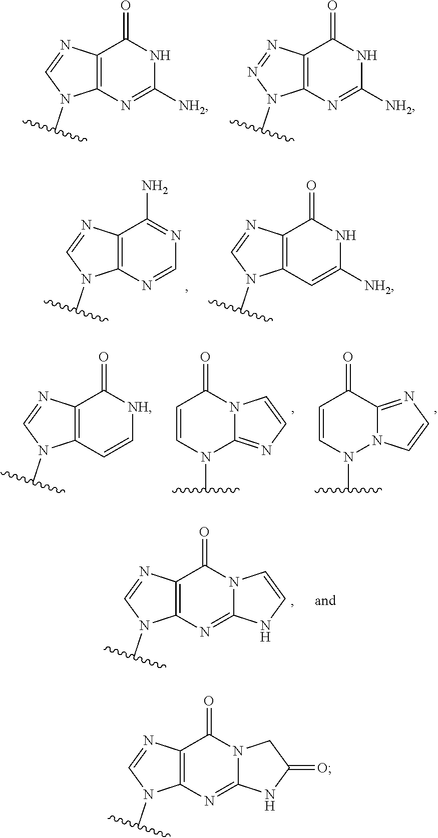

Base² is selected from the group consisting of

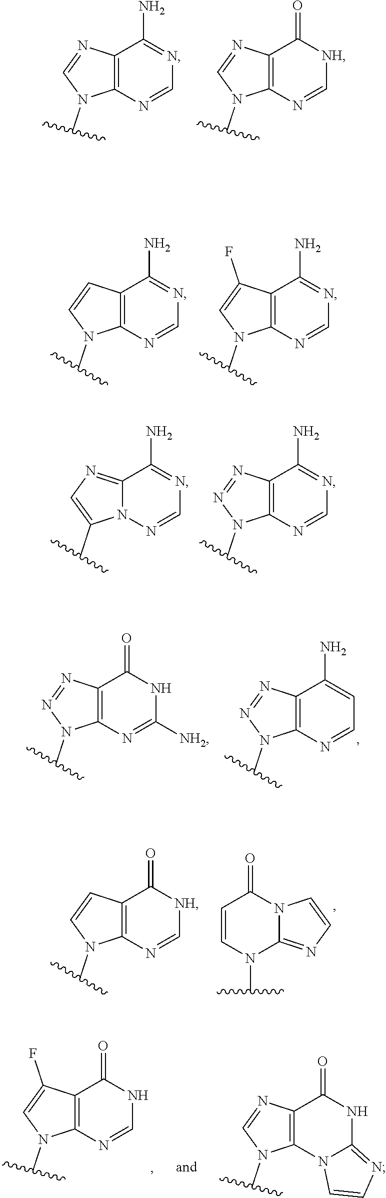

and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂; Y and Y^{a} are each independently selected from the group consisting of —O— and —S—; X^{a} and X^{a1} are each —O—; X^{b} and X^{b1} are each —O—; X^{c} and X^{c1} are each independently selected from the group consisting of —SH and —OH; X^{d} and X^{d1} are each independently selected from the group consisting of O and S; R¹ and R^{1a} are each H; R^{2a} is selected from the group consisting of H, F, and OH; R³ and R^{3a} are each independently selected from the group consisting of H, F, OH, —CH₃, —CH₂OH, —CH₂CH₃, —OCH₃, —OCH₂, and —OCH₂CH₃; R⁴ is selected from the group consisting of H, F, and OH; R^{4a} is H; R⁵ is selected from the group consisting of H, F, and OH; R⁶ and R^{6a} are each H; R⁷ and R^{7a} are each H; and R⁸ and R^{8a} are each H. In this embodiment, all other groups are as provided in the general formula (I) above or in the first through fourteenth embodiments described above.

In a thirty-first embodiment, Base¹ is selected from the group consisting of

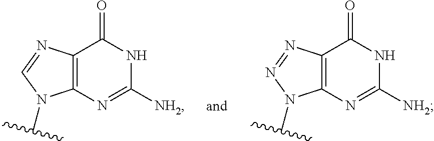 [SMILES: *n1cnc2c(=O)[nH]c(N)nc21 ; "and" ; *n1nnc2c(=O)[nH]c(N)nc21]

Base² is selected from the group consisting of

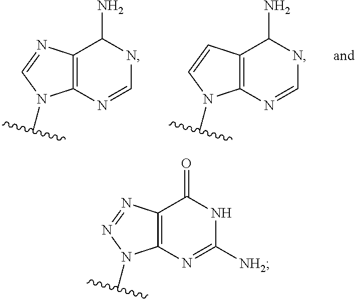

and Y and Y^{a} are each —O—; X^{a} and X^{a1} are each —O—; X^{b} and X^{b1} are each —O—; X^{c} and X^{c1} are each independently selected from the group consisting of —SH and —OH; X^{d} and X^{d1} are each independently selected from the group consisting of O and S; R¹ and R^{1a} are each H; R^{2a} is selected from the group consisting of H and F; R³ and R^{3a} are each independently selected from the group consisting of H, OH, —CH₃, —CH₂OH, —CH₂CH₃, —OCH₃, —OCH₂, and —OCH₂CH₃; R⁴ and R^{4a} are each H; R⁵ is selected from the group consisting of H, F, and OH; R⁶ and R^{6a} are each H; R⁷ and R^{7a} are each H; R⁸ and R^{8a} are each H; and R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position. In this embodiment, all other groups are as provided in the general formula (I) above or in the first through fourteenth embodiments described above.

A thirty-second embodiment relates to a compound selected from the group consisting of

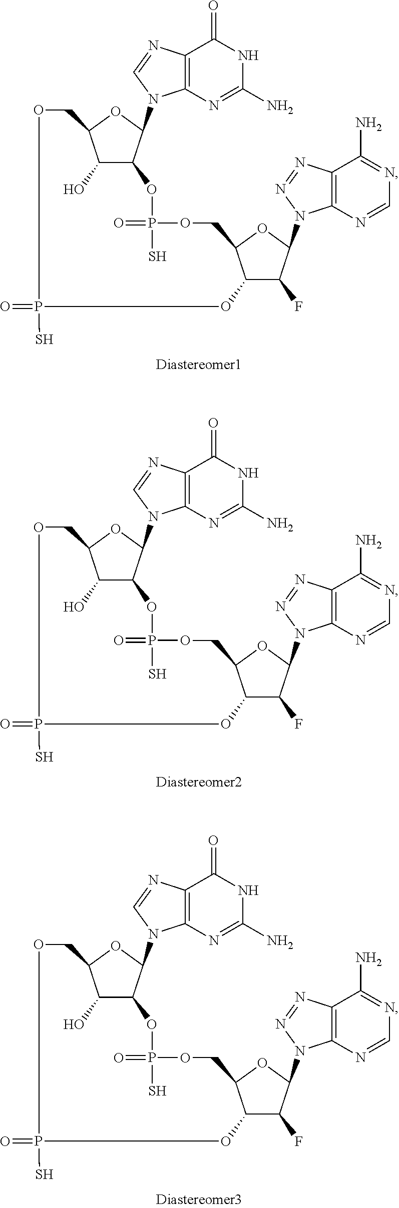 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3O)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3O)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3O)c(=O)[nH]1]

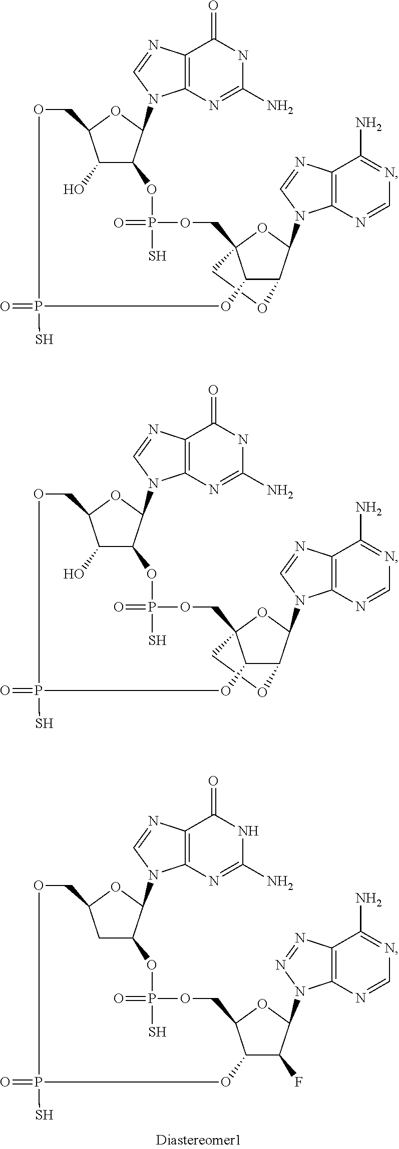 [SMILES: Nc1nc2c(ncn2[C@@H]2O[C@@H]3COP(=O)(S)O[C@H]4[C@H]5OC[C@]4(COP(=O)(S)O[C@H]2[C@@H]3O)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@H]2[C@@H]3O)O[C@H]5n2cnc3c(N)ncnc32)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1]

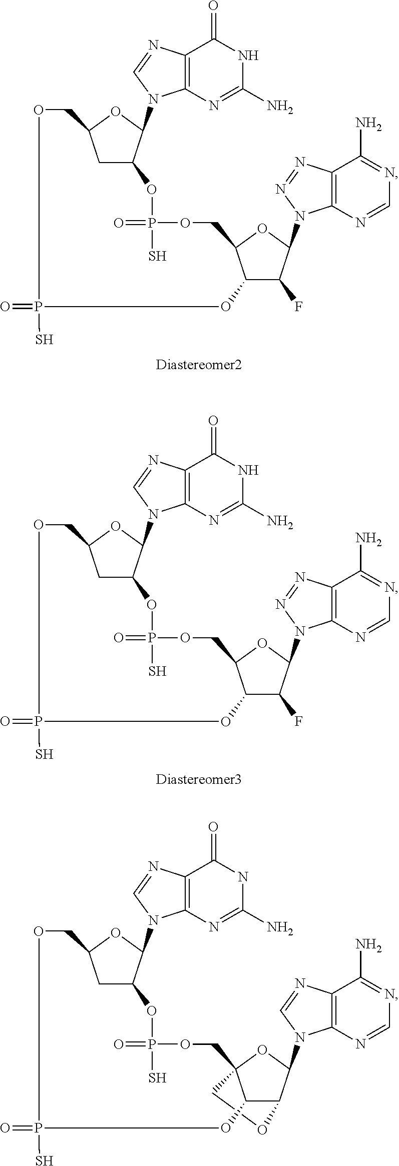 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1 ; Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1]

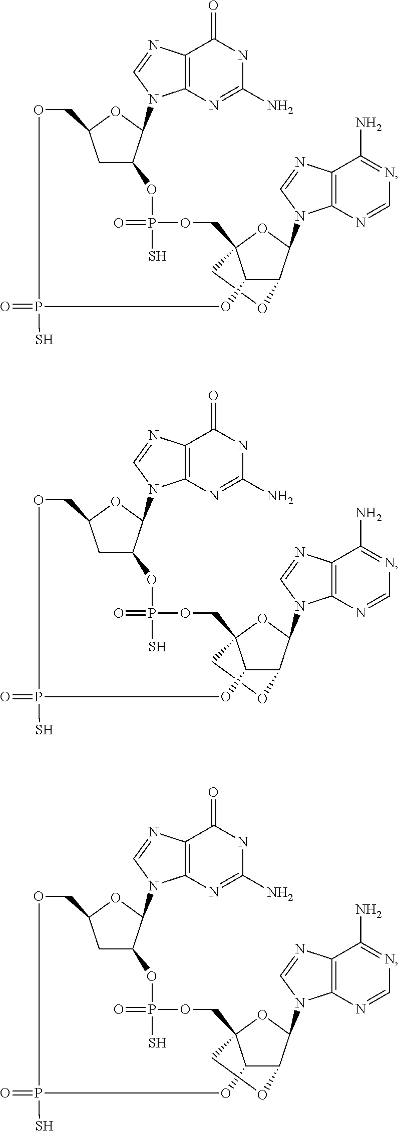 [SMILES: Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1]

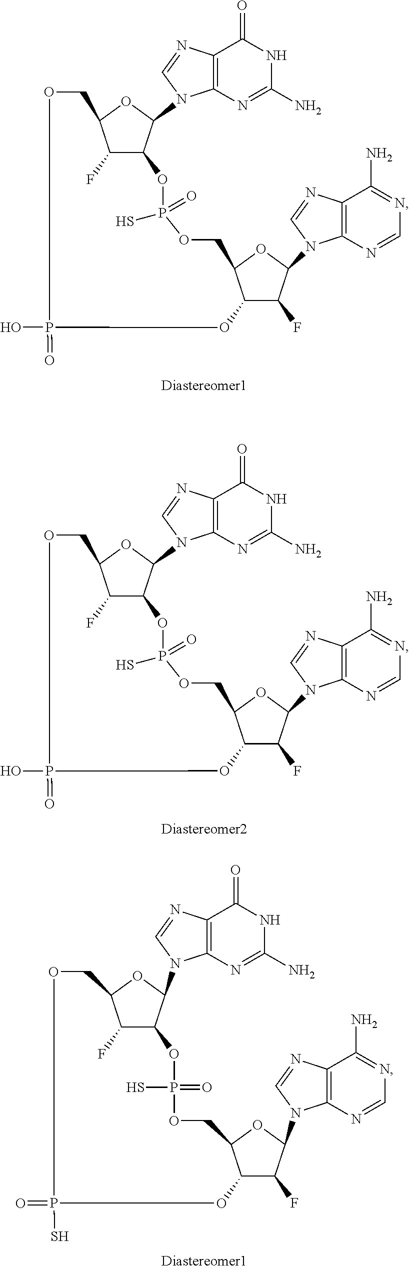 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1]

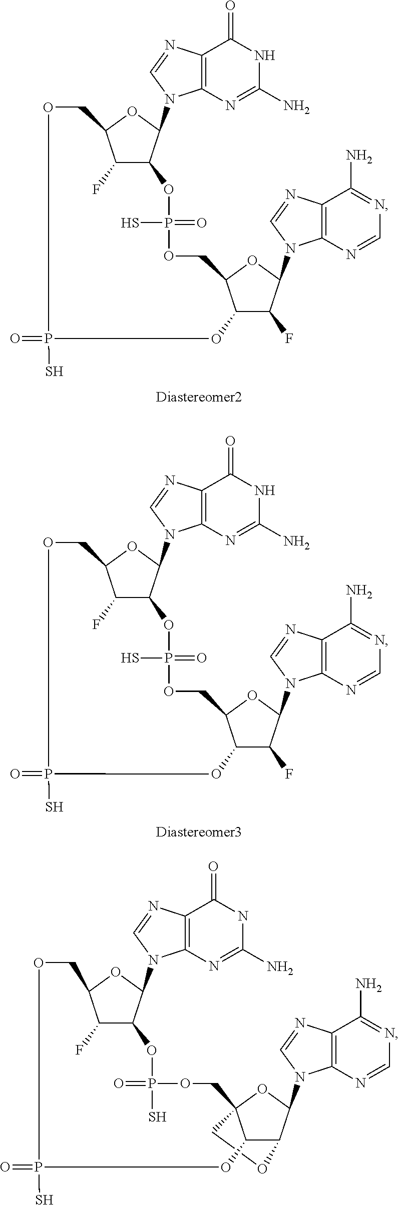 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(S)O[C@H]4[C@H]5OC[C@]4(COP(=O)(S)O[C@H]2[C@@H]3F)O[C@H]5n2cnc3c(N)ncnc32)c(=O)[nH]1]

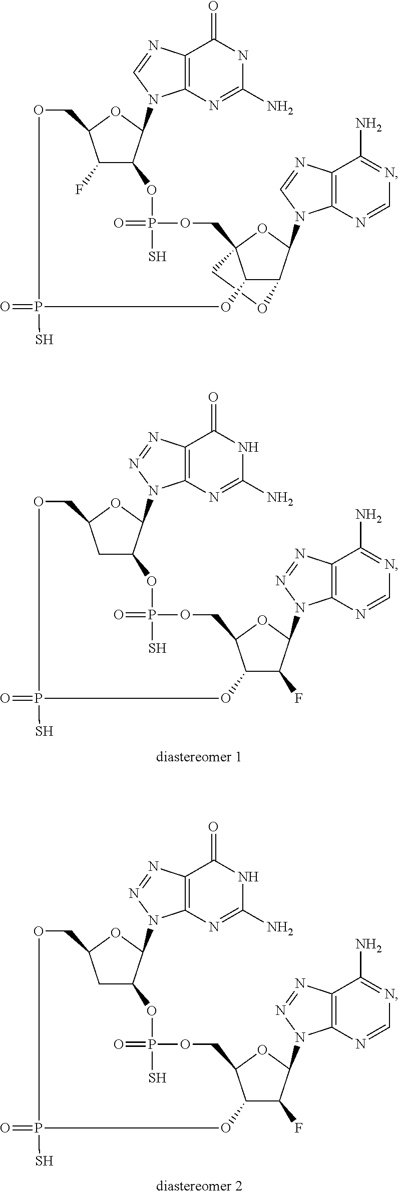 [SMILES: Nc1nc2c(ncn2[C@@H]2O[C@@H]3COP(=O)(S)O[C@H]4[C@H]5OC[C@]4(COP(=O)(S)O[C@H]2[C@@H]3F)O[C@H]5n2cnc3c(N)ncnc32)c(=O)[nH]1 ; Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1 ; Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1]

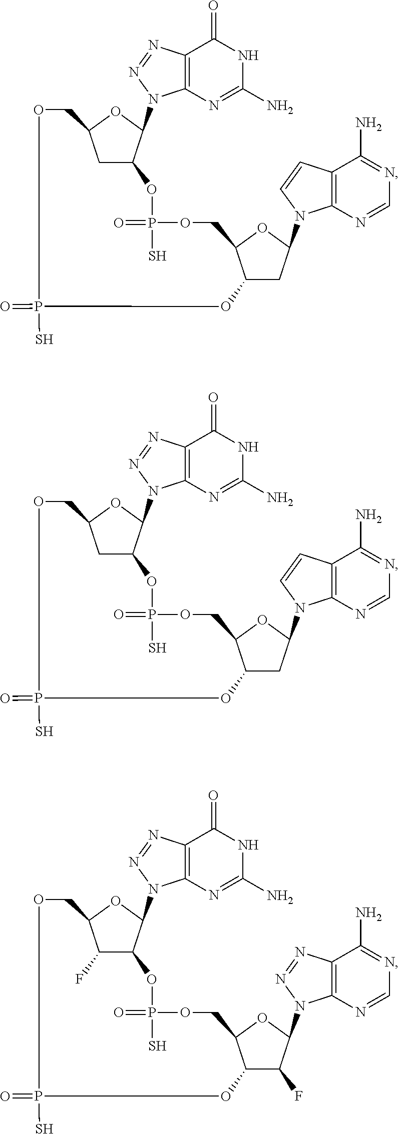 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2C3)c(=O)[nH]1 ; Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2C3)c(=O)[nH]1 ; Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3F)c(=O)[nH]1]

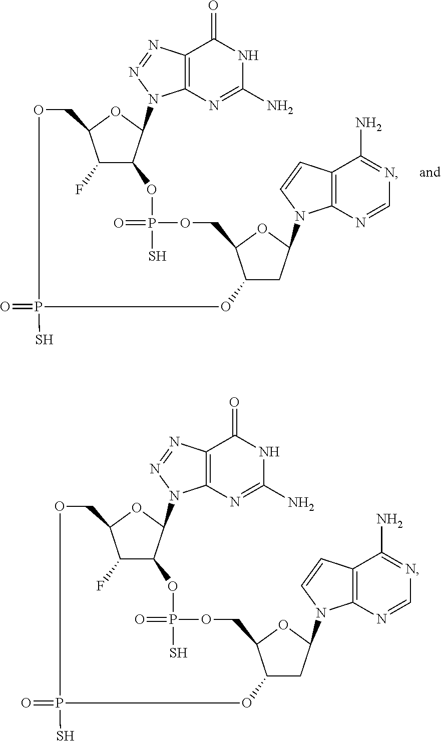 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2[C@@H]3F)c(=O)[nH]1 ; Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2[C@@H]3F)c(=O)[nH]1]

and pharmaceutically acceptable salts thereof.

A thirty-third embodiment relates to a pharmaceutical composition, said pharmaceutical composition comprising (a) a compound according to any one of general formula (I) above or the first through thirty-second embodiments above or a pharmaceutically acceptable salt thereof; and (b) a pharmaceutically acceptable carrier.

A thirty-fourth embodiment relates to methods of inducing an immune response in a subject, comprising administering a therapeutically effective amount of a compound according to any one of general formula (I) above or the first through thirty-second embodiments above or a pharmaceutically acceptable salt thereof to the subject.

A thirty-fifth embodiment relates to methods of inducing an immune response in a subject, comprising administering a therapeutically effective amount of a composition according to the thirty-third embodiment above to the subject.

A thirty-sixth embodiment relates to methods of inducing STING-dependent type I interferon production in a subject, comprising administering a therapeutically effective amount of a compound according to any one of general formula (I) above or the first through thirty-second embodiments above or a pharmaceutically acceptable salt thereof to the subject.

A thirty-seventh embodiment relates to methods of inducing STING-dependent type I interferon production in a subject, comprising administering a therapeutically effective amount of a composition according to the thirty-third embodiment above to the subject.

A thirty-eighth embodiment relates to methods of inducing STING-dependent cytokine production in a subject, comprising administering a therapeutically effective amount of a compound according to any one of general formula (I) above or the first through thirty-second embodiments above or a pharmaceutically acceptable salt thereof to the subject.

A thirty-ninth embodiment relates to methods of inducing a STING-dependent cytokine production in a subject, comprising administering a therapeutically effective amount of a composition according to the thirty-third embodiment above to the subject.

A fortieth embodiment relates to a compound selected from the exemplary species depicted in Examples 1 through 26 shown below.

A forty-first embodiment relates to a pharmaceutical composition, said pharmaceutical composition comprising (a) a compound according to the fortieth embodiment above or a pharmaceutically acceptable salt thereof; and (b) a pharmaceutically acceptable carrier.

A forty-second embodiment relates to methods of inducing an immune response in a subject, comprising administering a therapeutically effective amount of a compound according to the fortieth embodiment above or a pharmaceutically acceptable salt thereof to the subject.

A forty-third embodiment relates to methods of inducing an immune response in a subject, comprising administering a therapeutically effective amount of a composition according to the forty-first embodiment above to the subject.

A forty-fourth embodiment relates to methods of inducing STING-dependent type I interferon production in a subject, comprising administering a therapeutically effective amount of a compound according to the fortieth embodiment above or a pharmaceutically acceptable salt thereof to the subject.

A forty-fifth embodiment relates to methods of inducing STING-dependent type I interferon production in a subject, comprising administering a therapeutically effective amount of a composition according to the forty-first embodiment above to the subject.

A forty-sixth embodiment relates to methods of inducing STING-dependent cytokine production in a subject, comprising administering a therapeutically effective amount of a compound according to the fortieth embodiment above or a pharmaceutically acceptable salt thereof to the subject.

A forty-seventh embodiment relates to methods of inducing STING-dependent cytokine production in a subject, comprising administering a therapeutically effective amount of a composition according to the forty-first embodiment above to the subject.

Other embodiments of the present disclosure include the following:

(a) A pharmaceutical composition comprising an effective amount of a compound of general formula (I), or a pharmaceutically acceptable salt thereof, and a pharmaceutically acceptable carrier.

(b) The pharmaceutical composition of (a), further comprising an active agent, or a pharmaceutically acceptable salt thereof, selected from the group consisting of STING agonist compounds, anti-viral compounds, antigens, adjuvants, CTLA-4 and PD-1 pathway antagonists and other immunomodulatory agents, lipids, liposomes, peptides, anti-cancer and chemotherapeutic agents.

(c) A pharmaceutical combination that is (i) a compound of general formula (I), or a pharmaceutically acceptable salt thereof, and (ii) an active agent, or a pharmaceutically acceptable salt thereof, selected from the group consisting of STING agonist compounds, anti viral compounds, antigens, adjuvants, CTLA-4 and PD-1 pathway antagonists and other immunomodulatory agents, lipids, liposomes, peptides, anti-cancer and chemotherapeutic agents; wherein the compound of general formula (I), or pharmaceutically acceptable salt thereof, and the active agent are each employed in an amount that renders the combination effective for inducing an immune response in a patient.

(d) A method of inducing an immune response in a patient, which comprises administering to the subject a therapeutically effective amount of a compound of general formula (I), or a pharmaceutically acceptable salt thereof.

(e) A method of inducing an immune response in a patient, which comprises administering to the subject a therapeutically effective amount of a composition of (a), a composition of (b), or a combination of (c).

(f) A method of inducing STING-dependent type I interferon production in a patient, which comprises administering to the subject a therapeutically effective amount of a compound of general formula (I), or a pharmaceutically acceptable salt thereof.

(g) A method of inducing STING-dependent type I interferon production in a patient, which comprises administering to the subject a therapeutically effective amount of a composition of (a), a composition of (b), or a combination of (c).

(h) A method of inducing STING-dependent cytokine production in a patient, which comprises administering to the subject a therapeutically effective amount of a compound of general formula (I), or a pharmaceutically acceptable salt thereof.

(i) A method of inducing STING-dependent cytokine production in a patient, which comprises administering to the subject a therapeutically effective amount of a composition of (a), a composition of (b), or a combination of (c).

(j) A method of treating a cell proliferation disorder in a subject, said method comprising administering a therapeutically effective amount of a compound of general formula (I), or a pharmaceutically acceptable salt thereof to the subject;

(k) The method of (j), wherein the cell proliferation disorder is cancer.

(l) A method of treating a cell proliferation disorder in a subject, said method comprising administering a therapeutically effective amount of a composition of (a), a composition of (b), or a combination of (c) to the subject.

The present disclosure also includes a compound of the present disclosure for use (i) in, (ii) as a medicament for, or (iii) in the preparation of a medicament for: (a) inducing an immune response in a patient, or (b) inducing STING-dependent cytokine production in a patient. In these uses, the compounds of the present disclosure can optionally be employed in combination with one or more active agents selected from STING agonist compounds, anti-viral compounds, antigens, adjuvants, CTLA-4 and PD-1 pathway antagonists and other immunomodulatory agents, lipids, liposomes, peptides, anti-cancer agents, and chemotherapeutic agents.

Additional embodiments of the disclosure include the pharmaceutical compositions, combinations and methods set forth in (a) through (1) above and the uses set forth in the preceding paragraph, wherein the compound of the present disclosure employed therein is a compound of one of the embodiments, aspects, instances, occurrences, or features of the compounds described above. In all of these embodiments, the compound may optionally be used in the form of a pharmaceutically acceptable salt, as appropriate.

In the embodiments of the compound provided above, it is to be understood that each embodiment may be combined with one or more other embodiments, to the extent that such a combination provides a stable compound and is consistent with the description of the embodiments. It is further to be understood that the embodiments of compositions and methods provided as (a) through (1) above are understood to include all embodiments of the compounds, including such embodiments as result from combinations of embodiments.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), Lagomorpha (rabbits), rodents (e.g., rats or mice), Procyon lotor (e.g., raccoons). In particular embodiments, the subject is human.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell proliferation, immune cell differentiation, and cytokine expression. In certain embodiments, a compound of general formula (I), or a pharmaceutically acceptable salt of the foregoing, is administered in conjunction with one or more additional therapeutic agents including anti-viral compounds, vaccines intended to stimulate an immune response to one or more predetermined antigens, adjuvants, CTLA-4 and PD-1 pathway antagonists and other immunomodulatory agents, lipids, liposomes, peptides, anti-cancer agents, and chemotherapeutic agents, etc. In certain embodiments, a compound of general formula (I), or a pharmaceutically acceptable salt of the foregoing, is administered in conjunction with one or more additional compositions including anti-viral compounds, vaccines intended to stimulate an immune response to one or more predetermined antigens, adjuvants, CTLA-4 and PD-1 pathway antagonists and other immunomodulatory agents, lipids, liposomes, peptides, anti-cancer agents, and chemotherapeutic agents, etc.

### Compounds

The term “alkyl” refers to a monovalent straight or branched chain, saturated aliphatic hydrocarbon radical having a number of carbon atoms in the specified range. Thus, for example, “C₁₋₆ alkyl” (or “C₁-C₆ alkyl”) refers to any of the hexyl alkyl and pentyl alkyl isomers as well as n-, iso-, sec- and tert-butyl, n- and iso-propyl, ethyl, and methyl. As another example, “C₁₋₄ alkyl” refers to n-, iso-, sec- and tert-butyl, n- and isopropyl, ethyl, and methyl.

As used herein, the term “alkylene” refers to a bivalent straight chain, saturated aliphatic hydrocarbon radical having a number of carbon atoms in the specified range.

As used herein, the term “alkenyl” refers to a monovalent straight or branched chain, unsaturated aliphatic hydrocarbon radical having a number of carbon atoms in the specified range and including one or more double bond.

As used herein, the term “alkenylene” refers to a bivalent straight chain, unsaturated aliphatic hydrocarbon radical having a number of carbon atoms in the specified range and including one or more double bond.

As used herein, the term “alkynyl” refers to a monovalent straight or branched chain, unsaturated aliphatic hydrocarbon radical having a number of carbon atoms in the specified range and including one or more triple bond.

As used herein, the term “alkynylene” refers to a bivalent straight chain, unsaturated aliphatic hydrocarbon radical having a number of carbon atoms in the specified range and including one or more triple bond.

The term “halogen” (or “halo”) refers to fluorine, chlorine, bromine, and iodine (alternatively referred to as fluoro, chloro, bromo, and iodo or F, Cl, Br, and I).

The term “haloalkyl” refers to an alkyl group as defined above in which one or more of the hydrogen atoms have been replaced with a halogen. Thus, for example, “C₁₋₆ haloalkyl” (or “C₁-C₆ haloalkyl”) refers to a C₁ to C₆ linear or branched alkyl group as defined above with one or more halogen substituents. The term “fluoroalkyl” has an analogous meaning except the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.).

As used herein, the term “haloalkenyl” refers to an alkenyl group as defined above in which one or more of the hydrogen atoms have been replaced with a halogen.

As used herein, the term “haloalkynyl” refers to an alkynyl group as defined above in which one or more of the hydrogen atoms have been replaced with a halogen.

As used herein, the term “alkoxy” as used herein, alone or in combination, includes an alkyl group connected to the oxy connecting atom. The term “alkoxy” also includes alkyl ether groups, where the term “alkyl” is defined above, and “ether” means two alkyl groups with an oxygen atom between them. Examples of suitable alkoxy groups include methoxy, ethoxy, n-propoxy, i-propoxy, n-butoxy, s-butoxy, and t-butoxy.

As used herein, the term “cycloalkyl” refers to a saturated hydrocarbon containing one ring having a specified number of carbon atoms. Examples of cycloalkyl include cyclopropyl, cyclobutyl, cyclopentyl, and cyclohexyl.

As used herein, the term “heterocycle”, “heterocyclyl”, or “heterocyclic”, as used herein, represents a stable 3- to 6-membered monocyclic that is either saturated or unsaturated, and that consists of carbon atoms and from one to two heteroatoms selected from the group consisting of N, O, and S. The heterocyclic ring may be attached at any heteroatom or carbon atom which results in the creation of a stable structure. The term includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofiuyl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofiuyl, thienothienyl, triazolyl, and thienyl.

As used herein, the term “spirocycle” or “spirocyclic ring” refers to a pendant cyclic group formed by substituents on a single atom.

The term “compound” refers to the compound and, in certain embodiments, to the extent they are stable, any hydrate or solvate thereof.

A “stable” compound is a compound that can be prepared and isolated and whose structure and properties remain or can be caused to remain essentially unchanged for a period of time sufficient to allow use of the compound for the purposes described herein (e.g., therapeutic administration to a subject). The compounds of the present invention are limited to stable compounds embraced by general formula (I), or pharmaceutically acceptable salts thereof.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations off 1%, ±2%, ±3%, ±4%, ±5%, ±10%, ±15%, and ±20% and their numerical equivalents.

As used herein, the term “one or more” item includes a single item selected from the list as well as mixtures of two or more items selected from the list.

In the compounds of general formula (I), and pharmaceutically acceptable salts of the foregoing, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of general formula (I), and pharmaceutically acceptable salts of the foregoing. For example, different isotopic forms of hydrogen (H) include protium (¹H), deuterium (²H), and tritium (³H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within general formula (I), and the pharmaceutically acceptable salts of the foregoing, can be prepared without undue experimentation by conventional techniques well known to those skilled in the art or by processes analogous to those described in the Schemes and Examples herein using appropriate isotopically-enriched reagents and/or intermediates.

In particular embodiments of the compounds of general formula (I), and/or pharmaceutically acceptable salts of the foregoing, the compounds are isotopically enriched with deuterium. In aspects of these embodiments, one or more of Base¹, Base², Y, Y^{a}, X^{a}, X^{a1}, X^{b}, X^{b1}, R¹, R^{1a}, R^{2a}, R³, R^{3a}, R⁴, R^{4a}, R⁵, R⁶, R^{6a}, R⁷, R^{7a}, R⁸, R^{8a}, and R⁹ may include deuterium.

As shown in the general structural formulas and the structures of specific compounds as provided herein, a straight line at a chiral center includes both (R) and (S) stereoisomers and mixtures thereof.

Recitation or depiction of a specific compound in the claims (i.e., a species) without a specific stereoconfiguration designation, or with such a designation for less than all chiral centers, is intended to encompass, for such undesignated chiral centers, the racemate, racemic mixtures, each individual enantiomer, a diastereoisomeric mixture and each individual diastereomer of the compound where such forms are possible due to the presence of one or more asymmetric centers.

The invention includes all possible enantiomers and diastereomers and mixtures of two or more stereoisomers, for example mixtures of enantiomers and/or diastereomers, in all ratios.

Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism, the invention includes both the cis form and the trans form, as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of general formula (I), or a pharmaceutically acceptable salt of the foregoing, or it can be done on a final racemic product. Absolute stereochemistry may be determined by X-ray crystallography of crystalline products or crystalline intermediates that are derivatized, if necessary, with a reagent containing a stereogenic center of known configuration. Unless a particular isomer, salt, solvate (including hydrates) or solvated salt of such racemate, enantiomer, or diastereomer is indicated, the present invention includes all such isomers, as well as salts, solvates (including hydrates), and solvated salts of such racemates, enantiomers, diastereomers, and mixtures thereof.

Those skilled in the art will recognize that chiral compounds, and in particular sugars, can be drawn in a number of different ways that are equivalent. Those skilled in the art will further recognize that the identity and regiochemical position of the substituents on ribose can vary widely and that the same principles of steroechemical equivalence apply regardless of substituent. Non-limiting examples of such equivalence include those exemplified below.

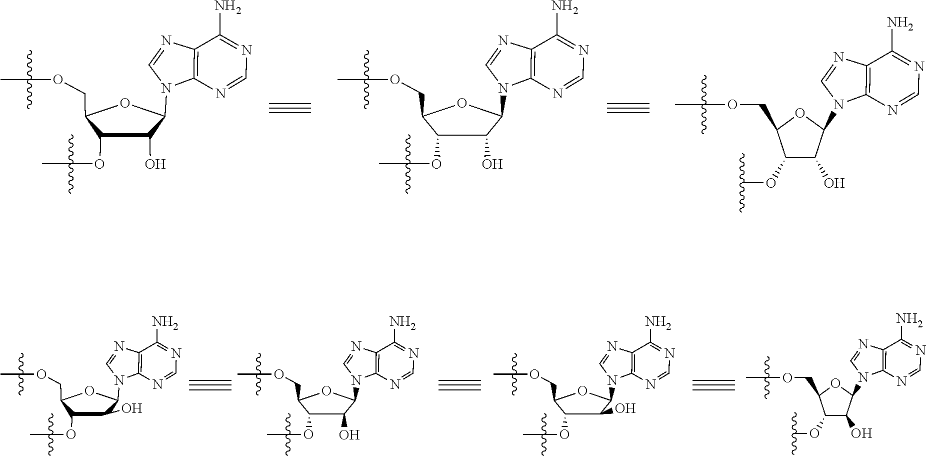

### Salts

As indicated above, the compounds of the present invention can be employed in the form of pharmaceutically acceptable salts. Those skilled in the art will recognize those instances in which the compounds of the invention may form salts. Examples of such compounds are described herein by reference to possible salts. Such reference is for illustration only. Pharmaceutically acceptable salts can be used with compounds for treating patients. Non-pharmaceutical salts may, however, be useful in the preparation of intermediate compounds.

The term “pharmaceutically acceptable salt” refers to a salt (including an inner salt such as a zwitterion) that possesses effectiveness similar to the parent compound and that is not biologically or otherwise undesirable (e.g., is neither toxic nor otherwise deleterious to the recipient thereof). Thus, an embodiment of the invention provides pharmaceutically acceptable salts of the compounds of the invention. The term “salt(s)”, as employed herein, denotes any of the following: acidic salts formed with inorganic and/or organic acids, as well as basic salts formed with inorganic and/or organic bases. Salts of compounds of the invention may be formed by methods known to those of ordinary skill in the art, for example, by reacting a compound of the invention with an amount of acid or base, such as an equivalent amount, in a medium such as one in which the salt precipitates or in aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.), Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts), and salts formed with suitable organic ligands such as quaternary ammonium salts. Also, in the case of an acid (such as —COOH) or alcohol group being present, pharmaceutically acceptable esters can be employed to modify the solubility or hydrolysis characteristics of the compound.

All such acid salts and base salts are intended to be pharmaceutically acceptable salts within the scope of the invention and all acid and base salts are considered equivalent to the free forms of the corresponding compounds for purposes of the invention.

In addition, when a compound of the invention contains both a basic moiety, such as, but not limited to an aliphatic primary, secondary, tertiary or cyclic amine, an aromatic or heteroaryl amine, pyridine or imidazole, and an acidic moiety, such as, but not limited to tetrazole or carboxylic acid, zwitterions (“inner salts”) may be formed and are included within the terms “salt(s)” as used herein. It is understood that certain compounds of the invention may exist in zwitterionic form, having both anionic and cationic centers within the same compound and a net neutral charge. Such zwitterions are included within the invention.

### Methods of Preparing Compounds

Exemplary methods for preparing the compounds of general formula (I), and pharmaceutically acceptable salts of the foregoing, are described in the following Schemes and Examples. Starting materials and intermediates are purchased from commercial sources, made from known procedures, or are otherwise illustrated. In some cases the order of carrying out the steps of the reaction schemes may be varied to facilitate the reaction or to avoid unwanted reaction products.

In the following Methods and Schemes, PG₁ or PG₂ represents a protecting group for an amino group in a nucleobase, which may be a phenyl carbonyl group including benzoyl group, an alkyl carbonyl group including isobutyl carbonyl group and 2-(4-(tert-butyl)phenoxy) acetyl group or a formamidine group including N,N-dimethyl-formamidine. All other variables have the same meaning as provided above.

### Method 1

One method for the preparation of examples of Formula (I) of the instant invention is detailed in Scheme 1. This procedure was adequately modified from the previously reported procedure for cyclic dinucleotide synthesis (Barbara L. Gaffney et al., One-Flask Syntheses of c-di-GMP and the [Rp,Rp] and [Rp,Sp] Thiophosphate Analogues, 12 ORG. LETT. 3269-3271 (2010)). The sequence starts with modified ara-nucleoside with DMTr ether at 5′-0 position and a nucleobase of which amino group was appropriately protected as described above if necessary. It was treated with diphenyl phosphite and then, aqueous trimethylamine to introduce an H-phosphonate group. Then, DMTr ether was removed under acidic condition. The resulting 5′-hydroxyl group was reacted with 3′-phosphoramidites of fully protected second modified nucleotide to give a linear dimer compound. It was immediately treated with (E)-N,N-dimethyl-N′-(3-thioxo-3H-1,2,4-dithiazol-5-yl)formimidamide or tert-butyl hydroperoxide. Then, the 5′-hydroxyl group of the second nucleotide was deprotected with dichloroacetic acid. Using diphenyl chlorophosphate as a coupling reagent, the H-phosphonate at 2′-O of the first nucleotide was reacted with 5′-OH of the second nucleotide to give a cyclic product. It was immediately sulfurized with 3H-1,2-benzodithiol-3-one. Treatment with ammonium hydroxide, t-butylamine, or methylamine plus fluoride anion in case silyl protection was used provided the desired cyclic dinucleotide 1G.

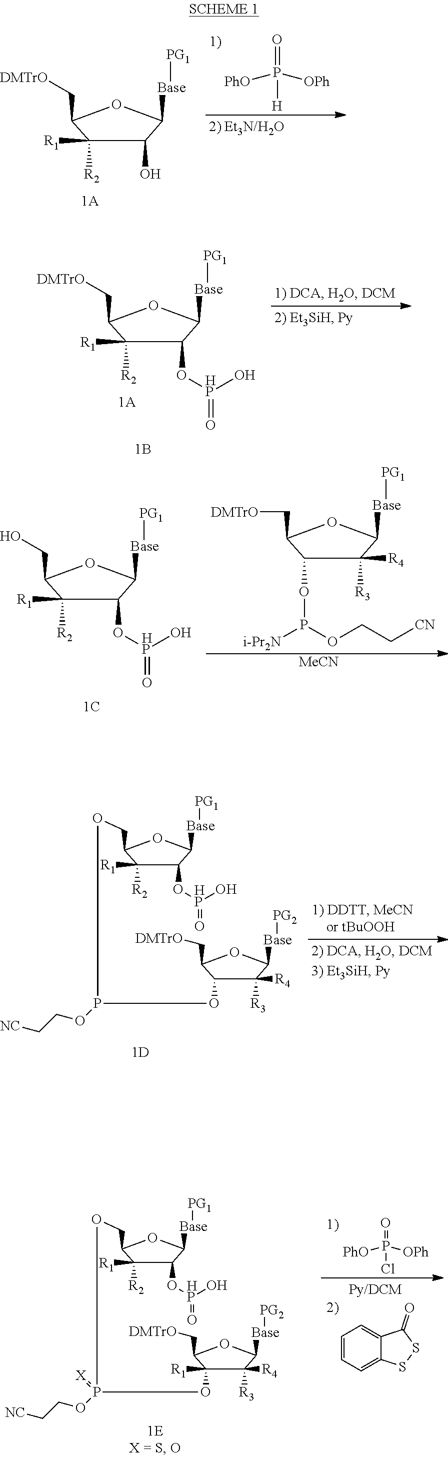

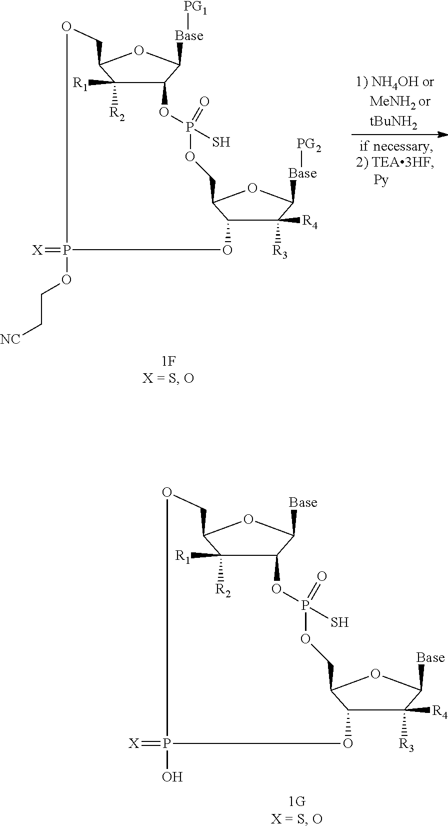

### Methods of Use

Compounds described herein having therapeutic applications, such as the compounds of general formula (I), the compounds of the Examples 1 through 26, and pharmaceutically acceptable salts, hydrates, and solvates thereof, of the foregoing, may be administered to a patient for the purpose of inducing an immune response, inducing STING-dependent cytokine production and/or inducing anti-tumor activity. The term “administration” and variants thereof (e.g., “administering” a compound) means providing the compound to the individual in need of treatment. When a compound is provided in combination with one or more additional active agents (e.g., antiviral agents useful for treating HCV infection or anti-tumor agents for treating cancers), “administration” and its variants are each understood to include concurrent and sequential provision of the compound or salt and other agents.

The compounds disclosed herein may be STING agonists. These compounds are potentially useful in treating diseases or disorders including, but not limited to, cell proliferation disorders. Cell-proliferation disorders include, but are not limited to, cancers, benign papillomatosis, gestational trophoblastic diseases, and benign neoplastic diseases, such as skin papilloma (warts) and genital papilloma.

In specific embodiments, the disease or disorder to be treated is a cell proliferation disorder. In certain embodiments, the cell proliferation disorder is cancer. In particular embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary (i.e., cancers in which a metastasized cancer is found but the original cancer site is not known). In particular embodiments, the cancer is present in an adult patient; in additional embodiments, the cancer is present in a pediatric patient. In particular embodiments, the cancer is AIDS-related.

In specific embodiments, the cancer is selected from brain and spinal cancers. In particular embodiments, the cancer is selected from the group consisting of anaplastic astrocytomas, glioblastomas, astrocytomas, and estheosioneuroblastomas (also known as olfactory blastomas). In particular embodiments, the brain cancer is selected from the group consisting of astrocytic tumor (e.g., pilocytic astrocytoma, subependymal giant-cell astrocytoma, diffuse astrocytoma, pleomorphic xanthoastrocytoma, anaplastic astrocytoma, astrocytoma, giant cell glioblastoma, glioblastoma, secondary glioblastoma, primary adult glioblastoma, and primary pediatric glioblastoma), oligodendroglial tumor (e.g., oligodendroglioma, and anaplastic oligodendroglioma), oligoastrocytic tumor (e.g., oligoastrocytoma, and anaplastic oligoastrocytoma), ependymoma (e.g., myxopapillary ependymoma, and anaplastic ependymoma); medulloblastoma, primitive neuroectodermal tumor, schwannoma, meningioma, atypical meningioma, anaplastic meningioma, pituitary adenoma, brain stem glioma, cerebellar astrocytoma, cerebral astorcytoma/malignant glioma, visual pathway and hypothalmic glioma, and primary central nervous system lymphoma. In specific instances of these embodiments, the brain cancer is selected from the group consisting of glioma, glioblastoma multiforme, paraganglioma, and suprantentorial primordial neuroectodermal tumors (sPNET).

In specific embodiments, the cancer is selected from cancers of the head and neck, including nasopharyngeal cancers, nasal cavity and paranasal sinus cancers, hypopharyngeal cancers, oral cavity cancers (e.g., squamous cell carcinomas, lymphomas, and sarcomas), lip cancers, oropharyngeal cancers, salivary gland tumors, cancers of the larynx (e.g., laryngeal squamous cell carcinomas, rhabdomyosarcomas), and cancers of the eye or ocular cancers. In particular embodiments, the ocular cancer is selected from the group consisting of intraocular melanoma and retinoblastoma.

In specific embodiments, the cancer is selected from leukemia and cancers of the blood. In particular embodiments, the cancer is selected from the group consisting of myeloproliferative neoplasms, myelodysplastic syndromes, myelodysplastic/myeloproliferative neoplasms, acute myeloid leukemia (AML), myelodysplastic syndrome (MDS), chronic myelogenous leukemia (CML), myeloproliferative neoplasm (MPN), post-MPN AML, post-MDS AML, del(5q)-associated high risk MDS or AML, blast-phase chronic myelogenous leukemia, angioimmunoblastic lymphoma, acute lymphoblastic leukemia, Langerans cell histiocytosis, hairy cell leukemia, and plasma cell neoplasms including plasmacytomas and multiple myelomas. Leukemias referenced herein may be acute or chronic.

In specific embodiments, the cancer is selected from skin cancers. In particular embodiments, the skin cancer is selected from the group consisting of melanoma, squamous cell cancers, and basal cell cancers.

In specific embodiments, the cancer is selected from cancers of the reproductive system. In particular embodiments, the cancer is selected from the group consisting of breast cancers, cervical cancers, vaginal cancers, ovarian cancers, prostate cancers, penile cancers, and testicular cancers. In specific instances of these embodiments, the cancer is a breast cancer selected from the group consisting of ductal carcinomas and phyllodes tumors. In specific instances of these embodiments, the breast cancer may be male breast cancer or female breast cancer. In specific instances of these embodiments, the cancer is a cervical cancer selected from the group consisting of squamous cell carcinomas and adenocarcinomas. In specific instances of these embodiments, the cancer is an ovarian cancer selected from the group consisting of epithelial cancers.

In specific embodiments, the cancer is selected from cancers of the gastrointestinal system. In particular embodiments, the cancer is selected from the group consisting of esophageal cancers, gastric cancers (also known as stomach cancers), gastrointestinal carcinoid tumors, pancreatic cancers, gallbladder cancers, colorectal cancers, and anal cancer. In instances of these embodiments, the cancer is selected from the group consisting of esophageal squamous cell carcinomas, esophageal adenocarcinomas, gastric adenocarcinomas, gastrointestinal carcinoid tumors, gastrointestinal stromal tumors, gastric lymphomas, gastrointestinal lymphomas, solid pseudopapillary tumors of the pancreas, pancreatoblastoma, islet cell tumors, pancreatic carcinomas including acinar cell carcinomas and ductal adenocarcinomas, gallbladder adenocarcinomas, colorectal adenocarcinomas, and anal squamous cell carcinomas.

In specific embodiments, the cancer is selected from liver and bile duct cancers. In particular embodiments, the cancer is liver cancer (also known as hepatocellular carcinoma). In particular embodiments, the cancer is bile duct cancer (also known as cholangiocarcinoma); in instances of these embodiments, the bile duct cancer is selected from the group consisting of intrahepatic cholangiocarcinoma and extrahepatic cholangiocarcinoma.

In specific embodiments, the cancer is selected from kidney and bladder cancers. In particular embodiments, the cancer is a kidney cancer selected from the group consisting of renal cell cancer, Wilms tumors, and transitional cell cancers. In particular embodiments, the cancer is a bladder cancer selected from the group consisting of urethelial carcinoma (a transitional cell carcinoma), squamous cell carcinomas, and adenocarcinomas.

In specific embodiments, the cancer is selected from bone cancers. In particular embodiments, the bone cancer is selected from the group consisting of osteosarcoma, malignant fibrous histiocytoma of bone, Ewing sarcoma, chordoma (cancer of the bone along the spine).

In specific embodiments, the cancer is selected from lung cancers. In particular embodiments, the lung cancer is selected from the group consisting of non-small cell lung cancer, small cell lung cancers, bronchial tumors, and pleuropulmonary blastomas.

In specific embodiments, the cancer is selected from malignant mesothelioma. In particular embodiments, the cancer is selected from the group consisting of epithelial mesothelioma and sarcomatoids.

In specific embodiments, the cancer is selected from sarcomas. In particular embodiments, the sarcoma is selected from the group consisting of central chondrosarcoma, central and periosteal chondroma, fibrosarcoma, clear cell sarcoma of tendon sheaths, and Kaposi's sarcoma.

In specific embodiments, the cancer is selected from lymphomas. In particular embodiments, the cancer is selected from the group consisting of Hodgkin lymphoma (e.g., Reed-Stemberg cells), non-Hodgkin lymphoma (e.g., diffuse large B-cell lymphoma, follicular lymphoma, mycosis fungoides, Sezary syndrome, primary central nervous system lymphoma), cutaneous T-cell lymphomas, primary central nervous system lymphomas. In specific embodiments, the cancer is selected from glandular cancers. In particular embodiments, the cancer is selected from the group consisting of adrenocortical cancer (also known as adrenocortical carcinoma or adrenal cortical carcinoma), pheochromocytomas, paragangliomas, pituitary tumors, thymoma, and thymic carcinomas.

In specific embodiments, the cancer is selected from thyroid cancers. In particular embodiments, the thyroid cancer is selected from the group consisting of medullary thyroid carcinomas, papillary thyroid carcinomas, and follicular thyroid carcinomas.

In specific embodiments, the cancer is selected from germ cell tumors. In particular embodiments, the cancer is selected from the group consisting of malignant extracranial germ cell tumors and malignant extragonadal germ cell tumors. In specific instances of these embodiments, the malignant extragonadal germ cell tumors are selected from the group consisting of nonseminomas and seminomas.

In specific embodiments, the cancer is selected from heart tumors. In particular embodiments, the heart tumor is selected from the group consisting of malignant teratoma, lymphoma, rhabdomyosacroma, angiosarcoma, chondrosarcoma, infantile fibrosarcoma, and synovial sarcoma.

In specific embodiments, the cell-proliferation disorder is selected from benign papillomatosis, benign neoplastic diseases and gestational trophoblastic diseases. In particular embodiments, the benign neoplastic disease is selected from skin papilloma (warts) and genital papilloma. In particular embodiments, the gestational trophoblastic disease is selected from the group consisting of hydatidiform moles, and gestational trophoblastic neoplasia (e.g., invasive moles, choriocarcinomas, placental-site trophoblastic tumors, and epithelioid trophoblastic tumors).

As used herein, the terms “treatment” and “treating” refer to all processes in which there may be a slowing, interrupting, arresting, controlling, or stopping of the progression of a disease or disorder described herein. The terms do not necessarily indicate a total elimination of all disease or disorder symptoms.

The terms “administration of” and or “administering” a compound should be understood to include providing a compound described herein, or a pharmaceutically acceptable salt thereof, and compositions of the foregoing to a subject.

The amount of a compound administered to a subject is an amount sufficient to induce an immune response and/or to induce STING-dependent type I interferon production in the subject. In an embodiment, the amount of a compound can be an “effective amount” or “therapeutically effective amount,” such that the subject compound is administered in an amount that will elicit, respectively, a biological or medical (i.e., intended to treat) response of a tissue, system, animal, or human that is being sought by a researcher, veterinarian, medical doctor, or other clinician. An effective amount does not necessarily include considerations of toxicity and safety related to the administration of a compound.

An effective amount of a compound will vary with the particular compound chosen (e.g., considering the potency, efficacy, and/or half-life of the compound); the route of administration chosen; the condition being treated; the severity of the condition being treated; the age, size, weight, and physical condition of the subject being treated; the medical history of the subject being treated; the duration of the treatment; the nature of a concurrent therapy; the desired therapeutic effect; and like factors and can be routinely determined by the skilled artisan.

The compounds disclosed herein may be administered by any suitable route including oral and parenteral administration. Parenteral administration is typically by injection or infusion and includes intravenous, intramuscular, intratumoral, and subcutaneous injection or infusion.

The compounds disclosed herein may be administered once or according to a dosing regimen where a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound disclosed herein depend on the pharmacokinetic properties of that compound, such as absorption, distribution and half-life, which can be determined by a skilled artisan. In addition, suitable dosing regimens, including the duration such regimens are administered, for a compound disclosed herein depend on the disease or condition being treated, the severity of the disease or condition, the age and physical condition of the subject being treated, the medical history of the subject being treated, the nature of concurrent therapy, the desired therapeutic effect, and like factors within the knowledge and expertise of the skilled artisan. It will be further understood by such skilled artisans that suitable dosing regimens may require adjustment given an individual subject's response to the dosing regimen or over time as the individual subject needs change. Typical daily dosages may vary depending upon the particular route of administration chosen.

One embodiment of the present disclosure provides for a method of treating a cell proliferation disorder comprising administration of a therapeutically effective amount of a compound of general formula (I), or a pharmaceutically acceptable salt of the foregoing, to a subject in need of treatment thereof. In embodiments, the disease or disorder to be treated is a cell proliferation disorder. In aspects of these embodiments, the cell proliferation disorder is cancer. In further aspects of these embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary.

In one embodiment, disclosed herein is the use of a compound of general formula (I), or a pharmaceutically acceptable salt of the foregoing, in a therapy. The compound may be useful in a method of inducing an immune response and/or inducing STING-dependent type I interferon production in a subject, such as a mammal in need of such inhibition, comprising administering an effective amount of the compound to the subject.

In one embodiment, disclosed herein is a pharmaceutical composition comprising at least one compound of general formula (I), or at least one pharmaceutically acceptable salt of the foregoing, for use in potential treatment to induce an immune response and/or to induce STING-dependent type I interferon production.

One embodiment disclosed herein is the use of a compound of general formula (I), or a pharmaceutically acceptable salt of the foregoing, in the manufacture of a medicament to induce an immune response and/or to induce STING-dependent type I interferon production. In embodiments, the disease or disorder to be treated is a cell proliferation disorder. In aspects of these embodiments, the cell proliferation disorder is cancer. In further aspects of these embodiments, the cancer is selected from brain and spinal cancers, cancers of the head and neck, leukemia and cancers of the blood, skin cancers, cancers of the reproductive system, cancers of the gastrointestinal system, liver and bile duct cancers, kidney and bladder cancers, bone cancers, lung cancers, malignant mesothelioma, sarcomas, lymphomas, glandular cancers, thyroid cancers, heart tumors, germ cell tumors, malignant neuroendocrine (carcinoid) tumors, midline tract cancers, and cancers of unknown primary.

### Compositions

The term “composition” as used herein is intended to encompass a dosage form comprising a specified compound in a specified amount, as well as any dosage form that results, directly or indirectly, from combination of a specified compound in a specified amount. Such term is intended to encompass a dosage form comprising a compound of general formula (I), or a pharmaceutically acceptable salt, hydrate, solvate, or prodrug of the foregoing, and one or more pharmaceutically acceptable carriers or excipients. In embodiments, the dosage form comprises compounds of general structural formula (I), or a pharmaceutically acceptable salt, hydrate, or solvate thereof, and one or more pharmaceutically acceptable carriers or excipients. In specific embodiments, the dosage form comprises compounds of general structural formula (I), or a pharmaceutically acceptable salt thereof, and one or more pharmaceutically acceptable carriers or excipients. Accordingly, the compositions of the present disclosure encompass any composition made by admixing a compound of the present disclosure and one or more pharmaceutically acceptable carrier or excipients. By “pharmaceutically acceptable”, it is meant the carriers or excipients are compatible with the compound disclosed herein and with other ingredients of the composition.

For the purpose of inducing an immune response and/or inducing STING-dependent type I interferon production, the compounds of general formula (I), or pharmaceutically acceptable salts of the foregoing, can be administered by means that produces contact of the active agent with the agent's site of action. The compounds can be administered by conventional means available for use in conjunction with pharmaceuticals, either as individual therapeutic agents or in a combination of therapeutic agents. The compounds can be administered alone, but typically are administered with a pharmaceutical carrier selected on the basis of the chosen route of administration and standard pharmaceutical practice.

In one embodiment, disclosed herein is a composition comprising a compound of general formula (I), or a pharmaceutically acceptable salt of the foregoing, and one or more pharmaceutically acceptable carriers or excipients. The composition may be prepared and packaged in bulk form in which a therapeutically effective amount of a compound of the disclosure can be extracted and then given to a subject, such as with powders or syrups. Alternatively, the composition may be prepared and packaged in unit dosage form in which each physically discrete unit contains a therapeutically effective amount of a compound of general formula (I), or a pharmaceutically acceptable salt of the foregoing.

The compounds disclosed herein and a pharmaceutically acceptable carrier or excipient(s) will typically be formulated into a dosage form adapted for administration to a subject by a desired route of administration. For example, dosage forms include those adapted for (1) oral administration, such as tablets, capsules, caplets, pills, troches, powders, syrups, elixirs, suspensions, solutions, emulsions, sachets, and cachets; and (2) parenteral administration, such as sterile solutions, suspensions, and powders for reconstitution. Suitable pharmaceutically acceptable carriers or excipients will vary depending upon the particular dosage form chosen. In addition, suitable pharmaceutically acceptable carriers or excipients may be chosen for a particular function that they may serve in the composition. For example, certain pharmaceutically acceptable carriers or excipients may be chosen for their ability to facilitate the production of uniform dosage forms. Certain pharmaceutically acceptable carriers or excipients may be chosen for their ability to facilitate the production of stable dosage forms. Certain pharmaceutically acceptable carriers or excipients may be chosen for their ability to facilitate the carrying or transporting of a compound disclosed herein, once administered to the subject, from one organ or portion of the body to another organ or another portion of the body. Certain pharmaceutically acceptable carriers or excipients may be chosen for their ability to enhance patient compliance.

Suitable pharmaceutically acceptable excipients include the following types of excipients: diluents, lubricants, binders, disintegrants, fillers, glidants, granulating agents, coating agents, wetting agents, solvents, co-solvents, suspending agents, emulsifiers, sweeteners, flavoring agents, flavor masking agents, coloring agents, anti-caking agents, hemectants, chelating agents, plasticizers, viscosity increasing agents, antioxidants, preservatives, stabilizers, surfactants, and buffering agents.

A skilled artisan possesses the knowledge and skill in the art to select suitable pharmaceutically acceptable carriers and excipients in appropriate amounts for the use in the compositions of the disclosure. In addition, there are a number of resources available to the skilled artisan, which describe pharmaceutically acceptable carriers and excipients and may be useful in selecting suitable pharmaceutically acceptable carriers and excipients. Examples include REMINGTON'S PHARMACEUTICAL SCIENCES (Mack Publishing Company), THE HANDBOOK OF PHARMACEUTICAL ADDITIVES (Gower Publishing Limited), and THE HANDBOOK OF PHARMACEUTICAL EXCIPIENTS (the American Pharmaceutical Association and the Pharmaceutical Press).

The compositions of the disclosure are prepared using techniques and methods known to those skilled in the art. Some methods commonly used in the art are described in REMINGTON'S PHARMACEUTICAL SCIENCES (Mack Publishing Company).

In one embodiment, the disclosure is directed to a solid oral dosage form such as a tablet or capsule comprising a therapeutically effective amount of a compound of general formula (I), or a pharmaceutically acceptable salt of the foregoing, and a diluent or filler. Suitable diluents and fillers include lactose, sucrose, dextrose, mannitol, sorbitol, starch (e.g., corn starch, potato starch, and pre-gelatinized starch), cellulose and its derivatives, (e.g., microcrystalline cellulose), calcium sulfate, and dibasic calcium phosphate. The solid oral dosage form may further comprise a binder. Suitable binders include starch (e.g., corn starch, potato starch, and pre-gelatinized starch) gelatin, acacia, sodium alginate, alginic acid, tragacanth, guar gum, povidone, and cellulose and its derivatives (e.g., microcrystalline cellulose). The solid oral dosage form may further comprise a disintegrant. Suitable disintegrants include crospovidone, sodium starch glycolate, croscarmelose, alginic acid, and sodium carboxymethyl cellulose. The solid oral dosage form may further comprise a lubricant. Suitable lubricants include stearic acid, magnesium stearate, calcium stearate, and talc.

Where appropriate, dosage unit formulations for oral administration can be microencapsulated. The composition can also be prepared to prolong or sustain the release as, for example, by coating or embedding particulate material in polymers, wax, or the like.

The compounds disclosed herein may also be coupled with soluble polymers as targetable drug carriers. Such polymers can include polyvinylpyrrolidone, pyrancopolymer, polyhydroxypropylmethacrylamidephenol, polyhydroxyethylaspartamidephenol, or polyethyleneoxidepolylysine substituted with palmitoyl residues. Furthermore, the compounds of the disclosure may be coupled to a class of biodegradable polymers useful in achieving controlled release of a drug, for example polylactic acid, polepsilon caprolactone, polyhydroxy butyric acid, polyorthoesters, polyacetals, polydihydropyrans, polycyanacrylates, and cross-linked or amphipathic block copolymers of hydrogels.

In one embodiment, the disclosure is directed to a liquid oral dosage form. Oral liquids such as solutions, syrups, and elixirs can be prepared in dosage unit form so that a given quantity contains a predetermined amount of a compound or a pharmaceutically acceptable salt thereof disclosed herein. Syrups can be prepared by dissolving the compound of the disclosure in a suitably flavored aqueous solution; elixirs are prepared through the use of a non-toxic alcoholic vehicle. Suspensions can be formulated by dispersing a compound disclosed herein in a non-toxic vehicle. Solubilizers and emulsifiers such as ethoxylated isostearyl alcohols and polyoxy ethylene sorbitol ethers, preservatives, flavor additives such as peppermint oil, or other natural sweeteners or saccharin or other artificial sweeteners and the like can also be added.

In one embodiment, the disclosure is directed to compositions for parenteral administration. Compositions adapted for parenteral administration include aqueous and non-aqueous sterile injection solutions that may contain anti-oxidants, buffers, bacteriostats, and solutes that render the formulation isotonic with the blood of the intended recipient; and aqueous and non-aqueous sterile suspensions that may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

### Combinations

The compounds of general formula (I), and/or pharmaceutically acceptable salts of the foregoing, may be administered in combination with one or more additional active agents. In embodiments, one or more compounds of general formula (I), or one or more pharmaceutically acceptable salts of the foregoing, and the one or more additional active agents may be co-administered. The additional active agent(s) may be administered in a single dosage form with the compound of general formula (I), or pharmaceutically acceptable salt of the foregoing, or the additional active agent(s) may be administered in separate dosage form(s) from the dosage form containing the compound of general formula (I), or pharmaceutically acceptable salt of the foregoing. The additional active agent(s) may be one or more agents selected from the group consisting of STING agonist compounds, anti-viral compounds, antigens, adjuvants, anti-cancer agents, CTLA-4, LAG-3, and PD-1 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines. It will be understood the descriptions of the above additional active agents may be overlapping. It will also be understood that the treatment combinations are subject to optimization, and it is understood that the best combination to use of the compounds of general formula (I), or pharmaceutically acceptable salts of the foregoing, and one or more additional active agents will be determined based on the individual patient needs.

A compound disclosed herein may be used in combination with one or more other active agents, including but not limited to, other anti-cancer agents that are used in the prevention, treatment, control, amelioration, or reduction of risk of a particular disease or condition (e.g., cell proliferation disorders). In one embodiment, a compound disclosed herein is combined with one or more other anti-cancer agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the present disclosure.

When a compound disclosed herein is used contemporaneously with one or more other active agents, a composition containing such other active agents in addition to the compound disclosed herein is contemplated. Accordingly, the compositions of the present disclosure include those that also contain one or more other active ingredients, in addition to a compound disclosed herein. A compound disclosed herein may be administered either simultaneously with, or before or after, one or more other active agent(s). A compound disclosed herein may be administered separately, by the same or different route of administration, or together in the same pharmaceutical composition as the other agent(s).

Products provided as combinations may include a composition comprising a compound of general formula (I), or a pharmaceutically acceptable salt of the foregoing, and one or more other active agent(s) together in the same pharmaceutical composition, or may include a composition comprising a compound of general formula (I), or a pharmaceutically acceptable salt of the foregoing, and a composition comprising one or more other active agent(s) in separate form, e.g. in the form of a kit or in any form designed to enable separate administration either concurrently or on separate dosing schedules.

The weight ratio of a compound of general formula (I), or a pharmaceutically acceptable salt of the foregoing, to a second active agent may be varied and will depend upon the therapeutically effective dose of each agent. Generally, a therapeutically effective dose of each will be used. Combinations of a compound disclosed herein and other active agents will generally also be within the aforementioned range, but in each case, a therapeutically effective dose of each active agent should be used. In such combinations, the compound disclosed herein and other active agents may be administered separately or in conjunction. In addition, the administration of one element may be prior to, concurrent to, or subsequent to the administration of other agent(s).

In one embodiment, this disclosure provides a composition comprising a compound of general formula (I), or a pharmaceutically acceptable salt thereof, and at least one other active agent as a combined preparation for simultaneous, separate or sequential use in therapy. In one embodiment, the therapy is the treatment of a cell proliferation disorder, such as cancer.

In one embodiment, the disclosure provides a kit comprising two or more separate pharmaceutical compositions, at least one of which contains a compound of general formula (I), or a pharmaceutically acceptable salt of the foregoing. In one embodiment, the kit comprises means for separately retaining said compositions, such as a container, divided bottle, or divided foil packet. An example of such a kit is a blister pack, as typically used for the packaging of tablets, capsules, and the like.

A kit of this disclosure may be used for administration of different dosage forms, for example, oral and parenteral, for administration of the separate compositions at different dosage intervals, or for titration of the separate compositions against one another. To assist with compliance, a kit of the disclosure typically comprises directions for administration.

Disclosed herein is a use of a compound of general formula (I), or a pharmaceutically acceptable salt of the foregoing, for treating a cell proliferation disorder, where the medicament is prepared for administration with another active agent. The disclosure also provides the use of another active agent for treating a cell proliferation disorder, where the medicament is administered with a compound of general formula (I), or a pharmaceutically acceptable salt of the foregoing.

The disclosure also provides the use of a compound of general formula (I), or a pharmaceutically acceptable salt of the foregoing, for treating a cell proliferation disorder, where the patient has previously (e.g., within 24 hours) been treated with another active agent. The disclosure also provides the use of another active agent for treating a cell proliferation disorder, where the patient has previously (e.g., within 24 hours) been treated with a compound of general formula (I), or a pharmaceutically acceptable salt of the foregoing. The second agent may be administered a week, several weeks, a month, or several months after the administration of a compound disclosed herein.

STING agonist compounds that may be used in combination with the compounds of general formula (I), or pharmaceutically acceptable salts of the foregoing, disclosed herein include but are not limited to cyclic di-nucleotide compounds.

Anti-viral compounds that may be used in combination with the compounds of general formula (I), or pharmaceutically acceptable salts of the foregoing, disclosed herein include hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors.

Antigens and adjuvants that may be used in combination with the compounds of general formula (I), or the pharmaceutically acceptable salts of the foregoing, include B7 costimulatory molecule, interleukin-2, interferon-y, GM-CSF, CTLA-4 antagonists, OX-40/0X-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryllipid A, lipoteichoic acid, imiquimod, resiquimod, and in addition retinoic acid-inducible gene I (RIG-I) agonists such as poly I:C, used separately or in combination with the described compositions are also potential adjuvants.

CLTA-4 and PD-1 pathways are important negative regulators of immune response. Activated T-cells up-regulate CTLA-4, which binds on antigen-presenting cells and inhibits T-cell stimulation, IL-2 gene expression, and T-cell proliferation; these anti-tumor effects have been observed in mouse models of colon carcinoma, metastatic prostate cancer, and metastatic melanoma. PD-1 binds to active T-cells and suppresses T-cell activation; PD-1 antagonists have demonstrated anti-tumor effects as well. CTLA-4 and PD-1 pathway antagonists that may be used in combination with the compounds of general formula (Ia), the compounds of general formula (Ib), the compounds of general formula (I), or the pharmaceutically acceptable salts of the foregoing, disclosed herein, include ipilimumab, tremelimumab, nivolumab, pembrolizumab, CT-011, AMP-224, and MDX-1106.

“PD-1 antagonist” or “PD-1 pathway antagonist” means any chemical compound or biological molecule that blocks binding of PD-L1 expressed on a cancer cell to PD-1 expressed on an immune cell (T-cell, B-cell, or NKT-cell) and preferably also blocks binding of PD-L2 expressed on a cancer cell to the immune-cell expressed PD-1. Alternative names or synonyms for PD-1 and its ligands include: PDCD1, PD1, CD279, and SLEB2 for PD-1; PDCD1L1, PDL1, B7H1, B7-4, CD274, and B7-H for PD-L1; and PDCD1L2, PDL2, B7-DC, Btdc, and CD273 for PD-L2. In any of the treatment method, medicaments and uses of the present disclosure in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L 1 and PD-L2 amino acid sequences can be found in NCBI Locus No.: NP_054862 and NP_079515, respectively.

PD-1 antagonists useful in any of the treatment method, medicaments and uses of the present disclosure include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody, or a chimeric antibody and may include a human constant region. In some embodiments, the human constant region is selected from the group consisting of IgG1, IgG2, IgG3, and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv, and Fv fragments.

Examples of mAbs that bind to human PD-1, and useful in the treatment method, medicaments and uses of the present disclosure, are described in U.S. Pat. Nos. 7,488,802, 7,521,051, 8,008,449, 8,354,509, and 8,168,757, PCT International Patent Application Publication Nos. WO2004/004771, WO2004/072286, and WO2004/056875, and U.S. Patent Application Publication No. US2011/0271358.

Examples of mAbs that bind to human PD-L1, and useful in the treatment method, medicaments and uses of the present disclosure, are described in PCT International Patent Application Nos. WO2013/019906 and WO2010/077634 A1 and in U.S. Pat. No. 8,383,796. Specific anti-human PD-L1 mAbs useful as the PD-1 antagonist in the treatment method, medicaments and uses of the present disclosure include MPDL3280A, BMS-936559, MEDI4736, MSB0010718C, and an antibody that comprises the heavy chain and light chain variable regions of SEQ ID NO:24 and SEQ ID NO:21, respectively, of WO2013/019906.

Other PD-1 antagonists useful in any of the treatment method, medicaments, and uses of the present disclosure include an immune-adhesion that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immune-adhesion molecules that specifically bind to PD-1 are described in PCT International Patent Application Publication Nos. WO2010/027827 and WO2011/066342. Specific fusion proteins useful as the PD-1 antagonist in the treatment method, medicaments, and uses of the present disclosure include AMP-224 (also known as B7-DCIg), which is a PD-L2-FC fusion protein and binds to human PD-1.

Examples of cytotoxic agents that may be used in combination with the compounds of general formula (I) or pharmaceutically acceptable salts thereof, include, but are not limited to, arsenic trioxide (sold under the tradename TRISENOX®), asparaginase (also known as L-asparaginase, and Erwinia L-asparaginase, sold under the tradenames ELSPAR® and KIDROLASE®).

Chemotherapeutic agents that may be used in combination with the compounds of general formula (I), or pharmaceutically acceptable salts of the foregoing, disclosed herein include abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-prolyl-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea andtaxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, nivolumab, onapristone, paclitaxel, pembrolizumab, prednimustine, procarbazine, RPR109881, stramustine phosphate, tamoxifen, tasonermin, taxol, tretinoin, vinblastine, vincristine, vindesine sulfate, and vinflunine.

Examples of vascular endothelial growth factor (VEGF) receptor inhibitors include, but are not limited to, bevacizumab (sold under the trademark AVASTIN by Genentech/Roche), axitinib (described in PCT International Patent Publication No. WO01/002369), Brivanib Alaninate ((S)-((R)-1-(4-(4-Fluoro-2-methyl-1H-indol-5-yloxy)-5-methylpyrrolo[2,1-f][1,2,4]triazin-6-yloxy)propan-2-yl)₂-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide. and described in PCT International Patent Application Publication No. WO02/068470), pasireotide (also known as SO 230, and described in PCT International Patent Publication No. WO02/010192), and sorafenib (sold under the tradename NEXAVAR).

Examples of topoisomerase II inhibitors, include but are not limited to, etoposide (also known as VP-16 and Etoposide phosphate, sold under the tradenames TOPOSAR, VEPESID, and ETOPOPHOS), and teniposide (also known as VM-26, sold under the tradename VUMON).

Examples of alkylating agents, include but are not limited to, 5-azacytidine (sold under the trade name VIDAZA), decitabine (sold under the trade name of DECOGEN), temozolomide (sold under the trade names TEMODAR and TEMODAL by Schering-Plough/Merck), dactinomycin (also known as actinomycin-D and sold under the tradename COSMEGEN), melphalan (also known as L-PAM, L-sarcolysin, and phenylalanine mustard, sold under the tradename ALKERAN), altretamine (also known as hexamethylmelamine (HMM), sold under the tradename HEXALEN), carmustine (sold under the tradename BCNU), bendamustine (sold under the tradename TREANDA), busulfan (sold under the tradenames BUSULFEX® and MYLERAN®), carboplatin (sold under the tradename PARAPLATIN®), lomustine (also known as CCNU, sold under the tradename CEENU®), cisplatin (also known as CDDP, sold under the tradenames PLATINOL® and PLATINOL®-AQ), chlorambucil (sold under the tradename LEUKERAN®), cyclophosphamide (sold under the tradenames CYTOXAN® and NEOSAR®), dacarbazine (also known as DTIC, DIC and imidazole carboxamide, sold under the tradename DTIC-DOME®), altretamine (also known as hexamethylmelamine (HMM) sold under the tradename HEXALEN®), ifosfamide (sold under the tradename IFEX®), procarbazine (sold under the tradename MATULANE®), mechlorethamine (also known as nitrogen mustard, mustine and mechloroethamine hydrochloride, sold under the tradename MUSTARGEN®), streptozocin (sold under the tradename ZANOSAR®), thiotepa (also known as thiophosphoamide, TESPA and TSPA, and sold under the tradename THIOPLEX®.

Examples of anti-tumor antibiotics include, but are not limited to, doxorubicin (sold under the tradenames ADRIAMYCIN® and RUBEX®), bleomycin (sold under the tradename LENOXANE®), daunorubicin (also known as dauorubicin hydrochloride, daunomycin, and rubidomycin hydrochloride, sold under the tradename CERUBIDINE®), daunorubicin liposomal (daunorubicin citrate liposome, sold under the tradename DAUNOXOME®), mitoxantrone (also known as DHAD, sold under the tradename NOVANTRONE®), epirubicin (sold under the tradename ELLENCE™), idarubicin (sold under the tradenames IDAMYCIN®, IDAMYCIN PFS®), and mitomycin C (sold under the tradename MUTAMYCIN®).

Examples of anti-metabolites include, but are not limited to, claribine (2 chlorodeoxyadenosine, sold under the tradename LEUSTATIN®), 5-fluorouracil (sold under the tradename ADRUCIL®), 6-thioguanine (sold under the tradename PURINETHOL®), pemetrexed (sold under the tradename ALIMTA®), cytarabine (also known as arabinosylcytosine (Ara-C), sold under the tradename CYTOSAR-U®), cytarabine liposomal (also known as Liposomal Ara-C, sold under the tradename DEPOCYT™), decitabine (sold under the tradename DACOGEN®), hydroxyurea and (sold under the tradenames HYDREA®, DROXIA™ and MYLOCEL™), fludarabine (sold under the tradename FLUDARA®), floxuridine (sold under the tradename FUDR®), cladribine (also known as 2-chlorodeoxyadenosine (2-CdA) sold under the tradename LEUSTATIN™), methotrexate (also known as amethopterin, methotrexate sodium (MTX), sold under the tradenames RHEUMATREX® and TREXALL™), and pentostatin (sold under the tradename NIPENT®).

Examples of retinoids include, but are not limited to, alitretinoin (sold under the tradename PANRETIN®), tretinoin (all-trans retinoic acid, also known as ATRA, sold under the tradename VESANOID®), Isotretinoin (13-c/s-retinoic acid, sold under the tradenames ACCUTANE®, AMNESTEEM®, CLARAVIS®, CLARUS®, DECUTAN®, ISOTANE®, IZOTECH®, ORATANE®, ISOTRE®, and SOTRET®), and bexarotene (sold under the tradename TARGRETIN®).

### Activity: STING Biochemical [³H]cGAMP Competition Assay

The individual compounds described in the Examples herein are defined as STING agonists by (1) binding to the STING protein as evidenced by a reduction in binding of tritiated cGAMP ligand to the STING protein by at least 20% at 20 uM (concentration of compound being tested) in a STING Biochemical [³H]cGAMP Competition Assay and (ii) demonstrating interferon production with a 6% or greater induction of IFN-β secretion at 30 uM in the THP1 cell assay (where induction caused by cGAMP at 30 uM was set at 100%).

The ability of compounds to bind STING is quantified by the ability to compete with tritiated cGAMP ligand for human STING receptor membrane using a radioactive filter-binding assay. The binding assay employs STING receptor obtained from Hi-Five cell membranes overexpressing full-length HAQ STING prepared in-house and tritiated cGAMP ligand also purified in-house.

The following experimental procedures detail the preparation of specific examples of the instant disclosure. The compounds of the examples are drawn in their neutral forms in the procedures and tables below. In some cases, the compounds were isolated as salts depending on the method used for their final purification and/or intrinsic molecular properties. The examples are for illustrative purposes only and are not intended to limit the scope of the instant disclosure in any way.

### Abbreviations

Å Angstrom
Ac₂O Acetic anhydride
AcOH Acetic acid
AMPDA Adenosine monophosphate deaminase
aq aqueous
atm Atmosphere, a unit of pressure defined as 101325 Pa (1.01325 bar)
BzCl Benzoyl chloride
Ci Curie, a non-standard unit of radioactivity; 1 Ci=3.7×10¹⁰ Bq, where Bq is Becquerel, the SI unit of radioactivity, equivalent to disintegration per second (dps)
DCM, CH₂Cl₂ Dichloromethane
DDTT (E)-N,N-dimethyl-N′-(3-thioxo-3H-1,2,4-dithiazol-5-yl)formimidamide, N′-(3-thioxo-3H-1,2,4-dithiazol-5-yl)-N,N-dimethylmethanimidamide
DIAD (Z)-diisopropyl diazene-1,2-dicarboxylate
DMA N,N-dimethylacetamide
DMSO Dimethylsulfoxide
DMTr 4,4′-dimethoxytrityl
DMTrCl 4,4′-(chloro(phenyl)methylene)bis(methoxybenzene), 4,4′-dimethoxytrityl chloride
DPCP Diphenyl phosporochloridate
EC₅₀ half maximal effective concentration, concentration of a drug, antibody or toxicant that induces a response halfway between the baseline and maximum after a specified exposure time
eq Equivalents
ES Electron spray
Et₂O Diethyl ether
EtOAc Ethyl acetate
EtOH Ethyl alcohol, ethanol
g Gram
h Hour
Hex Hexanes
HPLC High performance liquid chromatography
Hz Hertz
i-PrOH Isopropanol, isopropyl alcohol
LCMS Liquid chromatography-mass spectroscopy
M Molar, moles per liter
m/e Mass/charge
mCi Millicurie
MeCN, ACN, CH₃CN Acetonitrile
MeNH₂, CH₃NH₂ Methylamine
MeOH, CH₃OH Methyl alcohol, methanol
MOI Multiplicity of infection
NaBH₄ Sodium borohydride
Na₂SO₄ Sodium sulfate
NaHCO₃Sodium bicarbonate
NaOMe Sodium methoxide
NH₄HCO₃ Ammonium bicarbonate
PDC Pyridium dichromate
PPh₃ Triphenylphosphine
Py Pyridine
RPM, rpm Revolutions per minute
RT, rt Room temperature, approximately 25° C.
sat Saturated
SnCl₄ Tin(IV) chloride
TBAF, Bu₄NF, Tetra-n-butylammonium fluoride (CH₃CH₂CH₂CH₂)₄NF
TEA, Et₃N Triethylamine
TEA.3HF, Et₃N.3HF Triethylamine trihydrofluoride
TEMPO (2,2,6,6-Tetramethylpiperidin-1-yl)oxyl
TES, Et₃SiH Triethylsilane
TFA Trifluoroacetic acid
THE Tetrahydrofuran
TIPSOTf Triisopropylsilyl trifluoromethanesulfonate
TMSCl Trimethylsilyl chloride
T_{R} Retention time
TrisCl Tris(hydroxymethyl)aminomethane hydrochloride
v/v Volume/volume
λₑₘ Emission wavelength
λₑₓ Excitation wavelength

### PREPARATIONS

The following experimental procedures detail the preparation of specific examples of the instant disclosure. The compounds of the examples are drawn in their neutral forms in the procedures and tables below. In some cases, the compounds were isolated as salts depending on the method used for their final purification and/or intrinsic molecular properties. The examples are for illustrative purposes only and are not intended to limit the scope of the instant disclosure in any way.

### Preparation 1: N-(9-((2R,3S,4S,5R)-5-((bis(4-methoxyphenyl)(phenyl)methoxy) methyl)-4-((tert-butyldimethylsilyl)oxy)-3-hydroxytetrahydrofuran-2-yl)-6-oxo-6,9-dihydro-1H-purin-2-yl)isobutyramide

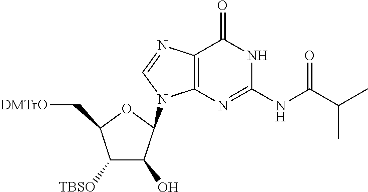

### Step 1: N-(9-((2R,4R,5R)-5-((bis(4-methoxyphenyl)(phenyl)methoxy)methyl)-4-((tert-butyldimethylsilyl)oxy)-3-oxotetrahydrofuran-2-yl)-6-oxo-6,9-dihydro-1H-purin-2-yl)isobutyramide

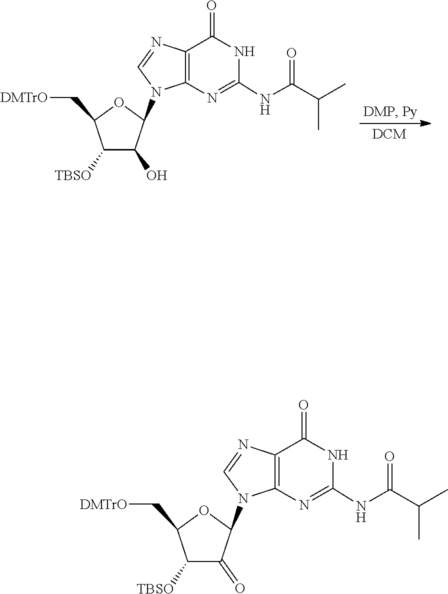

N-(9-((2R,3R,4S,5R)-5-((bis(4-methoxyphenyl)(phenyl)methoxy)methyl)-4-((tert-butyldimethylsilyl)oxy)-3-hydroxytetrahydrofuran-2-yl)-6-oxo-6,9-dihydro-1H-purin-2-yl)isobutyramide was prepared according to Bioorg. Med. Chem. 2010, 18, 4485-4497. To a stirred solution of Dess-Martin periodinane (5.51 g, 13.0 mmol) in DCM (30 mL) at RT was added Py (1.44 g, 18.2 mmol) and N-(9-((2R,3R,4S,5R)-5-((bis(4-methoxy-phenyl)(phenyl) methoxy)methyl)-4-((tert-butyldimethylsilyl)oxy)-3-hydroxytetrahydrofuran-2-yl)-6-oxo-6,9-dihydro-1H-purin-2-yl)isobutyramide (4.00 g, 5.20 mmol). The mixture was stirred at RT for 1 h. Then, the reaction was cooled to 0° C. and quenched with cold sat aq NaHCO₃ (150 mL). The layers were separated, and the aq layer was extracted with DCM (250 mL). The combined organic layer was washed with brine (100 mL), dried (Na₂O₄), and concentrated to give a mixture containing the product. The crude was used for next reaction step directly without further purification. LCMS (ES, m/z): 768.34 [M+H]⁺.

### Step 2: N-(9-((2R,3S,4S,5R)-5-((bis(4-methoxyphenyl)(phenyl)methoxy)methyl)-4-((tert-butyldimethylsilyl)oxy)-3-hydroxytetrahydrofuran-2-yl)-6-oxo-6,9-dihydro-1H-purin-2-yl)isobutyramide

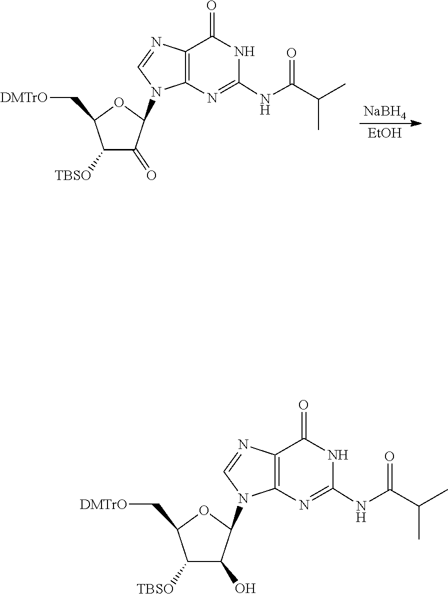

To a stirred solution of the crude from Step 1 (5.2 g, ˜6.7 mmol) in EtOH (30 mL) at 0° C. under Ar was added NaBH₄ (0.790 g, 20.8 mmol). After stirring at 0° C. for 1 h, the volatile components were removed under reduced pressure, and the residue was partitioned between EtOAc (100 mL) and cold sat aq NH₄Cl (100 mL). The layers were separated, and the organic layer was washed with brine (2×100 mL), dried (Na₂SO₄) and concentrated. It was purified by silica gel column chromatography eluted with 0 to 60% EtOAc in Hex to the product. LCMS (ES, m/z): 770.4 [M+H]⁺. ¹H-NMR (400 MHz, DMSO-d₆): δ 12.12 (s, 1H), 11.73 (s, 1H), 8.01 (s, 1H), 7.40-7.37 (m, 2H), 7.34-7.23 (m, 7H), 6.88-6.84 (m, 4H), 6.15 (d, J=5.3 Hz, 1H), 5.85 (d, J=5.4 Hz, 1H), 4.29 (t, J=5.3 Hz, 1H), 4.22 (q, J=5.3 Hz, 1H), 3.91-3.87 (m, 1H), 3.74 (d, J=1.3 Hz, 6H), 3.32-3.28 (m, 1H), 3.25-3.20 (m, 1H), 2.80 (p, J=0.9 Hz, 1H), 1.14 (d, J=6.8 Hz, 6H), 0.80 (s, 9H), 0.07 (s, 3H), −0.02 (s, 3H).

### Preparation 2: (2R,3R,4S,5R)-5-(7-benzamido-3H-[1,2,3]triazolo[4,5-d]pyrimidin-3-yl)-2-((bis(4-methoxy)(phenyl)(phenyl)methoxy)methyl)-4-fluorotetrahydrofuran-3-yl (2-cyanoethyl) diisopropylphosohoramidite

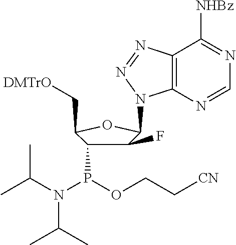

### Step 1: (2R,3R,4S,5R)-5-(7-amino-3H-[1,2,3]triazolo[4,5-d]pyrimidin-3-yl)-2-((benzoyloxy)methyl)-4-fluorotetrahydrofuran-3-yl Benzoate

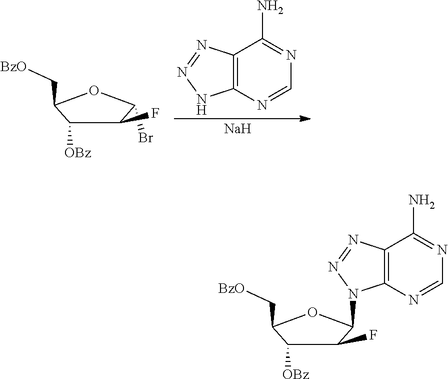

To a mixture of 3H-[1,2,3]triazolo[4,5-d]pyrimidin-7-amine (2.36 g, 17.4 mmol) in N-methyl-2-pyrrolidone (50 mL) was added NaH (60%, 0.744 g, 18.6 mmol). The mixture was vigorously stirred and after 1 h, generation of bubbles had completely ceased. The mixture was added to ((2R,3R,4S,5R)-3-(benzoyloxy)-5-bromo-4-fluorotetrahydrofuran-2-yl)methyl benzoate (neat, 5.25 g, 12.4 mmol) in one portion. It was stirred for 18 h. Then, EtOAc (70 mL) and water (70 mL) were added to the reaction. The layers were separated, and the organic layer was washed with half-saturated brine (3×10 mL) and brine (lx 10 mL), dried (MgSO₄), and concentrated. The crude was purified by silica column chromatography eluted with 0 to 50% EtOAc in Hex to give the product. LCMS (ES, m/z): 479.3 [M+H]⁺. ¹H-NMR (500 MHz, DMSO-d₆): δ 8.62 (s, 1H), 8.34 (s, 1H), 8.28 (s, 1H), 8.10-8.04 (m, 2H), 7.97-7.90 (m, 2H), 7.77-7.69 (m, 1H), 7.67-7.55 (m, 3H), 7.49-7.42 (m, 2H), 6.97 (dd, J=0.5, 3.1 Hz, 1H), 6.49 (dt, J=17.6, 6.9 Hz, 1H), 6.16 (dt, J=56, 6.6 Hz, 1H), 4.76-4.62 (m, 3H).

### Step 2: (2R,3R,4S,5R)-5-(7-amino-3H-[1,2,3]triazolo[4,5-d]pyrimidin-3-yl)-4-fluoro-2-(hydroxymethyl)tetrahydrofuran-3-ol

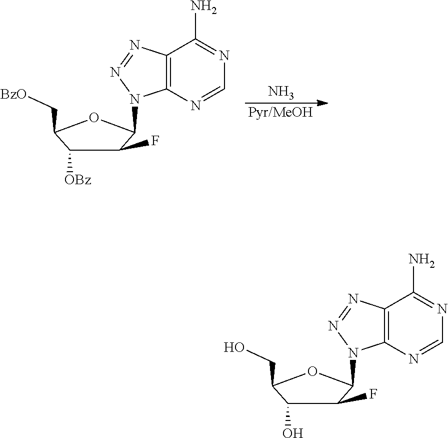

To a solution of the product of Step 1 (2.00 g, 4.18 mmol) in pyridine (10 mL) at RT was added NH₃ in MeOH (7N, 20 mL, 140 mmol). It was stirred for 48 h. LCMS showed completion of the reaction (m/e=271). It was concentrated and purified by silica column chromatography eluted with 10% MeOH in CH₂Cl₂ to give the desired product LCMS (ES, m/z): 271.1 [M+H]⁺. ¹H-NMR (500 MHz, DMSO-d₆): δ 8.54 (s, 1H), 8.33 (s, 1H), 8.22 (s, 1H), 6.73 (dd, J=0.5, 2.6 Hz, 1H), 6.00 (d, J=5.4 Hz, 1H), 5.51 (ddd, J=53, 7.2, 6.5 Hz, 1H), 4.93 (t, J=5.8 Hz, 1H), 4.86-4.74 (m, 1H), 3.91-3.83 (m, 1H), 3.77-3.61 (m, 2H).

### Step 3: N-(3-((2R,3S,4R,5R)-3-fluoro-4-hydroxy-5-(hydroxymethyl)tetrahydrofuran-2-yl)-3H-[1,2,3]triazolo[4,5-d]pyrimidin-7-yl)benzamide

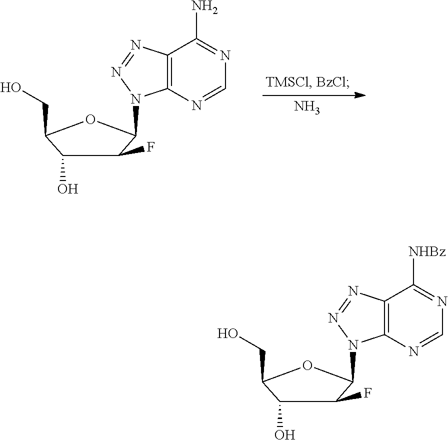

To a solution of the product of Step 2 (1.34 g, 4.96 mmol) in Py (30 mL) at 0° C. was added TMSCl (1.46 mL, 11.4 mmol). It was warmed to RT and stirred for 1 h. Then, it was re-cooled to 0° C. and BzCl (0.921 mL, 7.93 mmol) was added dropwise. The reaction was slowly warmed to RT over 2 h. Then, water (3 mL) was added. It was cooled to 0° C. and NH₃ in MeOH (7N, 2.8 mL, 20 mmol) was added. After 1 h, the reaction mixture was concentrated. It was purified by silica column chromatography eluted with 0 to 10% MeOH in CH₂Cl₂ to give the product LCMS (ES, m/z): 375.2 [M+H]⁺. ¹H-NMR (500 MHz, DMSO-d₆): δ 11.95 (s, 1H), 8.98 (s, 1H), 8.10 (d, J=7.6 Hz, 2H), 7.73-7.66 (m, 1H), 7.59 (t, J=7.7 Hz, 2H), 6.91 (d, J=6.2 Hz, 1H), 6.06 (d, J=5.6 Hz, 1H), 5.59 (t, J=53, 6.8 Hz, 1H), 4.90 (t, J=5.8 Hz, 1H), 4.82 (dq, J=19.8, 7.0 Hz, 1H), 3.92 (td, J=7.6, 2.9 Hz, 1H), 3.75 (ddd, J=12.1, 5.6, 3.0 Hz, 1H), 3.66 (dt, J=12.0, 6.6 Hz, 1H).

### Step 4: N-(3-((2R,3S,4R,5R)-5-((bis(4-methoxyphenyl)(phenyl)methoxy)methyl)-3-fluoro-4-hydroxytetrahydrofuran-2-yl)-3H-[1,2,3]triazolo[4,5-d]pyrimidin-7-yl)benzamide

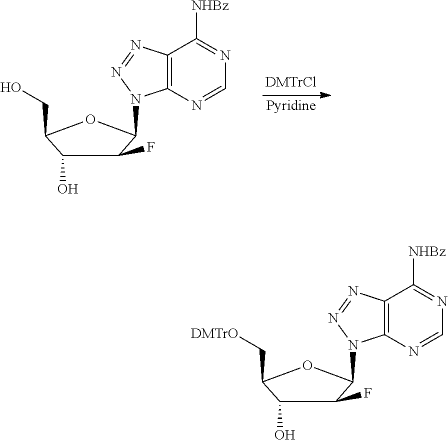

To a solution of the product of Step 3 (1.25 g, 3.34 mmol) in Py (15 mL) at 0° C. was added DMTrCl (1.58 g, 4.68 mmol). It was stirred at RT for 1 h. Then, it was partly concentrated (to 5 mL), and EtOAc (20 mL) and water (10 mL) were added. Layers were separated, and the aq layer was extracted with EtOAc (2×10 mL). The combined organics were washed with brine (5 mL), dried (MgSO₄), concentrated and purified by silica column chromatography eluted with 0 to 60% EtOAc in Hex to give the product. LCMS (ES, m/z): 675.5 [M−H]⁻. ¹H-NMR (500 MHz, DMSO-d₆): δ 8.13-8.07 (m, 2H), 7.69 (t, J=7.4 Hz, 1H), 7.59 (t, J=7.6 Hz, 2H), 7.35-7.29 (m, 2H), 7.23-7.10 (m, 6H), 6.97 (d, J=6.5 Hz, 1H), 6.81-6.74 (m, 2H), 6.74-6.67 (m, 2H), 6.07 (d, J=5.7 Hz, 1H), 5.62 (dt, J=53, 7.0 Hz, 1H), 4.91-4.79 (m, 1H), 4.15-4.07 (m, 1H), 3.69 (s, 3H), 3.67 (s, 3H), 3.44 (dd, J=10.4, 8.0 Hz, 1H), 3.21 (dd, J=10.3, 2.4 Hz, 1H).

### Step 5: (2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydrofuran-3-yl (2-cyanoethyl) Diisopropylphosphoramidite

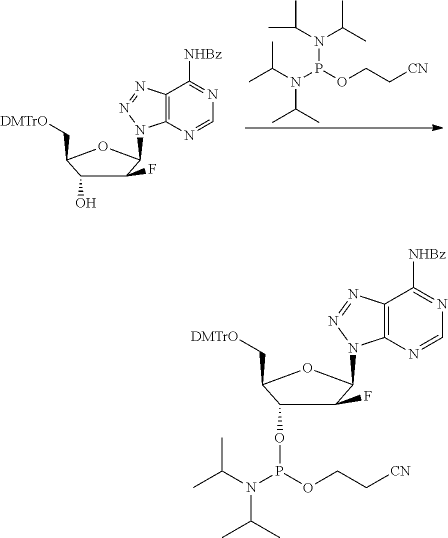

To a solution of 3-((bis(diisopropylamino)phosphino)oxy)propanenitrile (8.02 g, 26.6 mmol) in ACN (90 mL) at RT was added pyridin-1-ium 2,2,2-trifluoroacetate (3.85 g, 19.95 mmol) and a solution of the product of Step 4 (9.00 g, 13.3 mmol) in ACN (90 mL). The resulting mixture was stirred for 1 h. Then, it was concentrated, and the residue was dissolved in CH₂Cl₂ (1000 mL). It was washed with aq NaHCO₃ (1%, 2×300 mL), water (300 mL) and brine (300 mL), dried (Na₂SO₄), concentrated, and purified by reverse phase (C18) chromatography eluted with 0 to 95% ACN in water to give the product. LCMS (ES, m/z): 877.5 [M+H]⁺. ¹H-NMR: (400 MHz, DMSO-d₆): δ 12.01 (s, 1H), 8.92 (s, 1H), 8.11 (d, J=7.6 Hz, 2H), 7.66 (dt, J=42.3, 7.5 Hz, 3H), 7.32 (td J=7.2, 6.6, 2.9 Hz, 2H), 7.22-7.00 (m, 9H), 6.83-6.63 (m, 4H), 5.86 (ddt, J=52.8, 17.6, 6.9 Hz, 1H), 5.16 (td, J=17.7, 17.2, 8.8 Hz, 1H), 3.78-3.63 (m, 7H), 3.59-3.35 (m, 5H), 2.74 (t, J=5.9 Hz, 1H), 2.63 (t, J=5.9 Hz, 1H), 1.23-0.99 (m, 10H), 0.91 (d, J=6.7 Hz, 2H). ³¹P-NMR: (162 MHz, DMSO-d₆): δ 150.26, 149.60.

### Preparation 3: N-(3-((2R,3S,5S)-5-((bis(4-methoxy)(phenyl)(phenyl)methoxy) methyl)-3-hydroxytetrahydrofuran-2-yl)-7-oxo-6,7-dihydro-3H-[1,2,3]triazolo[4,5-d]pyrimidin-5-yl)isobutyramide

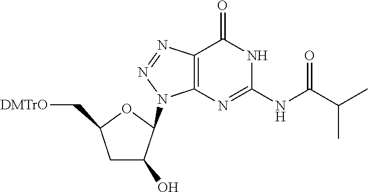

### Step 1: N-(3-((2R,5S)-5-((bis(4-methoxyphenyl)(phenyl)methoxy)methyl)-3-oxotetrahydrofuran-2-yl)-7-oxo-6,7-dihydro-3H-[1,2,3]triazolo[4,5-d]pyrimidin-5-yl) isobutyramide

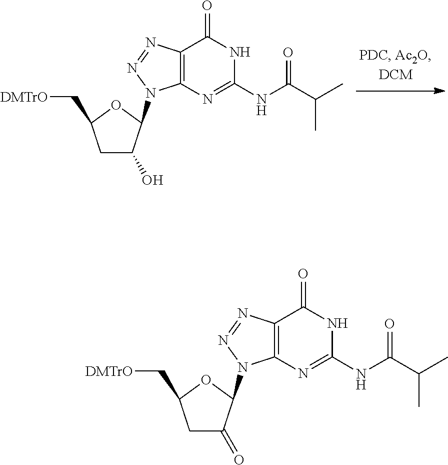

To a solution of N-(3-((2R,3R,5S)-5-((bis(4-methoxyphenyl)(phenyl)methoxy) methyl)-3-hydroxytetrahydrofuran-2-yl)-7-oxo-6,7-dihydro-3H-[1,2,3]triazolo[4,5-d]pyrimidin-5-yl)isobutyramide (8.10 g, 12.5 mmol) in DCM (50 mL), were added activated 4 Å molecular sieves (1.0 g), PDC (3.54 g, 8.75 mmol) and Ac₂O (4.46 g, 43.7 mmol). The reaction mixture was stirred at RT for 2 h. Then, EtOAc (50 mL) was added, and the mixture was filtered. The filtrate was concentrated to give a crude product, which was used for the next reaction step directly without further purification. LCMS (ES, m/z): 639.3 [M+H]⁺.

### Step 2: N-(3-((2R,3S,5S)-5-((bis(4-methoxyphenyl)(phenyl)methoxy)methyl)-3-hydroxytetrahydrofuran-2-yl)-7-oxo-6,7-dihydro-3H-[1,2,3]triazolo[4,5-d]pyrimidin-S-yl)isobutyramide

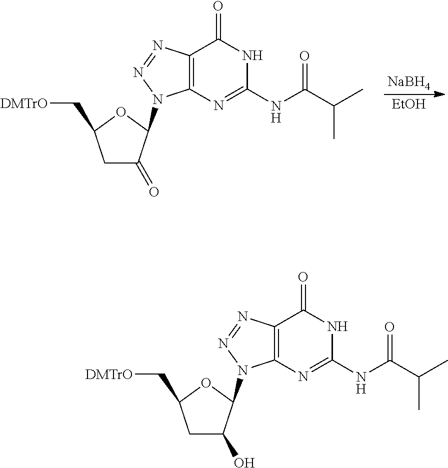

To a suspension of NaBH₄ (0.710 g, 18.8 mmol) in THE (50 mL) at 0° C. under Ar was added a solution of the product of Step 1 in EtOH (50 mL). After stirring at 0° C. for 1 h, the volatile components were removed under reduced pressure, and the residue was partitioned between EtOAc (100 mL) and cold sat aq NH₄Cl (100 mL). The layers were separated, and the organic layer was washed with brine (2×100 mL), dried (Na₂SO₄) and concentrated. It was purified by silica gel column chromatography eluted with 0 to 60% EtOAc in Hex and supercritical fluid chromatography (Lux 5 μm Cellulose-3) eluted with CO₂ and i-PrOH to give the product. LCMS (ES, m/z): 770.4 [M−H]⁻. ¹H-NMR (400 MHz, DMSO-d₆): δ 12.20 (s, 1H), 11.80 (s, 1H), 7.39-7.25 (m, 2H), 7.24-7.07 (m, 7H), 6.80-6.66 (m, 4H), 6.22 (d, J=6.2 Hz, 1H), 5.45 (d, J=5.4 Hz, 1H), 4.73 (dt, J=12.6, 5.8 Hz, 1H), 4.36-4.31 (m, 1H), 3.67 (d, J=4.5 Hz, 6H), 3.49 (t, J=8.9 Hz, 1H), 3.01 (dd, J=10.0, 3.2 Hz, 1H), 2.75 (p, J=6.8 Hz, 1H), 2.51-2.45 (m, 1H), 2.31-2.08 (m, 2H), 1.10 (d, J=6.8 Hz, 6H).

### Preparation 4: N-(3-((2R,3R,4S,5R)-5-((bis(4-methoxyphenyl)(phenyl) methoxy)methyl)-4-fluoro-3-hydroxytetrahydrofuran-2-yl)-7-Oxo-6,7-dihydro-3H-[1,2,3]triazolo[4,5-d]pyrimidin-5-yl)isobutyramide

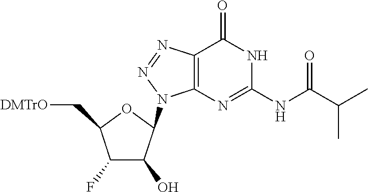

### Step 1: ((2R,3R,4S,5R)-5-(5-amino-7-oxo-6,7-dihydro-3H-[1,2,3]triazolo[4,5-d]pyrimidin-3-yl)-4-(benzoyloxy)-3-fluorotetrahydrofuran-2-yl)methyl Benzoate

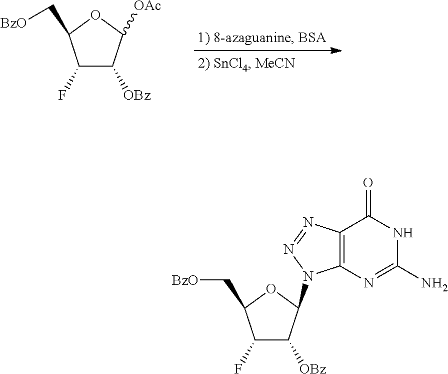

To a stirred suspension of 5-amino-3H-[1,2,3]triazolo[4,5-d]pyrimidin-7(6H)-one (9.83 g, 64.6 mmol) in ACN (180 mL) at RT was added (E)-trimethylsilyl-N-(trimethylsilyl) acetimidate (26.3 g, 129 mmol) over 5 min and then, the mixture was heated at 70° C. for 2 h. The reaction mixture was cooled to RT. To the mixture, were added a solution of ((2R,3R,4S)-5-acetoxy-4-(benzoyloxy)-3-fluorotetrahydrofuran-2-yl)methyl benzoate (13.0 g, 32.3 mmol) in ACN (40 mL) and SnCl₄ in DCM (1.0M, 129 mL, 129 mmol). It was stirred at 70° C. for 2 h. Then, it was concentrated, and to the residue was added EtOAc (1 L) and saturated aq NaHCO₃ (1 L). The layers were separated, and the aq layer was extracted with EtOAc (2×2500 mL). The organic layer were combined, washed with water (500 mL) and brine (500 mL), dried (Na₂SO₄), and concentrated to give a crude product. LCMS (ES, m/z): 495.2 [M+H]⁺.

### Step 2: ((2R,3R,4S,5R)-4-(benzoyloxy)-3-fluoro-5-(5-isobutyramido-7-oxo-6,7-dihydro-3H-[1,2,3]triazolo[4,5-d]pyrimidin-3-yl)tetrahydrofuran-2-yl)methyl Benzoate

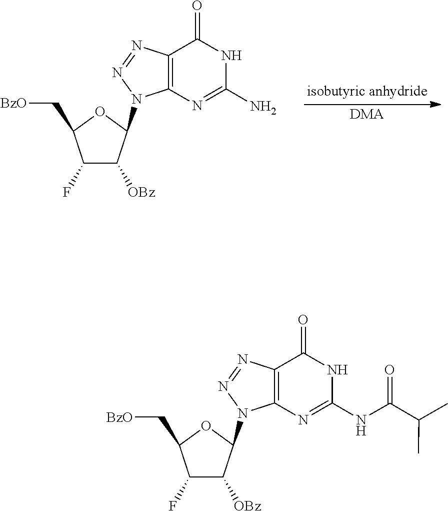

To a solution of the crude from Step 1 in DMA (80 mL) at RT was added isobutyric anhydride (7.66 g, 48.5 mmol) and then, the mixture was heated at 140° C. for 4 h. It was cooled to RT. EtOAc (1 L) and sat aq NH₄Cl (1 L) were added. The layers were separated, and the organic layer was washed with water (4×1 L) and brine (500 mL), dried (Na₂SO₄), and concentrated. The residue was purified by flash chromatography on silica gel eluted with 0 to 50% EtOAc in Hex to give the product LCMS (ES, m/z): 565.2 [M+H]⁺. ¹H-NMR (400 MHz, Chloroform-d): δ 12.20 (s, 1H), 9.45 (s, 1H), 8.03-8.00 (m, 4H), 7.62-7.59 (m, 2H), 7.57-7.41 (m, 4H), 6.65 (d, J=8.0 Hz, 1H), 6.25 (dt, J=15.1, 5.6 Hz, 1H), 5.85-5.65 (m, 1H), 4.89-4.82 (m, 2H), 4.73-4.68 (m, 1H), 2.78-2.74 (m, 1H), 1.29-1.18 (m, 6H).

### Step 3: N-(3-((2R,3S,4S,5R)-4-fluoro-3-hydroxy-5-(hydroxymethyl)tetrahydrofuran-2-yl)-7-oxo-6,7-dihydro-3H-[1,2,3]triazolo[4,5-d]pyrimidin-5-yl)isobutyramide

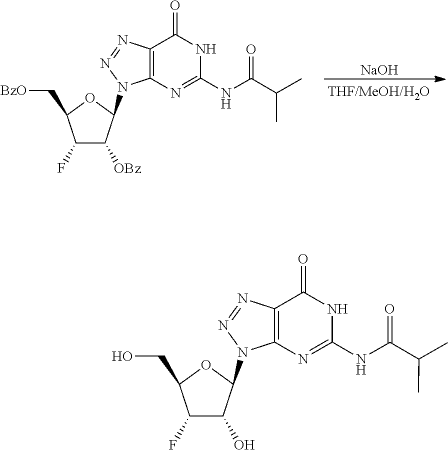

To a solution of the product of Step 2 (15.0 g, 26.6 mmol) in MeOH (52 mL), THE (65 mL) and water (13 mL) at 0° C. was added aq NaOH (2M, 40 mL, 80 mmol) dropwise over 15 min. After 15 min, it was neutralized with aq HCl (2M) and concentrated. The residue was purified by flash chromatography on silica gel eluted with 0 to 10% MeOH in DCM to give the product. LCMS (ES, m/z): 357.1 [M+H]⁺. ¹H-NMR (300 MHz, MeOH-d₄) δ: 6.17 (d, J=5.8 Hz, 1H), 5.31-5.19 (m, 1H), 5.11 (dd, J=4.4, 2.0 Hz, 1H), 4.40-4.29 (m, 1H), 3.73 (d, J=4.9 Hz, 2H), 2.73 (p, J=6.9 Hz, 1H), 1.20 (d, J=6.9 Hz, 6H).

### Step 4: N-(3-((2R,3S,4S,5R)-5-((bis(4-methoxyphenyl)(phenyl)methoxy)methyl)-4-fluoro-3-hydroxytetrahydrofuran-2-yl)-7-oxo-6,7-dihydro-3H-[1,2,3]triazolo[4,5-d]pyrimidin-5-yl)isobutyramide

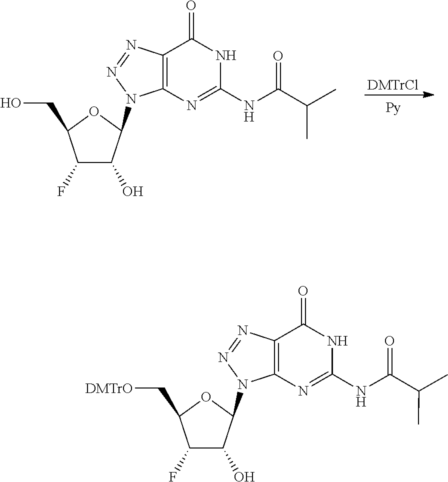

To a solution of the product of Step 3 (8.8 g, 25 mmol) in Py (125 mL) at 0° C. under Ar was added DMTrCl (10.0 g, 29.6 mmol). The reaction mixture was stirred at RT for 2 h. Then, it was concentrated and purified by flash chromatography on silica gel eluted with 0 to 10% MeOH in DCM to give the product. LCMS (ES, m/z): 659.3 [M+H]⁺. ¹H-NMR (300 MHz, MeOH-d₄) δ: 7.37-7.35 (m, 2H), 7.25 (q, J=1.9 Hz, 2H), 7.22 (d, J=1.4 Hz, 2H), 7.20-7.12 (m, 3H), 6.73 (dq, J=8.1, 3.1 Hz, 4H), 6.20 (d, J=6.0 Hz, 1H), 5.27 (ddd, J=19.4, 6.3, 4.3 Hz, 1H), 5.24-5.05 (m, 1H), 4.39 (dq, J=24.5, 3.6, 2.7 Hz, 1H), 3.70-3.67 (m, 6H), 3.36-3.18 (m, 2H), 2.63 (dq, J=13.7, 6.8 Hz, 1H), 1.24-1.13 (m, 6H).

### Step 5: N-(3-((2R,4R,5R)-5-((bis(4-methoxyphenyl)(phenyl)methoxy)methyl)-4-fluoro-3-oxotetrahydrofuran-2-yl)-7-oxo-6,7-dihydro-3H-[1,2,3]triazolo[4,5-d]pyrimidin-5-yl)isobutyramide

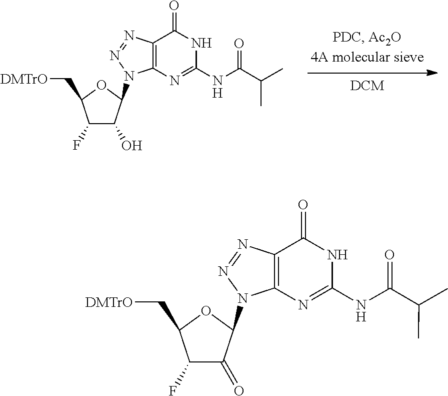

The product of Step 4 (12.4 g, 18.8 mmol) was dissolved in DCM (190 mL) at RT, and activated powdered 4 Å molecular sieves (5 g), PDC (4.96 g, 13.2 mmol) and Ac₂O (6.73 g, 65.9 mmol) were added. It was stirred at RT for 2 h and then, concentrated to give a crude product, which was used for the next reaction step directly without further purification. LCMS (ES, m/z): 657.2 [M+H]⁺.

### Step 6: N-(3-((2R,3R,4S,5R)-5-((bis(4-methoxyphenyl)(phenyl)methoxy)methyl)-4-fluoro-3-hydroxytetrahydrofuran-2-yl)-7-oxo-6,7-dihydro-3H-[1,2,3]triazolo[4,5-d]pyrimidin-5-yl)isobutyramide

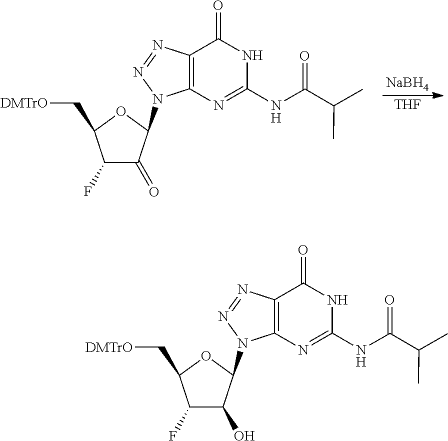

To a solution of the product of Step 5 in THE (95 mL) at 0° C. under Ar was added NaBH₄ (2.85 g, 75.0 mmol). After 1 h, it was concentrated, and the residue was partitioned between EtOAc (1.5 L) and cold sat aq NH₄Cl (1 L). The layers were separated, and the organic layer was washed with brine (2×1.5 L), dried (Na₂SO₄), and concentrated. The residue was purified by reverse phase chromatography (C18) eluted with 0 to 95% ACN in aq NH₄HCO₃ (5 mM) to give the product. LCMS (ES, m/z): 659.7 [M−H]⁻. ¹H-NMR (300 MHz, Methanol-d₄) δ: 7.35 (dt, J=7.6, 1.5 Hz, 2H), 7.28-7.21 (m, 5H), 7.15 (d, J=7.4 Hz, 2H), 6.80-6.65 (m, 4H), 6.51 (d, J=7.0 Hz, 1H), 5.64 (t, J=6.9 Hz, 0.5H), 5.45 (t, J=7.0 Hz, 0.5H), 4.99 (t, J=7.1 Hz, 0.5H), 4.90 (t, J=7.1 Hz, 0.5H), 4.44-4.25 (m, 1H), 3.76-3.62 (m, 7H), 3.57 (dd, J=10.3, 8.4 Hz, 1H), 2.71 (p, J=6.8 Hz, 1H), 1.20 (dt, J=5.6, 2.8 Hz, 6H).

### Preparation 5: (2R,3S,4R,5R)-5-(6-benzamido-9H-purin-9-yl)-2-((bis(4-methoxyphenyl)(phenyl)methoxy)methyl)-4-((triisopropylsilyl)oxy)tetrahydrofuran-3-yl phosohenate, Ammonia Salt

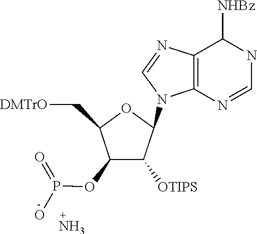

### Step 1: N-(9-((2R,3S,5R)-5-((bis(4-methoxyphenyl)(phenyl)methoxy)methyl)-4-oxo-3-((triisopropylsilyl)oxy)tetrahydrofuran-2-yl)-9H-purin-6-yl)benzamide

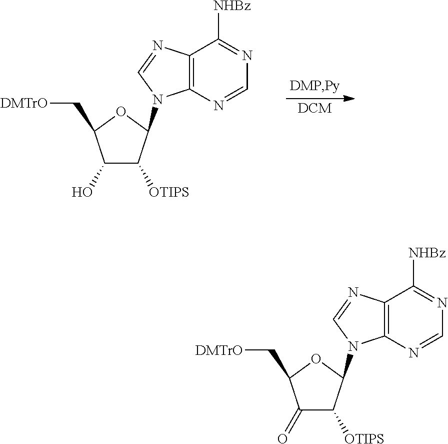

To a solution of N-(9-((2R,3R,4R,5R)-5-((bis(4-methoxyphenyl)(phenyl)methoxy) methyl)-4-hydroxy-3-((triisopropylsilyl)oxy)tetrahydrofuran-2-yl)-9H-purin-6-yl)benzamide (1.50 g, 1.81 mmol) in DCM (10 mL) at RT was added Dess-Martin periodinane (1.53 g, 3.61 mmol). It was stirred at RT for 16 h and then, concentrated. The residue was purified by column chromatography on silica gel eluted with 0 to 50% EtOAc in Hex to give the product LCMS (ES, m/z): 829.2 [M+H]⁺.

### Step 2: N-(9-((2R,3R,4S,5R)-5-((bis(4-methoxyphenyl)(phenyl)methoxy)methyl)-4-hydroxy-3-((triisopropylsilyl)oxy)tetrahydrofuran-2-yl)-9H-purin-6-yl)benzamide

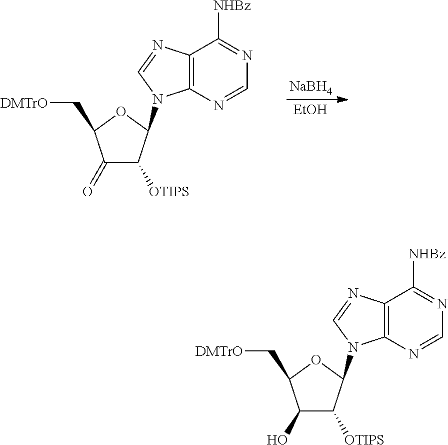

To a suspension of the product of Step 1 (3.00 g, 3.62 mmol) in EtOH (50 ml) at RT under Ar was added NaBH₄ (0.548 g, 14.5 mmol), and it was stirred for 16 h. Then, the reaction mixture was concentrated, and the residue was partitioned between EtOAc (100 mL) and brine (50 mL). The layers were separated, and the organic layer was washed with aq NaHCO₃ (5%, 50 mL), dried (Na₂SO₄), concentrated, and purified by column chromatography on silica gel eluted with 0 to 100% EtOAc in Hex to give the product. LCMS (ES, m/z): 831.2 [M+H]⁺.

### Step 3: (2R,3S,4R,5R)-5-(6-benzamido-9H-purin-9 yl)-2-((bis(4-methoxyphenyl) (phenyl)methoxy)methyl)-4-((triisopropylsilyl)oxy)tetrahydrofuran-3-yl Phenyl Phosphonate

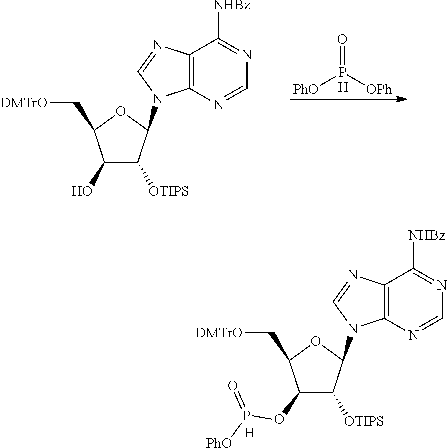

The product of Step 2 (0.830 g, 1.00 mmol) was co-evaporated with Py (3×2 ml) and dissolved in Py (8 ml). To the mixture at RT under Ar was added diphenyl phosphonate (1.17 g, 5.00 mmol), and it was stirred for 20 min. The reaction solution was used for the next step directly.

### Step 4: (2R,3S,4R,5R)-5-(6-benzamido-9H-purin-9 yl)-2-((bis(4-methoxyphenyl) (phenyl) methoxy)methyl)-4-((triisopropylsilyl)oxy)tetrahydrofuran-3-yl phosphenate, Ammonia Salt

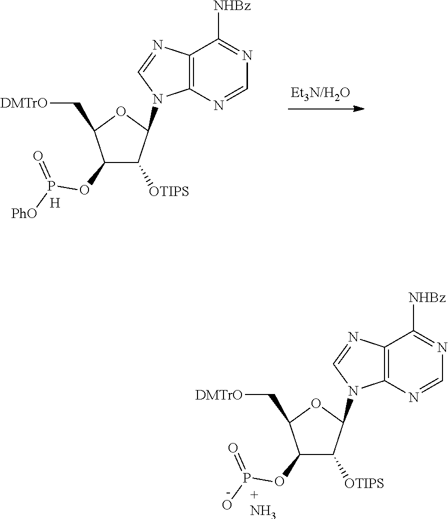

To the reaction mixture from Step 3 was added water (4 ml) and Et₃N (4 mL), which was stirred at RT for 20 min. Then, it was concentrated and purified by reverse phase chromatography (AQ C18) eluted with 0 to 95% ACN in aq NH₄HCO₃ (5 mM) to give the product. LCMS (ES, m/z): 894.4 [M+H]⁺. ¹H-NMR: (300 MHz, DMSO-d₆) δ 11.17 (s, 1H), 8.72 (s, 1H), 8.55 (s, 1H), 8.06-7.96 (m, 2H), 7.67-7.42 (m, 4H), 7.37 (d, J=7.6 Hz, 2H), 7.22 (td, J=9.9, 9.3, 4.6 Hz, 7H), 6.87-6.74 (m, 4H), 6.14 (s, 1H), 4.78 (s, 1H), 4.42 (dd, J=9.1, 3.1 Hz, 1H), 4.28 (d, J=3.4 Hz, 1H), 4.07 (d, J=5.3 Hz, 1H), 3.68 (d, J=2.2 Hz, 6H), 3.41 (t, J=9.0 Hz, 1H), 3.29 (q, J=6.2, 5.2 Hz, 2H), 3.13 (d, J=4.4 Hz, 4H), 1.23-1.12 (m, 1H), 1.11 (td, J=6.8, 2.5 Hz, 2H), 0.99 (t, J=7.7 Hz, 19H). ³¹P-NMR: (121 MHz, DMSO-d₆) δ 0.01 (s).

### Preparation 6: Ammonium (2R,3S,4S,5R)-5-(6-benzamido-9H-purin-9-yl)-2-((bis(4-methoxyphenyl)(phenyl)methoxy)methyl)-4-fluorotetrahydrofuran-3-yl phosphonate

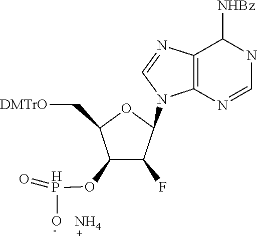

### Step 1: N-(9-((2R,3R,5R)-5-((bis(4-methoxyphenyl)(phenyl)methoxy)methyl)-3-fluoro-4-oxotetrahydrofuran-2-yl)-9H-purin-6-yl)benzamide

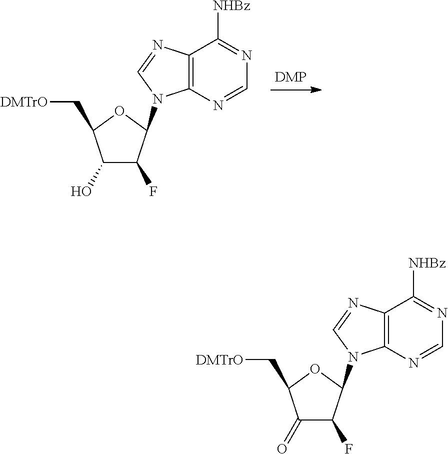

To a solution of N-(9-((2R,3S,4R,5R)-5-((bis(4-methoxyphenyl)(phenyl)methoxy) methyl)-3-fluoro-4-hydroxytetrahydrofuran-2-yl)-9H-purin-6-yl)benzamide (3.00 g, 4.44 mmol) in DCM (80 mL) at RT was added Dess-Martin periodinane (3.39 g, 7.99 mmol). It was stirred at RT for 1 h, and then, sat aq NaHCO₃ (20 ml) was added. The layers were separated, and the organic layer was washed with water (3×25 ml), dried (Na₂SO₄), and concentrated to give a crude product. LCMS (ES, m/z): 674.7 [M+H]⁺.

### Step 2: N-(9-((2R,3S,4S,5R)-5-((bis(4-methoxyphenyl)(phenyl)methoxy)methyl)-3-fluoro-4-hydroxytetrahydrofuran-2-yl)-9H-purin-6-yl)benzamide

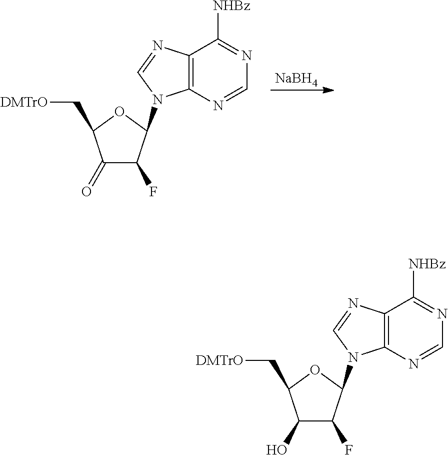

To a solution of the product of Step 1 in EtOH (80 ml) at 0° C. was added NaBH₄ (0.672 g, 17.8 mmol). It was stirred for 30 min as it warmed up to RT. Then, the reaction mixture was concentrated, and sat aq NH₄Cl (25 mL) and EtOAc (25 ml) were added. The layers were separated, and the aq layer was extracted with EtOAc (3×25 ml). The combined organics were dried (Na₂SO₄), concentrated, and purified by reverse phase chromatography (C18) eluted with 20 to 95% ACN in aq NH₄HCO₃ (5 mM) to give the product. LCMS (ES, m/z): 676.7 [M+H]⁺.

### Step 3: (2R,3S,4S,5R)-5-(6-benzamido-9H-purin-9-yl)-2-((bis(4-methoxyphenyl) (phenyl)methoxy)methyl)-4-fluorotetrahydrofuran-3-yl phenyl Phosphonate

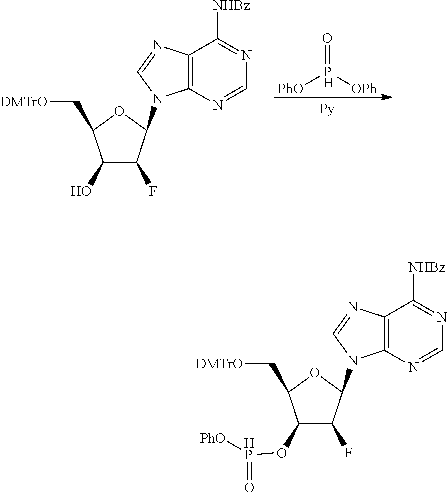

The product of Step 2 (0.68 g, 1.0 mmol) was co-evaporated with Py (3×2 mL) and dissolved in Py (8 ml). To the mixture at 0° C. under Ar was added diphenyl phosphonate (0.96 mL, 5.0 mmol) over 3 min, and it was stirred for 30 min. The reaction mixture was used for the next step directly. LCMS (ES, m/z): 816.3 [M+H]⁺.

### Step 4: Ammonium (2R,3S,4S,5R)-5-(6-benzamido-9H-purin-9-yl)-2-((bis(4-methoxyphenyl)(phenyl)methoxy)methyl)-4-fluorotetrahydrofuran-3-yl Phosphonate

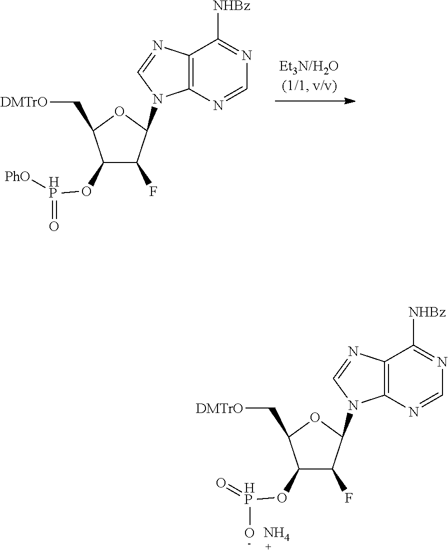

To the reaction mixture from Step 3 at 0° C. was added water (1 ml) and Et₃N (1 mL). The mixture was stirred at RT for 30 min. Then, it was concentrated, and the residue was partitioned between DCM (50 mL) and aq NaHCO₃ (5%, 50 mL). The organic layer was washed with aq NaHCO₃ (5%, 40 mL), dried (Na₂SO₄), concentrated and purified by reverse phase chromatography (AQ C18) eluted with 0 to 95% ACN in aq NH₄HCO₃ (5 mM) to give the product. LCMS (ES, m/z): 740.2 [M+H]⁺. ¹H-NMR: (400 MHz, DMSO-d₆) δ 11.23 (br s, 1H), 8.79 (s, 1H), 8.34 (d, J=2.0 Hz, 1H), 8.07 (d, J=7.5 Hz, 2H), 7.66 (t, J=7.7 Hz, 1H), 7.57 (t, J=7.6 Hz, 2H), 7.44-7.38 (m, 2H), 7.33-7.24 (m, 7H), 7.21 (dd, J=8.3, 6.1 Hz, 0.5H), 6.85 (t, J=9.1 Hz, 4H), 6.68 (dd, J=8.6, 5.5 Hz, 1H), 5.81 (d, J=2.5 Hz, 0.5H), 5.53 (dt, J=51.5, 5.0 Hz, 1H), 5.01 (dq, J=10.2, 5.4, 4.9 Hz, 1H), 4.42 (dt, J=7.9, 3.7 Hz, 1H), 3.73 (d, J=3.4 Hz, 6H), 3.41-3.27 (m, 2H). ³¹P-NMR: (162 MHz, DMSO-d₆) δ −0.43 (s).

### Preparation 7: (2R,3R,5R)-5-(4-benzamido-7H-pyrrolo[2,3d]pyrimidin-7-yl)-2-((bis(4-methoxyphenyl)(phenyl)(methoxy)methyl)tetrahydrofuran-3-yl (2-cyanoethyl) diisopropylphosohoramidite

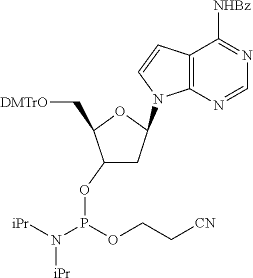

### Step 1: (2R,3R,5R)-5-(4-benzamido-7H-pyrrolo[2,3-d]pyrimidin-7-yl)-2-((bis(4-methoxyphenyl)(phenyl)methoxy)methyl)tetrahydrofuran-3-yl 4-nitrobenzoate

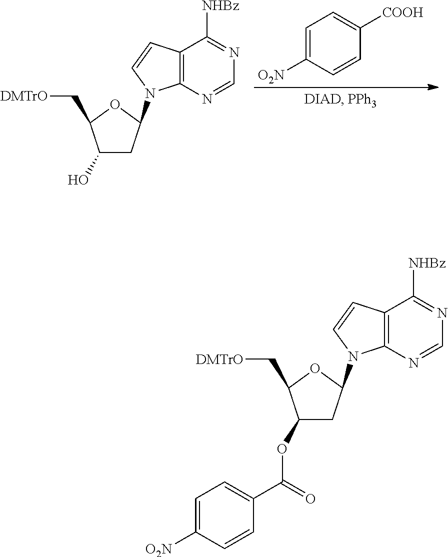

To a solution of N-(7-((2R,4S,5R)-5-((bis(4-methoxyphenyl)(phenyl)methoxy) methyl)-4-hydroxytetrahydrofuran-2-yl)-7H-pyrrolo[2,3-d]pyrimidin-4-yl)benzamide (4.00 g, 6.09 mmol) (co-evaporated with THF 3×15 mL before being used) in THF (180 ml) at RT under Ar were added 4-nitrobenzoic acid (2.04 g, 12.2 mmol), PPh₃ (3.20 g, 12.2 mmol) and DIAD (2.46 g, 12.2 mmol). The resulting mixture was stirred at RT for 2.5 h. Then, it was concentrated and purified by flash chromatography on silica gel eluted with 0 to 64% EtOAc in Hex to give the product. LCMS (ES, m/z): 806.8 [M+H]⁺.

### Step 2: N-(7-((2R,4R,5R)-5-((bis(4-methoxyphenyl)(phenyl)methoxy)methyl)-4-hydroxytetrahydrofuran-2-yl)-7H pyrrolo[2,3-d]pyrimidin-4-yl)benzamide

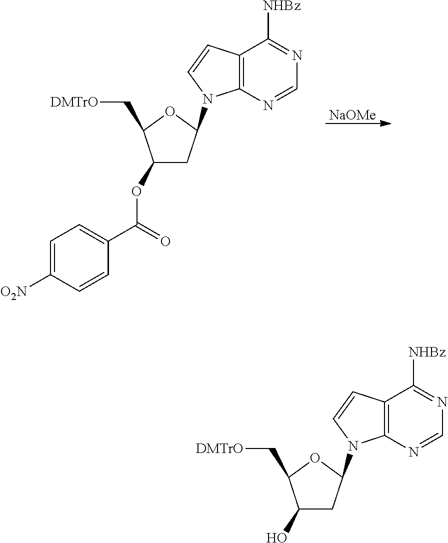

To a solution of the product of Step 1 (4.6 g, 5.7 mmol) in MeOH (175 ml) at 0° C. was added NaOMe in MeOH (0.20M, 20 ml, 4.0 mmol). It was stirred as it warmed to RT over 4 h. Then, the solution was neutralized with aq HCl and concentrated. The residue was purified by reverse phase chromatography (C18) eluted with 0 to 95% ACN in aq NH₄HCO₃ (5 mM) to give the product. LCMS (ES, m/z): 657.7 [M+H]⁺.

### Step 3: (2R,3R,5R)-5-(4-benzamido-7H pyrrolo[2,3-d]pyrimidin-7-yl)-2-((bis(4-methoxyphenyl) (phenyl)methoxy)methyl)tetrahydrofuran-3-yl (2-cyanoethyl) diisopropylphosphoramidite

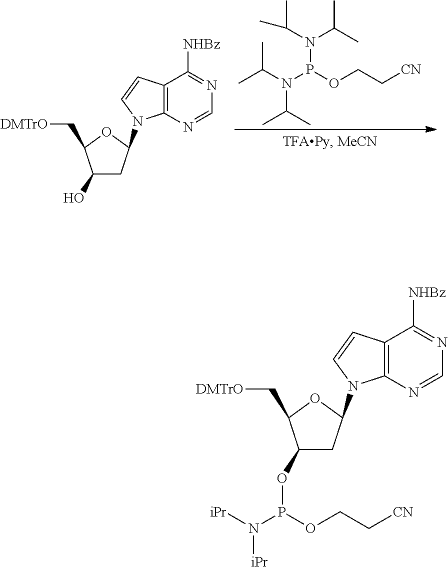

To a solution of 3-((bis(diisopropylamino)phosphino)oxy)propanenitrile (129 mg, 0.429 mmol) in ACN (5 mL) at RT under Ar was added TFA.Py (62 mg, 0.32 mmol) and a solution of the dry product of Step 2 (750 mg, 1.14 mmol co-evaporated with ACN (3×3 mL) before being used) in ACN (5 mL) over 2 min. The mixture was stirred at RT for 1 h. Then, it was concentrated, and aq NaHCO₃ (1%, 30 mL) and DCM (50 mL) were added. The layers were separated, and the organic layer was washed with aq NaHCO₃ (1%, 2×30 mL), water (30 mL) and brine (30 mL), dried (Na₂SO₄) and purified by reverse phase chromatography (C18) eluted with 0 to 100% ACN in water to give the product. LCMS (ES, m/z): 857.4 [M+H]⁺. ¹H-NMR: (300 MHz, DMSO-d₆) δ 11.10 (s, 1H), 8.60-8.59 (m, 1H), 8.05-8.01 (m, 2H), 7.65-7.57 (m, 1H), 7.56-7.47 (m, 2H), 7.39-7.31 (m, 2H), 7.14-7.25 (m, 7H), 6.82-6.73 (m, 5H), 6.63-6.52 (m, 1H), 4.53-4.49 (m, 1H), 4.36-4.33 (m, 1H), 3.69 (s, 3H), 3.68 (s, 3H), 3.63-3.54 (m, 1H), 3.52-3.44 (m, 1H), 3.40-3.31 (m, 3H), 2.26-3.08 (m, 1H), 2.94-2.84 (m, 1H), 2.66-2.48 (m, 2H), 2.41-2.35 (m, 1H), 2.29-2.23 (m, 1H), 1.05-1.01 (m, 6H), 0.91-0.84 (m, 6H). ³¹P-NMR: (121 MHz, DMSO-d₆) δ 149.80 (s), 145.19 (s).

### EXAMPLES

### Examples 1, 2 and 3: 2-amino-9-[(5R,7R,5S,12aR,14R,15S,15aR,16R)-14-(7-amino-3H-[1,2,3]triazolo[4,5-d]pyrimidin-3-yl)-15-fluor-16-hydroxy-2,10-dioxido-2,10-disulfanyl octahydro-12H-5,8-methanofuro[3,2-1][3,6,9,11,2,10]pentaoxadiphospha-cyclotetradecin-7-yl]-1,9-dihydro-6H-purin-6-one (Diastereomer 1), 2-amino-9-[(5R,7R,8S,12aR,14R,15S,15aR,16R)-14-(7-amino-3H-[1,2,3]triazolo[4,5-d]pyrimidin-3-yl)-15-fluoro-16-hydroxy-2,10-dioxido-2,10-disulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 2) and 2-amino-9-[(5R,7R,5S,12aR,14R,15S,15aR,16R)-14-(7-amino-3H-[1,2,3]triazolo[4,5-d]pyrimidin-3-yl)-15-fluoro-16-hydroxy-2,10-dioxido-2,10-disulfanyloctahydro-12H-5,8-methanofuro[3,2-1][1,3,6,9,11,2,10]pentaoxadiphosphacyclo-tetradecin-7-yl]-19-dihydro-6H-purin-6-one (Diastereomer 3)

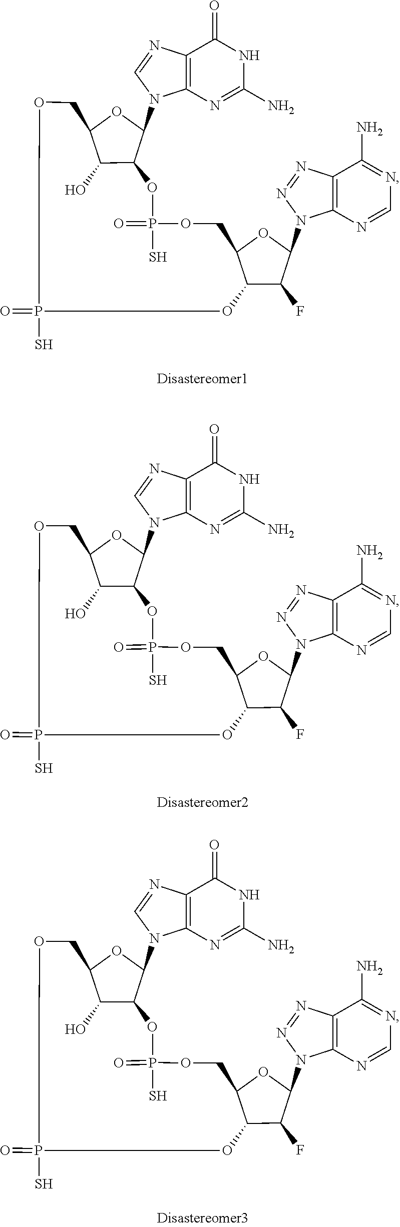 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3O)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3O)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3O)c(=O)[nH]1]

### Step 1: (2R,3S,4R,5R)-5-((bis(4-methoxyphenyl)(phenyl)methoxy)methyl)-4-((tert-butyldimethylsilyl)oxy)-2-(2-isobutyramido-6-oxo-1,6-dihydro-9H-purin-9-yl)tetrahydrofuran-3-yl Phenyl Phosphonate

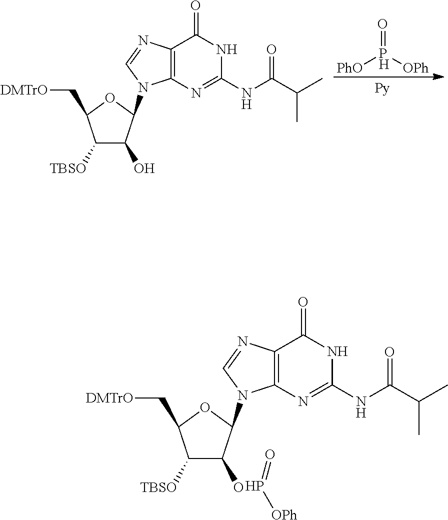

Preparation 1 (440 mg, 0.571 mmol) was co-evaporated with Py (3×4 mL) and dissolved in Py (2 ml). To the mixture at RT under Ar was added diphenyl phosphonate (548 mg, 2.86 mmol), and it was stirred for 15 min. The reaction solution was used for the next step directly. LCMS (ES, m/z): 910.1 [M−H]⁻.

### Step 2: triethylammonium (2R,3S,4R,5R)-5-((bis(4-methoxyphenyl)(phenyl) methoxy)methyl)-4-((tert-butyldimethylsilyl)oxy)-2-(2-isobutyramido-6-oxo-1,6-dihydro-9H-purin-9-yl)tetrahydrofuran-3-yl Phosphonate

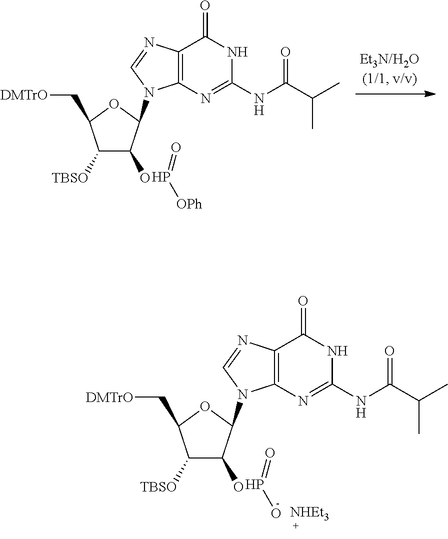

To the reaction mixture from Step 1 was added water (1 ml) and Et₃N (1 mL). The mixture was stirred at RT for 15 min. Then, it was concentrated, and the residue was partitioned between DCM (60 mL) and aq NaHCO₃ (5%, 80 mL). The layers were separated, and the organic layer was dried (Na₂SO₄) and concentrated. It was purified by chromatography on silica gel eluted with 0 to 10% MeOH in DCM (containing 0.2% of Et₃N) to give the product. LCMS (ES, m/z): 832.2 [M−H]⁻. ¹H-NMR (400 MHz, DMSO-d₆): δ 12.11 (s, 1H), 11.78 (s, 1H), 10.00 (s, 1H), 8.04 (s, 1H), 7.35 (d, J=7.1 Hz, 2H), 7.23 (t, J=12.4 Hz, 7H), 6.83 (d, J=8.3 Hz, 4H), 6.21 (d, J=5.4 Hz, 1H), 4.68 (s, 1H), 4.48 (m, 1H), 3.87 (s, 1H), 3.73 (s, 6H), 3.06-2.98 (m, 12H), 2.84-2.75 (m, 1H), 1.26 (m, 6H), 1.15 (m, 19H), 0.79 (s, 9H), 0.09 (s, 3H), 0.00 (s, 3H). ³¹P-NMR: (162 MHz, DMSO-d₆): δ 0.11 (s).

### Step 3: pyridin-1-ium (2R,3S,4R,5R)-4-((tert-butyldimethylsilyl)oxy)-5-(hydroxyl-methyl)-2-(2-isobutyramido-6-oxo-1,6-dihydro-9H-purin-9-yl)tetrahydrofuran-3-yl phosphonate

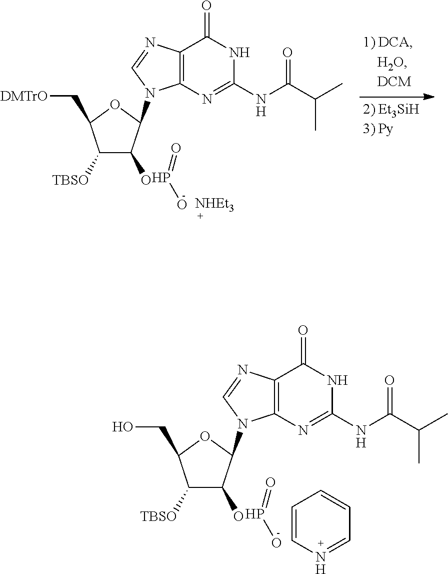

To a stirred solution of the product of Step 1 (388 mg, 0.466 mmol) in DCM (6.5 ml) at RT was added water (75 mg, 4.2 mmol) and 2,2-dichloroacetic acid in DCM (6%, 6.2 ml, 3.7 mmol). After 10 min, Et₃SiH (8 mL) was added, and it was stirred at RT for 1 h. Then, Py (0.7 mL) was added. It was concentrated to give a crude product LCMS (ES, m/z): 532.3 [M+H]⁺.

### Step 4: Pyridin-1-ium (2R,3S,4R,5R)-5-((((((2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydro-furan-3-yl)oxy)(2-cyanoethoxy)phosphanyl)oxy)methyl)-4-((tert-butyldimethylsilyl)oxy)-2-(2-isobutyramido-6-oxo-1,6-dihydro-9H-purin-9 yl)tetrahydrofuran-3-yl Phosphonate

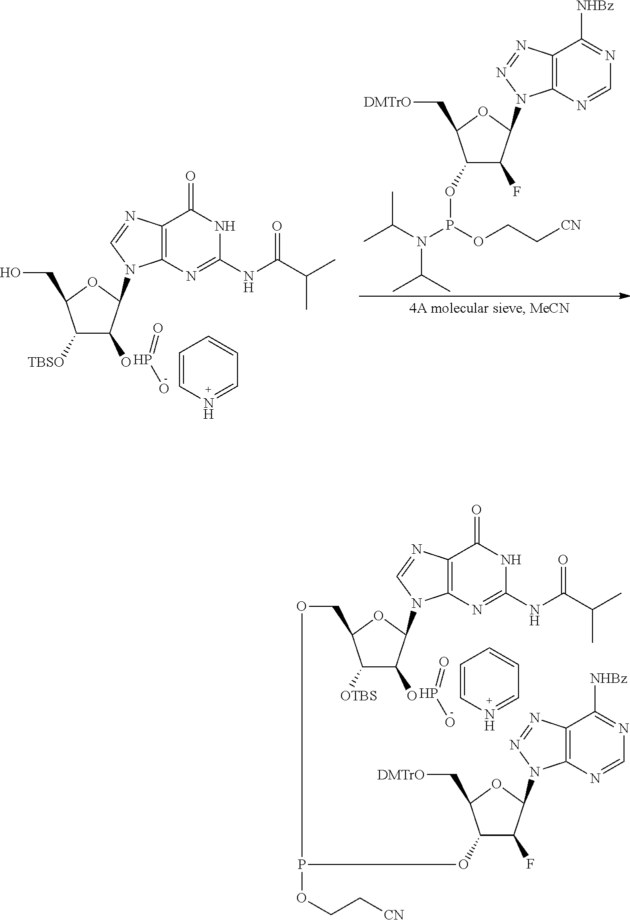

(2R,3R, 4S, 5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydrofuran-3-yl (2-cyanoethyl) diisopropylphosphoramidite (0.40 g, 0.46 mmol) was co-evaporated with ACN (3×2 mL) and dissolved in ACN (3 mL). Activated 4 Å molecular sieves (200 mg) were added to the solution. The product from Step 3 was co-evaporated with ACN (3×2 mL) and dissolved in ACN (3 mL). Activated 4 Å molecular sieves (200 mg) were added to the solution. After 30 min, to the solution under Ar was added the solution containing (2R,3R,4S,5R)-5-(7-benzamido-3H-[1,2,3]triazolo[4,5-d]pyrimidin-3-yl)-2-((bis(4-methoxyphenyl)(phenyl) methoxy)methyl)-4-fluorotetrahydro-furan-3-yl (2-cyanoethyl) diisopropylphosphoramidite, and it was stirred at RT for 30 min. The reaction mixture was used in the next step without purification. LCMS (ES, m/z): 1305.2 [M−H]⁻.

### Step 5: pyridin-1-ium (2R,3S,4R,5R)-5-((((((2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydro-furan-3-yl)oxy)(2-cyanoethoxy)phosphorothioyl)oxymethyl)-4-((tert-butyldimethylsilyl)oxy)-2-(2-isobutyramido-6-oxo-1,6-dihydro-9H-purin-9-yl)tetrahydrofuran-3-yl Phosphonate

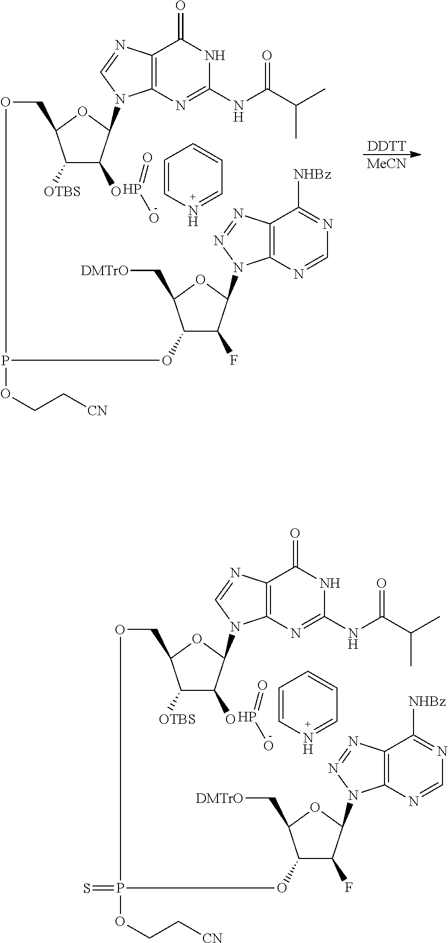

To the reaction mixture from Step 4 at RT was added (E)-N,N-dimethyl-N-(3-thioxo-3H-1,2,4-dithiazol-5-yl)formimidamide (0.094 g, 0.46 mmol), and it was stirred for 45 min. Then, the mixture was concentrated to give a crude product, which was used for the next reaction step directly without further purification. LCMS (ES, m/z): 1137.1 [M−H]⁻.

### Step 6: ammonium (2R,3S,4R,5R)-5-((((((2R,3R,4S,5R)-5-(7-benzamido-3H-[1,2,3]triazolo[4,5-d]pyrimidin-3-yl)-4-fluoro-2-(hydroxymethyl)tetrahydrofuran-3-yl)oxy)(2-cyanoethoxy)phosphorothioyl)oxy)methyl)-4-((tert-butyldimethylsilyl)oxy)-2-(2-isobutyramido-6-oxo-1,6-dihydro-9H-purin-9 yl)tetrahydrofuran-3-yl Phosphonate

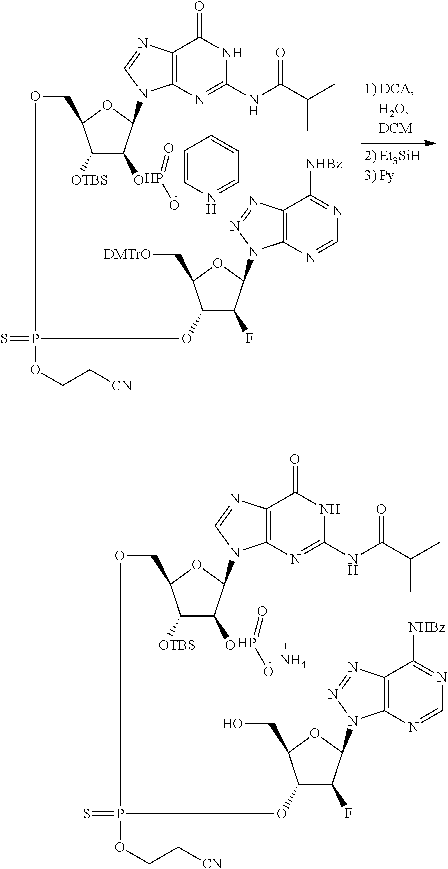

To the crude from Step 5 in DCM (6.5 mL) at RT was added water (75 mg, 4.2 mmol) and 2,2-dichloroacetic acid in DCM (0.6M, 6.2 ml, 3.7 mmol). After stirring for 10 min, Et₃SiH (8 mL) was added, and it was stirred for 1 h. Then, Py (700 mg) was added to the reaction. It was concentrated, and the crude was purified by reverse phase chromatography (C18) eluted with 0 to 95% ACN in aq NH₄HCO₃ (5 mM) to give the product. LCMS (ES, m/z): 1037.3 [M+H]⁺. ³¹P-NMR: (162 MHz, MeOH-d₄): δ 67.98 (s), 2.22-2.16 (m).

### Step 7: pyridinium (5R,7R,8S,12aR,14R,15S,15aR,16R)-16-{[tert-butyl(dimethyl) silyl]oxy}-2-(2-cyanoethoxy)-15-fluoro-7-{2 [(2-methylpropanoyl)amino]-6-oxo-1,6-dihydro-9H-purin-9-yl}-14-{7-[(phenylcarbonyl)amino]-3H-[1,2,3]triazolo[4,5-d]pyrimidin-3-yl}octahydro-12H-5,8-methanofuro[3.2-1][1,3,6,9,11,2,10]pentaoxadiphosphacyclotetradecin-10-olate 2-sulfide

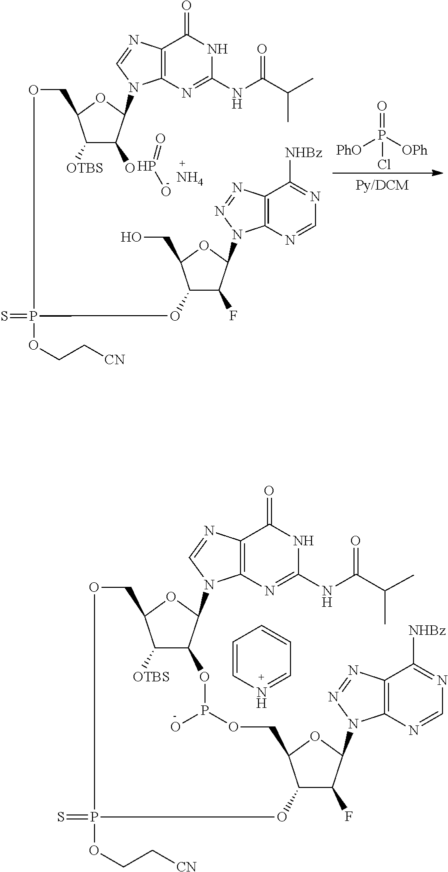

To Py (10 mL) at −40° C. under Ar was added diphenyl phosphorochloridate (32 mg, 0.12 mmol), and a solution of the product of Step 6 (113 mg, 0.107 mmol) in Py (7 mL) dropwise over 10 min. The resulting mixture was stirred at −40° C. to −20° C. for 40 min. The solution containing the product was used for the next step directly. LCMS (ES, m/z): [M+H]⁺ 1019.3.

### Step 8: ammonium (5R,7R,8S,12aR,14R,15S,15aR,16R)-16-{[tert-butyl(dimethyl) silyl]oxy}-2-(2-cyanoethoxy)-15-fluoro-7-{2-[(2-methylpropanoyl)amino]-6-oxo-1,6-dihydro-9H-purin-9-yl}-14-(7-[(phenylcarbonyl)amino]-3H-[1,2,3]triazolo[4,5-d]pyrimidin-3-yl) octahydro-12H-5,8-methanofuro[3,2-1][1,3,6,9,11,2,10]pentaoxadiphosphacyclotetradecin-10-olate 2,10-disulfide

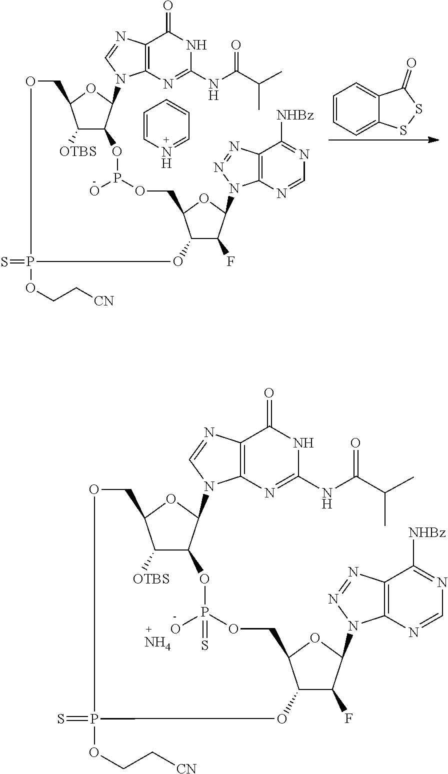

To the reaction mixture from Step 7 at −40° C. was added 3H-benzo[c][1,2]dithiol-3-one (0.027 g, 0.16 mmol) and water (0.096 g, 5.4 mmol). It was stirred at RT for 40 min. Then, the solution was concentrated. It was co-evaporated with toluene (2×10 mL) and ACN (10 mL). The crude was purified by reverse phase chromatography (C18) eluted with 0 to 95% ACN in aq NH₄HCO₃ (5 mM) to give the product. LCMS (ES, m/z): 1051.3 [M+H]⁺. ³¹P-NMR: (162 MHz, MeOH-d₄) δ 68.79-56.76 (m).

### Step 9: N-{9 [(5R,7R,8S,12aR,14R,15S,15aR,16R)-14-(7-amino-3H-[1,2,3]triazolo[4,5-d]pyrimidin-3-yl)-16-{[tert-butyl(dimethyl)silyl]oxy}-15-fluoro-2,10-dihydroxy-2,10-disulfidooctahydro-12H-5,8-methanofuro[3,2-1][1,3,6,9,11,2,10]pentaoxadiphosphacyclo-tetradecin-7-yl]-6-oxo-6,9-dihydro-1H-purin-2-yl}-2-methylpropanamide Diammoniate

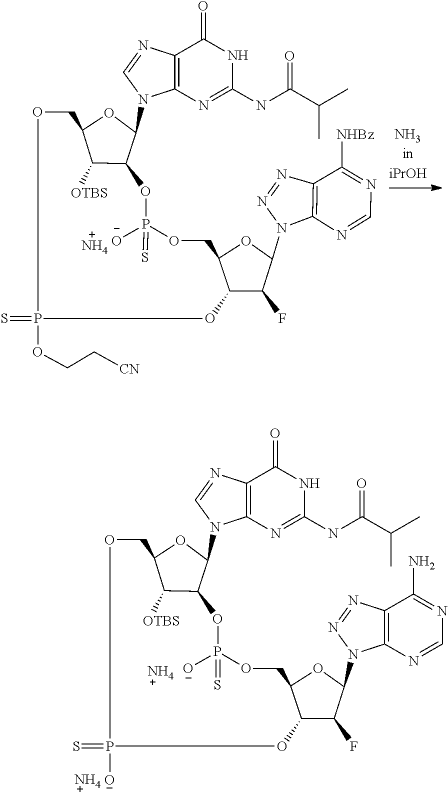

To a solution of NH₃ in i-PrOH (2M, 22 ml) at RT was added the product of Step 8 (110 mg, 0.104 mmol). The reaction container was sealed, and the reaction was stirred at 50° C. for 3 h. Then, it was concentrated to give a crude product, which was directly used for next step without further purification. LCMS (ES, m/z): 894.2 [M+H]⁺.

### Step 10: 2-amino-9-[(5R,7R,8S,12aR,14R,15S,15aR,16R)-14-(7-amino-3H-[1,2,3]triazolo[4,5-d]pyrimidin-3-yl)-16-{[tert-butyl(dimethyl)silyl]oxy}-15-fluoro-2,10-dihydroxy-2,10-disulfidooctahydro-12H-5,8-methanofuro[3,2-1][1,3,6,9,11,2,10]pentaoxadiphosphacyclo-tetradecin-7-yl]-1,9-dihydro-6H-purin-6-one-methanamine (1:2)

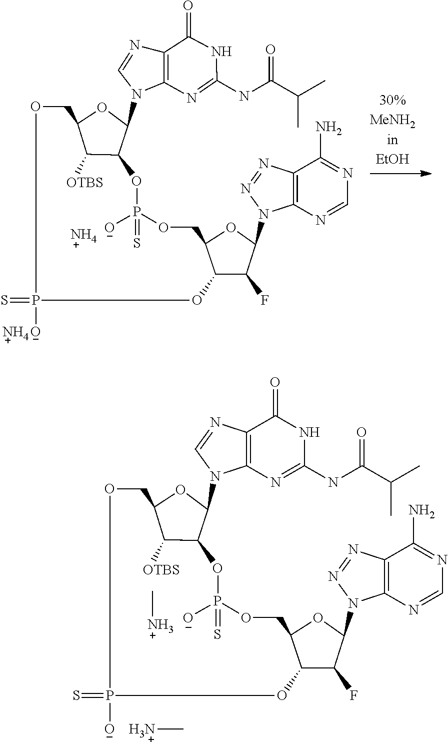

The crude from Step 9 was dissolved in a solution of MeNH₂ in EtOH (33% by weight, 10 mL). The resulting solution was stirred at RT for 3 h. Then, it was concentrated, and the crude product was used for the next reaction step directly. LCMS (ES, m/z): 824.2 [M+H]⁺.

### Step 11: 2-amino-9-[(5R,7R,8S,12aR,14R,15S,15aR,16R)-14-(7-amino-3H-[1,2,3]triazolo[4,5-d]pyrimidin-3-yl)-15-fluoro-16-hydroxy-2,10-dioxido-2,10-disulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 1-3)

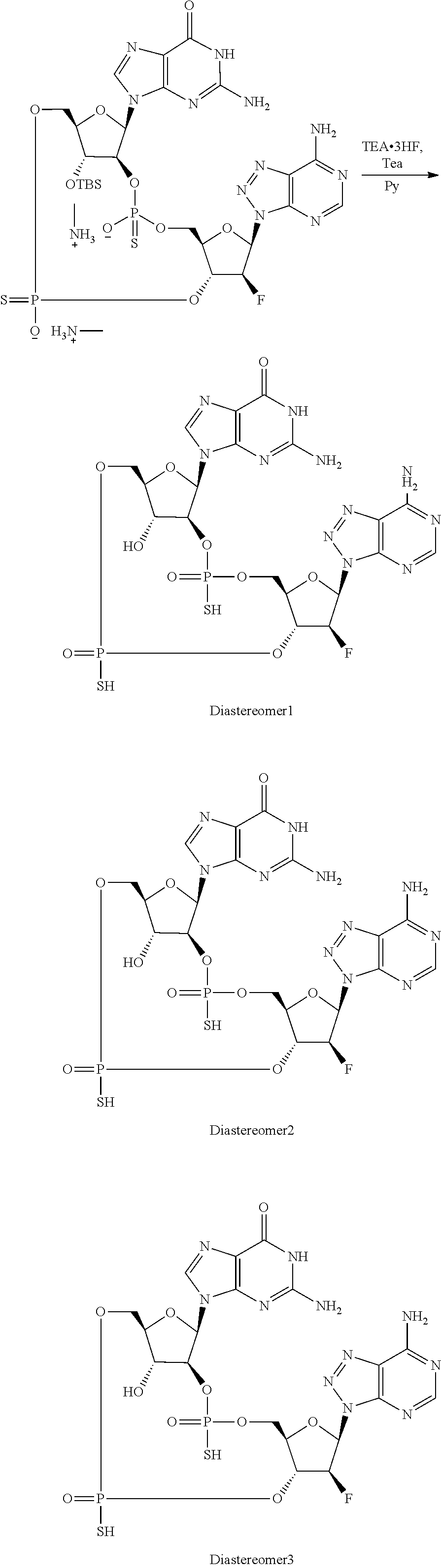

To the crude product of Step 10 was added Py (5 mL) and Et₃N (2 mL). The mixture was concentrated, and this process was repeated twice. To the residue was added Py (1.5 ml), TEA (1.11 g, 11.0 mmol) and TEA.3HF (0.887 g, 5.50 mmol). The mixture was stirred at 50° C. for 16 h. Then, it was concentrated and purified by prep-HPLC (Atlantis Prep T3 OBD Column) eluted with 0 to 16% ACN in aq NH₄HCO₃ (10 mM) over 16 min to give the products.

Example 1 (T_{R}=10.48 min, diastereomer 1): LCMS (ES, m/z): 710.1 [M+H]⁺. ¹H-NMR: (400 MHz, D₂O): δ 8.31 (s, 2H), 6.76 (dd, J=8.9, 5.3 Hz, 1H), 6.27 (s, 1H), 5.84 (t, J=5.1 Hz, 0.5H), 5.71 (t, J=5.1 Hz, 0.5H), 5.57-5.63 (m, 1H), 4.66 (d, J=2.3 Hz, 2H), 4.42 (dd, J=0.5, 3.6 Hz, 1H), 4.32-4.13 (m, 5H). ³¹P-NMR: (162 MHz, D₂O): δ 57.17 (s), 55.08 (s).

Example 2 (T_{R}=12.50 min, diastereomer 2): LCMS (ES, m/z): 710.1 [M+H]⁺. ¹H-NMR: (400 MHz, D₂O): δ 8.33 (s, 1H), 8.17 (s, 1H), 6.79 (t, J=6.5 Hz, 1H), 6.34 (s, 1H), 5.87 (m, 0.5H), 5.74 (m, 0.5H), 5.63 (m, 1H), 4.91 (d, J=11.4 Hz, 1H), 4.66 (m, 1H), 4.44 (m, 1H), 4.26 (m, 4H), 4.13 (m, 1H). ³¹P-NMR: (162 MHz, D₂O): δ 56.69 (s), 55.90 (s).

Fractions at T_{R}=13.47 min were further purified by reverse phase chromatography (Xbridge Prep Phenyl OBD) eluted with 0 to 95% ACN in aq NH₄HCO₃ (10 mM) over 16 min to give the product. Example 3 (T_{R}=9.67 min, diastereomer 3): LCMS (ES, m/z): 710.1 [M+H]⁺. ¹H-NMR: (400 MHz, D₂O): δ 8.35 (s, 1H), 8.09 (s, 1H), 6.79 (m, 1H), 6.30 (s, 1H), 5.86 (m, 0.5H), 5.73 (m, 0.5H), 5.57 (m, 1H), 4.88 (dd, J=11.4, 3.3 Hz, 1H), 4.45 (d, J=5.9 Hz, 1H), 4.29 (s, 2H), 4.24-4.14 (m, 4H). ³¹P-NMR: (162 MHz, D₂O): δ 55.97 (s), 54.85 (s).

Examples 4 and 5, as shown in Table 1 below, were prepared according to procedures analogous to those outlined in Examples 1, 2, and 3 above using appropriate monomers, described as Preparations or as obtained from commercial sources, in the coupling step.

TABLE 1

| | | | Mass |
|---|---|---|---|
| Ex. | Structure | Name | [M + H]⁺ |
| 4 | 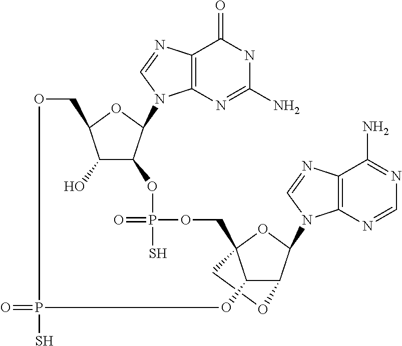 [SMILES: Nc1nc2c(ncn2[C@@H]2O[C@@H]3COP(=O)(S)O[C@H]4[C@H]5OC[C@]4(COP(=O)(S)O[C@H]2[C@@H]3O)O[C@H]5n2cnc3c(N)ncnc32)c(=O)[nH]1] | 2-amino-9-[(5R,7R,8S,12aR,14R,15R,15aS, 18R)-14-(6-amino-9H-purin-9-yl)-18-hydroxy- 2,10-dioxido-2,10-disulfanylhexahydro-14H- 15,12a-(epoxymethano)-5,8-methanofuro[3,2- l][1,3,6, 9,11,2,10]pentaoxadiphospha- cyclotetradecin-7(12H)-yl]-1,9-dihydro-6H- purin-6-one (diastereomer 1) | 719 |
| 5 | 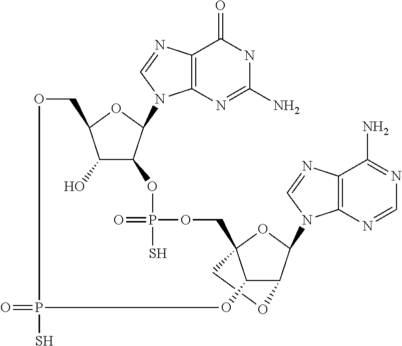 [SMILES: Nc1nc2c(ncn2[C@@H]2O[C@@H]3COP(=O)(S)O[C@H]4[C@H]5OC[C@]4(COP(=O)(S)O[C@H]2[C@@H]3O)O[C@H]5n2cnc3c(N)ncnc32)c(=O)[nH]1] | 2-amino-9-[(5R,7R,8S,12aR,14R,15R,15aS, 18R)-14-(6-amino-9H-purin-9-yl)-18-hydroxy- 2,10-dioxido-2,10-disulfanylhexahydro-14H- 15,12a-(epoxymethano)-5,8-methanofuro[3,2- l][1,3,6, 9,11,2,10]pentaoxadiphospha- cyclotetradecin-7(12H)-yl]-1,9-dihydro-6H- purin-6-one (diastereomer 2) | 719 |

Examples 6, 7, and 8: 2-amino-9-[(5S,7R,8S,12aR,14R,15S,15aR)-14-(7-amino-3H-[1,2,3]triazolo[4,5-d]pyrimidin-3-yl)-15-fluoro-2,10-dioxido-2,10-disulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 1), 2-amino-9-[(5S,7R,8S,12aR,14R,15S,15aR)-14-(7-amino-3H-[1,2,3]triazolo[4,5-d]pyrimidin-3-yl)-15-fluoro-2,10-dioxido-2,10-disulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 2) and 2-amino-9-[(5S,7R,8S,12aR,14R,15S,15aR)-14-(7-amino-3H-[1,2,3]triazolo[4,5-d]pyrimidin-3-yl)-15-fluoro-2,10-dioxido-2,10-disulfanyl-octahydro-12H-5,8-methanofuro[3,2-1][1,3,6,9,11,2,10]pentaoxadiphosphacyclotetradecin-7-yl]-1,9-dihydro-6H-purin-6-one (Diastereomer 3)

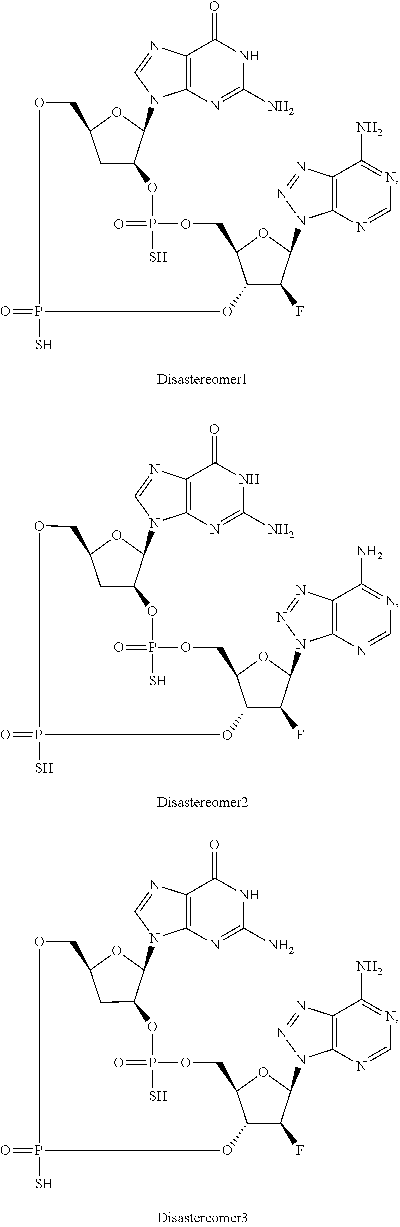 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1]

### Step 1: (2R,3S,5S)-5-((bis(4-methoxyphenyl)(phenyl)methoxy)methyl)-2-(2-isobutyramido-6-oxo-1,6-dihydro-9H-purin-9-yl)tetrahydrofuran-3-yl phenyl Phosphonate

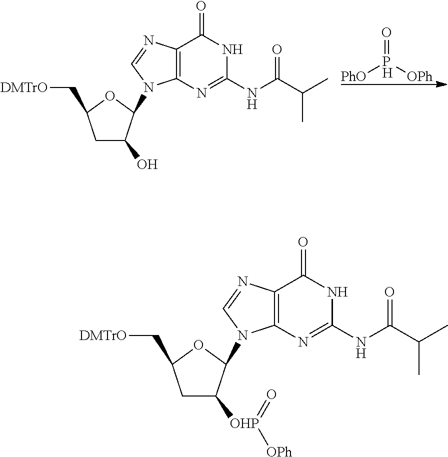

To a solution of N-(9-((2R,3S,5S)-5-((bis(4-methoxyphenyl)(phenyl)methoxy) methyl)-3-hydroxy-tetrahydrofuran-2-yl)-6-oxo-6,9-dihydro-1H-purin-2-yl)isobutyramide (prepared according to Nucleosides Nucleotides Nucleic Acids 2001, 20, 1783-1796; 702.9 mg, 1.1 mmol) in Py (11 mL) under Ar was added diphenyl phosphonate (1.29 g, 5.51 mmol), and it was stirred at RT for 20 min. Then, it was used for the next step directly without purification. LCMS (ES, m/z): 780.3 [M+H]⁺.

### Step 2: triethylammonium (2R,3S,5S)-5-((bis(4-methoxyphenyl)(phenyl)methoxy) methyl)-2-(2-isobutyramido-6-oxo-1,6-dihydro-9H-purin-9-yl)tetrahydrofuran-3-yl Phosphonate

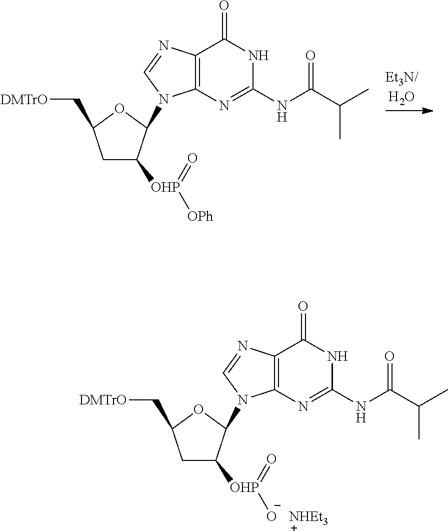

To the reaction mixture from Step 1 at RT was added water (1.1 ml) and Et₃N (1.1 ml), and the mixture was stirred at RT for 20 min. Then, it was concentrated, and the residue was partitioned between DCM (50 mL) and aq NaHCO₃ (5%, 20 mL). The layers were separated, and the organic layer was washed with aq NaHCO₃ (5%, 2×20 mL), dried (Na₂SO₄), and concentrated. It was purified by silica gel column chromatography eluted with 0 to 7.5% MeOH in DCM (with 1% Et₃N) to give the product. LCMS (ES, m/z): 704.2 [M+H]⁺. ¹H-NMR (400 MHz, DMSO-d₆): δ 12.05 (br s, 1H), 11.72 (br s, 1H), 8.00 (s, 1H), 7.33-7.28 (m, 2H), 7.22-7.18 (m, 7.5H), 6.77-6.73 (m, 4H), 6.08 (d, J=6.0 Hz, 1H), 5.38 (s, 0.5H), 4.93 (p, J=8.0 Hz, 1H), 4.23-4.19 (m, 1H), 3.68 (s, 6H), 3.35-3.3 (m, 2H), 2.76-2.73 (m, 1H), 2.35-2.32 (m, 1H), 2.16-2.12 (m, 1H), 1.07 (t, J=7.4 Hz, 6H). ³¹P-NMR (121 MHz, DMSO-d₆): δ −0.64 (s).

### Step 3: pyridin-1-ium (2R,3S,5S)-5-(hydroxymethyl)-2-(2-isobutyramido-6-oxo-1,6-dihydro-9H-purin-9-yl)tetrahydrofuran-3-yl Phosphonate

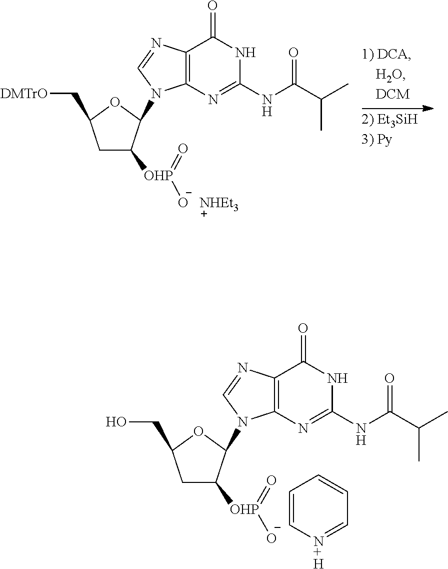

To a stirred solution of the product of Step 2 (620 mg, 0.77 mmol) in DCM (10 mL) at RT was added water (0.12 g, 6.6 mmol) and 2,2-dichloroacetic acid in DCM (0.63M, 9.5 mL, 5.9 mmol). It was stirred at RT for 30 min, and then Et₃SiH (12 mL) was added. After 40 min, Py (1 mL) was added, and it was stirred for 5 min. Then, it was concentrated to give a crude product, which was used for the next reaction step directly. LCMS (ES, m/z): 832.2 [M+H]⁺.

### Step 4: Pyridin-1-ium (2R,3S,5S)-5-((((((2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydrofuran-3-yl)oxy)(2-cyanoethoxy)phosphanyl)oxy)methyl)-2-(2-isobutyramido-6-oxo-1,6-dihydro-9H-purin-9 yl)tetrahydrofuran-3-yl Phosphonate

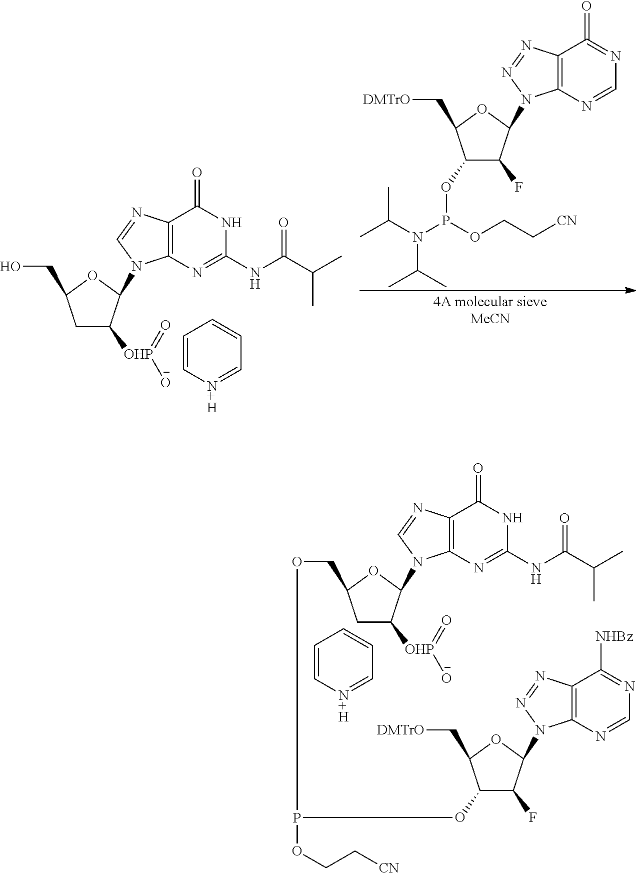

(2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydrofuran-3-yl (2-cyanoethyl) diisopropyl phosphoramidite (0.40 g, 0.46 mmol) was co-evaporated with ACN (3×2 mL) and dissolved in ACN (3 mL). Activated 4 Å molecular sieves (200 mg) were added to the solution. The crude from Step 3 was co-evaporated with ACN (3×3 mL) and dissolved in ACN (4 mL). Activated 4 Å molecular sieves (200 mg) were added to the solution. After 30 min, to the solution under Ar was added the solution containing (2R,3R,4S,5R)-5-(7-benzamido-3H-[1,2,3]triazolo[4,5-d]pyrimidin-3-yl)-2-((bis(4-methoxyphenyl)(phenyl)methoxy) methyl)-4-fluorotetrahydrofuran-3-yl (2-cyanoethyl) diisopropylphosphoramidite, and it was stirred at RT for 30 min. The reaction mixture was used in the next reaction step without purification. LCMS (ES, m/z): 1177.4 [M+H]⁺.

### Step 5: pyridin-1-ium (2R,3S,5S)-5-((((((2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydrofuran-3-yl)oxy)(2-cyanoethoxy)phosphorothioyl)oxy)methyl)-2-(2-isobutyramido-6-oxo-1,6-dihydro-9H-purin-9-yl)tetrahydrofuran-3-yl Phosphonate

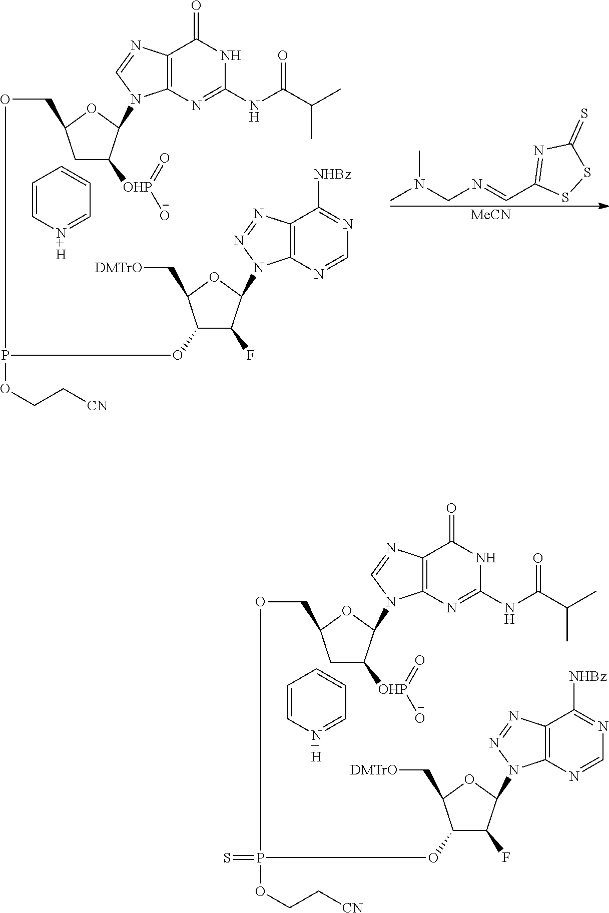

To the reaction mixture from Step 4 at RT was added (E)-N,N-dimethyl-N-(3-thioxo-3H-1,2,4-dithiazol-5-yl)formimidamide (0.227 g, 1.10 mmol), and it was stirred for 30 min. Then, the mixture was concentrated to give a crude product, which was used for the next reaction step directly without further purification. LCMS (ES, m/z): 1209.1 [M+H]⁺.

### Step 6: ammonium (2R,3S,5S)-5-((((((2R,3R,4S,5R)-5-(7-benzamido-3H-[1,2,3]triazolo[4,5-d]pyrimidin-3-yl)-4-fluoro-2-(hydroxymethyl)tetrahydrofuran-3-yl)oxy)(2-cyanoethoxy)phosphorothioyl)oxy)methyl)-2-(2-isobutyramido-6-oxo-1,6-dihydro-9H-purin-9-yl)tetrahydrofuran-3-yl Phosphonate

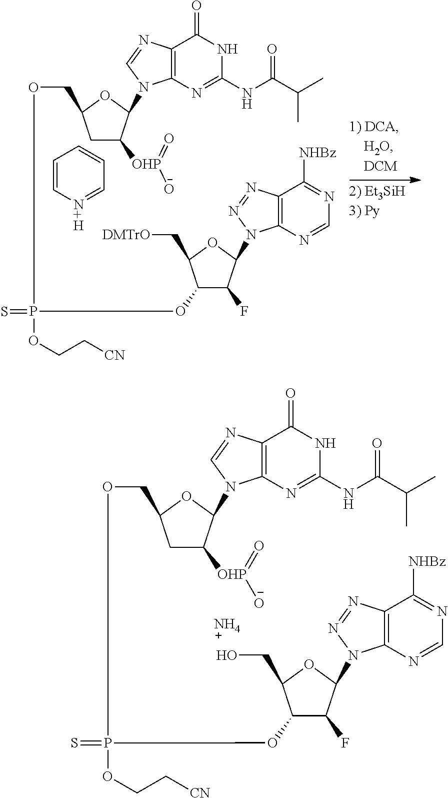

To the crude from Step 5 in DCM (6.5 mL) at RT was added water (181 mg, 10.0 mmol) and 2,2-dichloroacetic acid in DCM (0.63M, 12 ml, 1.0 mmol). After stirring for 15 min, Et₃SiH (12 mL) was added, and it was stirred for 1 h. Then, Py (1 ml) was added to the reaction. It was concentrated, and the crude was purified by reverse phase chromatography (C18) eluted with 0 to 40% ACN in aq NH₄HCO₃ (5 mM) to give the product. LCMS (ES, m/z): 907.2 [M+H]⁺. ¹H-NMR: ¹H NMR (300 MHz, CD₃OD) δ 8.82-8.79 (m, 1H), 8.26-8.14 (m, 1H), 8.13-8.09 (m, 2H), 7.63-7.44 (m, 3H), 7.01-6.98 (m, 1H), 6.27-6.24 (m, 1H), 6.03-5.72 (m, 1H), 5.43-5.40 (m, 1H), 5.10-5.05 (m, 2H), 4.43-4.29 (m, 4H), 3.88-3.78 (m, 2H), 2.98-2.80 (m, 3H), 2.70-2.65 (m, 1H), 2.40-2.30 (m, 1H), 1.29-1.07 (m, 6H). ³¹P-NMR: (121 MHz, CD₃OD) δ 68.09-68.03 (m), 2.39 (s).

### Step 7: Pyridinium (5S,7R,8S,12aR,14R,15S,15aR)-2-(2-cyanoethoxy)-15 fluoro-7-{2-[(2-methylpropanoyl)amino]-6-oxo-1,6-dihydro-9H-purin-9-yl}-14-{7 [(phenylcarbonyl) amino]-3H-[1,2,3]triazolo[4,5-d]pyrimidin-3-yl}octahydro-12H-5,8-methanofuro[3,2-1][1,3,6,9,11,2,10]pentaoxadiphosphacyclotetradecin-10-olate 2-sulfide

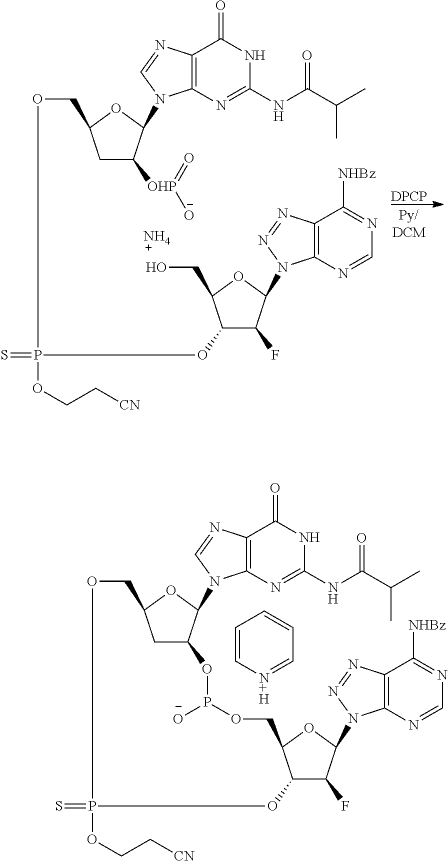

To Py (15 mL) at −40° C. under Ar was added DICP (2.21 g, 8.23 mmol), and a solution of the product of Step 6 (380 mg, 0.41 mmol) in Py (15 mL) dropwise over 20 min. The resulting mixture was stirred at −40° C. for 20 min. The solution containing the product was used for the next reaction step directly. LCMS (ES, m/z): [M+H]⁺ 889.2.

### Step 8: ammonium (5S,7R,8S,12aR,14R,15S,15aR)-2-(2-cyanoethoxy)-15-fluoro-7-{2-[(2-methylpropanoyl)amino]-6-oxo-1,6-dihydro-9H-purin-9-yl}-14-{7 [(phenylcarbonyl) amino]-3H-[1,2,3]triazolo[4,5-d]pyrimidin-3-yl}octahydro-12H-5,8-methanofuro[3,2-1][1,3,6,9,11,2,10]pentaoxadiphosphacyclotetradecine-10-thiolate 10-oxide 2-sulfide

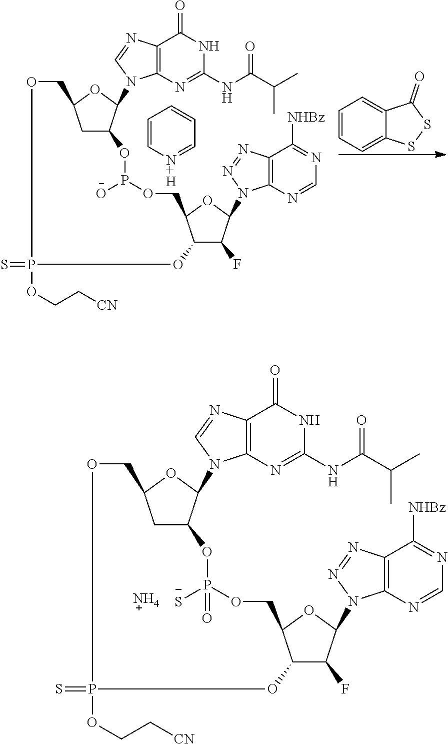

To the reaction mixture from Step 7 at −40° C. was added 3H-benzo[c][1,2]dithiol-3-one (104 mg, 0.615 mmol) and water (74 mg, 4.1 mmol). It was stirred at RT for 40 min. Then, the solution was concentrated. It was co-evaporated with toluene (2×10 mL) and ACN (10 mL). The crude was purified by reverse phase chromatography (C18) eluted with 0 to 95% ACN in aq NH₄HCO₃ (5 mM) to give the product. LCMS (ES, m/z): 921.2 [M+H]⁺. ³¹P-NMR: (162 MHz, CD₃OD) δ 69.04-63.64 (m); 57.73-56.84 (m).

### Step 9: diammonium (5S,7R,8S,12aR,14R,15S,15aR)-14-(7-amino-3H-[1,2,3]triazolo[4,5-d]pyrimidin-3-yl)-15-fluoro-7-{2-[(2-methylpropanoyl)amino]-6-oxo-1,6-dihydro-9H-purin-9-yl}-10-sulfidooctahydro-12H-5,8-methanofuro[3,2-1][1,3,6,9,11,2,10]pentaoxadiphosphacyclotetradecin-2-olate 10-oxide 2-sulfide

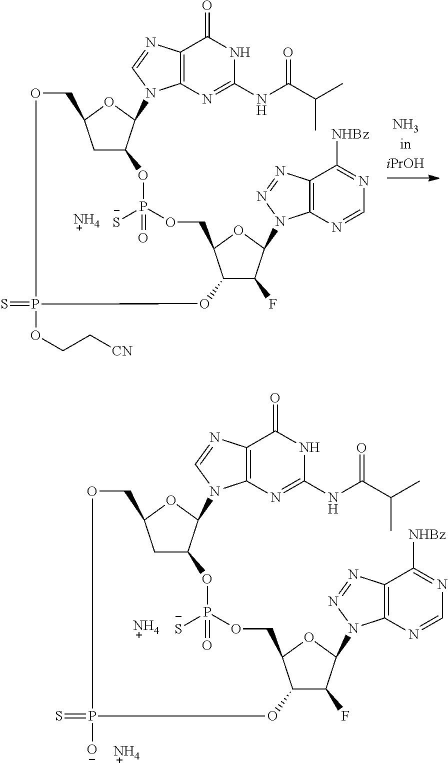

To a solution of NH₃ in i-PrOH (2M, 6 ml) at RT was added the product of Step 8 (150 mg, 0.194 mmol). The reaction container was sealed, and it was stirred at 50° C. for 3 h. Then, the solution was concentrated to give a crude product, which was directly used for next step without further purification. LCMS (ES, m/z): 764.1 [M+H]⁺.

### Step 10: 2-amino-9 [(5S,7R,8S,12aR,14R,15S,15aR)-14-(7-amino-3H-[1,2,3]triazolo[4,5-d]pyrimidin-3-yl)-15-fluoro-2,10-dioxido-2,10-disulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 1-3)

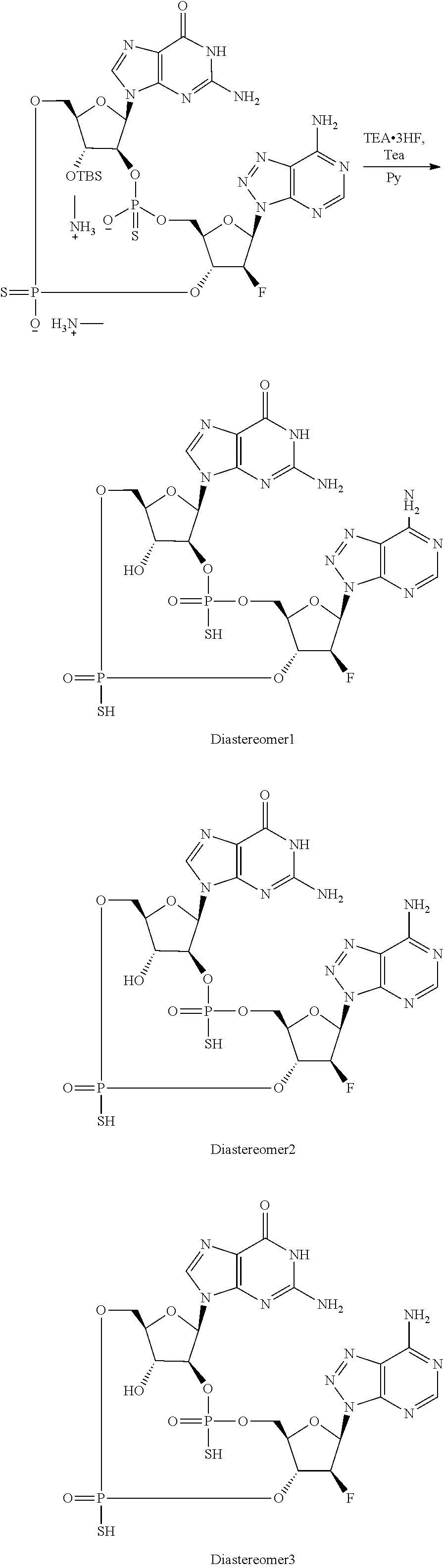

At RT, the crude from Step 9 was dissolved in a solution of methylamine in EtOH (30%, 15 mL). The resulting solution was stirred at RT for 3 h. Then, it was concentrated, and the crude product was purified by prep-HPLC (XBridge Shield RP18 OBD) eluted with 4 to 15% ACN in aq NH₄HCO₃ (10 mM) over 20 min to give the product.

Example 6 (T_{R}=10.08 min, diastereomer 1): LCMS (ES, m/z): 694.1 [M+H]⁺. ¹H-NMR: (400 MHz, D₂O): δ 8.32 (s, 1H), 8.17 (s, 1H), 6.76 (dd, J=10.0, 5.1 Hz, 1H), 6.08 (dd, J=3.4, 1.6 Hz, 1H), 5.82-5.62 (m, 2H), 4.97-4.83 (m, 1H), 4.46-4.40 (m, 2H), 4.34-4.18 (m, 3H), 4.16-3.99 (m, 1H), 2.72-2.65 (m, 1H), 2.49-2.43 (m, 1H). ³¹P-NMR (162 MHz, D₂O): δ 56.51 (s); 54.92 (s).

Fractions at T_{R}=12.67 min were further purified by reverse phase chromatography (Xbridge Prep Phenyl OBD) eluted with 2 to 20% ACN in aq NH₄HCO₃ (10 mM) over 14 min to give the product.

Example 7 (T_{R}=12.2 min, diastereomer 2): LCMS (ES, m/z): 694.1 [M+H]⁺. ¹H-NMR (400 MHz, D₂O): δ 8.27 (s, 1H), 8.09 (s, 1H), 6.79-6.68 (m, 1H), 6.09 (t, J=2.8 Hz, 1H), 5.85-5.55 (m, 2H), 5.05-5.01 (m, 1H), 4.42-4.22 (m, 3H), 4.17-4.13 (m, 2H), 3.99-3.89 (m, 1H), 2.64-2.37 (m, 2H). ³¹P-NMR (121 MHz, D₂O): δ 56.67(s); 54.96(s).

Example 8 (T_{R}=17.38 min, diastereomer 3): LCMS (ES, m/z): 694.1 [M+H]⁺. ¹H-NMR: (400 MHz, D₂O): δ 8.37 (s, 1H), 8.32 (s, 1H), 6.82-6.77 (m, 1H), 6.16 (d, J=3.3 Hz, 1H), 5.78 (dt, J=51.5, 5.4 Hz, 1H), 5.63-5.57 (m, 1H), 5.12-5.04 (m, 1H), 4.45 (m, 2H), 4.38-4.16 (m, 3H), 4.09 (s, 1H), 2.66-2.62 (m, 1H), 2.54-2.48 (m, 1H). ³¹P-NMR (162 MHz, D₂O): δ 55.17 (s); 54.89 (s).

Examples 9 through 12, as shown in Table 2 below, were prepared according to procedures analogous to those outlined in Examples 6, 7, and 8 above using appropriate monomers, described as Preparations or as obtained from commercial sources, in the coupling step.

TABLE 2

| | | | Mass |
|---|---|---|---|
| Ex. | Structure | Name | [M + H]⁺ |
| 9 | 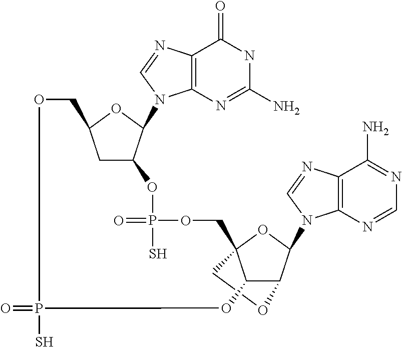 [SMILES: Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1] | 2-amino-9-[(5S,7R,8S,12aR,14R,15R,15aS)- 14-(6-amino-9H-purin-9-yl)-2,10-dioxido-2,10- disulfanylhexahydro-14H-15,12a- (epoxymethano)-5,8-methanofuro[3,2- l][1,3,6,9,11,2,10]pentaoxadi- phosphacyclotetradecin-7(12H)-yl]-1,9- dihydro-6H-purin-6-one (diastereomer 1) | 703 |
| 10 | 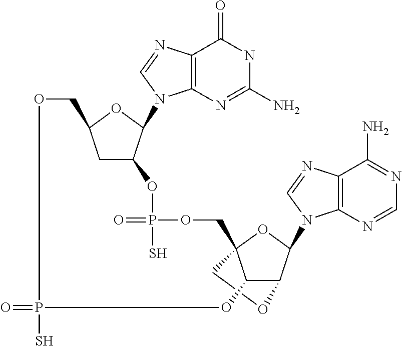 [SMILES: Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1] | 2-amino-9-[(5S,7R,8S,12aR,14R,15R,15aS)- 14-(6-amino-9H-purin-9-yl)-2,10-dioxido-2,10- disulfanylhexahydro-14H-15,12a- (epoxymethano)-5,8-methanofuro[3,2- l][1,3,6,9,11,2,10]pentaoxadi- phosphacyclotetradecin-7(12H)-yl]-1,9- dihydro-6H-purin-6-one (diastereomer 2) | 703 |
| 11 | 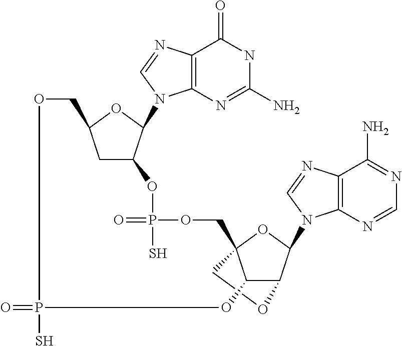 [SMILES: Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1] | 2-amino-9-[(5S,7R,8S,12aR,14R,15R,15aS)- 14-(6-amino-9H-purin-9-yl)-2,10-dioxido-2,10- disulfanylhexahydro-14H-15,12a- (epoxymethano)-5,8-methanofuro[3,2- l][1,3,6,9,11,2,10]pentaoxadi- phosphacyclotetradecin-7(12H)-yl]-1,9- dihydro-6H-purin-6-one (diastereomer 3) | 703 |
| 12 | 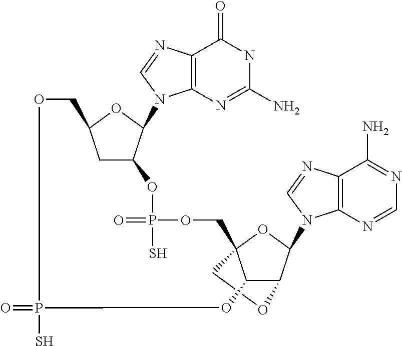 [SMILES: Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1] | 2-amino-9-[(5S,7R,8S,12aR,14R,15R,15aS)- 14-(6-amino-9H-purin-9-yl)-2,10-dioxido-2,10- disulfanylhexahydro-14H-15,12a- (epoxymethano)-5,8-methanofuro[3,2- l][1,3,6,9,11,2,10]pentaoxadi- phosphacyclotetradecin-7(12H)-yl]-1,9- dihydro-6H-purin-6-one (diastereomer 4) | 703 |

### Examples 13 and 14: 2-amino-9-[(5R,7R,8S,12aR,14R,15S,15aR,16R)-14-(6-amino-9H-purin-9-yl)-15,16-difluoro-2-hydroxy-2-oxido-10-sulfanyl-10-sulfidooctahydro-12H-5,8-methanofuro[3.2-1][1,3,6,9,11,2,10]pentaoxadiphosphacyclotetradecin-7-yl]-1,9-dihydro-6H-purin-6-one (Diastereomer 1) and 2-amino-9-[(5R,7R,8R,12aR,14R,15S,15aR,16R)-14-(6-amino-9H-purin-9-yl)-15,16-difluoro-2-hydroxy-2,10-dioxido-10-sulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 2)

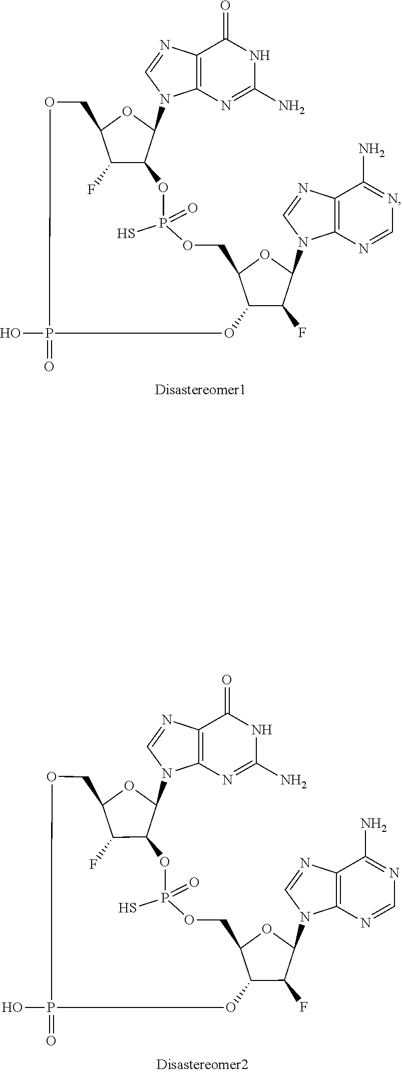 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(N)ncnc65)O[C@@H]4COP(=O)(S)O[C@H]2[C@@H]3F)c(=O)[nH]1]

### Step 1: N-(9-((2R,3S,4S,5R)-5-(((tert-butyldimethylsilyl)oxy)methyl)-4-fluoro-3-hydroxytetrahydrofuran-2-yl)-6-oxo-6,9-dihydro-1H-purin-2-yl)isobutyramide

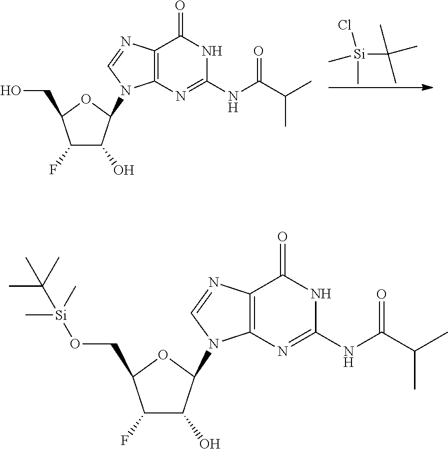

To a solution of N-(9-((2R,3S,4S,5R)-4-fluoro-3-hydroxy-5-(hydroxymethyl) tetrahydrofuran-2-yl)-6-oxo-6,9-dihydro-1H-purin-2-yl)isobutyramide (1.0 g, 2.81 mmol) in Py (20 ml) at RT was added tert-butyldimethylsilyl chloride in THE (1.0M, 3.4 ml, 3.4 mmol) dropwise over 5 min. The mixture was stirred at RT for 16 h. Then, Et₂₀ (40 ml) was added, and it was stirred for 10 min. Precipitates were removed by filtration. The solid was washed with Et₂O (20 ml) and the combined filtrate was concentrated to give a crude product. LCMS (ES, m/z): 470 [M+H]⁺.

### Step 2: N-(9-((2R,4R,5R)-5-(((tert-butyldimethylsilyl)oxy)methyl)-4-fluoro-3-oxotetrahydrofuran-2-yl)-6-oxo-6,9-dihydro-1H-purin-2-yl)isobutyramide

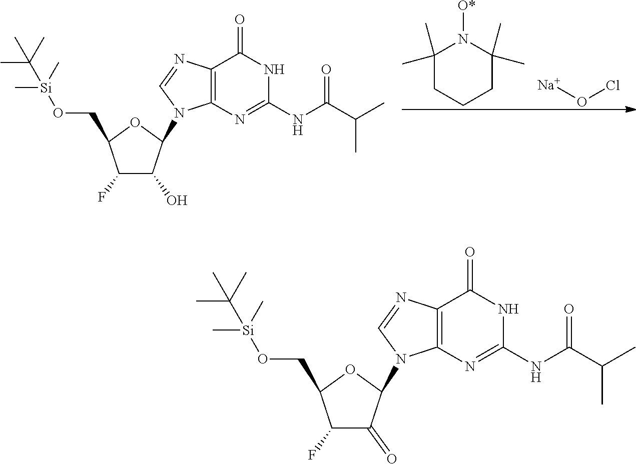

Sodium hypochlorite (15.5 ml, 11.2 mmol) was added to a mixture of the product of Step 1 (2.11 g, 4.49 mmol), KBr (0.535 g, 4.49 mmol), TEMPO (0.140 g, 0.899 mmol) and NaHCO₃ (1.13 g, 13.5 mmol) in DCM (20 ml) and water (5 ml) at 0° C. It was stirred for 3 h. Then, the reaction mixture was partitioned between EtOAc (100 ml) and aq Na₂S₂O₃ (10%, 50 ml). The layers were separated, and the organic layer was washed again with aq Na₂S₂O₃ (10%, 50 ml), dried (MgSO₄), and concentrated. The crude product was used in the next step without purification. LCMS (ES, m/z): 486 [M+H₂O+H]⁺.

### Step 3: N-(9-((2R,3R,4S,5R)-5-(((tert-butyldimethylsilyl)oxy)methyl)-4-fluoro-3-hydroxytetrahydrofuran-2-yl)-6-oxo-6,9-dihydro-1H-purin-2 yl)isobutyramide

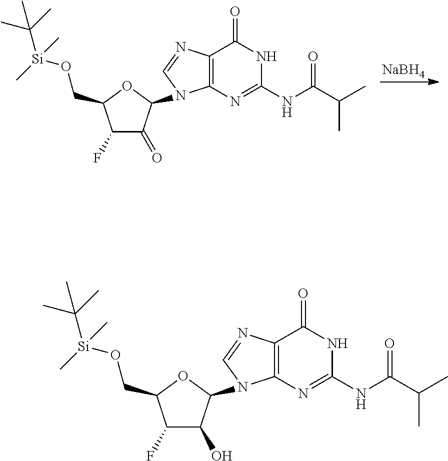

To a mixture of the product of Step 2 (2.15 g, 4.60 mmol) and NH₄Cl (0.246 g, 4.60 mmol) in EtOH (40 ml) at 0° C. was added NaBH₄ (0.435 g, 11.5 mmol), and it was stirred as it warmed to RT over 18 h. Then, it was partitioned between EtOAc (100 ml) and sat aq NaHCO₃ (50 ml). The layers were separated, and the organic layer was dried (Na₂SO₄) and concentrated. The residue was purified by column chromatography on silica gel eluted with 0 to 3% MeOH in DCM to give the product. LCMS (ES, m/z): 470 [M+H]⁺.

### Step 4: O-(((2R,3R,4S,5R)-5-(6-benzamido-9H-purin-9-yl)-4-fluoro-3-hydroxytetrahydrofuran-2-yl)methyl)O-((2R,3R,4R,5R)-5-(((tert-butyldimethylsilyl)oxy)methyl)-4-fluoro-2-(2-isobutyramido-6-oxo-1,6-dihydro-9H-purin-9-yl)tetrahydrofuran-3-yl) O-(2-cyanoethyl) Phosphorothioate

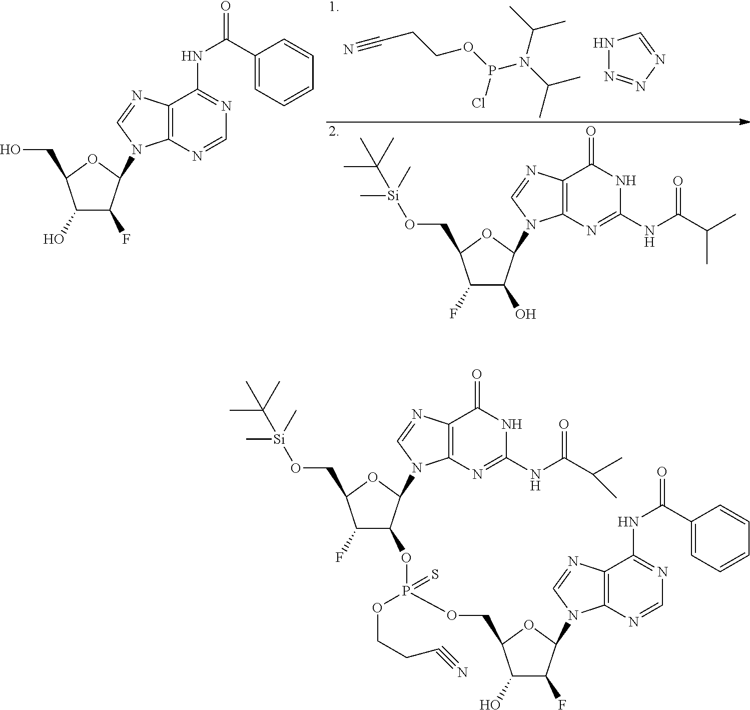

To a solution of N-(9-((2R,3S,4R,5R)-3-fluoro-4-hydroxy-5-(hydroxymethyl) tetrahydrofuran-2-yl)-9H-purin-6-yl)benzamide (398 mg, 1.07 mmol) in DMF (8 ml) at 0° C. was added N,N-diisopropylethyamine (0.558 ml, 3.19 mmol), activated molecular sieves (4 Å, 200 mg) and 2-cyanoethyl N,N-diisopropylchlorophosphoramidite (312 mg, 1.28 mmol) in DCM (1 ml). The resulting mixture was stirred at 0° C. for 2 h and then, gradually warmed to RT and stirred for 18 h. Then, TMSCl in THE (1.0M, 1.6 ml, 1.6 mmol) was added dropwise. It was stirred at RT for 4 h, and then, the product of Step 3 (250 mg, 0.532 mmol) and 1H-tetrazole (448 mg, 6.39 mmol) were added. The mixture was stirred at RT for 2 h, and DDTT (284 mg, 1.38 mmol) was added. It was stirred for 1 h. Then, it was partitioned between EtOAc (30 ml) and water (10 ml). The layers were separated, and the organic layer was dried (Na₂SO₄) and concentrated. The residue was purified by column chromatography on silica gel eluted with 0 to 6% MeOH in DCM to give the product. LCMS (ES, m/z): 975 [M+H]⁺.

### Step 5: O-(((2R,3R,4S,5R)-5-(6-benzamido-9H-purin-9-yl)-4-fluoro-3-hydroxytetrahydrofuran-2-yl)methyl) O-((2R,3R,4R,5R)-4-fluoro-5-(hydroxymethyl)-2-(2-isobutyramido-6-oxo-1,6-dihydro-9H-purin-9-yl)tetrahydrofuran-3-yl) O-hydrogen Phosphorothioate

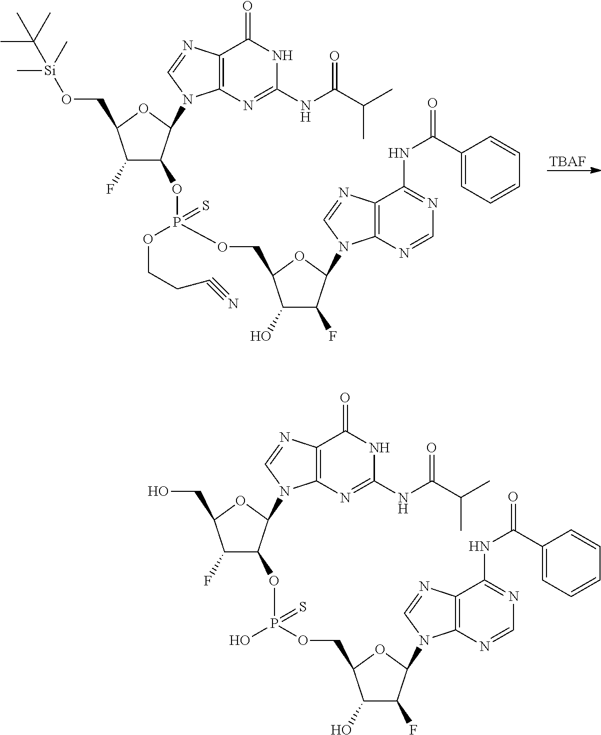

To a solution of the product of Step 4 (250 mg, 0.257 mmol) in THF (10 ml) was added tetra-n-butylammonium fluoride in THF (1.0M, 0.51 ml, 0.51 mmol). It was stirred at RT for 2 h and concentrated. The residue was purified by column chromatography on silica gel eluted with 0 to 12% MeOH in DCM to give the product. LCMS (ES, m/z): 807 [M+H]⁺.

### Step 6: N-{9-[(5R,7R,8R,12aR,14R,15S,15aR,16R)-2-(2-cyanoethoxy)-15,16-difluoro-10-hydroxy-7-(2 [(2-methylpropanoyl)amino]-6-oxo-1,6-dihydro-9H-purin-9-yl}-2-oxido-10-sulfidooctahydro-12H-5,8-methanofuro[3,2-1][1,3,6,9,11,2,10]pentaoxadiphosphacyclotetradecin-14-yl]-9H-purin-6-yl)benzamide

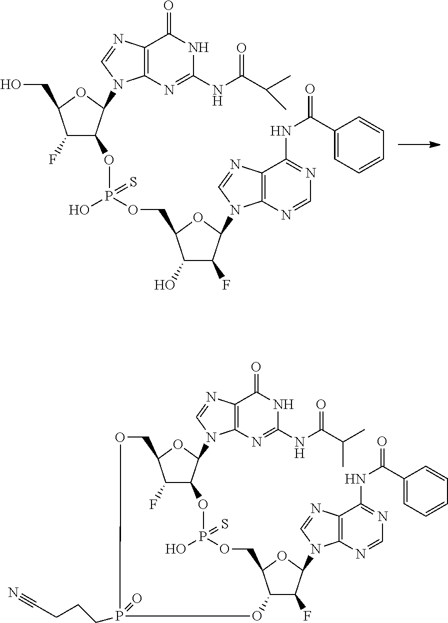 [SMILES: CC(C)C(=O)Nc1nc2c(ncn2[C@@H]2O[C@H](CO)[C@@H](F)[C@@H]2OP(O)(=S)OC[C@H]2O[C@@H](n3cnc4c(NC(=O)c5ccccc5)ncnc43)[C@@H](F)[C@@H]2O)c(=O)[nH]1 ; CC(C)C(=O)Nc1nc2c(ncn2[C@@H]2O[C@@H]3COP(=O)(CCCC#N)O[C@H]4[C@H](F)[C@H](n5cnc6c(NC(=O)c7ccccc7)ncnc65)O[C@@H]4COP(O)(=S)O[C@H]2[C@@H]3F)c(=O)[nH]1]

The product of Step 5 (57.3 mg, 0.335 mmol) and 1H-tetrazole (3.1 mg, 0.045 mmol) were co-evaporated with ACN (3×10 ml) and dissolved in DMF (1 ml) and ACN (7 ml). To the solution was added activated molecular sieves (4 Å, 200 mg) and a solution of 2-cyanoethyl N,N,N′,N′-tetraisopropylphosphorodiamidite (99 mg, 0.31 mmol) in ACN (1 ml). The resulting mixture was stirred at RT for 3 h, and then, 1H-tetrazole (78 mg, 1.1 mmol) was added. After 1 h, t-butyl hydroperoxide in decane (5.0M, 0.22 ml, 1.1 mmol) was added. It was stirred for 1 h and then, concentrated to give a crude product. LCMS (ES, m/z): 920 [M−H]⁻.

### Step 6: 2-amino-9-[(5R,7R,8R,12aR,14R,15S,15aR,16R)-14-(6-amino-9H-purin-9-yl)-15,16-difluoro-2-hydroxy-2,10-dioxido-10-sulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 1-2)

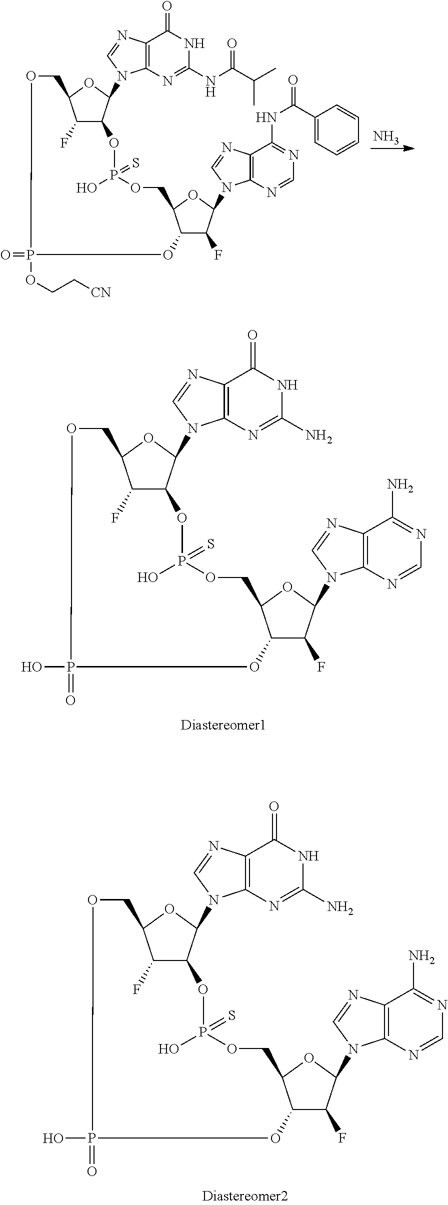

The crude product from Step 5 and ammonia in MeOH (7.0M, 2.0 ml, 14 mmol) were sealed in a tube. It was heated to 50° C. and stirred for 4 h. Then, it was concentrated, and the residue was purified by reverse phase prep HPLC (X-Bridge BEH 150 Prep C18) eluted with 2 to 15% ACN in aq tetraethylammonium acetate (0.1M) over 18 min to give the products.

Example 13 (T_{R}=8.51 min, diastereomer 1): LCMS (ES, m/z): 693 [M−H]⁻. ¹H NMR (D₂O, 500 MHz): δ 8.33 (1H, d, J=2.8 Hz), 8.22 (1H, s), 8.05 (1H, s), 6.50 (1H, dd, J=18.4, 3.9 Hz), 6.36 (1H, s), 5.55 (1H, d, J=13.1 Hz), 5.45 (1H, d, J=14.3 Hz), 5.13-5.08 (2H, m), 4.48 (1H, d, J=30.7 Hz), 4.31-4.20 (2H, m), 4.14 (2H, dd, J=12.3, 6.7 Hz), 4.06 (1H, d, J=10.5 Hz). ³¹P NMR: (D₂O, 202 MHz): δ 56.2, −0.5.

Example 14 (T_{R}=15.01 min, diastereomer 2): LCMS (ES, m/z): 693 [M−H]⁻. ¹H NMR (D₂O, 500 MHz): δ 8.37 (1H, d, J=2.8 Hz), 8.22 (1H, s), 8.06 (1H, s), 6.52 (1H, dd, J=18.4, 3.9 Hz), 6.35 (1H, s), 5.50 (1H, d, J=7.3 Hz), 5.13-5.07 (2H, m), 4.94 (1H, td, J=13.0, 3.5 Hz), 4.48 (1H, s), 4.31-4.20 (2H, m), 4.15-4.12 (1H, m), 4.06 (1H, d, J=10.5 Hz) 3.99 (1H, q, J=6.4 Hz). ³¹P NMR: (D₂O, 202 MHz): δ 58.6, −0.1.

### Examples 15, 16, and 17: 2-amino-9-[(5R,7R,8R,12aR,14R,15S,15aR,16R)-14-(6-amino-9H-purin-9-yl)-15,16-difluoro-2,10-dioxido-2,10-disulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 1), 2-amino-9-[(5R,7R,8R,12aR,14R,15S,15aR,16R)-14-(6-amino-9H-purin-9-yl)-15,16-difluoro-2,10-dioxido-2,10-disulfanyl octahydro-12H-5,8-methanofuro[3,2-1][1,3,6,9,11,2,10]pentaoxadiphosphacyclotetradecin-7-yl]-1,9-dihydro-6H-purin-6-one (Diastereomer 2) and 2-amino-9-[(5R,7R,8R,12aR,14R,15S,15aR,16R)-14-(6-amino-9H-purin-9-yl)-15,16-difluoro-2,10-dioxido-2,10-disulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 3)

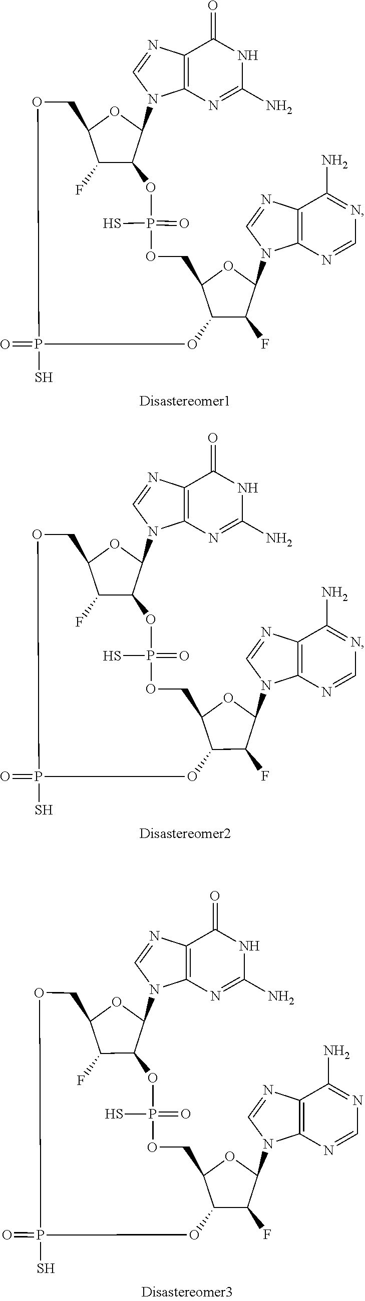 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1]

### Step 1: N-{9-[(5R,7R,8R,12aR,14R,15S,15aR,16R)-2-(2-cyanoethoxy)-15,16-difluoro-10-hydroxy-7-{2 [(2-methylpropanoyl)amino]-6-oxo-1,6-dihydro-9H-purin-9-yl}-2,10-disulfidooctahydro-12H-5,8-methanofuro[3.2-1][1,3,6,9,11,2,10]pentaoxadiphosphacyclo-tetradecin-14-yl]-9H-purin-6-yl}benzamide

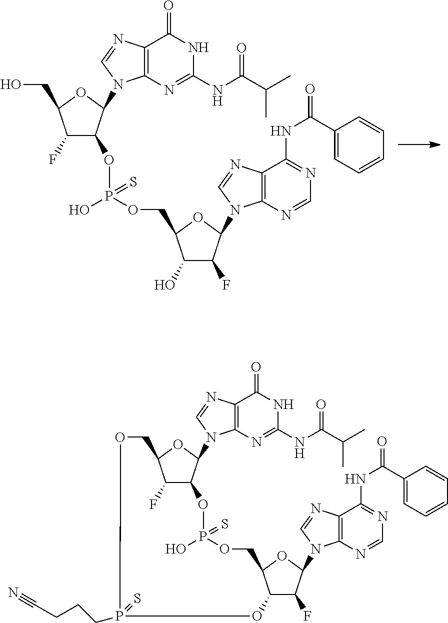 [SMILES: CC(C)C(=O)Nc1nc2c(ncn2[C@@H]2O[C@H](CO)[C@@H](F)[C@@H]2OP(O)(=S)OC[C@H]2O[C@@H](n3cnc4c(NC(=O)c5ccccc5)ncnc43)[C@@H](F)[C@@H]2O)c(=O)[nH]1 ; CC(C)C(=O)Nc1nc2c(ncn2[C@@H]2O[C@@H]3COP(=S)(CCCC#N)O[C@H]4[C@H](F)[C@H](n5cnc6c(NC(=O)c7ccccc7)ncnc65)O[C@@H]4COP(O)(=S)O[C@H]2[C@@H]3F)c(=O)[nH]1]

The product according to Step 4 of Examples 13 and 14 (170 mg, 0.211 mmol), diisopropylammonium tetrazolide (72 mg, 0.42 mmol) and 1H-tetrazole (3.0 mg, 0.042 mmol) were co-evaporated with ACN (3×10 ml) and dissolved in DMF (2 ml) and ACN (7 ml). To the solution was added activated molecular sieves (4 Å, 200 mg) and a solution of 2-cyanoethyl N,N,N′,N′-tetraisopropylphosphorodiamidite (94 mg, 0.30 mmol) in ACN (1 ml). The resulting mixture was stirred at RT for 20 min, and then, 1H-tetrazole (74 mg, 1.1 mmol) was added. After 1 h, DDTT (65 mg, 0.32 mmol) was added. It was stirred for 1 h and then, concentrated and partly purified by column chromatography on silica gel eluted with 0 to 10% MeOH in DCM to give a crude product. LCMS (ES, m/z): 938 [M+H]⁺.

### Step 2: 2-amino-9-[(5R,7R,8R,12aR,14R,15S,15aR,16R)-14-(6-amino-9H-purin-9-yl)-15,16-difluoro-2,10-dioxido-2,10-disulfanyloctahydro-12H-5,8-methanofuro[3,2-1][1,3,6,9,11,2,10]pentaoxadiphosphacyclotetradecin-7-yl]-1,9-dihydro-6H-purin-6-one (Diastereomer 1-3)

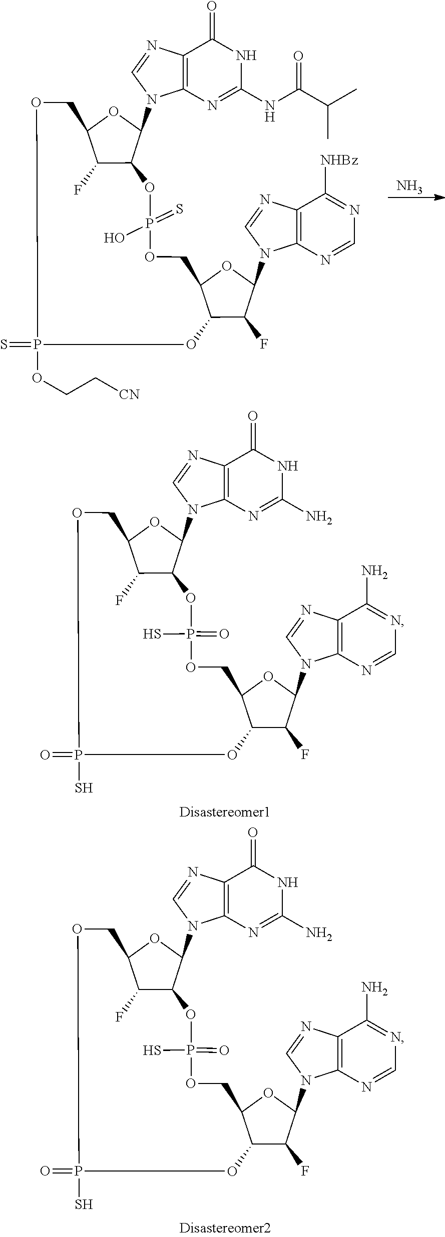

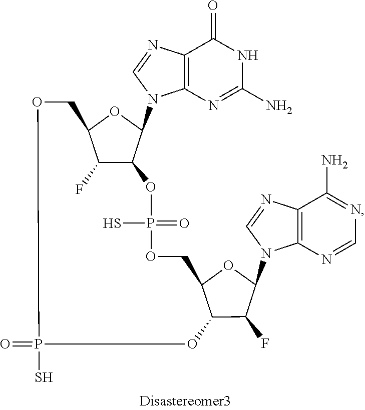 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1]

The crude from Step 1 and ammonia in MeOH (7.0M, 3.0 ml, 21 mmol) were sealed in a tube. It was heated to 50° C. and stirred for 4 h. Then, it was concentrated and purified by reverse phase prep HPLC (X-Bridge BEH 150 Prep C18) eluted with 2 to 20% ACN in aq tetraethylammonium acetate (0.1M) over 10 min to give the products.

Example 15 (T_{R}=13.67 min, diastereomer 1): LCMS (ES, m/z): 709 [M−H]⁻. ¹H NMR (D₂O, 500 MHz): δ 8.41 (1H, d, J=2.9 Hz), 8.24 (2H, d, J=12.0 Hz), 6.48 (1H, dd, J=18.4, 3.9 Hz), 6.34 (1H, s), 5.76 (1H, d, J=7.3 Hz), 5.42-5.27 (2H, m), 4.89 (1H, t), 4.46 (1H, s), 4.29 (2H, s), 4.16-4.23 (1H, m), 4.06 (1H, d, J=10.5 Hz) 3.99 (1H, q, J=6.4 Hz). ³¹P NMR: (D₂O, 202 MHz): δ 59.0, 56.7.

Example 16 (T_{R}=15.45 min, diastereomer 2): LCMS (ES, m/z): 709 [M−H]⁻. ¹H NMR (D₂O, 500 MHz): δ 8.41 (1H, d, J=2.9 Hz), 8.23 (1H, s), 8.19 (1H, s), 6.49 (1H, dd, J=19.6, 3.7 Hz), 6.35 (1H, s), 5.67 (1H, d, J=50.1 Hz), 5.47 (1H, d, J=49.8 Hz), 5.24 (1H, dd, J=21.2, 11.7 Hz), 5.09-5.04 (1H, m), 4.49 (1H, d, J=31.4 Hz), 4.41-4.29 (3

Example 17 (T_{R}=18.26 min, diastereomer 3): LCMS (ES, m/z): 709 [M−H]⁻. ¹H NMR (D₂O, 500 MHz): δ 8.38 (1H, d, J=2.9 Hz), 8.23 (1H, s), 7.99 (1H, s), 6.51 (1H, dd, J=19.3, 3.7 Hz), 6.34 (1H, s), 5.60 (1H, d, J=18.4 Hz), 5.50 (1H, d, J=18.9 Hz), 5.24 (1H, dd, J=21.2, 11.7 Hz), 5.08 (1H, td, J=13.0, 3.5 Hz), 4.50-4.44 (2H, m), 4.37 (3H, dd, J=19.9, 6.2 Hz), 4.26-4.23 (1H, m). ³¹P NMR: (D₂O, 202 MHz): δ 56.4, 54.8.

Examples 18 and 19, as shown in Table 3 below, were prepared according to procedures analogous to those outlined in Examples 15, 16, and 17 above using appropriate monomers, described as Preparations or as obtained from commercial sources, in the coupling step.

TABLE 3

| | | | Mass |
|---|---|---|---|
| Ex. | Structure | Name | [M + H]⁺ |
| 18 | 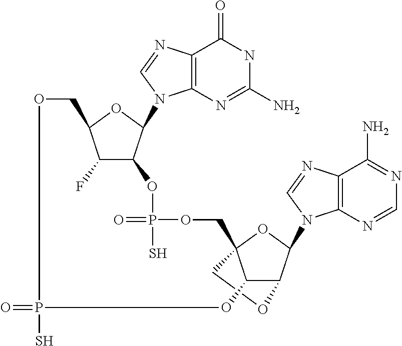 [SMILES: Nc1nc2c(ncn2[C@@H]2O[C@@H]3COP(=O)(S)O[C@H]4[C@H]5OC[C@]4(COP(=O)(S)O[C@H]2[C@@H]3F)O[C@H]5n2cnc3c(N)ncnc32)c(=O)[nH]1] | 2-amino-9-[(5R,7R,8R,12aR,14R,15R,15aS, 18R)-14-(6-amino-9H-purin-9-yl)-18-fluoro- 2,10-dioxido-2,10-disulfanylhexahydro-14H- 15,12a-(epoxymethano)-5,8-methanofuro[3,2- l][1,3,6,9,11,2,10]pentaoxadiphosphacyclotetra- decin-7(12H)-yl]-1,9-dihydro-6H-purin-6-one (diastereomer 1) | 719 |
| 19 | 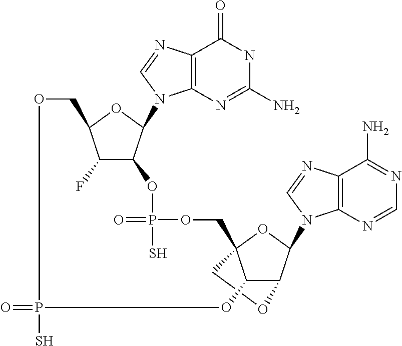 [SMILES: Nc1nc2c(ncn2[C@@H]2O[C@@H]3COP(=O)(S)O[C@H]4[C@H]5OC[C@]4(COP(=O)(S)O[C@H]2[C@@H]3F)O[C@H]5n2cnc3c(N)ncnc32)c(=O)[nH]1] | 2-amino-9-[(5R,7R,8R,12aR,14R,15R,15aS, 18R)-14-(6-amino-9H-purin-9-yl)-18-fluoro- 2,10-dioxido-2,10-disulfanylhexahydro-14H- 15,12a-(epoxymethano)-5,8-methanofuro[3,2- l][1,3,6,9,11,2,10]pentaoxadiphosphacyclotetra- decin-7(12H)-yl]-1,9-dihydro-6H-purin-6-one (diastereomer 2) | 719 |

### Examples 20 and 21: 5-amino-3-{(5S,7R,8S,12aR,14R,15S,15aR)-14-(7-amino-3H-[1,2,3]triazolo[4,5-d]pyrimidin-3-yl)-15-fluoro-2,10-dioxido-2,10-disulfanyloctahydro-12H-5,8-methanofuro[3,2-1][1,3,6,9,11,2,10]pentaoxadiphosphacyclotetradecin-7-yl}-3,6-dihydro-7H-[1,2,3]triazolo[4,5-d]pyrimidin-7-one (Diastereomer 1) and 5-amino-3-[(5S,7R,8S,12aR,14R,15S,15aR)-14-(7-amino-3H-[1,2,3]triazolo[4,5-d]pyrimidin-3-yl)-15-fluoro-2,10-dioxido-2,10-disulfanyloctahydro-12H-5,8-methanofuro[3,2-1][1,3,6,9,11,2,10]pentaoxadiphosphacyclotetradecin-7-yl]-3,6-dihydro-7H-[1,2,3]triazolo[4,5-d]pyrimidin-7-one (Diastereomer 2)

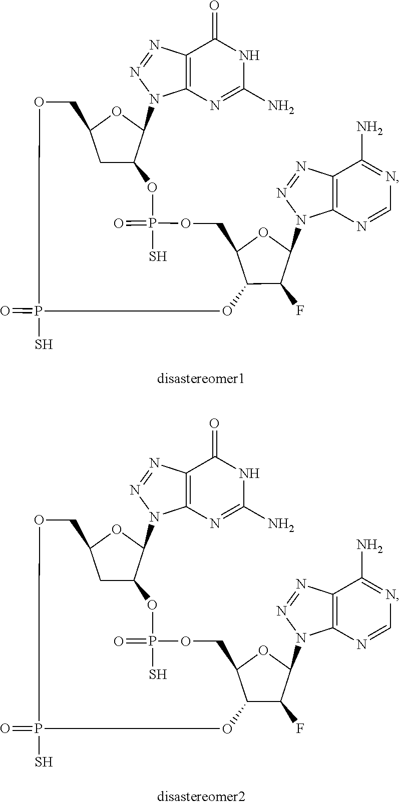 [SMILES: Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1 ; Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1]

### Step 1: (2R,3S,5S)-5-((bis(4-methoxyphenyl)(phenyl)methoxy)methyl)-2-(5-isobutyramido-7-oxo-6,7-dihydro-3H-[1,2,3]triazolo[4,5-d]pyrimidin-3-yl)tetrahydrofuran-3-yl phenyl Phosphonate

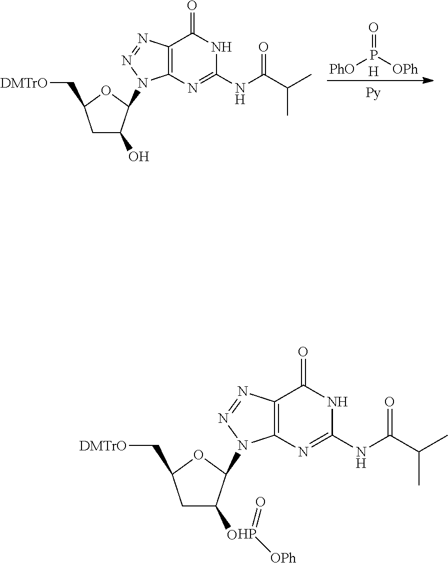

The compound of Preparation 3 (450 mg, 0.702 mmol) was co-evaporated with Py (3×4 mL) and dissolved in Py (3.5 ml). To the mixture at RT under Ar was added diphenyl phosphonate (822 mg, 3.51 mmol), and it was stirred for 20 min. The reaction solution was used for the next step directly. LCMS (ES, m/z): 779.0 [M−H]⁻.

### Step 2: triethylammonium (2R,3S,5S)-5-((bis(4-methoxyphenyl)(phenyl)methoxy) methyl)-2-(5-isobutyramido-7-oxo-6,7-dihydro-3H-[1,2,3]triazolo[4,5-d]pyrimidin-3-yl) tetrahydrofuran-3-yl Phosphonate

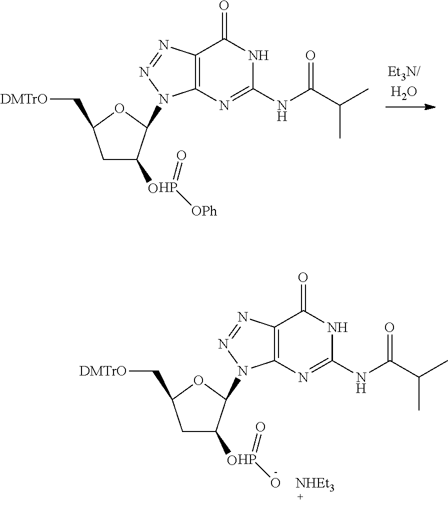

To the reaction mixture from Step 1 was added water (0.2 ml) and Et₃N (0.2 mL). The mixture was stirred at RT for 20 min. Then, it was concentrated, and the residue was partitioned between DCM (150 mL) and aq NaHCO₃ (2%, 30 mL). The layers were separated, and the organic layer was washed with aq NaHCO₃ (2%, 2×30 mL) and water (30 mL), dried (Na₂SO₄) and concentrated. It was purified by chromatography on silica gel eluted with 0 to 10% MeOH in DCM (containing 0.2% of Et₃N) to give the product. LCMS (ES, m/z): 703.0 [M−H]⁻. ¹H-NMR (400 MHz, DMSO-d₆): δ 7.38-7.28 (m, 2H), 7.25-7.13 (m, 7.5H), 6.86-6.66 (m, 4H), 6.35 (d, J=6.4 Hz, 1H), 5.73 (s, 0.5H), 5.17-5.01 (m, 1H), 4.41 (q, J=9.9, 8.4 Hz, 1H), 3.71 (d, J=0.8 Hz, 6H), 3.49 (t, J=9.1 Hz, 1H), 3.04 (dd, J=10.1, 3.2 Hz, 1H), 2.90 (q, J=7.3 Hz, 9H), 2.79 (p, J=6.8 Hz, 1H), 2.36 (dd, J=13.6, 7.2 Hz, 2H), 1.19-1.10 (m, 21H). ³¹P-NMR: (162 MHz, DMSO-d₆): δ −0.50 (s).

### Step 3: pyridin-1-ium (2R,3S,5S)-5-(hydroxymethyl)-2-(5-isobutyramido-7-oxo-6,7-dihydro-3H-[1,2,3]triazolo[4,5-d]pyrimidin-3-yl)tetrahydrofuran-3-yl Phosphonate

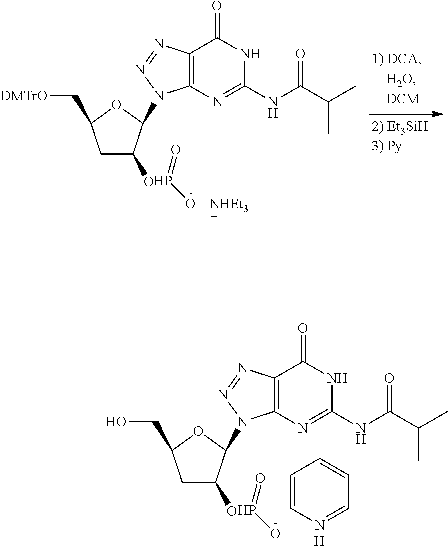

To a stirred solution of the product of Step 2 (460 mg, 0.571 mmol) in DCM (9 ml) at RT was added water (103 mg, 5.71 mmol) and 2,2-dichloroacetic acid in DCM (6%, 8.6 mL, 5.1 mmol). After 10 min, Et₃SiH (12 mL) was added, and it was stirred at RT for 1 h. Then, Py (1 g) was added. It was concentrated to give a crude product. LCMS (ES, m/z): 401.1 [M−H]⁻.

### Step 4: Pyridin-1-ium (2R,3S,5S)-5-((((((2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydrofuran-3-yl)oxy)(2-cyanoethoxy)phosphanyl)oxy)methyl)-2-(5-isobutyramido-7-oxo-6,7-dihydro-3H-[1,2,3]triazolo[4,5-d]pyrimidin-3-yl)tetrahydrofuran-3-yl Phosphonate

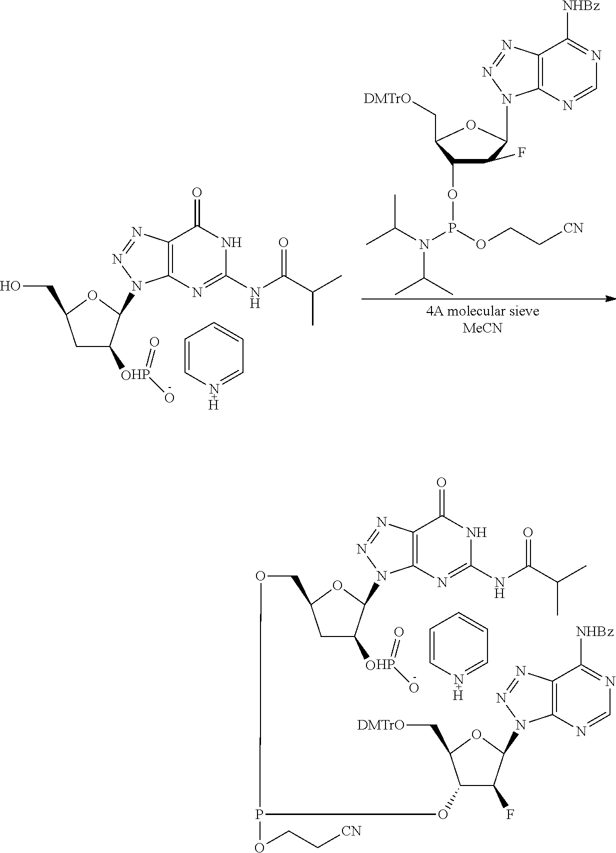

(2R,3R,4R,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydrofuran-3-yl (2-cyanoethyl) diisopropylphosphoramidite (0.579 g, 0.660 mmol) was co-evaporated with ACN (3×2 mL) and dissolved in ACN (3 mL). Activated 4 Å molecular sieves (200 mg) were added to the solution. The crude product from Step 3 (0.3 g, ˜0.6 mmol) was co-evaporated with ACN (3×2 mL) and dissolved in ACN (3 mL). Activated 4 Å molecular sieves (200 mg) were added to the solution. After 30 min, to the solution under Ar was added the solution containing (2R,3R,4R,5R)-5-(7-benzamido-3H-[1,2,3]triazolo[4,5-d]pyrimidin-3-yl)-2-((bis(4-methoxyphenyl)(phenyl)methoxy) methyl)-4-fluorotetrahydrofuran-3-yl (2-cyanoethyl) diisopropylphosphoramidite, and it was stirred at RT for 30 min. The reaction mixture was used in the next reaction step without purification. LCMS (ES, m/z): 1178.4 [M+H]⁺.

### Step 5: pyridin-1-ium (2R,3S,5S)-5-((((((2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydrofuran-3-yl)oxy)(2-cyanoethoxy)phosphorothioyl)oxy)methyl)-2-(5-isobutyramido-7-oxo-6,7-dihydro-3H-[1,2,3]triazolo[4,5-d]pyrimidin-3-yl)tetrahydrofuran-3-yl Phosphonate

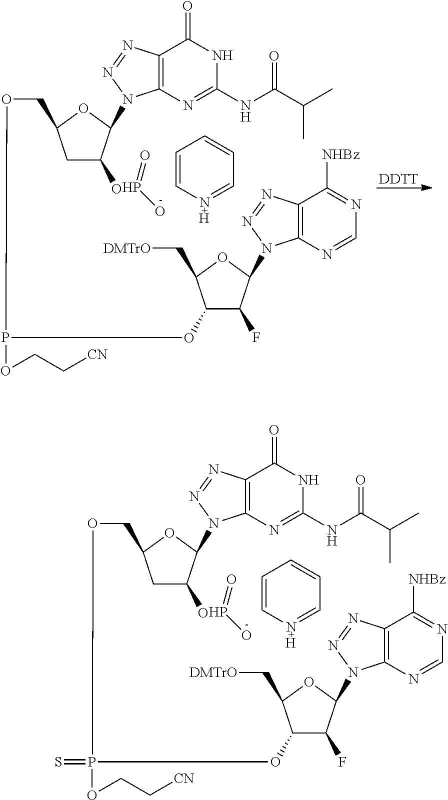

To the reaction mixture from Step 4 at RT was added (E)-N,N-dimethyl-N-(3-thioxo-3H-1,2,4-dithiazol-5-yl)formimidamide (0.136 g, 0.660 mmol), and it was stirred for 1 h. Then, the mixture was concentrated to give a crude product, which was used for the next reaction step directly without further purification. LCMS (ES, m/z): 1210.3 [M+H]⁺.

### Step 6: ammonium (2R,3S,5S)-5-((((((2R,3R,4S,5R)-5-(7-benzamido-3H-[1,2,3]triazolo[4,5-d]pyrimidin-3-yl)-4-fluoro-2-(hydroxymethyl)tetrahydrofuran-3-yl)oxy)(2-cyanoethoxy)phosphorothioyl)oxy)methyl)-2-(5-isobutyramido-7-oxo-6,7-dihydro-3H-[1,2,3]triazolo[4,5-d]pyrimidin-3-yl)tetrahydrofuran-3-yl Phosphonate

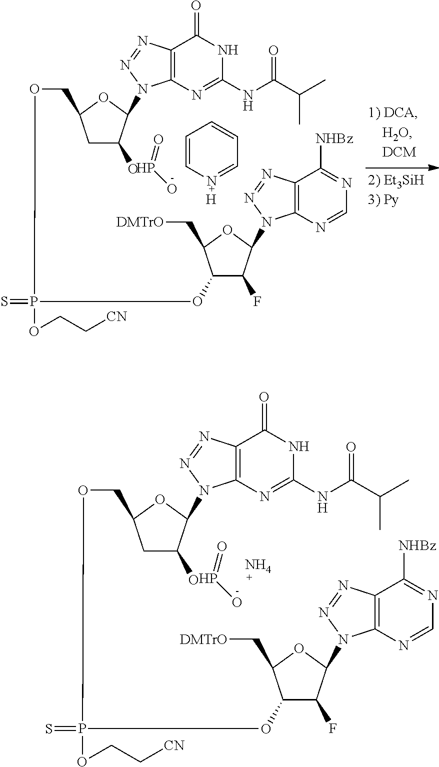

To the crude from Step 5 in DCM (6 mL) at RT was added water (0.11 g, 6.0 mmol) and 2,2-dichloroacetic acid in DCM (0.6M, 9.0 ml, 5.4 mmol). After stirring for 10 min, Et₃SiH (4 mL) was added, and it was stirred for 1 h. Then, Py (0.8 g) was added to the reaction. After 5 min, it was concentrated, and the crude was purified by reverse phase chromatography (C18) eluted with 0 to 95% ACN in aq NH₄HCO₃ (5 mM) to give the product. LCMS (ES, m/z): 908.2 [M+H]⁺. ¹H-NMR: (300 MHz, MeOH-d₄) δ 8.84 (d, J=3.2 Hz, 1H), 8.09 (t, J=7.0 Hz, 2H), 7.72-7.51 (m, 4H), 6.94 (d, J=5.6 Hz, 1H), 6.49 (t, J=6.1 Hz, 1H), 5.99-5.61 (m, 2H), 5.29-5.09 (m, 1H), 4.68-4.49 (m, 2H), 4.44-4.12 (m, 4H), 4.04-3.78 (m, 2H), 3.16 (t, J=7.3 Hz, 1H), 2.87-2.83 (m, 2H), 2.75-2.48 (m, 3H), 1.34-1.08 (m, 6H). ³¹P-NMR: (121 MHz, Methanol-d₄) δ 67.52-67.37 (m), 2.57-2.54 (m).

### Step 7: Pyridinium (5S,7R,8S,12aR,14R,15S,15aR)-2-(2-cyanoethoxy)-15-fluoro-7-{5-[(2-methylpropanoyl)amino]-7-oxo-6,7-dihydro-3H-[1,2,3]triazolo[4,5-d]pyrimidin-3-yl}-14-{7-[(phenylcarbonyl)amino]-3H-[1,2,3]triazolo[4,5-d]pyrimidin-3-yl}octahydro-12H-5,8-methanofuro[3,2-1][1,3,6,9,11,2,10]pentaoxadiphosphacyclotetradecin-10-olate 2-sulfide

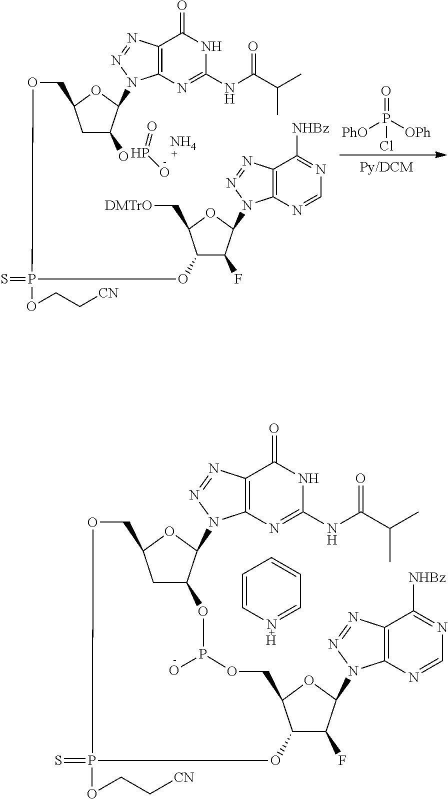

To Py (16 mL) at −40° C. under Ar was added diphenyl phosphorochloridate (1.3 g, 5.0 mmol), and a solution of the product of Step 6 (0.23 g, 0.25 mmol) in Py (9 mL) in DCM (25 ml) dropwise over 30 min. The resulting mixture was stirred at −40° C. to −20° C. for 40 min. The solution containing the product was used for the next step directly. LCMS (ES, m/z): [M+H]⁺ 889.2.

### Step 8: ammonium (5S,7R,8S,12aR,14R,15S,15aR)-2-(2-cyanoethoxy)-15-fluoro-7-{5-[(2-methylpropanoyl)amino]-7-oxo-6,7-dihydro-3H-[1,2,3]triazolo[4,5-d]pyrimidin-3-yl}-14-(7-[(phenylcarbonyl)amino]-3H-[1,2,3]triazolo[4,5-d]pyrimidin-3-yl)octahydro-12H-5,8-methanofuro[3.2-1][1,3,6,9,11,2,10]pentaoxadiphosphacyclotetradecine-10-thiolate 10-oxide 2-sulfide

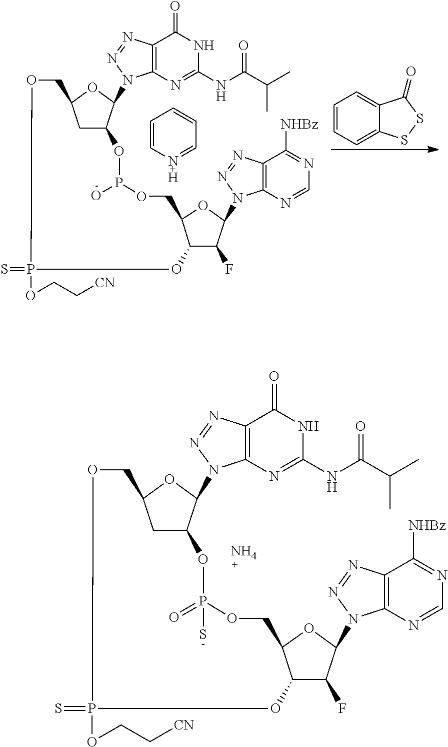

To the reaction mixture from Step 7 at −40° C. was added 3H-benzo[c][1,2]dithiol-3-one (0.063 g, 0.38 mmol) and water (0.09 ml, 5 mmol). It was stirred at RT for 40 min. Then, the solution was concentrated. The crude was purified by reverse phase chromatography (C18) eluted with 0 to 95% ACN in aq NH₄HCO₃ (5 mM) to give the product. LCMS (ES, m/z): 922.2 [M+H]⁺. ³¹P-NMR: (121 MHz, MeOH-d₄) δ 68.42-55.92 (m).

### Step 9: 5-amino-3-[(5S,7R,8S,12aR,14S,15S,15aR)-14-(7-amino-3H-[1,2,3]triazolo[4,5-d]pyrimidin-3-yl)-15-fluoro-2,10-dioxido-2,10-disulfanyloctahydro-12H-5,8-methanofuro[3,2-1][1,3,6,9,11,2,10]pentaoxadiphosphacyclotetradecin-7-yl]-3,6-dihydro-7H-[1,2,3]triazolo[4,5-d]pyrimidin-7-one (Diastereomer 1-2)

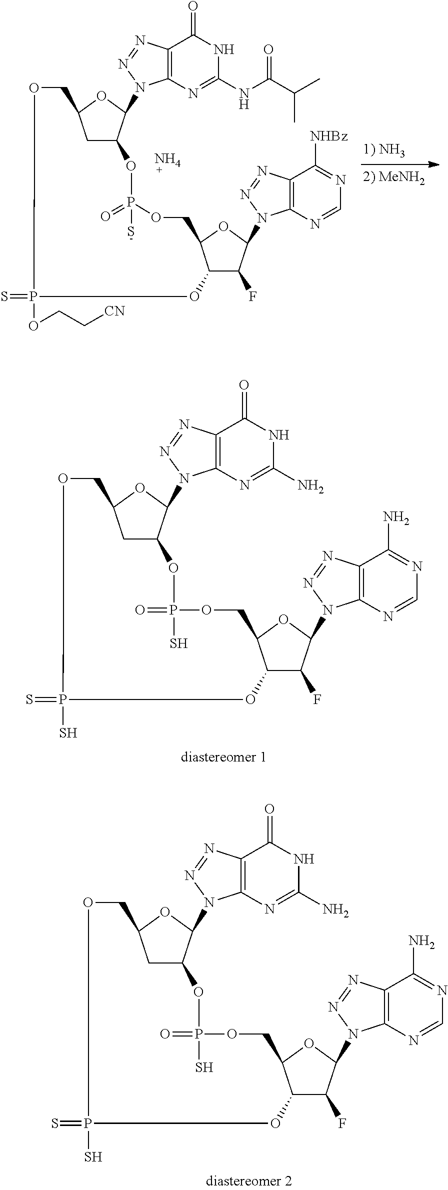

To a tube at −60° C. was added the product of Step 8 (200 mg, 0.213 mmol) and NH₃ in i-PrOH (50% by volume, 10 ml). The tube was tightly sealed and heated at 50° C. for 3 h. Then, the reaction mixture was cooled, and it was concentrated. The residue was dissolved in MeNH₂ in EtOH (30%, 5 ml), and it was stirred at RT for 1 h. It was concentrated and purified by reverse phase chromatography (C18) eluted with 0 to 12% ACN in aq NH₄HCO₃ (30 mM) to give a crude product.

Faster fractions were further purified by reverse phase chromatography (Xbridge Prep C18 OBD) eluted with 2 to 12% ACN in aq NH₄HCO₃ (20 mM) over 16 min to give the product. Example 20 (T_{R}=10.07 min, diastereomer 1): LCMS (ES, m/z): 695.0 [M+H]⁺. ¹H-NMR: (300 MHz, D₂O) δ 8.26 (s, 1H), 6.71 (dd, J=8.6, 5.2 Hz, 1H), 6.35 (dd, J=3.9, 1.5 Hz, 1H), 5.85-5.40 (m, 2H), 5.01 (dt, J=0.8, 4.9 Hz, 1H), 4.45 (d, J=5.2 Hz, 1H), 4.36-4.18 (m, 2H), 4.13-4.05 (m, 3H), 2.73-2.54 (m, 1H), 2.48 (d, J=4.3 Hz, 1H). ³¹P-NMR: (121 MHz, D₂O) δ 55.67 (s), 55.39 (s).

Slower fractions were further purified by reverse phase chromatography (Xbridge Prep C18 OBD) eluted with 2 to 12% ACN in aq NH₄HCO₃ (20 mM) over 16 min to give the product. Example 21 (T_{R}=15.75 min, diastereomer 2): LCMS (ES, m/z): 695.0 [M+H]⁺. ¹H-NMR: (300 MHz, D₂O) δ 8.30 (s, 1H), 6.75 (t, J=6.2 Hz, 1H), 6.40 (d, J=4.0 Hz, 1H), 5.82 (t, J=5.5 Hz, 1H), 5.65 (t, J=5.5 Hz, 1H), 5.48-5.21 (m, 1H), 4.57-4.38 (m, 2H), 4.35-4.23 (m, 2H), 4.11 (s, 2H), 2.55 (d, J=14.2 Hz, 2H). ³¹P-NMR: (121 MHz, D₂O) δ 55.31 (s), 53.84 (s).

Examples 22 and 23, as shown in Table 4 below, were prepared according to procedures analogous to those outlined in Examples 20 and 21 above using appropriate monomers, described as Preparations or as obtained from commercial sources, in the coupling step.

TABLE 4

| | | | Mass |
|---|---|---|---|
| Ex. | Structure | Name | [M + H]⁺ |
| 22 | 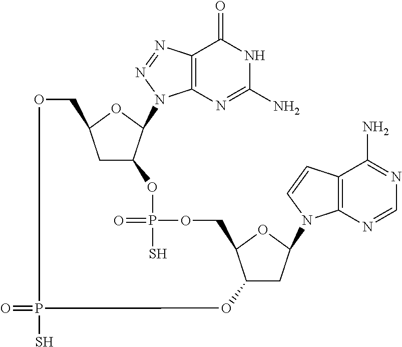 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2C3)c(=O)[nH]1] | 5-amino-3-[(5S,7R,8S,12aR,14R,15aS)-14-(4- amino-7H-pyrrolo[2,3-d]pyrimidin-7-yl)-2,10- dioxido-2,10-disulfanyloctahydro-12H-5,8- methanofuro[3,2-l][1,3,6,9,11,2,10]penta- oxadiphosphacyclotetradecin-7-yl]-3,6-dihydro- 7H-[1,2,3]triazolo[4,5-d]pyrimidin-7-one (diastereomer 1) | 675 |
| 23 | 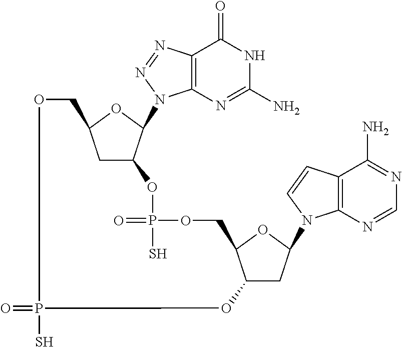 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2C3)c(=O)[nH]1] | 5-amino-3-[(5S,7R,8S,12aR,14R,15aS)-14-(4- amino-7H-pyrrolo[2,3-d]pyrimidin-7-yl)-2,10- dioxido-2,10-disulfanyloctahydro-12H-5,8- methanofuro[3,2-l][1,3,6,9,11,2,10]penta- oxadiphosphacyclotetradecin-7-yl]-3,6-dihydro- 7H-[1,2,3]triazolo[4,5-d]pyrimidin-7-one (diastereomer 2) | 675 |

### Example 24: 5-amino-3-[(5R,7R,8R,12aR,14R,15S,15aR,16R)-14-(7-amino-3H-[1,2,3]triazolo[4,5-d]pyrimidin-3-yl)-15,16-difluoro-2,10-dioxido-2,10-disulfanyloctahydro-12H-5,8-methanofuro[3,2-1][1,3,6,9,11,2,10]pentaoxadiphosphacyclotetradecin-7-yl]-3,6-dihydro-7H-[1,2,3]triazolo[4,5-d]pyrimidin-7-one

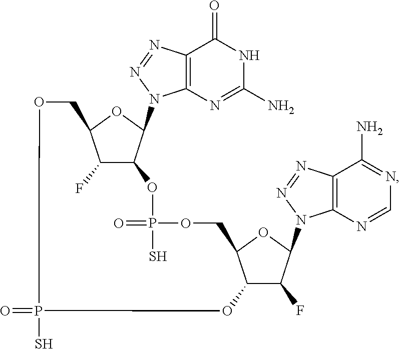 [SMILES: Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3F)c(=O)[nH]1]

### Step 1: (2R,3R,4R,5R)-5-((bis(4-methoxyphenyl)(phenyl)methoxy)methyl)-4-fluoro-2-(5-isobutyramido-7-oxo-6,7-dihydro-3H-[1,2,3]triazolo[4,5-d]pyrimidin-3-yl)tetrahydrofuran-3-yl phenyl Phosphonate

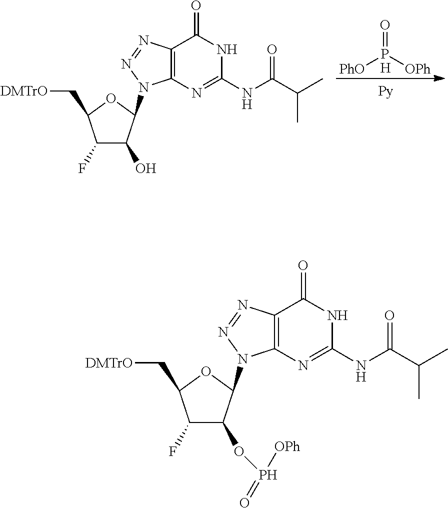

The compound of Preparation 4 (0.527 g, 0.801 mmol) was co-evaporated with Py (3×3 mL) and dissolved in Py (4 ml). To the mixture at RT under Ar was added diphenyl phosphonate (0.562 g, 2.40 mmol), and it was stirred for 20 min. The reaction solution was used for the next step directly. LCMS (ES, m/z): 797.3 [M−H]⁻.

### Step 2: Ammonium (2R,3R,4R,5R)-5-((bis(4-methoxyphenyl)(phenyl)methoxy) methyl)-4-fluoro-2-(5-isobutyramido-7-oxo-6,7-dihydro-3H [1,2,3]triazolo[4,5-d]pyrimidin-3-yl)tetrahydrofuran-3-yl Phosphonate

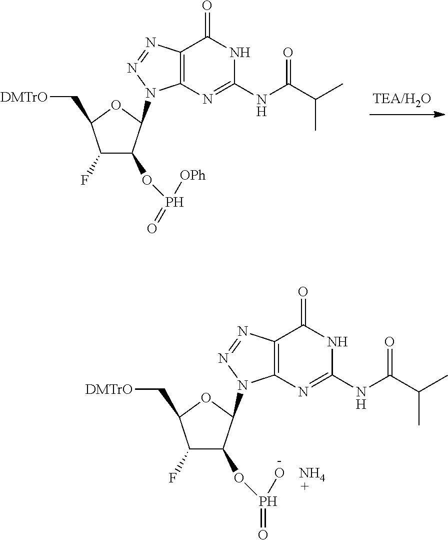

To the reaction mixture from Step 1 at 0° C. was added water (2 ml) and TEA (2 mL). The mixture was stirred at RT for 20 min. Then, it was concentrated and purified by reverse phase chromatography (AQ C18) eluted with 0 to 95% ACN in aq NH₄HCO₃ (5 mM) to give the product. LCMS (ES, m/z): 721.0 [M−H]⁻. ¹H-NMR (300 MHz, DMSO-d₆) δ 7.36 (s, 0.5H), 7.29-7.20 (m, 2H), 7.18-7.08 (m, 9H), 6.74-6.69 (m, 2H), 6.68-6.62 (m, 2H), 6.46 (d, J=7.2 Hz, 1H), 5.78 (t, J=7.2 Hz, 1H), 5.58 (t, J=6.9 Hz, 0.5H), 5.40 (s, 0.5H), 5.29 (ddt, J=24.2, 10.2, 7.3 Hz, 1H), 4.30 (dt, J=19.0, 9.2 Hz, 1H), 3.67 (s, 3H), 3.65 (s, 3H), 3.52 (t, J=9.6 Hz, 1H), 3.20-3.10 (m, 1H), 2.68 (p, J=6.9 Hz, 1H), 1.07 (dd, J=6.9, 2.2 Hz, 6H). ³¹P-NMR: (121 MHz, DMSO-d₆) δ 0.67 (s).

### Step 3: Pyridin-1-ium (2R,3R,4R,5R)-4-fluoro-5-(hydroxymethyl)-2-(5-isobutyramido-7-oxo-6,7-dihydro-3H-[1,2,3]triazolo[4,5-d]pyrimidin-3-yl)tetrahydrofuran-3-yl Phosphonate

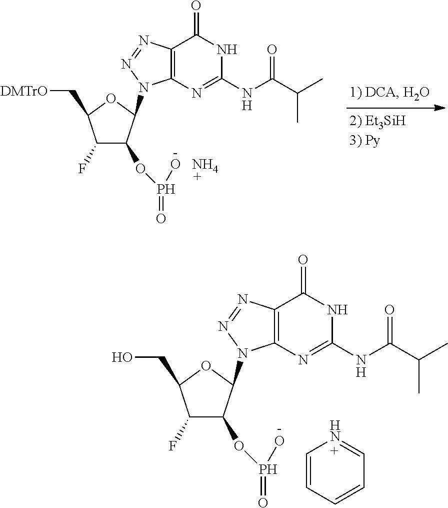

To a stirred solution of the product of Step 2 (460 mg, 0.622 mmol) in DCM (6 ml) at RT was added water (112 mg, 6.22 mmol) and 2,2-dichloroacetic acid in DCM (0.60M, 9.3 mL, 5.6 mmol). After 10 min, Et₃SiH (2 mL) was added, and it was stirred at RT for 1 h. Then, Py (0.9 g) was added. After 5 min, it was concentrated to give a crude product. LCMS (ES, m/z): 421.1 [M−H]⁻.

### Step 4: Pyridin-1-ium (2R,3R,4R,5R)-5-((((((2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydro-furan-3-yl)oxy) Q-cyanoethoxy)phosphanyl)oxymethyl)-4-fluoro-2-(5-isobutyramido-7-oxo-6,7-dihydro-3H-[1,2,3]triazolo[4,5-d]pyrimidin-3-yl)tetrahydrofuran-3-yl Phosphonate

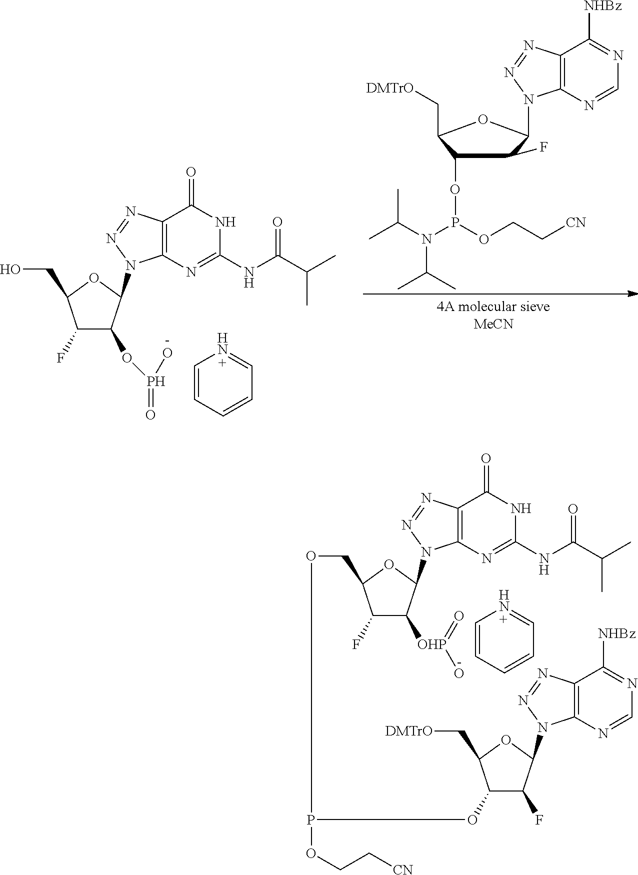

(2R,3R,4R,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydrofuran-3-yl (2-cyanoethyl) diisopropylphosphoramidite (0.545 g, 0.622 mmol) was co-evaporated with ACN (3×2 mL) and dissolved in ACN (3 mL). Activated 4 Å molecular sieves (50 mg) were added to the solution. The crude product from Step 3 was co-evaporated with ACN (3×2 mL) and dissolved in ACN (3 mL). Activated 4 Å molecular sieves (50 mg) were added to the solution. After 30 min, to the solution under Ar was added the solution containing (2R,3R,4R,5R)-5-(7-benzamido-3H-[1,2,3]triazolo[4,5-d]pyrimidin-3-yl)-2-((bis(4-methoxyphenyl)(phenyl) methoxy)methyl)-4-fluorotetrahydro-furan-3-yl (2-cyanoethyl) diisopropylphosphoramidite, and it was stirred at RT for 30 min. The reaction mixture was used in the next reaction step without purification. LCMS (ES, m/z): 1196.3 [M+H]⁺.

### Step 5: Pyridin-1-ium (2R,3R,4R,5R)-5-((((((2R,3R,4S,5R)-5-(7-benzamido-3H-[1,2,3]triazolo[4,5-d]pyrimidin-3-yl)-2-((bis(4-methoxyphenyl)(phenyl)methoxy)methyl)-4-fluorotetrahydro-furan-3-yl)oxyl(2-cyanoethoxy)phosphorothioyl)oxymethyl)-4-fluoro-2-(5-isobutyramido-7-oxo-6,7-dihydro-3H-[1,2,3]triazolo[4,5-d]pyrimidin-3-yl)tetrahydrofuran-3-yl Phosphonate

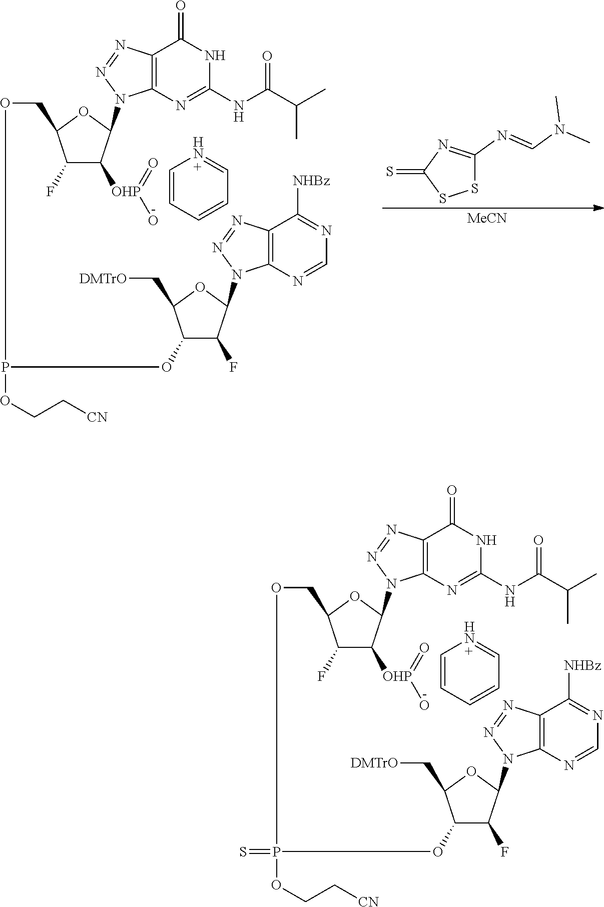

To the reaction mixture from Step 4 at RT was added (E)-N,N-dimethyl-N-(3-thioxo-3H-1,2,4-dithiazol-5-yl)formimidamide (0.140 g, 0.684 mmol), and it was stirred for 1 h. Then, the mixture was concentrated to give a crude product, which was used for the next step directly without further purification. LCMS (ES, m/z): 1226.3 [M−H]⁻.

### Step 6: ammonium (2R,3R,4R,5R)-5-((((((2R,3R,4S,5R)-5-(7-benzamido-3H-[1,2,3]triazolo[4,5-d]pyrimidin-3-yl)-4-fluoro-2-(hydroxymethyl)tetrahydrofuran-3-yl)oxy)(2-cyanoethoxy) phosphorothioyl)oxy)methyl)-4-fluoro-2-(5-isobutyramido-7-oxo-6,7-dihydro-3H-[1,2,3]triazolo[4,5-d]pyrimidin-3-yl)tetrahydrofuran-3-yl Phosphonate

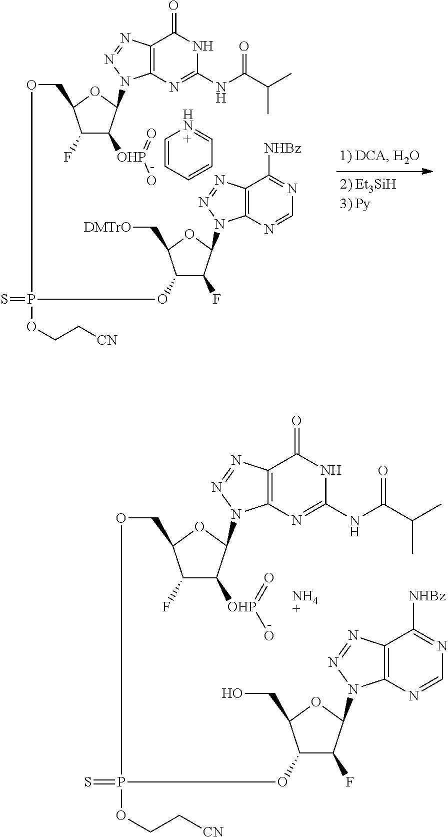

To the crude from Step 5 in DCM (6 mL) at RT was added water (0.11 g, 6.2 mmol) and 2,2-dichloroacetic acid in DCM (0.6M, 9.0 ml, 5.4 mmol). After stirring for 10 min, Et₃SiH (2 mL) was added, and it was stirred for 1 h. Then, Py (0.89 g, 11 mmol) was added to the reaction. After 5 min, it was concentrated, and the crude was purified by reverse phase chromatography (C18) eluted with 0 to 95% ACN in aq NH₄HCO₃ (5 mM) to give the product. LCMS (ES, m/z): 926.2 [M+H]⁺. ¹H-NMR: (300 MHz, MeOH-d₄) δ 8.84 (d, J=1.2 Hz, 1H), 8.09 (td, J=8.0, 7.5, 1.7 Hz, 2H), 7.70-7.56 (m, 3.5H), 6.96 (td, J=5.9, 4.1 Hz, 1H), 6.62 (t, J=6.5 Hz, 1H), 5.99-5.62 (m, 3H), 5.57-5.46 (m, 1.5H), 4.54-4.47 (m, 3H), 4.27-4.22 (m, 3H), 3.90-3.86 (m, 2H), 2.83 (dt, J=11.3, 5.9 Hz, 2H), 2.73-2.60 (m, 1H), 1.24-1.10 (m, 6H). ³¹P NMR: (121 MHz, MeOH-d₄) δ 67.75 67.68 (m), 3.02-2.46 (m).

### Step 7: pyridinium (5R,7R,8R,12aR,14R,15S,15aR,16R)-2-(2-cyanoethoxy)-15,16-difluoro-7-{5-[(2-methylpropanoyl)amino]-7-oxo-6,7-dihydro-3H-[1,2,3]triazolo[4,5-d]pyrimidin-3-yl}-14-(7-[(phenylcarbonyl)amino]-3H-[1,2,3]triazolo[4,5-d]pyrimidin-3-yl)octahydro-12H-5,8-methanofuro[3.2-1][1,3,6,9,11,2,10]pentaoxadiphosphacyclotetradecin-10-olate 2-sulfide

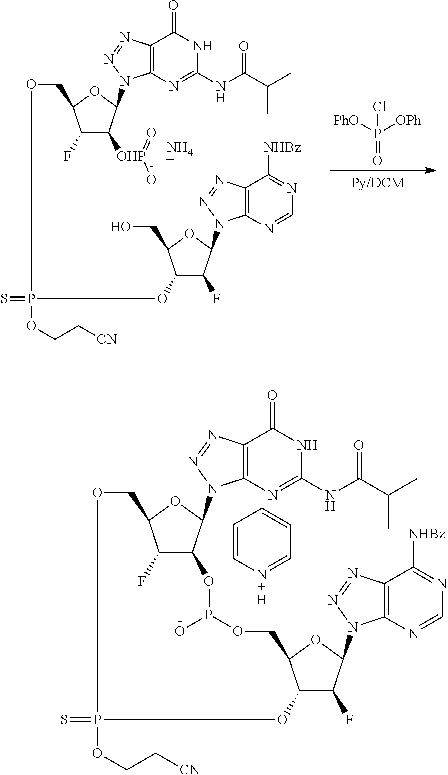

To Py (30 mL) at −40° C. under Ar was added diphenyl phosphorochloridate (2.05 g, 7.64 mmol), and a solution of the product of Step 6 (0.36 g, 0.38 mmol) in Py (9 mL) in DCM (25 ml) dropwise over 30 min. The resulting mixture was stirred at −40° C. for 40 min. The solution containing the product was used for the next reaction step directly.

### Step 8: ammonium (5R,7R,8R,12aR,14R,15S,15aR,16R)-2-(2-cyanoethoxy)-15,16-difluoro-7-{5-[(2-methylpropanoyl)amino]-7-oxo-6,7-dihydro-3H-[1,2,3]triazolo[4,5-d]pyrimidin-3-yl}-14-{7-[(phenylcarbonyl)amino]-3H-[1,2,3]triazolo[4,5-d]pyrimidin-3-yl}octahydro-12H-5,8-methanofuro[3,2-1][1,3,6,9,11,2,10]pentaoxadiphosphacyclotetradecine-10-thiolate 10-oxide 2-sulfide

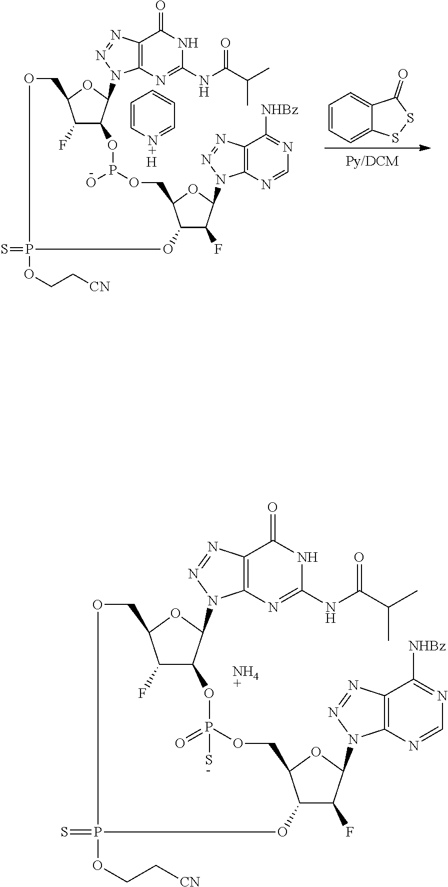

To the reaction mixture from Step 7 at −40° C. was added 3H-benzo[c][1,2]dithiol-3-one (96 mg, 0.57 mmol) and water (0.14 ml, 7.6 mmol). It was stirred at RT for 40 min. Then, the solution was concentrated. The crude was purified by reverse phase chromatography (C18) eluted with 0 to 95% ACN in aq NH₄HCO₃ (5 mM) to give the product. LCMS (ES, m/z): 940.1 [M+H]⁺. ³¹P-NMR: (121 MHz, MeOH-d₄) δ 68.91-56.85 (m).

### Step 9: 5-amino-3-[(5R,7R,8R,12aR,14R,15S,15aR,16R)-14-(7-amino-3H-[1,2,3]triazolo[4,5-d]pyrimidin-3-yl)-15,16-difluoro-2,10-dioxido-2,10-disulfanyloctahydro-12H-5,8-methanofuro[3,2-1][1,3,6,9,11,2,10]pentaoxadiphosphacyclotetradecin-7-yl]-3,6-dihydro-7H-triazolo[4,5-d]pyrimidin-7-one

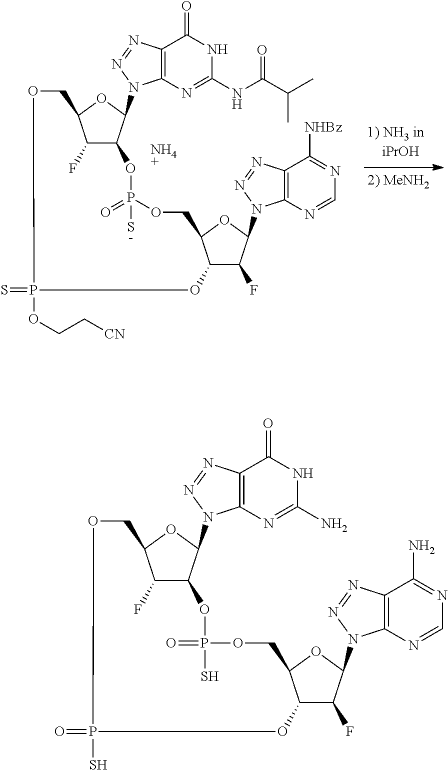

To a tube at −60° C. was added the product of Step 8 (280 mg, 0.293 mmol) and NH₃ in i-PrOH (50%, 10 ml). The tube was tightly sealed and heated at 50° C. for 3 h. Then, the reaction mixture was cooled, and it was concentrated. The residue was dissolved in MeNH₂ in EtOH (30%, 10 ml), and it was stirred at RT for 1 h. It was concentrated and purified by reverse phase chromatography (AQ C18) eluted with 0 to 15% ACN in aq NH₄HCO₃ (30 mM) over 40 min to give a crude product.

Fractions around T_{R}=32.2 min were further purified by reverse phase chromatography (AQ C18) eluted with 0 to 15% ACN in aq NH₄HCO₃ (30 mM) over 40 min to give the product. Example 24 (T_{R}=28.1 min): LCMS (ES, m/z): 713.0 [M+H]⁺. ¹H-NMR: (300 MHz, D₂O) δ 8.23 (s, 1H), 6.75 (dd, J=0.6, 4.9 Hz, 1H), 6.32 (d, J=8.6 Hz, 1H), 5.99-5.77 (m, 2H), 5.74-5.59 (m, 1H), 5.46 (dd, J=53.3, 3.5 Hz, 1H), 4.75 (d, J=8.3 Hz, 1H), 4.46-4.40 (m, 2H), 4.21-4.06 (m, 3H). ³¹P-NMR: (121 MHz, D₂O) δ 54.52 (s), 53.45 (s).

Examples 25 and 26, as shown in Table 5 below, were prepared according to procedures analogous to those outlined in Example 24 above using appropriate monomers, described as Preparations or as obtained from commercial sources, in the coupling step.

TABLE 5

| | | | Mass |
|---|---|---|---|
| Ex. | Structure | Name | [M + H]⁺ |
| 25 | 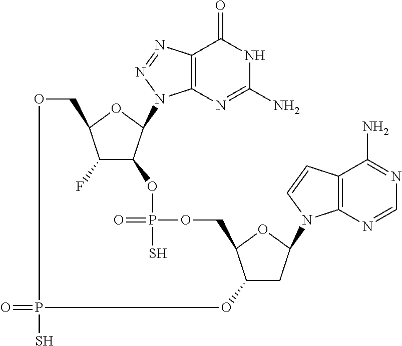 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2[C@@H]3F)c(=O)[nH]1] | 5-amino-3-[(5R,7R,8R,12aR,14R,15aS,16R)- 14-(4-amino-7H-pyrrolo[2,3-d]pyrimidin-7-yl)- 16-fluoro-2,10-dioxido-2,10- disulfanyloctahydro-12H-5,8-methanofuro[3,2- l][1,3,6,9,11,2,10]penta- oxadiphosphacyclotetradecin-7-yl]-3,6-dihydro- 7H-[1,2,3]triazolo[4,5-d]pyrimidin-7-one (diastereomer 1) | 693 |
| 26 | 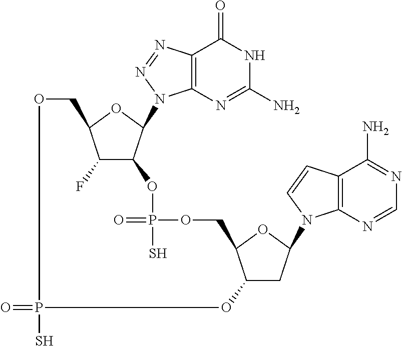 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2[C@@H]3F)c(=O)[nH]1] | 5-amino-3-[(5R,7R,8R,12aR,14R,15aS,16R)- 14-(4-amino-7H-pyrrolo[2,3-d]pyrimidin-7-yl)- 16-fluoro-2,10-dioxido-2,10- disulfanyloctahydro-12H-5,8-methanofuro[3,2- l][1,3,6,9,11,2,10]penta- oxadiphosphacyclotetradecin-7-yl]-3,6-dihydro- 7H-[1,2,3]triazolo[4,5-d]pyrimidin-7-one (diastereomer 2) | 693 |

### Biological Evaluation

The individual compounds described in the Examples herein are defined as STING agonists by (i) binding to the STING protein as evidenced by a reduction in binding of tritiated cGAMP ligand to the STING protein by at least 20% at 20 uM (concentration of compound being tested) in a STING Biochemical [3H]cGAMP Competition Assay and (ii) demonstrating interferon production with a 6% or greater induction of IFN-β secretion at 30 uM in the THP1 cell assay (where induction caused by cGAMP at 30 uM was set at 100%).

### [³H]-cGAMP Synthesis

2.3 mL of buffer solution containing 80 mM TrisCl, 200 mM MgCl₂, and 20 mM NaCl followed by 0.32 mL of 10 mM aq solution of GTP (guanosine 5′-triphosphate) was added to a plastic 50 mL AMICON tube. A solution of [³H]ATP (Adenosine 5′-triphosphate, 21 Ci/mmol, 45 mCi) in 0.5 mL H₂O was then added followed by 1 mL of 1 mg/mL solution of DNA (Herring testes activator DNA, Sigma, #D6898) and 53 uL of 47 mM solution of cGAS enzyme. Additional H₂O was added to bring the total volume to 10 mL.

The reaction was stirred for 2 h at 37° C. and then added directly to an Amicon Ultra-15 10K centrifuge tube and spun for 1 h at 4,000 g. The collected solution was then purified on a semi-prep Mono Q column using the following mobile phases:

A: 0.05M TrisCl pH 8.5 adjusted with 1M NaOH

B: 0.05M TrisCl, 0.5M NaCl pH 8.5 adjusted with 1M NaOH

Gradient: 100% A for 5 min followed by a linear gradient to 50:50 (A:B) over 25 min, 3 mL/min, 254 nm.

The collected product fractions were pooled and adjusted to a total volume of 30 mL with buffer A. A total yield of 15.5 mCi of [³H]cGAMP was isolated at a radiochemical purity of 98.0% at a specific activity of 21.5 Ci/mmol.

cGAS Enzyme

A recombinant DNA vector was chemically synthesized to express the truncated human cGAS enzyme (residues 161-522). To aid in expression and purification, the amino terminus contains a hexahistidine tag, SUMO tag and TEV cleavage site. The recombinant enzyme was overexpressed in ROSETTA™ 2(DE3) Single Competent Cells (Novagen). Affinity purification was carried out using HIS-Select HF Nickel Affinity Gel (Sigma) followed by size exclusion chromatography using a Hi-Load 26/60 SUPERDEX200 prep grade column (GE Healthcare). Fractions were pooled, concentrated, flash-frozen in liquid nitrogen and stored at −80° C. until needed.

### 3H-cGAMP Filtration Binding Assay (HAQ STING)

The ability of compounds to bind STING is quantified by their ability to compete with tritiated cGAMP ligand for human STING receptor membrane using a radioactive filter-binding assay. The binding assay employs STING receptor obtained from Trichoplusia ni cell membranes (T.ni; Expression Systems, cat #94-002F, www.expressionsystems.com) overexpressing full-length HAQ STING and tritiated cGAMP ligand.

The basic HAQ STING filtration assay protocol is as follows:

The compounds were serially titrated by the Hamilton STARPlus CORE in a 96-well plate (Greiner, #651201) using a 1:3 ten-point dose response format. After compound preparation, a 2.2 ug/ml working concentration of STING membrane (SEQ. ID. No. 1) was prepared by diluting concentrated membrane into assay buffer (lx PBS; Invitrogen #SH30028.02) and douncing 7× using a manual tissue homogenizer (Wheaton, #357546). 148 uL of prepared membrane was then manually added to each well of a 96-well deep-well polypropylene plate (Fisher Scientific, #12-566-121). Following membrane addition, 2 uL of either titrated test compound, DMSO control (Sigma #276855), or cold cGAMP control was added to the appropriate wells using a BIOMEK FX. Compound and membrane then preincubated for 60 min at RT to allow compound binding to equilibrate. Following equilibration, 8 nM of [³H]c-GAMP ligand was prepared by diluting into assay buffer, and 50 uL of this working stock was then manually added to each well of the assay plate. Plates were then incubated at RT for 60 min, and the contents of each assay plate were then filtered through a 96-well GF/B filter plate (PerkinElmer, #6005250) using a TOMTEC MACH III Cell Harvester equipped with 20 mM HEPES buffer (Fisher Scientific, #BP299500). The filter plates were then dried at 55° C. for 30 min using a pressurized oven before 30 uL of ULTIMA GOLD F scintillate was added to each well. Tritium levels for each reaction well were then measured using a PerkinElmer TopCount plate reader.

After normalization to controls, the percent activity for each compound concentration was calculated by measuring the amount of remaining radioactivity. The plot of percent activity versus the log of compound concentration was fit with a 4-parameter dose response equation to calculate EC₅₀ values.

The final reaction conditions were:

| Component | Volume (uL) | Final Concentration | |
|---|---|---|---|
| STING membrane | 148 | 1.5 | ug/ml |
| ³H-cGAMP | 50 | 2.0 | nM |
| Low Control (cold cGAMP) | 2 | 10 | uM |
| Test compound/DMSO | 2 | 10 | uM |

Compound concentrations tested were 20.000, 637.00, 2.200, 0.740, 0.247, 0.082, 0.027, 0.009, 0.003, and 0.001 μM with 1.0% residual DMSO.

### Full-Length STING (HAQ) Virus Generation

STING virus was generated using an insect cell baculovirus system. Spodoptera frugiperda Sf21 cells (Kempbio, Inc.) were diluted to 5e5 cells/ml in Sf-900II SFM media (LifeTechnologies #10902088) without antibiotics. The cell suspension was added to each well of a treated 6-well plate (2 mL per well, 1e6 cells total), and the cells were allowed to adhere for at least 30 min. Meanwhile, a 1 mL co-transfection mix was assembled by combining 500 ng of HAQ STING [STING(1-379)R71H,G230A,H232R,R293Q-GG-AviTag-GS-HRV3C-HIS8/pBAC1] DNA (Genewiz custom synthesis) with 1 mL Sf-900II SFM media containing 10 μL Cellfectin® II Reagent (Invitrogen #10362100) and 100 ng viral backbone BestBac 2.0, v-cath/chiA Deleted Linearized Baculovirus DNA (Expression Systems #91-002). The transfection mixtures were allowed to incubate for 30 min. After incubation, media was gently removed from the adhered cells in the 6-well plate, the 1 mL transfection mixtures were added (1 mL per well), and the plate was placed in a humidified incubator at 27° C. The following day, 1 mL Sf-900II SFM media (no antibiotics) was added to each well of the 6-well plate. After media addition, the cells were allowed to incubate with DNA (SEQ. ID. No. 2) at 27° C. for 5-7 days to generate the P0 viral stock. To generate P1 viral stocks, 0.5 mL of P0 viral supernatant was added to 50 mL uninfected Sf21 cells (seeded the day prior to infection at a density of 5×10⁵ cells/mL to allow for one overnight doubling) in Sf-900II SFM media containing 5 μg/mL gentamicin (Invitrogen #15710072). The infected cells were then incubated at 27° C. for 3 days while shaking at 110 rpm (ATR Biotech Multitron Infors HT #AJ118). On day 3, P1 cultures were counted using a ViCell XR (Beckman Coulter Life Sciences #383556) to confirm infection had occurred (cell size ≥3 μm larger than uninfected cells and viability approximately 85-95%). Cultures were harvested in 50 mL conical tubes and centrifuged at 2000×g for 10 min at 4° C. The P1 viral supernatants were poured off into clean 50 ml centrifuge tubes, and the remaining P1 cell pellets were used to generate Baculovirus Infected Insect Cells (BIICs). Cryopreservation media containing Sf-900II SFM media with 10% heat inactivated FBS, 10% DMSO (Sigma #D2650), and 5 μg/ml gentamicin was prepared and sterilized through 0.22 μM filter immediately prior to use. P1 cell pellets were resuspended to a density of 2e7 cells/ml and aliquoted into cryovials (1 mL per vial). Cryovials were placed in MR. FROSTY™ cell freezers O/N at −80° C. and transferred to liquid nitrogen for long term storage the following day. To generate P2 viral stock, 0.5 mL of the P1 viral supernatant was added to 50 mL uninfected Sf21 cells (seeded the day prior to infection at a density of 5×10⁵ cells/mL to allow for one overnight doubling) in Sf-900II SFM media containing 5 μg/mL gentamicin. These cells were incubated at 27° C. for 3 days while shaking at 110 rpm before harvesting P2 stock with centrifugation at 2000×g for 10 min at 4° C. The P2 viral supernatants were poured off and discarded, while the P2 cell pellets were used to generate P2 BIICs following the same protocol described above. The baculovirus generation protocol has been validated to consistently produce P1/P2 BIICs with titers of 2e9 pfu/mL (2e7 cells/mL×100 pfu/cell).

### Full-Length STING (HAQ) Expression

To generate STING membranes, P1/P2 BIICs were amplified overnight by adding thawed BIICs to Sf21 cells seeded at a density of 1.0×10⁶ cells/mL. The volume of BIIC used to infect the culture was calculated using an assumed BIIC titer of 2e9 pfu/ml to achieve an MOI of 10 in the overnight amplification. After culturing overnight, the cells were counted on a ViCell XR to confirm infection had occurred (cell size ≥3 μm larger than uninfected cells and viability approximately 80-90%). The volume of infected Sf21 cells from the overnight amplification used to infect the large-scale expression of Trichoplusia ni (T.ni; Expression Systems, cat #94-002F, www.expressionsystems.com) seeded at a density of 1.0×10⁶ in cell media (ESF921 SFM containing 5 μg/mL gentamicin) at MOI=2.0 was calculated based on (100 pfu/infected Sf21 cell). The cells were allowed to express for 48 h at 27° C. before harvesting the cell pellet, by centrifugation at 3,400×g for 10 min at 4° C. T. ni cells were counted on a ViCell XR to confirm infection had occurred (cell size ≥3 μm larger than uninfected cells and viability approximately 80-90%) prior to harvest.

### Full-Length STING (HAQ) Membrane Generation

Buffer stock reagents:

1) 1M HEPES pH 7.5, Teknova, Cat #H1035

2) 5M NaCl, Sigma Aldrich, Cat #S5150-1L

3) KCl, Sigma Aldrich, Cat #319309-500ML

4) Complete EDTA-free protease inhibitor tablets, Roche Diagnostics, Cat #11873580001

5) Benzonase, Universal Nuclease, Pierce, Cat #88702

Lysis buffer [25 mM HEPES pH 7.5, 10 mM MgCl₂, 20 mM KCl, (Benzonase 1:5000, Complete Protease Inhibitor tab/50 mL)] was added to the pellet of cells expressing full-length STING (HAQ) prepared above at 5 mL Lysis buffer per g of cell pellet. The pellet was resuspended and dounced twenty times using a Wheaton Dounce Homogenizer to disrupt the cell membrane. Homogenized lysate was then passed through the EMULSIFLEX-C5 microfluidizer at a pressure close to 5000 PSI. The resuspended pellet was centrifuged at 36,000 rpm (100,000×g) in a 45 Ti rotor ultra-high speed centrifuge for 45 min, 4° C. The supernatant was removed. The pellet then was resuspended in wash buffer [(25 mM HEPES pH7.5, 1 mM MgCl₂, 20 mM KCl, 1M NaCl (Complete Protease Inhibitor tab/50 mL)] at a volume of 50 mL pellet/centrifuge tube. The pellet/wash buffer mixture was then homogenized, using a glass homogenizer on ice (20 strokes), followed by centrifugation at 36,000 rpm for 45 min at 4° C. The supernatant was removed. The wash step was repeated once more. The resulting membrane was resuspended in 20 mM HEPES pH 7.5, 500 mM NaCl, 10% glycerol, EDTA-free Protease Inhibitors (1 tablet/50 mL). The protein concentration was measured by Bradford assay (Bio-Rad Protein Assay, Cat #500-0006), and protein enrichment was determined by SDS-PAGE and confirmed by Western blot. The resuspended membranes were stored at −80° C.

Full-Length HAQ STING [STING(1-379)R71H, G230A, H232R,
R293Q-GG-AviTag-GS-HRV3C-HIS8] Amino Acid Sequence:
(SEQ. ID. No. 1)
MPHSSLHPSIPCPRGHGAQKAALVLLSACLVTLWGLGEPPEHTLRYLVLHLASLQLGLLLNGVCSLAEELHHIH
SRYRGSYWRTVRACLGCPLRRGALLLLSIYFYYSLPNAVGPPFTWMLALLGLSQALNILLGLKGLAPAEISAVC
EKGNFNVAHGLAWSYYIGYLRLILPELQARIRTYNQHYNNLLRGAVSQRLYILLPLDCGVPDNLSMADPNIRFL
DKLPQQTADRAGIKDRVYSNSIYELLENGQRAGTCVLEYATPLQTLFAMSQYSQAGFSREDRLEQAKLFCQTLE
DILADAPESQNNCRLIAYQEPADDSSFSLSQEVLRHLRQEEKEEVTVGSLKTSAVPSTSTMSQEPELLISGMEK
PLPLRTDFSGGGLNDIFEAQKIEWHEGSLEVLFQGPHHHHHHHH
Full-length HAQ [STING(1-379)R71H, G230A, H232R,
R293Q-GG-AviTag-GS-HRV3C-HIS8/pBAC1] Plasmid DNA Sequence:
(SEQ. ID. No. 2)
GGAACGGCTCCGCCCACTATTAATGAAATTAAAAATTCCAATTTTAAAAAACGCAGCAAGAGAAACATTTGTAT
GAAAGAATGCGTAGAAGGAAAGAAAAATGTCGTCGACATGCTGAACAACAAGATTAATATGCCTCCGTGTATAA
AAAAAATATTGAACGATTTGAAAGAAAACAATGTACCGCGCGGCGGTATGTACAGGAAGAGGTTTATACTAAAC
TGTTACATTGCAAACGTGGTTTCGTGTGCCAAGTGTGAAAACCGATGTTTAATCAAGGCTCTGACGCATTTCTA
CAACCACGACTCCAAGTGTGTGGGTGAAGTCATGCATCTTTTAATCAAATCCCAAGATGTGTATAAACCACCAA
ACTGCCAAAAAATGAAAACTGTCGACAAGCTCTGTCCGTTTGCTGGCAACTGCAAGGGTCTCAATCCTATTTGT
AATTATTGAATAATAAAACAATTATAAATGCTAAATTTGTTTTTTATTAACGATACAAACCAAACGCAACAAGA
ACATTTGTAGTATTATCTATAATTGAAAACGCGTAGTTATAATCGCTGAGGTAATATTTAAAATCATTTTCAAA
TGATTCACAGTTAATTTGCGACAATATAATTTTATTTTCACATAAACTAGACGCCTTGTCGTCTTCTTCTTCGT
ATTCCTTCTCTTTTTCATTTTTCTCTTCATAAAAATTAACATAGTTATTATCGTATCCATATATGTATCTATCG
TATAGAGTAAATTTTTTGTTGTCATAAATATATATGTCTTTTTTAATGGGGTGTATAGTACCGCTGCGCATAGT
TTTTCTGTAATTTACAACAGTGCTATTTTCTGGTAGTTCTTCGGAGTGTGTTGCTTTAATTATTAAATTTATAT
AATCAATGAATTTGGGATCGTCGGTTTTGTACAATATGTTGCCGGCATAGTACGCAGCTTCTTCTAGTTCAATT
ACACCATTTTTTAGCAGCACCGGATTAACATAACTTTCCAAAATGTTGTACGAACCGTTAAACAAAAACAGTTC
ACCTCCCTTTTCTATACTATTGTCTGCGAGCAGTTGTTTGTTGTTAAAAATAACAGCCATTGTAATGAGACGCA
CAAACTAATATCACAAACTGGAAATGTCTATCAATATATAGTTGCTGATCAGATCTGATCATGGAGATAATTAA
AATGATAACCATCTCGCAAATAAATAAGTATTTTACTGTTTTCGTAACAGTTTTGTAATAAAAAAACCTATAAA
TATAGGATCCATGCCCCACTCCAGCCTGCATCCATCCATCCCGTGTCCCAGGGGTCACGGGGCCCAGAAGGCAG
CCTTGGTTCTGCTGAGTGCCTGCCTGGTGACCCTTTGGGGGCTAGGAGAGCCACCAGAGCACACTCTCCGGTAC
CTGGTGCTCCACCTAGCCTCCCTGCAGCTGGGACTGCTGTTAAACGGGGTCTGCAGCCTGGCTGAGGAGCTGCA
CCACATCCACTCCAGGTACCGGGGCAGCTACTGGAGGACTGTGCGGGCCTGCCTGGGCTGCCCCCTCCGCCGTG
GGGCCCTGTTGCTGCTGTCCATCTATTTCTACTACTCCCTCCCAAATGCGGTCGGCCCGCCCTTCACTTGGATG
CTTGCCCTCCTGGGCCTCTCGCAGGCACTGAACATCCTCCTGGGCCTCAAGGGCCTGGCCCCAGCTGAGATCTC
TGCAGTGTGTGAAAAAGGGAATTTCAACGTGGCCCATGGGCTGGCATGGTCATATTACATCGGATATCTGCGGC
TGATCCTGCCAGAGCTCCAGGCCCGGATTCGAACTTACAATCAGCATTACAACAACCTGCTACGGGGTGCAGTG
AGCCAGCGGCTGTATATTCTCCTCCCATTGGACTGTGGGGTGCCTGATAACCTGAGTATGGCTGACCCCAACAT
TCGCTTCCTGGATAAACTGCCCCAGCAGACCGCTGACCGTGCTGGCATCAAGGATCGGGTTTACAGCAACAGCA
TCTATGAGCTTCTGGAGAACGGGCAGCGGGCGGGCACCTGTGTCCTGGAGTACGCCACCCCCTTGCAGACTTTG
TTTGCCATGTCACAATACAGTCAAGCTGGCTTTAGCCGGGAGGATAGGCTTGAGCAGGCCAAACTCTTCTGCCA
GACACTTGAGGACATCCTGGCAGATGCCCCTGAGTCTCAGAACAACTGCCGCCTCATTGCCTACCAGGAACCTG
CAGATGACAGCAGCTTCTCGCTGTCCCAGGAGGTTCTCCGGCACCTGCGGCAGGAGGAAAAGGAAGAGGTTACT
GTGGGCAGCTTGAAGACCTCAGCGGTGCCCAGTACCTCCACGATGTCCCAAGAGCCTGAGCTCCTCATCAGTGG
AATGGAAAAGCCCCTCCCTCTCCGCACGGATTTCTCTGGCGGTGGCCTGAACGACATCTTCGAAGCCCAGAAAA
TCGAATGGCATGAAGGCAGCCTGGAAGTGCTGTTCCAGGGCCCACACCACCATCATCACCATCACCATTAATGA
GCGGCCGCACTCGAGCACCACCACCACCACCACTAACCTAGGTAGCTGAGCGCATGCAAGCTGATCCGGGTTAT
TAGTACATTTATTAAGCGCTAGATTCTGTGCGTTGTTGATTTACAGACAATTGTTGTACGTATTTTAATAATTC
ATTAAATTTATAATCTTTAGGGTGGTATGTTAGAGCGAAAATCAAATGATTTTCAGCGTCTTTATATCTGAATT
TAAATATTAAATCCTCAATAGATTTGTAAAATAGGTTTCGATTAGTTTCAAACAAGGGTTGTTTTTCCGAACCG
ATGGCTGGACTATCTAATGGATTTTCGCTCAACGCCACAAAACTTGCCAAATCTTGTAGCAGCAATCTAGCTTT
GTCGATATTCGTTTGTGTTTTGTTTTGTAATAAAGGTTCGACGTCGTTCAAAATATTATGCGCTTTTGTATTTC
TTTCATCACTGTCGTTAGTGTACAATTGACTCGACGTAAACACGTTAAATAGAGCTTGGACATATTTAACATCG
GGCGTGTTAGCTTTATTAGGCCGATTATCGTCGTCGTCCCAACCCTCGTCGTTAGAAGTTGCTTCCGAAGACGA
TTTTGCCATAGCCACACGACGCCTATTAATTGTGTCGGCTAACACGTCCGCGATCAAATTTGTAGTTGAGCTTT
TTGGAATTATTTCTGATTGCGGGCGTTTTTGGGCGGGTTTCAATCTAACTGTGCCCGATTTTAATTCAGACAAC
ACGTTAGAAAGCGATGGTGCAGGCGGTGGTAACATTTCAGACGGCAAATCTACTAATGGCGGCGGTGGTGGAGC
TGATGATAAATCTACCATCGGTGGAGGCGCAGGCGGGGCTGGCGGCGGAGGCGGAGGCGGAGGTGGTGGCGGTG
ATGCAGACGGCGGTTTAGGCTCAAATGTCTCTTTAGGCAACACAGTCGGCACCTCAACTATTGTACTGGTTTCG
GGCGCCGTTTTTGGTTTGACCGGTCTGAGACGAGTGCGATTTTTTTCGTTTCTAATAGCTTCCAACAATTGTTG
TCTGTCGTCTAAAGGTGCAGCGGGTTGAGGTTCCGTCGGCATTGGTGGAGCGGGCGGCAATTCAGACATCGATG
GTGGTGGTGGTGGTGGAGGCGCTGGAATGTTAGGCACGGGAGAAGGTGGTGGCGGCGGTGCCGCCGGTATAATT
TGTTCTGGTTTAGTTTGTTCGCGCACGATTGTGGGCACCGGCGCAGGCGCCGCTGGCTGCACAACGGAAGGTCG
TCTGCTTCGAGGCAGCGCTTGGGGTGGTGGCAATTCAATATTATAATTGGAATACAAATCGTAAAAATCTGCTA
TAAGCATTGTAATTTCGCTATCGTTTACCGTGCCGATATTTAACAACCGCTCAATGTAAGCAATTGTATTGTAA
AGAGATTGTCTCAAGCTCGGATCGATCCCGCACGCCGATAACAAGCCTTTTCATTTTTACTACAGCATTGTAGT
GGCGAGACACTTCGCTGTCGTCGAGGTTTAAACGCTTCCTCGCTCACTGACTCGCTGCGCTCGGTCGTTCGGCT
GCGGCGAGCGGTATCAGCTCACTCAAAGGCGGTAATACGGTTATCCACAGAATCAGGGGATAACGCAGGAAAGA
ACATGTGAGCAAAAGGCCAGCAAAAGGCCAGGAACCGTAAAAAGGCCGCGTTGCTGGCGTTTTTCCATAGGCTC
CGCCCCCCTGACGAGCATCACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTTACCGGATACCTGTCCG
CCTTTCTCCCTTCGGGAAGCGTGGCGCTTTCTCATAGCTCACGCTGTAGGTATCTCAGTTCGGTGTAGGTCGTT
CGCTCCAAGCTGGGCTGTGTGCACGAACCCCCCGTTCAGCCCGACCGCTGCGCCTTATCCGGTAACTATCGTCT
TGAGTCCAACCCGGTAAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGGACAGTATTTGGTATC
TGCGCTCTGCTGAAGCCAGTTACCTTCGGAAAAAGAGTTGGTAGCTCTTGATCCGGCAAACAAACCACCGCTGG
TAGCGGTGGTTTTTTTGTTTGCAAGCAGCAGATTACGCGCAGAAAAAAAGGATCTCAAGAAGATCCTTTGATCT
TTTCTACGGGGTCTGACGCTCAGTGGAACGAAAACTCACGTTAAGGGATTTTGGTCATGAGATTATCAAAAAGG
ATCTTCACCTAGATCCTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTTCATCCATAGTTGCCT
GACTCCCCGTCGTGTAGATAACTACGATACGGGAGGGCTTACCATCTGGCCCCAGTGCTGCAATGATACCGCGA
GACCCACGCTCACCGGCTCCAGATTTATCAGCAATAAACCAGCCAGCCGGAAGGGCCGAGCGCAGAAGTGGTCC
TGCAACTTTATCCGCCTCCATCCAGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTCGTTTGGTATGGCTTCATTCAGC
TCCGGTTCCCAACGATCAAGGCGAGTTACATGATCCCCCATGTTGTGCAAAAAAGCGGTTAGCTCCTTCGGTCC
TCCGATCGTTGTCAGAAGTAAGTTGGCCGCAGTGTTATCACTCATGGTTATGGCAGCACTGCATAATTCTCTTA
CTGTCATGCCATCCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCAGTTCGATGTAAC
CCACTCGTGCACCCAACTGATCTTCAGCATCTTTTACTTTCACCAGCGTTTCTGGGTGAGCAAAAACAGGAAGG
CAAAATGCCGCAAAAAAGGGAATAAGGGCGACACGGAAATGTTGAATACTCATACTCTTCCTTTTTCAATATTA
TTGAAGCATTTATCAGGGTTATTGTCTCATGAGCGGATACATATTTGAATGTATTTAGAAAAATAAACAAATAG
GGGTTCCGCGCACATTTCCCCGAAAAGTGCCACCTGACGCGCCCTGTAGCGGCGCATTAAGCGCGGCGGGTGTG
GTGGTTACGCGCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGATTTAGTGCTT
TACGGCACCTCGACCCCAAAAAACTTGATTAGGGTGATGGTTCACGTAGTGGGCCATCGCCCTGATAGACGGTT
TTTCGCCCTTTGACGTTGGAGTCCACGTTCTTTAATAGTGGACTCTTGTTCCAAACTGGAACAACACTCAACCC
TATCTCGGTCTATTCTTTTGATTTATAAGGGATTTTGCCGATTTCGGCCTATTGGTTAAAAAATGAGCTGATTT
AACAAAAATTTAACGCGAATTTTAACAAAATATTAACGTTTACAATTTCCCATTCGCCATTCAGGCTGCGCAAC
TGTTGGGAAGGGCGATCGGTGCGGGCCTCTTCGCTATTACGCCA

Certain compounds of the disclosure were evaluated in HAQ STING in vitro binding assay as described above. Table 6 tabulates the biological data for these compounds as EC₅₀ values.

TABLE 6
³H-cGAMP filtration binding assay for HAQ STING

| Compound | EC₅₀ (nM) |
|---|---|
| Example 1 | 1136 |
| Example 2 | 1128 |
| Example 3 | 51.3 |
| Example 4 | 1.69 |
| Example 5 | 1.87 |
| Example 6 | 241.1 |
| Example 7 | 3669 |
| Example 8 | 26 |
| Example 9 | 1.02 |
| Example 10 | 8.52 |
| Example 11 | 10.1 |
| Example 12 | 1.02 |
| Example 13 | 37.8 |
| Example 20 | 128.9 |
| Example 21 | 7.76 |
| Example 22 | 70 |
| Example 23 | 279.2 |
| Example 24 | 2.39 |
| Example 25 | 99.9 |
| Example 26 | 5.62 |

### 3H-cGAMP Filtration Binding Assay (WT STING)

The ability of compounds to bind STING is quantified by their ability to compete with tritiated cGAMP ligand for human STING receptor membrane using a radioactive filter-binding assay. The binding assay employs STING receptor obtained from Trichoplusia ni cell membranes (T.ni; Expression Systems, cat #94-002F, www.expressionsystems.com) overexpressing full-length WT STING and tritiated cGAMP ligand.

The basic WT STING filtration assay protocol is as follows:

16 nM of [³H] c-GAMP ligand was prepared by diluting into assay buffer, and 50 uL of this working stock was manually added to each well of the assay plate. After ligand addition, 2 uL of either titrated test compound, DMSO control (Sigma #276855), or cold cGAMP control was added to the appropriate wells using a BIOMEK FX. The serially titrated compound was prepared on a Hamilton STARPlus CORE in a 96-well plate (Greiner, #651201) using a 1:3 ten-point dose response format. Following compound addition, a 2.2 ug/ml working concentration of STING membrane (SEQ. ID. No. 3) was prepared by diluting concentrated membrane into assay buffer (1×PBS; Invitrogen #SH30028.02) and douncing 7× using a manual tissue homogenizer (Wheaton, #357546). 148 uL of this prepared membrane was then manually added to each well of a 96-well deep-well polypropylene plate (Fisher Scientific, #12-566-121). Compound, ligand, and membrane then incubated for 60 min at RT before the contents of each assay plate were filtered through a 96-well GFB filter plate (PerkinElmer, #6005250) using a TOMTEC MACH III Cell Harvester equipped with 20 mM HEPES buffer (Fisher Scientific, #BP299500). The filter plates were then dried at 55° C. for 30 min using a pressurized VWR oven before 30 uL of ULTIMA GOLD F scintillate was added to each well. Tritium levels for each reaction well were then measured using a PerkinElmer TopCount plate reader.

After normalization to controls, the percent activity for each compound concentration was calculated by measuring the amount of remaining radioactivity. The plot of percent activity versus the log of compound concentration was fit with a 4-parameter dose response equation to calculate EC₅₀ values.

The final reaction conditions were:

| Component | Volume (uL) | Final Concentration | |
|---|---|---|---|
| STING membrane | 148 | 1.5 | ug/ml |
| ³H-cGAMP | 50 | 4.0 | nM |
| Low Control (cold cGAMP) | 2 | 10 | uM |
| Test compound/DMSO | 2 | 10 | uM |

Compound concentrations tested were 20.000, 637.00, 2.200, 0.740, 0.247, 0.082, 0.027, 0.009, 0.003, and 0.001 μM with 1.0% residual DMSO.

### Full-Length STING (WT) Virus Generation

STING virus was generated using an insect cell baculovirus system. Spodoptera frugiperda Sf21 cells (Kempbio, Inc.) were diluted to 5e5 cells/ml in Sf-900II SFM media (LifeTechnologies #10902088) without antibiotics. The cell suspension was added to each well of a treated 6-well plate (2 mL per well, 1e6 cells total), and the cells were allowed to adhere for at least 30 min. Meanwhile, a 1 mL co-transfection mix was assembled by combining 500 ng of WT STING[STING(1-379)H232R-gg-AviTag-gs-HRV3C-HIS8/pBAC1] (Genewiz custom synthesis) with 1 mL Sf-900II SFM media containing 10 μL CELLFECTIN® II Reagent (Invitrogen #10362100) and 100 ng viral backbone BestBac 2.0, v-cath/chiA Deleted Linearized Baculovirus DNA (Expression Systems #91-002). The transfection mixtures were allowed to incubate for 30 min. After incubation, media was gently removed from the adhered cells in the 6-well plate, the 1 mL transfection mixtures were added (1 mL per well), and the plate was placed in a humidified incubator at 27° C. The following day, 1 mL Sf-900II SFM media (no antibiotics) was added to each well of the 6-well plate. After media addition, the cells were allowed to incubate with DNA [(SEQ. ID. No. 4) and linearized viral backbone BestBac 2.0] at 27° C. for 5-7 days to generate the P0 viral stock. To generate P1 viral stocks, 0.5 mL of P0 viral supernatant was added to 50 mL uninfected Sf21 cells (seeded the day prior to infection at a density of 5×10⁵ cells/mL to allow for one overnight doubling) in Sf-900II SFM media containing 5 μg/mL gentamicin (Invitrogen #15710072). The infected cells were then incubated at 27° C. for 3 days while shaking at 110 rpm (ATR Biotech Multitron Infors HT #AJ118). On day 3, P1 cultures were counted using a ViCell XR (Beckman Coulter Life Sciences #383556) to confirm infection had occurred (cell size ≥3 μm larger than uninfected cells and viability approximately 85-95%). Cultures were harvested in 50 mL conical tubes and centrifuged at 2000×g for 10 min at 4° C. The P1 viral supernatants were poured off into clean 50 ml centrifuge tubes, and the remaining P1 cell pellets were used to generate Baculovirus Infected Insect Cells (BIICs). Cryopreservation media containing Sf-900II SFM media with 10% heat inactivated FBS, 10% DMSO (Sigma #D2650), and 5 μg/ml gentamicin was prepared and sterilized through 0.22 μM filter immediately prior to use. P1 cell pellets were resuspended to a density of 2e7 cells/ml and aliquoted into cryovials (1 mL per vial). Cryovials were placed in MR. FROSTY™ cell freezers O/N at −80° C. and transferred to liquid nitrogen for long term storage the following day. To generate P2 viral stock, 0.5 mL of the P1 viral supernatant was added to 50 mL uninfected Sf21 cells (seeded the day prior to infection at a density of 5×10⁵ cells/mL to allow for one overnight doubling) in Sf-900II SFM media containing 5 μg/mL gentamicin. These cells were incubated at 27° C. for 3 days while shaking at 110 rpm before harvesting P2 stock with centrifugation at 2000×g for 10 min at 4° C. The P2 viral supernatants were poured off and discarded, while the P2 cell pellets were used to generate P2 BIICs following the same protocol described above. The baculovirus generation protocol has been validated to consistently produce P1/P2 BIICs with titers of 2e9 pfu/mL (2e7 cells/mL×100 pfu/cell).

### Full-Length STING (WT) Expression

To generate STING membranes, P1/P2 BIICs were amplified overnight by adding thawed BIICs to Sf21 cells seeded at a density of 1.0×10⁶ cells/mL. The volume of BIIC used to infect the culture was calculated using an assumed BIIC titer of 2e9 pfu/ml to achieve an MOI of 10 in the overnight amplification. After culturing overnight, the cells were counted on a ViCell XR to confirm infection had occurred (cell size ≥3 μm larger than uninfected cells and viability approximately 80-90%). The volume of infected Sf21 cells from the overnight amplification used to infect the large-scale expression of Trichoplusia ni (T.ni; Expression Systems, cat #94-002F, www.expressionsystems.com) seeded at a density of 1.0×10⁶ in cell media (ESF921 SFM containing 5 μg/mL gentamicin) at MOI=2.0 was calculated based on (100 pfu/infected Sf21 cell). The cells were allowed to express for 48 h at 27° C. before harvesting the cell pellet, by centrifugation at 3,400×g for 10 min at 4° C. T. ni cells were counted on a ViCell XR to confirm infection had occurred (cell size ≥3 μm larger than uninfected cells and viability approximately 80-90%) prior to harvest.

### Full-Length STING (WT) Membrane Generation

Buffer stock reagents:

1) 1 M HEPES pH 7.5, Teknova, Cat #H1035

2) 5 M NaCl, Sigma Aldrich, Cat #S5150-1L

3) KCl, Sigma Aldrich, Cat #319309-500ML

4) Complete EDTA-free protease inhibitor tablets, Roche Diagnostics, Cat #11873580001

5) Benzonase, Universal Nuclease, Pierce, Cat #88702

Lysis buffer [25 mM HEPES pH 7.5, 10 mM MgCl₂, 20 mM KCl, (Benzonase 1:5000, Complete Protease Inhibitor tab/50 mL)] was added to the pellet of cells expressing full-length STING (WT) prepared above at 5 mL Lysis buffer per g of cell pellet. The pellet was resuspended and dounced twenty times using a Wheaton Dounce Homogenizer to disrupt the cell membrane. Homogenized lysate was then passed through the emulsiflex-05 microfluidizer at a pressure close to 5000 PSI. The resuspended pellet was centrifuged at 36,000 rpm (100,000×g) in a 45 Ti rotor ultra-high speed centrifuge for 45 min, 4° C. The supernatant was removed. The pellet then was resuspended in wash buffer [(25 mM HEPES pH 7.5, 1 mM MgCl₂, 20 mM KCl, 1M NaCl (Complete Protease Inhibitor tab/50 mL)] at a volume of 50 mL/pellet/centrifuge tube. The pellet/wash buffer mixture was then homogenized, using a glass homogenizer on ice (20 strokes), followed by centrifugation at 36,000 rpm for 45 min at 4° C. The supernatant was removed. The wash step was repeated once more. The resulting membrane was resuspended in 20 mM HEPES pH 7.5, 500 mM NaCl, 10% glycerol, EDTA-free Protease Inhibitors (1 tablet/50 mL). The protein concentration was measured by Bradford assay (Bio-Rad Protein Assay, Cat #500-0006), and protein enrichment was determined by SDS-PAGE and confirmed by Western blot. The resuspended membranes were stored at −80° C.

Full-Length STING WT [STING(1-379)H232R-gg-AviTag-gs-HRV3C-HIS8]
Amino Acid Sequence:
(SEQ. ID. No. 3)
MPHSSLHPSIPCPRGHGAQKAALVLLSACLVTLWGLGEPPEHTLRYLVLHLASLQLGLLLNGVCSLAEELRHIHS
RYRGSYWRTVRACLGCPLRRGALLLLSIYFYYSLPNAVGPPFTWMLALLGLSQALNILLGLKGLAPAEISAVCEK
GNFNVAHGLAWSYYIGYLRLILPELQARIRTYNQHYNNLLRGAVSQRLYILLPLDCGVPDNLSMADPNIRFLDKL
PQQTGDRAGIKDRVYSNSIYELLENGQRAGTCVLEYATPLQTLFAMSQYSQAGFSREDRLEQAKLFCRTLEDILA
DAPESQNNCRLIAYQEPADDSSFSLSQEVLRHLRQEEKEEVTVGSLKTSAVPSTSTMSQEPELLISGMEKPLPLR
TDFSGGGLNDIFEAQKIEWHEGSLEVLFQGPHHHHHHHH
Full-length WT STING [STING(1-379)H232R-gg-AviTag-gs-HRV3C-HIS8/pBACI]
plasmid sequence:
(SEQ. ID. No. 4)
GGAACGGCTCCGCCCACTATTAATGAAATTAAAAATTCCAATTTTAAAAAACGCAGCAAGAGAAACATTTGTATG
AAAGAATGCGTAGAAGGAAAGAAAAATGTCGTCGACATGCTGAACAACAAGATTAATATGCCTCCGTGTATAAAA
AAAATATTGAACGATTTGAAAGAAAACAATGTACCGCGCGGCGGTATGTACAGGAAGAGGTTTATACTAAACTGT
TACATTGCAAACGTGGTTTCGTGTGCCAAGTGTGAAAACCGATGTTTAATCAAGGCTCTGACGCATTTCTACAAC
CACGACTCCAAGTGTGTGGGTGAAGTCATGCATCTTTTAATCAAATCCCAAGATGTGTATAAACCACCAAACTGC
CAAAAAATGAAAACTGTCGACAAGCTCTGTCCGTTTGCTGGCAACTGCAAGGGTCTCAATCCTATTTGTAATTAT
TGAATAATAAAACAATTATAAATGTCAAATTTGTTTTTTATTAACGATACAAACCAAACGCAACAAGAACATTTG
TAGTATTATCTATAATTGAAAACGCGTAGTTATAATCGCTGAGGTAATATTTAAAATCATTTTCAAATGATTCAC
AGTTAATTTGCGACAATATAATTTTATTTTCACATAAACTAGACGCCTTGTCGTCTTCTTCTTCGTATTCCTTCT
CTTTTTCATTTTTCTCTTCATAAAAATTAACATAGTTATTATCGTATCCATATATGTATCTATCGTATAGAGTAA
ATTTTTTGTTGTCATAAATATATATGTCTTTTTTAATGGGGTGTATAGTACCGCTGCGCATAGTTTTTCTGTAAT
TTACAACAGTGCTATTTTCTGGTAGTTCTTCGGAGTGTGTTGCTTTAATTATTAAATTTATATAATCAATGAATT
TGGGATCGTCGGTTTTGTACAATATGTTGCCGGCATAGTACGCAGCTTCTTCTAGTTCAATTACACCATTTTTTA
GCAGCACCGGATTAACATAACTTTCCAAAATGTTGTACGAACCGTTAAACAAAAACAGTTCACCTCCCTTTTCTA
TACTATTGTCTGCGAGCAGTTGTTTGTTGTTAAAAATAACAGCCATTGTAATGAGACGCACAAACTAATATCACA
AACTGGAAATGTCTATCAATATATAGTTGCTGATCAGATCTGATCATGGAGATAATTAAAATGATAACCATCTCG
CAAATAAATAAGTATTTTACTGTTTTCGTAACAGTTTTGTAATAAAAAAACCTATAAATATAGGATCCATGCCCC
ACTCCAGCCTGCATCCATCCATCCCGTGTCCCAGGGGTCACGGGGCCCAGAAGGCAGCCTTGGTTCTGCTGAGTG
CCTGCCTGGTGACCCTTTGGGGGCTAGGAGAGCCACCAGAGCACACTCTCCGGTACCTGGTGCTCCACCTAGCCT
CCCTGCAGCTGGGACTGCTGTTAAACGGGGTCTGCAGCCTGGCTGAGGAGCTGCGCCACATCCACTCCAGGTACC
GGGGCAGCTACTGGAGGACTGTGCGGGCCTGCCTGGGCTGCCCCCTCCGCCGTGGGGCCCTGTTGCTGCTGTCCA
TCTATTTCTACTACTCCCTCCCAAATGCGGTCGGCCCGCCCTTCACTTGGATGCTTGCCCTCCTGGGCCTCTCGC
AGGCACTGAACATCCTCCTGGGCCTCAAGGGCCTGGCCCCAGCTGAGATCTCTGCAGTGTGTGAAAAAGGGAATT
TCAACGTGGCCCATGGGCTGGCATGGTCATATTACATCGGATATCTGCGGCTGATCCTGCCAGAGCTCCAGGCCC
GGATTCGAACTTACAATCAGCATTACAACAACCTGCTACGGGGTGCAGTGAGCCAGCGGCTGTATATTCTCCTCC
CATTGGACTGTGGGGTGCCTGATAACCTGAGTATGGCTGACCCCAACATTCGCTTCCTGGATAAACTGCCCCAGC
AGACCGGTGACCGTGCTGGCATCAAGGATCGGGTTTACAGCAACAGCATCTATGAGCTTCTGGAGAACGGGCAGC
GGGCGGGCACCTGTGTCCTGGAGTACGCCACCCCCTTGCAGACTTTGTTTGCCATGTCACAATACAGTCAAGCTG
GCTTTAGCCGGGAGGATAGGCTTGAGCAGGCCAAACTCTTCTGCCGGACACTTGAGGACATCCTGGCAGATGCCC
CTGAGTCTCAGAACAACTGCCGCCTCATTGCCTACCAGGAACCTGCAGATGACAGCAGCTTCTCGCTGTCCCAGG
AGGTTCTCCGGCACCTGCGGCAGGAGGAAAAGGAAGAGGTTACTGTGGGCAGCTTGAAGACCTCAGCGGTGCCCA
GTACCTCCACGATGTCCCAAGAGCCTGAGCTCCTCATCAGTGGAATGGAAAAGCCCCTCCCTCTCCGCACGGATT
TCTCTGGCGGTGGCCTGAACGACATCTTCGAAGCCCAGAAAATCGAATGGCATGAAGGCAGCCTGGAAGTGCTGT
TCCAGGGCCCACACCACCATCATCACCATCACCATTAATGAGCGGCCGCACTCGAGCACCACCACCACCACCACT
AACCTAGGTAGCTGAGCGCATGCAAGCTGATCCGGGTTATTAGTACATTTATTAAGCGCTAGATTCTGTGCGTTG
TTGATTTACAGACAATTGTTGTACGTATTTTAATAATTCATTAAATTTATAATCTTTAGGGTGGTATGTTAGAGC
GAAAATCAAATGATTTTCAGCGTCTTTATATCTGAATTTAAATATTAAATCCTCAATAGATTTGTAAAATAGGTT
TCGATTAGTTTCAAACAAGGGTTGTTTTTCCGAACCGATGGCTGGACTATCTAATGGATTTTCGCTCAACGCCAC
AAAACTTGCCAAATCTTGTAGCAGCAATCTAGCTTTGTCGATATTCGTTTGTGTTTTGTTTTGTAATAAAGGTTC
GACGTCGTTCAAAATATTATGCGCTTTTGTATTTCTTTCATCACTGTCGTTAGTGTACAATTGACTCGACGTAAA
CACGTTAAATAGAGCTTGGACATATTTAACATCGGGCGTGTTAGCTTTATTAGGCCGATTATCGTCGTCGTCCCA
ACCCTCGTCGTTAGAAGTTGCTTCCGAAGACGATTTTGCCATAGCCACACGACGCCTATTAATTGTGTCGGCTAA
CACGTCCGCGATCAAATTTGTAGTTGAGCTTTTTGGAATTATTTCTGATTGCGGGCGTTTTTGGGCGGGTTTCAA
TCTAACTGTGCCCGATTTTAATTCAGACAACACGTTAGAAAGCGATGGTGCAGGCGGTGGTAACATTTCAGACGG
CAAATCTACTAATGGCGGCGGTGGTGGAGCTGATGATAAATCTACCATCGGTGGAGGCGCAGGCGGGGCTGGCGG
CGGAGGCGGAGGCGGAGGTGGTGGCGGTGATGCAGACGGCGGTTTAGGCTCAAATGTCTCTTTAGGCAACACAGT
CGGCACCTCAACTATTGTACTGGTTTCGGGCGCCGTTTTTGGTTTGACCGGTCTGAGACGAGTGCGATTTTTTTC
GTTTCTAATAGCTTCCAACAATTGTTGTCTGTCGTCTAAAGGTGCAGCGGGTTGAGGTTCCGTCGGCATTGGTGG
AGCGGGCGGCAATTCAGACATCGATGGTGGTGGTGGTGGTGGAGGCGCTGGAATGTTAGGCACGGGAGAAGGTGG
TGGCGGCGGTGCCGCCGGTATAATTTGTTCTGGTTTAGTTTGTTCGCGCACGATTGTGGGCACCGGCGCAGGCGC
CGCTGGCTGCACAACGGAAGGTCGTCTGCTTCGAGGCAGCGCTTGGGGTGGTGGCAATTCAATATTATAATTGGA
ATACAAATCGTAAAAATCTGCTATAAGCATTGTAATTTCGCTATCGTTTACCGTGCCGATATTTAACAACCGCTC
AATGTAAGCAATTGTATTGTAAAGAGATTGTCTCAAGCTCGGATCGATCCCGCACGCCGATAACAAGCCTTTTCA
TTTTTACTACAGCATTGTAGTGGCGAGACACTTCGCTGTCGTCGAGGTTTAAACGCTTCCTCGCTCACTGACTCG
CTGCGCTCGGTCGTTCGGCTGCGGCGAGCGGTATCAGCTCACTCAAAGGCGGTAATACGGTTATCCACAGAATCA
GGGGATAACGCAGGAAAGAACATGTGAGCAAAAGGCCAGCAAAAGGCCAGGAACCGTAAAAAGGCCGCGTTGCTG
GCGTTTTTCCATAGGCTCCGCCCCCCTGACGAGCATCACAAAAATCGACGCTCAAGTCAGAGGTGGCGAAACCCG
ACAGGACTATAAAGATACCAGGCGTTTCCCCCTGGAAGCTCCCTCGTGCGCTCTCCTGTTCCGACCCTGCCGCTT
ACCGGATACCTGTCCGCCTTTCTCCCTTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACTACGGCTACACTAGAAGGACA
GTATTTGGTATCTGCGCTCTGCTGAAGCCAGTTACCTTCGGAAAAAGAGTTGGTAGCTCTTGATCCGGCAAACAA
ACCACCGCTGGTAGCGGTGGTTTTTTTGTTTGCAAGCAGCAGATTACGCGCAGAAAAAAAGGATCTCAAGAAGAT
CCTTTGATCTTTTCTACGGGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCAATCTAAAGTATATATGAGTAA
ACTTGGTCTGACAGTTACCAATGCTTAATCAGTGAGGCACCTATCTCAGCGATCTGTCTATTTCGTTCATCCATA
GTTGCCTGACTCCCCGTCGTGTAGATAACTACGATACGGGAGGGCTTACCATCTGGCCCCAGTGCTGCAATGATA
CCGCGAGACCCACGCTCACCGGCTCCAGATTTATCAGCAATAAACCAGCCAGCCGGAAGGGCCGAGCGCAGAAGT
GGTCCTGCAACTTTATCCGCCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGTTAGCTCCTTCGGT
CCTCCGATCGTTGTCAGAAGTAAGTTGGCCGCAGTGTTATCACTCATGGTTATGGCAGCACTGCATAATTCTCTT
ACTGTCATGCCATCCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAACTTTAAAAGTGCTC
ATCATTGGAAAACGTTCTTCGGGGCGAAAACTCTCAAGGATCTTACCGCTGTTGAGATCCAGTTCGATGTAACCC
ACTCGTGCACCCAACTGATCTTCAGCATCTTTTACTTTCACCAGCGTTTCTGGGTGAGCAAAAACAGGAAGGCAA
AATGCCGCAAAAAAGGGAATAAGGGCGACACGGAAATGTTGAATACTCATACTCTTCCTTTTTCAATATTATTGA
AGCATTTATCAGGGTTATTGTCTCATGAGCGGATACATATTTGAATGTATTTAGAAAAATAAACAAATAGGGGTT
CCGCGCACATTTCCCCGAAAAGTGCCACCTGACGCGCCCTGTAGCGGCGCATTAAGCGCGGCGGGTGTGGTGGTT
ACGCGCAGCGTGACCGCTACACTTGCCAGCGCCCTAGCGCCCGCTCCTTTCGCTTTCTTCCCTTCCTTTCTCGCC
ACGTTCGCCGGCTTTCCCCGTCAAGCTCTAAATCGGGGGCTCCCTTTAGGGTTCCGATTTAGTGCTTTACGGCAC
CTCGACCCCAAAAAACTTGATTAGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACCCTATCTCGGTC
TATTCTTTTGATTTATAAGGGATTTTGCCGATTTCGGCCTATTGGTTAAAAAATGAGCTGATTTAACAAAAATTT
AACGCGAATTTTAACAAAATATTAACGTTTACAATTTCCCATTCGCCATTCAGGCTGCGCAACTGTTGGGAAGGG
CGATCGGTGCGGGCCTCTTCGCTATTACGCCA

Certain compounds of the disclosure were evaluated in WT STING in vitro binding assay as described above. The following table tabulates the biological data for these compounds as EC₅₀ values.

TABLE 7
³H-cGAMP filtration binding assay for WT STING

| Compound | EC₅₀ (nM) |
|---|---|
| Example 14 | 13.2 |
| Example 15 | 293 |
| Example 16 | 7.34 |
| Example 17 | 22.3 |
| Example 18 | 211 |
| Example 19 | 2.32 |

IFN-β secretion in THP1 cell culture (56)

The ability of compounds to stimulate the secretion of interferon-beta from THP1 cells was measured using a human IFN-β AlphaLISA kit (Perkin Elmer, Cat. No. AL265F). The basic protocol is as follows:

A Labcyte Echo 550 acoustic dispenser was used to transfer 120 nL of compound dissolved in DMSO into the wells of an empty, sterile 384-well microplate, (Corning, Cat. No. 3712). THP1 cells (American Type Culture Collection, Cat. No. TIB202) previously frozen in Recovery Medium (Life Technologies, Cat. No. 12648-010) were thawed and immediately diluted 10-fold into 37° C. assay medium (RPMI 1640+L-Glutamine & phenol red, Life Technologies, Cat. No. 11875-085; 0.5% heat inactivated fetal bovine serum, Sigma Aldrich, Cat. No. F4135; 1 mM Sodium Pyruvate, Life Technologies, Cat. No. 11360-070; lx non essential amino acids; Life Technologies, Cat. No. 11140-050). The cell viability and count was ascertained using a Beckman Coulter V-Cell XR cell counter. The cell suspension was centrifuged at 200×g for 5 min at RT. Cells were resuspended to a density of 0.8×10⁶/mL in 37° C. assay medium. Subsequent liquid transfers were performed using either a Matrix electronic multichannel pipette or an Agilent Bravo Automated Liquid Handling Platform.

The assay was started by dispensing 40 μL of the previously prepared cell suspension into the wells of the plate containing compounds. After 5 h incubation at 37° C., 5% CO₂ in a humidified atmosphere, the plate of cells and compounds was centrifuged at 200×g for 5 min at RT. From each well, 54 of supernatant was transferred into corresponding wells of a white 384-well plate (Perkin Elmer, Cat. No. 6005620). To these supernatant-containing wells was added 10 μL of 5× Anti-Analyte Acceptor beads (50 μg/mL of AlphaLISA HiBlock Buffer) and incubated for 30 min at RT while shaking on an orbital plate shaker. To each well was added 10 μL of 5× Biotinylated Antibody Anti-analyte (5 nM in AlphaLISA HiBlock Buffer) and incubated on an orbital plate shaker for 60 min at RT or overnight at 4° C. To each well was added 25 μL of 2× SA-Donor beads (8014/mL in AlphaLISA HiBlock Buffer) and incubated for 30-45 min at RT in the dark while shaking on an orbital plate shaker. The plate was then read on a Perkin Elmer Envision (λₑₓ=680 nm, λₑₘ=570 nm). The percent effect of the AlphaLISA signal at each compound concentration was calculated based on 30 uM cGAMP positive controls and 0.3% DMSO negative controls. The plot of percent effect versus the log of compound concentration was fit with a 4-parameter concentration response equation to calculate EC₅₀ values. The test compounds were tested at concentrations 30000, 10000, 3333, 1111, 370.4, 123.4, 41.2, 13.7, 4.6, and 1.5 nM with 0.3% residual DMSO. The control compound, cGAMP was tested at concentrations 100000, 33333, 11111, 3704, 1235, 412, 137, 46, and 15 nM with 0.3% residual DMSO.

Compounds of the disclosure were evaluated for IFN-β secretion in THP1 cell culture as described above. The following table tabulates the biological data for these compounds as percent activation relative to 2′3′-cGAMP at the 30 μM concentration.

TABLE 8
IFN-β secretion in THP1 cell culture (5 h)

| | % Effect at 30 μM relative |
|---|---|
| Compound | to 2′3′-cGAMP |
| Example 1 | 24 |
| Example 2 | 29 |
| Example 3 | 128 |
| Example 4 | 113 |
| Example 5 | 113 |
| Example 6 | 60 |
| Example 7 | 15 |
| Example 8 | 145 |
| Example 9 | 184 |
| Example 10 | 174 |
| Example 11 | 77 |
| Example 12 | 183 |
| Example 13 | 79 |
| Example 14 | 257 |
| Example 15 | 31 |
| Example 16 | 188 |
| Example 17 | 141 |
| Example 18 | 244 |
| Example 19 | 207 |
| Example 20 | 59 |
| Example 21 | 118 |
| Example 22 | 69 |
| Example 23 | 51 |
| Example 24 | 129 |
| Example 25 | 92 |
| Example 26 | 79 |

It will be appreciated that various of the above-discussed and other features and functions, or alternatives thereof, may be desirably combined into many other different systems or applications. It also will be appreciated that various presently unforeseen or unanticipated alternatives, modifications, variations or improvements therein may be subsequently made by those skilled in the art and are also intended to be encompassed by the following claims.

1. A compound of formula (I): 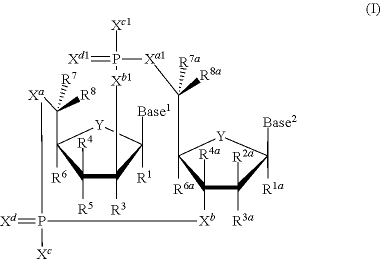
or a pharmaceutically acceptable salt thereof, wherein
Base¹ and Base² are each independently selected from the group consisting of 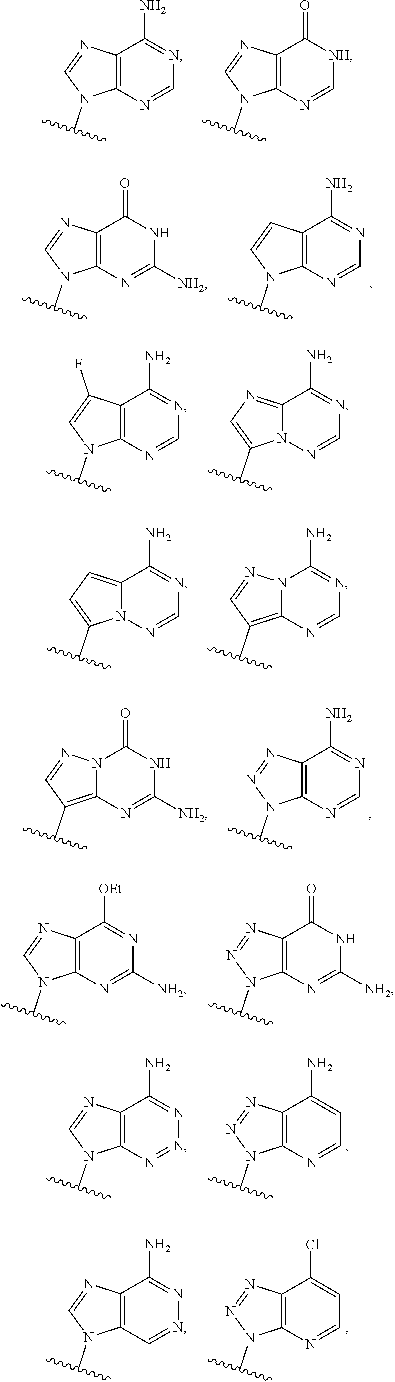 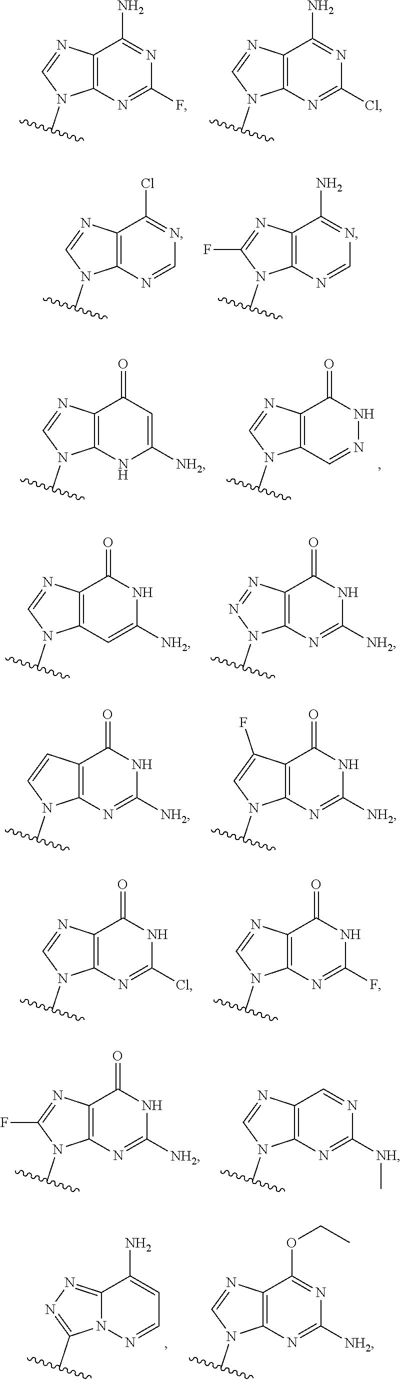 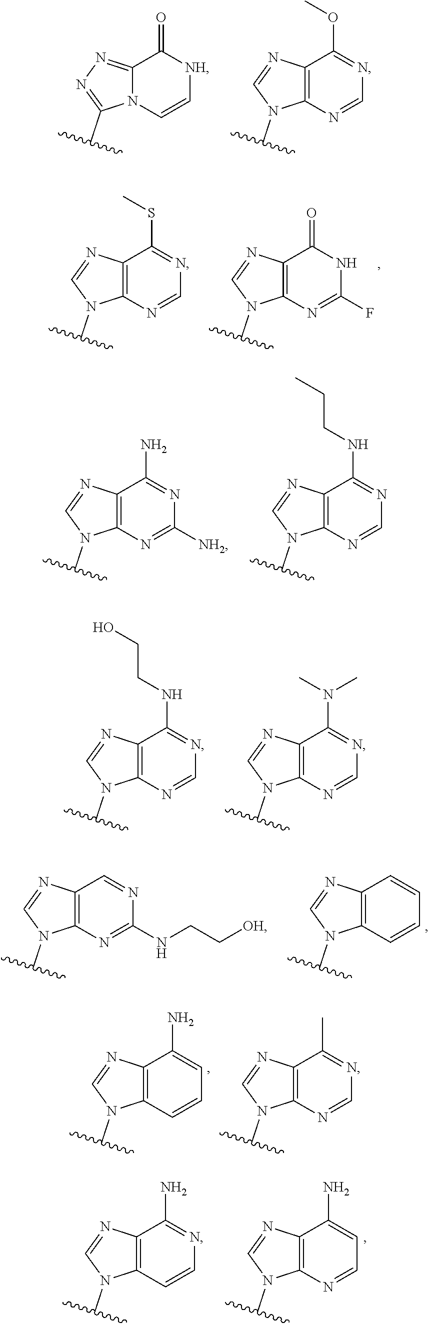 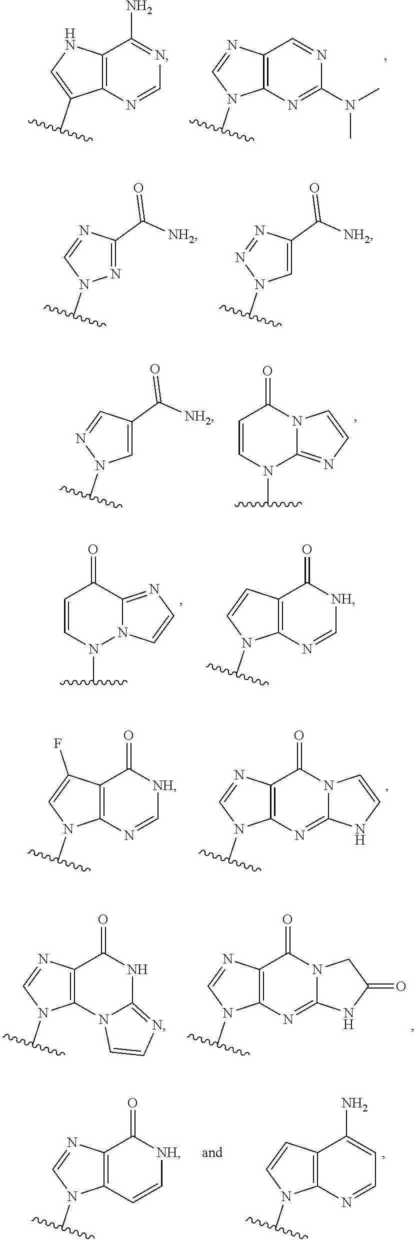
and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O—, —S—, —SO₂—, —CH₂—, and —CF₂—;
X^{a} and X^{a1} are each independently selected from the group consisting of —O—, —S—, and —CH₂—;
X^{b} and X^{b1} are each independently selected from the group consisting of —O—, —S—, and —CH₂—;
X^{c} and X^{c1} are each independently selected from the group consisting of —SR⁹, —OR⁹, and —NR⁹R⁹;
X^{d} and X^{d1} are each independently selected from the group consisting of O and S;
R¹ and R^{1a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R¹ and R^{1a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R^{2a} is selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R^{2a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R³ and R^{3a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R³ and R^{3a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R⁴ and R^{4a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁴ and R^{4a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R⁵ is selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁵ C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R⁶ and R^{6a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁶ and R^{6a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R⁷ and R^{7a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁷ and R^{7a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
R⁸ and R^{8a} are each independently selected from the group consisting of H, F, Cl, Br, I, OH, CN, N₃, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl, where said R⁸ and R^{8a} C₁-C₆ alkyl, C₂-C₆ alkenyl, C₂-C₆ alkynyl, C₁-C₆ haloalkyl, C₂-C₆ haloalkenyl, C₂-C₆ haloalkynyl, —O—C₁-C₆ alkyl, —O—C₂-C₆ alkenyl, and —O—C₂-C₆ alkynyl are substituted by 0 to 3 substituents selected from the group consisting of F, Cl, Br, I, OH, CN, and N₃;
each R⁹ is independently selected from the group consisting of H, C₁-C₂₀ alkyl, 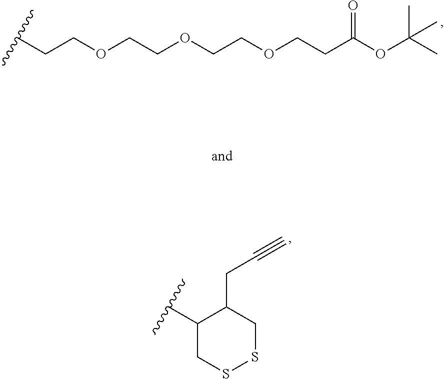
where each R⁹ C₁-C₂₀ alkyl is optionally substituted by 0 to 3 substituents independently selected from the group consisting of OH, —O—C₁-C₂₀ alkyl, —S—C(O)C₁-C₆ alkyl, and C(O)OC₁-C₆ alkyl;
optionally R^{1a} and R^{3a} are connected to form C₁-C₆ alkylene, C₂-C₆ alkenylene, —O—C₁-C₆ alkylene, or —O—C₂-C₆ alkenylene, such that where R^{1a} and R^{3a} are connected to form —O—C₁-C₆ alkylene, or —O—C₂-C₆ alkenylene, said O is bound at the R^{3a} position;
optionally R^{2a} and R^{3a} are connected to form C₁-C₆ alkylene, C₂-C₆ alkenylene, —O—C₁-C₆ alkylene, or —O—C₂-C₆ alkenylene, such that where R^{2a} and R^{3a} are connected to form —O—C₁-C₆ alkylene, or —O—C₂-C₆ alkenylene, said O is bound at the R^{3a} position;
optionally R^{3a} and R^{6a} are connected to form C₁-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene, or —O—C₂-C₆ alkenylene, said O is bound at the R^{3a} position;
optionally R⁴ and R⁵ are connected to form C₁-C₆ alkylene, C₂-C₆ alkenylene, —O—C₁-C₆ alkylene, or —O—C₂-C₆ alkenylene, such that where R⁴ and R⁵ are connected to form —O—C₁-C₆ alkylene, or —O—C₂-C₆ alkenylene, said O is bound at the R⁵ position;
optionally R⁵ and R⁶ are connected to form —O—C₁-C₆ alkylene, —O—C₂-C₆ alkenylene, or —O—C₂-C₆ alkynylene, such that where R⁵ and R⁶ are connected to form —O—C₁-C₆ alkylene, —O—C₂-C₆ alkenylene, or —O—C₂-C₆ alkynylene, said O is bound at the R⁵ position;
optionally R⁷ and R⁸ are connected to form C₁-C₆ alkylene or C₂-C₆ alkenylene; and
optionally R^{7a} and R^{8a} are connected to form C₁-C₆ alkylene or C₂-C₆ alkenylene.

2. The compound according to claim 1, or a pharmaceutically acceptable salt thereof, wherein
Y and Y^{a} are each independently selected from the group consisting of —O— and —S—;
X^{a} and X^{a1} are each independently selected from the group consisting of —O— and —S—;
X^{b} and X^{b1} are each independently selected from the group consisting of —O and —S—;
X^{c} and X^{c1} are each independently selected from the group consisting of —SR⁹, —OR⁹, and —NR⁹R⁹;
X^{d} and X^{d1} are each independently selected from the group consisting of O and S;
R¹ and R^{1a} are each H;
R^{2a} is selected from the group consisting of H, F, Cl, Br, I, OH, CN, N₃, C₁-C₆ alkyl, and C₁-C₆ haloalkyl, where said R^{2a} C₁-C₆ alkyl or C₁-C₆ haloalkyl are substituted by 0 to 3 substituents selected from the group consisting of OH, CN, and N₃;
R³ and R^{3a} are each independently selected from the group consisting of H, F, Cl, Br, I, OH, CN, N₃, C₁-C₆ alkyl, C₁-C₆ haloalkyl, —O—C₁-C₆ alkyl, —O—C₂-C₆ alkenyl, and —O—C₂-C₆ alkynyl, where said R³ and R^{3a} C₁-C₆ alkyl or C₁-C₆ haloalkyl are substituted by 0 to 3 substituents selected from the group consisting of OH, CN, and N₃;
R⁴ and R^{4a} are each independently selected from the group consisting of H, F, Cl, I, Br, CN, OH, N₃, C₁-C₆ alkyl, and C₁-C₆ haloalkyl, where said R⁴ and R^{4a} C₁-C₆ alkyl or C₁-C₆ haloalkyl are substituted by 0 to 3 substituents selected from the group consisting of OH, CN, and N₃;
R⁵ is selected from the group consisting of H, F, Cl, Br, I, OH, N₃, C₁-C₆ alkyl, and C₁-C₆ haloalkyl, where said R⁵ C₁-C₆ alkyl or C₁-C₆ haloalkyl are substituted by 0 to 3 substituents selected from the group consisting of F, Cl, Br, I, OH, CN, and N₃;
R⁶ and R^{6a} are each independently selected from the group consisting of H, F, Cl, I, Br, OH, CN, N₃, C₁-C₆ alkyl, C₂-C₆ alkenyl, and C₂-C₆ alkynyl, where said R⁶ and R^{6a} C₁-C₆ alkyl, C₂-C₆ alkenyl, and C₂-C₆ alkynyl are substituted by 0 to 3 substituents selected from the group consisting of F, Cl, Br, I, OH, CN, and N₃;
R⁷ and R^{7a} are each independently selected from the group consisting of H, C₁-C₆ alkyl, and C₁-C₆ haloalkyl, where said R⁷ and R^{7a} C₁-C₆ alkyl or C₁-C₆ haloalkyl are substituted by 0 to 3 substituents selected from the group consisting of OH, CN, and N₃;
R⁸ and R^{8a} are each independently selected from the group consisting of H, C₁-C₆ alkyl, and C₁-C₆ haloalkyl, where said R⁸ and R^{8a} C₁-C₆ alkyl or C₁-C₆ haloalkyl are substituted by 0 to 3 substituents selected from the group consisting of OH, CN, and N₃;
each R⁹ is independently selected from the group consisting of H, C₁-C₆ alkyl, 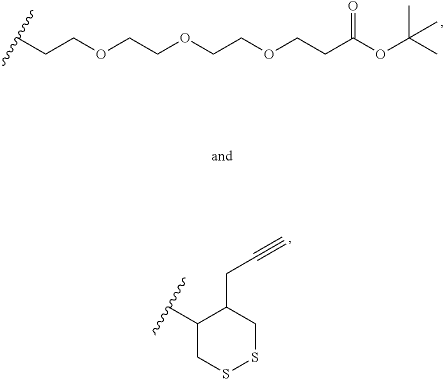
where each R⁹ C₁-C₆ alkyl is optionally substituted by 1 to 2 substituents independently selected from the group consisting of OH, —O—C₁-C₂₀ alkyl, —S—C(O)C₁-C₆ alkyl, and —C(O)OC₁-C₆ alkyl;
optionally R^{3a} and R^{6a} are connected to form C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position; and
optionally R⁵ and R⁶ are connected to form C₁-C₆ alkylene, C₂-C₆ alkenylene, —O—C₁-C₆ alkylene, or —O—C₂-C₆ alkenylene, such that where R⁵ and R⁶ are connected to form —O—C₁-C₆ alkylene or —O—C₂-C₆ alkenylene, said O is bound at the R⁵ position.

3. The compound according to claim 1, or a pharmaceutically acceptable salt thereof, wherein
Base¹ and Base² are each independently selected from the group consisting of 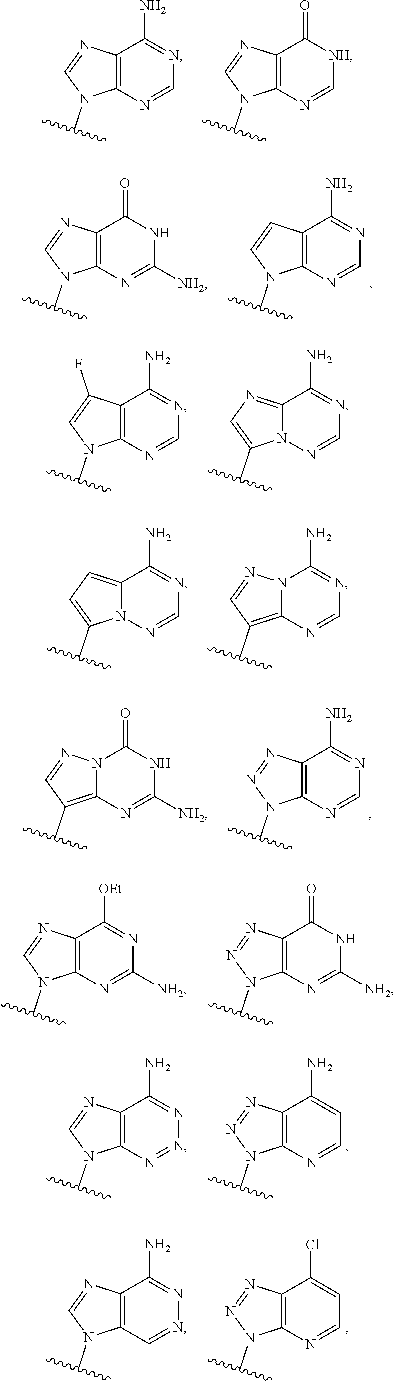 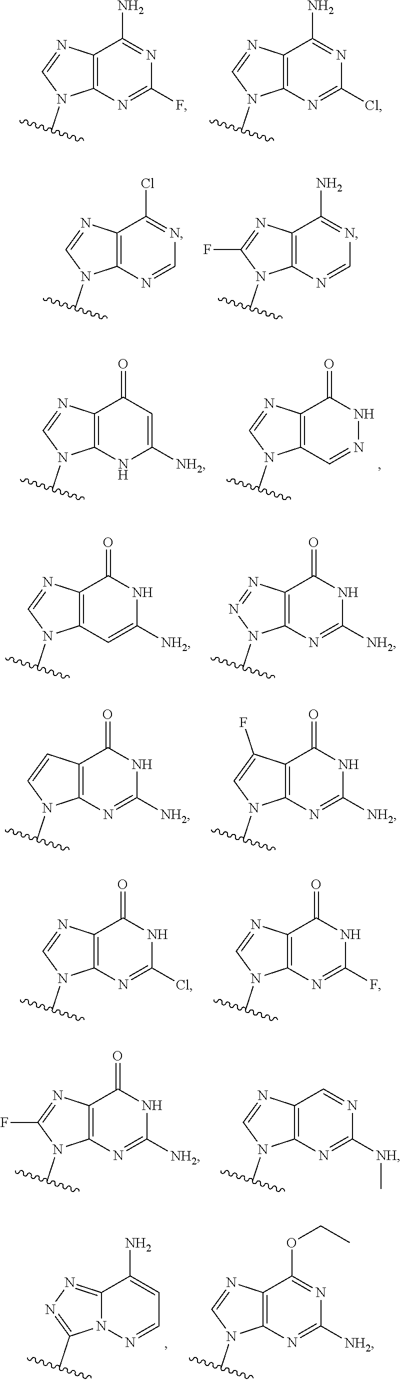 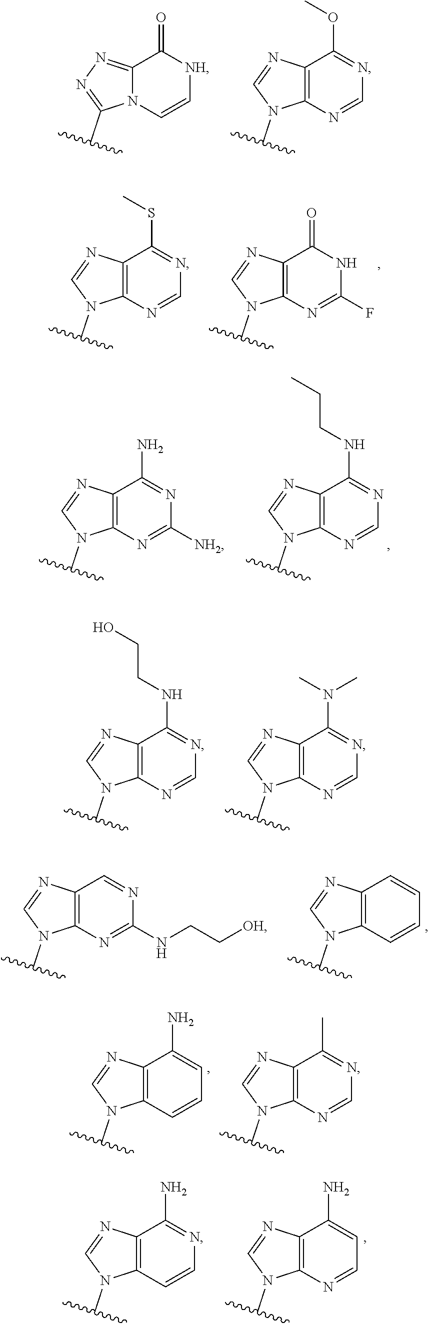 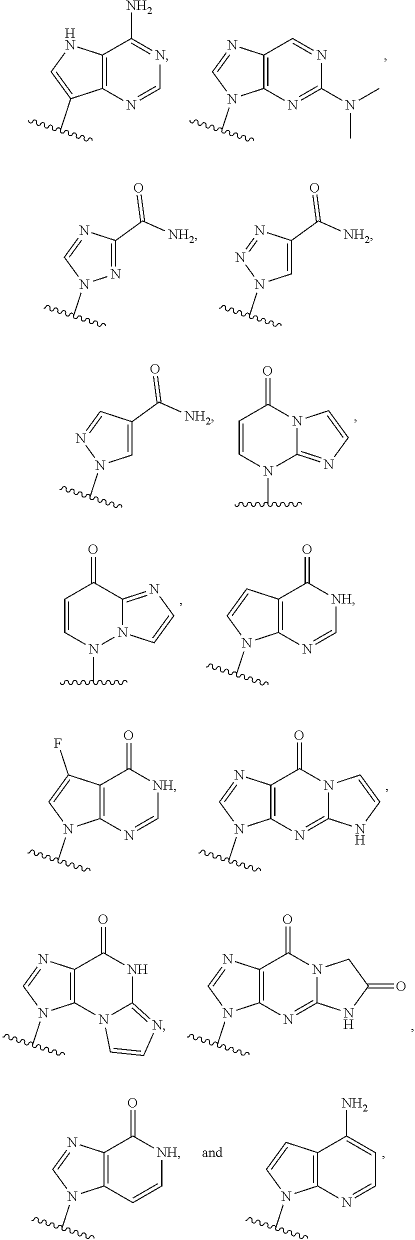
and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O— and —S—;
X^{a} and X^{a1} are each independently selected from the group consisting of —O— and —S—;
X^{b} and X^{b1} are each independently selected from the group consisting of —O— and —S—;
X^{c} and X^{c1} are each independently selected from the group consisting of —OH, —SH, 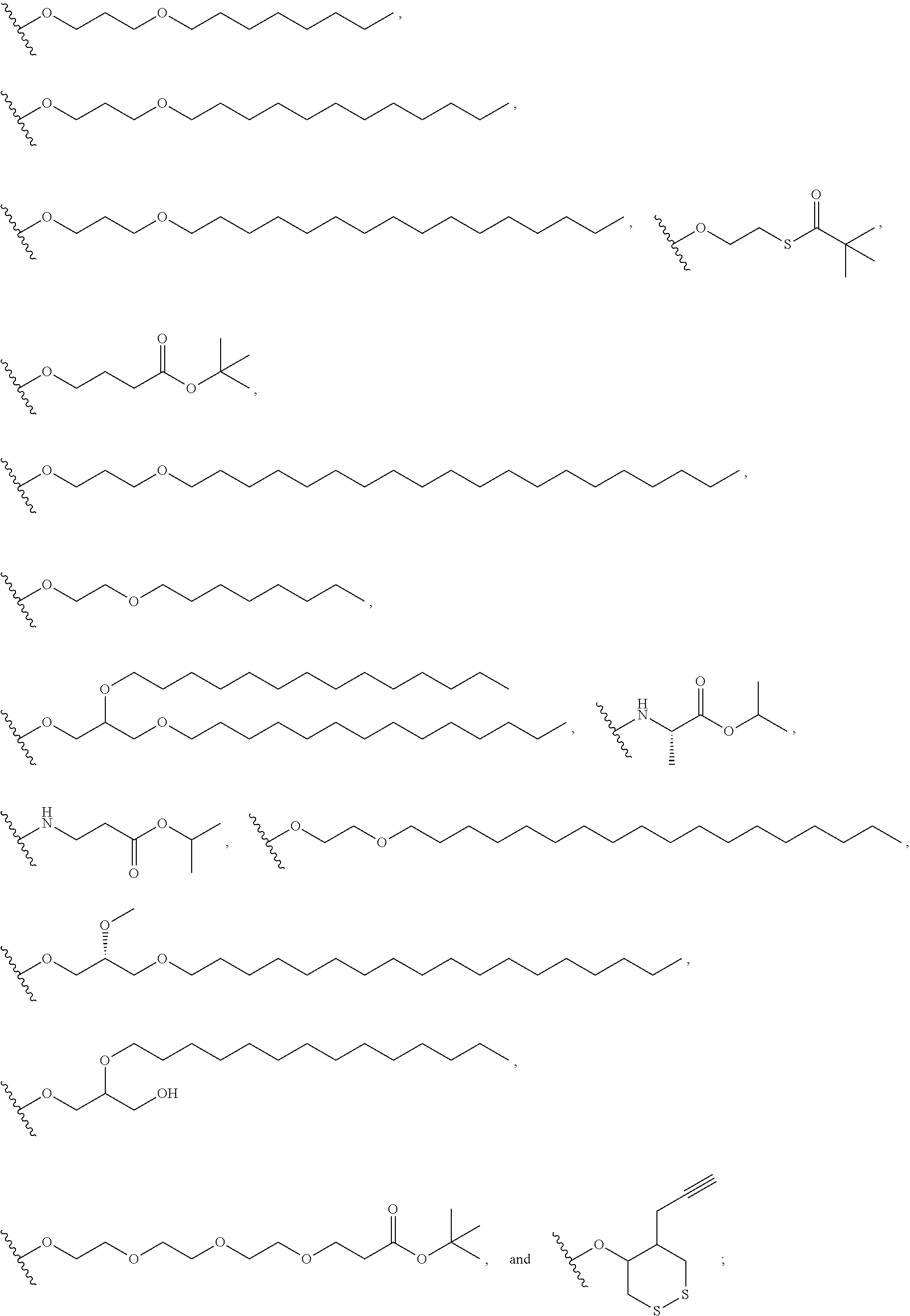
X^{d} and X^{d1} are each independently selected from the group consisting of O and S;
R¹ and R^{1a} are each H;
R^{2a} is selected from the group consisting of H, F, Cl, I, Br, OH, CN, N₃, —CF₃, —CH₃, —CH₂OH, and —CH₂CH₃;
R³ and R^{3a} are each independently selected from the group consisting of H, F, Cl, I, Br, OH, CN, N₃, —CF₃, —CH₃, —CH₂OH, —CH₂CH₃, —OCH₃, —OCH₂, and —OCH₂CH₃;
R⁴ and R^{4a} are each independently selected from the group consisting of H, F, Cl, I, Br, OH, CN, N₃, —CF₃, —CH₃, —CH₂OH, and —CH₂CH₃;
R⁵ is selected from the group consisting of H, F, Cl, I, Br, OH, CN, N₃, —CF₃, —CH₃, —CH₂OH, and —CH₂CH₃;
R⁶ and R^{6a} are each independently selected from the group consisting of H, F, Cl, I, Br, OH, CN, N₃, —CF₃, —CH₃, —CH₂OH, —CH₂CH₃, —C≡CH, and —C≡C—CH;
R⁷ and R^{7a} are each independently selected from the group consisting of H, —CF₃, —CH₃, and —CH₂CH₃;
R⁸ and R^{8a} are each independently selected from the group consisting of H, —CF₃, —CH₃, and —CH₂CH₃;
optionally R^{3a} and R^{6a} are connected to form C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position; and
optionally R⁵ and R⁶ are connected to form C₁-C₆ alkylene, C₂-C₆ alkenylene, —O—C₁-C₆ alkylene, or —O—C₂-C₆ alkenylene, such that where R⁵ and R⁶ are connected to form —O—C₁-C₆ alkylene or —O—C₂-C₆ alkenylene, said O is bound at the R⁵ position.

4. The compound according to claim 1, or a pharmaceutically acceptable salt thereof, wherein
Base¹ and Base² are each independently selected from the group consisting of 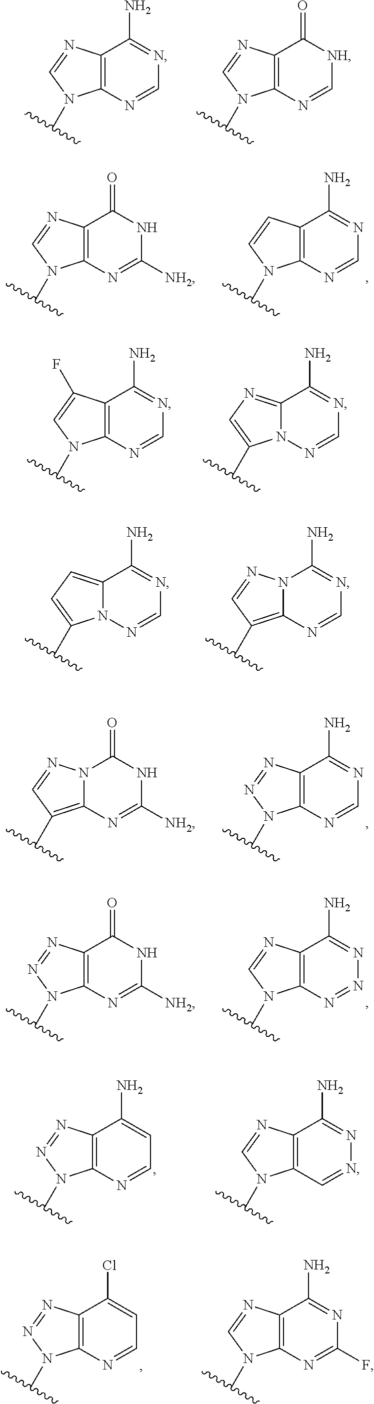 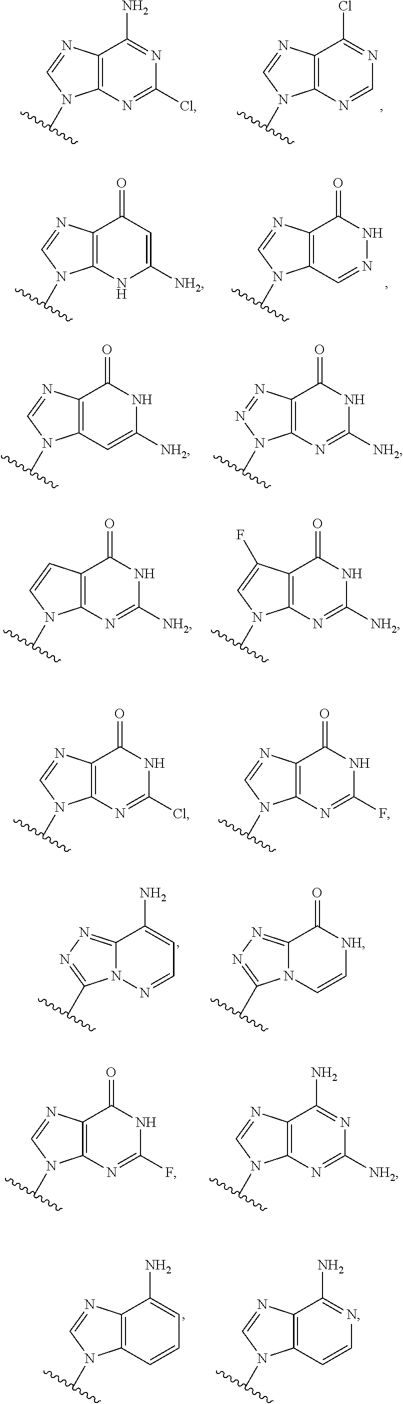 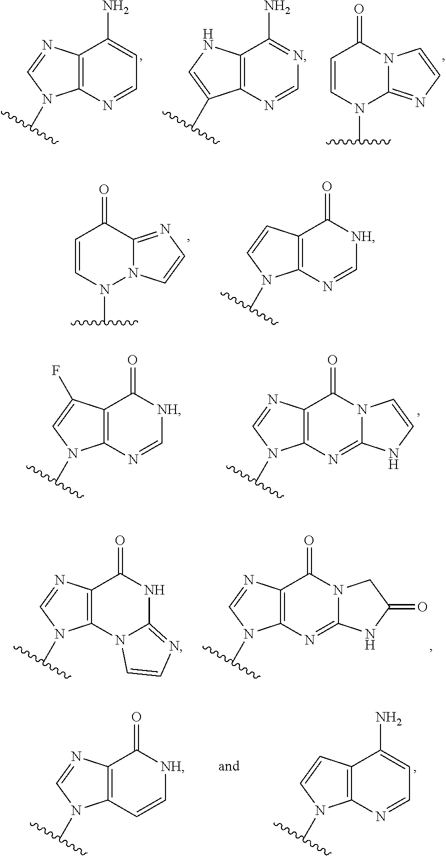
and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O— and —S—;
X^{a} and X^{a1} are each —O—;
X^{b} and X^{b1} are each —O—;
X^{c} and X^{c1} are each independently selected from the group consisting of —OH and —SH;
X^{d} and X^{d1} are each independently selected from the group consisting of O and S;
R¹ and R^{1a} are each H;
R^{2a} is selected from the group consisting of H, F, Cl, OH, CN, N₃, and —CH₃;
R³ and R^{3a} are each independently selected from the group consisting of H, F, Cl, OH, CN, N₃, —CH₃, —CH₂OH, —CH₂CH₃, —OCH₃, —OCH₂, and —OCH₂CH₃;
R⁴ and R^{4a} are each independently selected from the group consisting of H, F, Cl, OH, CN, N₃, and —CH₃;
R⁵ is selected from the group consisting of H, F, Cl, OH, CN, N₃, and —CH₃;
R⁶ and R^{6a} are each independently selected from the group consisting of H, F, CN, N₃, —CH₃, —CH═H₂, and —C≡CH;
R⁷ and R^{7a} are each H;
R⁸ and R^{8a} are each H;
optionally R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position; and
optionally R⁵ and R⁶ are connected to form C₁-C₆ alkylene, C₂-C₆ alkenylene, —O—C₁-C₆ alkylene, or —O—C₂-C₆ alkenylene, such that where R⁵ and R⁶ are connected to form —O—C₁-C₆ alkylene or —O—C₂-C₆ alkenylene, said O is bound at the R⁵ position.

5. The compound according to claim 1, or a pharmaceutically acceptable salt thereof, wherein
Base¹ and Base² are each independently selected from the group consisting of 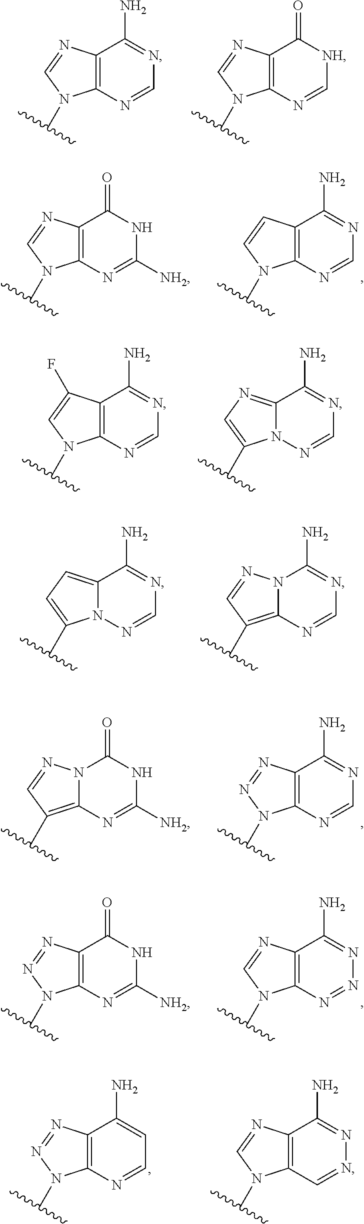 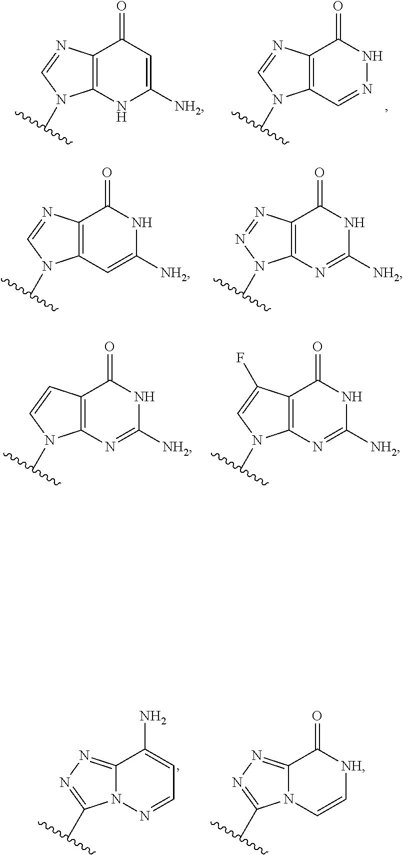 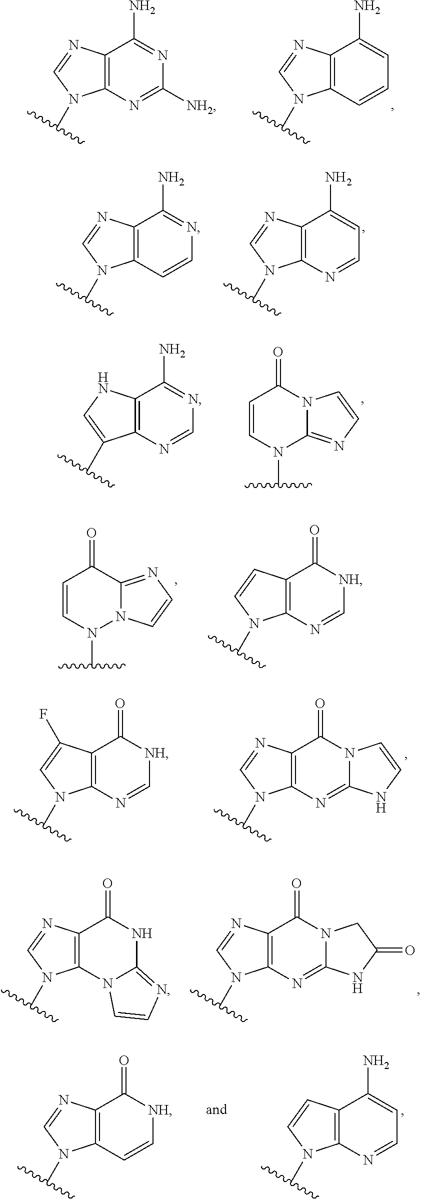
and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O— and —S—;
X^{a} and X^{a1} are each O;
X^{b} and X^{b1} are each O;
X^{c} and X^{c1} are each independently selected from the group consisting of —OH and —SH;
X^{d} and X^{d1} are each independently selected from the group consisting of O and S;
R¹ and R^{1a} are each H;
R^{2a} is selected from the group consisting of H, F, Cl, OH, CN, N₃, and —CH₃;
R³ and R^{3a} are each independently selected from the group consisting of H, F, Cl, OH, CN, N₃, —CH₃, —CH₂OH, —CH₂CH₃, —OCH₃, —OCH₂, and —OCH₂CH₃;
R⁴ and R^{4a} are each independently selected from the group consisting of H, F, Cl, OH, CN, N₃, and CH₃;
R⁵ is selected from the group consisting of H, F, Cl, OH, CN, N₃, and —CH₃;
R⁶ and R^{6a} are each independently selected from the group consisting of H, F, CN, N₃, —CH₃, —CH═H₂, and —C≡CH;
R⁷ and R^{7a} are each H;
R⁸ and R^{8a} are each H;
optionally R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position; and
optionally R⁵ and R⁶ are connected to form C₁-C₆ alkylene, C₂-C₆ alkenylene, —O—C₁-C₆ alkylene, or —O—C₂-C₆ alkenylene, such that where R⁵ and R⁶ are connected to form —O—C₁-C₆ alkylene or —O—C₂-C₆ alkenylene, said O is bound at the R⁵ position.

6. The compound according to claim 1, or a pharmaceutically acceptable salt thereof, wherein
Base¹ and Base² are each independently selected from the group consisting of 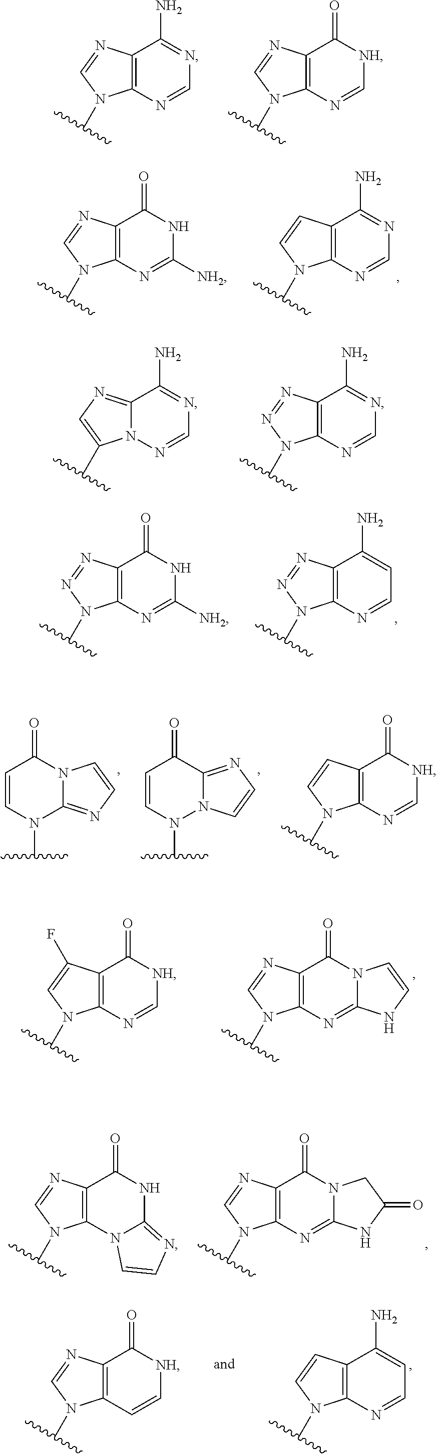
and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O— and —S—;
X^{a} and X^{a1} are each —O—;
X^{b} and X^{b1} are each —O—;
X^{c} and X^{c1} are each independently selected from the group consisting of —SH and —OH;
X^{d} and X^{d1} are each independently selected from the group consisting of O and S;
R¹ and R^{1a} are each H;
R^{2a} is selected from the group consisting of H, F, and OH;
R³ and R^{3a} are each independently selected from the group consisting of H, F, OH, —CH₃, —CH₂OH, —CH₂CH₃, —OCH₃, —OCH₂, and —OCH₂CH₃;
R⁴ and R^{4a} are each independently selected from the group consisting of H, F, and OH;
R⁵ is selected from the group consisting of H, F, and OH;
R⁶ and R^{6a} are each H;
R⁷ and R^{7a} are each H;
R⁸ and R^{8a} are each H; and
optionally R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position.

7. The compound according to claim 1, or a pharmaceutically acceptable salt thereof, wherein
Base¹ and Base² are each independently selected from the group consisting of 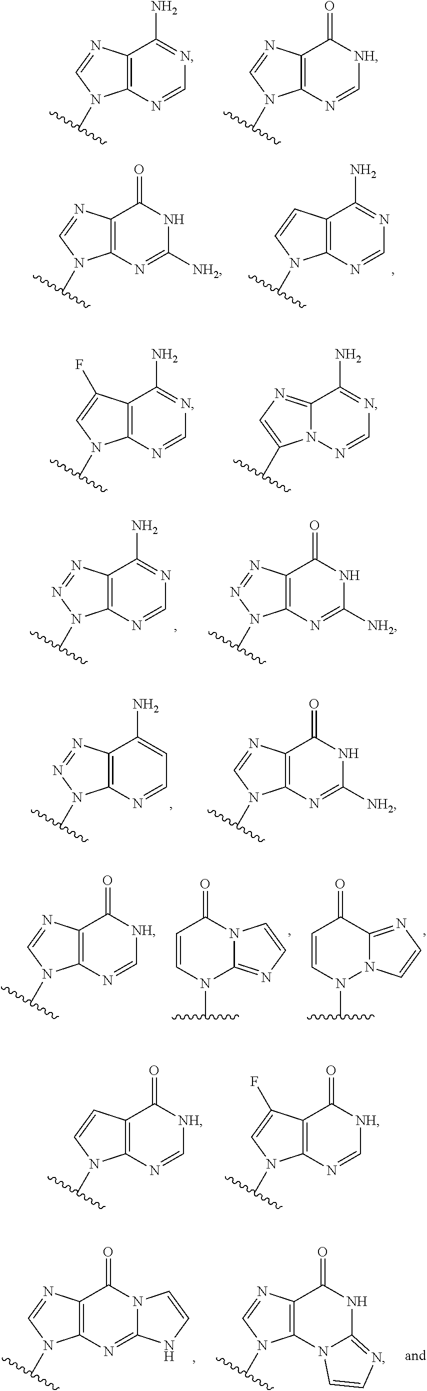 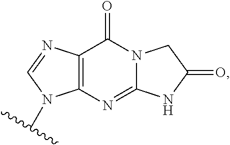 [SMILES: *n1cnc2c(=O)n3c(nc21)NC(=O)C3]
and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O— and —S—;
X^{a} and X^{a1} are each —O—;
X^{b} and X^{b1} are each —O—;
X^{c} and X^{c1} are each independently selected from the group consisting of —SH and —OH;
X^{d} and X^{d1} are each independently selected from the group consisting of O and S;
R¹ and R^{1a} are each H;
R^{2a} is selected from the group consisting of H, F, and OH;
R³ and R^{3a} are each independently selected from the group consisting of H, F, OH, —CH₃, —CH₂OH, —CH₂CH₃, —OCH₃, —OCH₂, and —OCH₂CH₃;
R⁴ is selected from the group consisting of H, F, and OH;
R^{4a} is H;
R⁵ is selected from the group consisting of H, F, and OH;
R⁶ and R^{6a} are each H;
R⁷ and R^{7a} are each H;
R⁸ and R^{8a} are each H; and
optionally R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position.

8. The compound according to claim 1, or a pharmaceutically acceptable salt thereof, wherein
Base¹ and Base² are each independently selected from the group consisting of 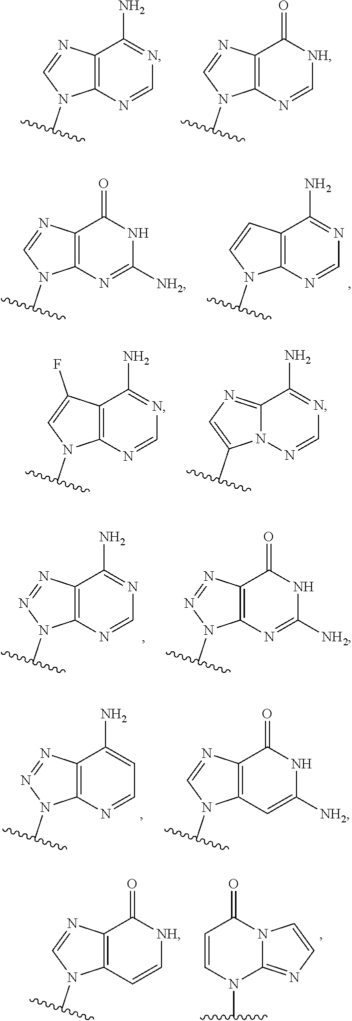 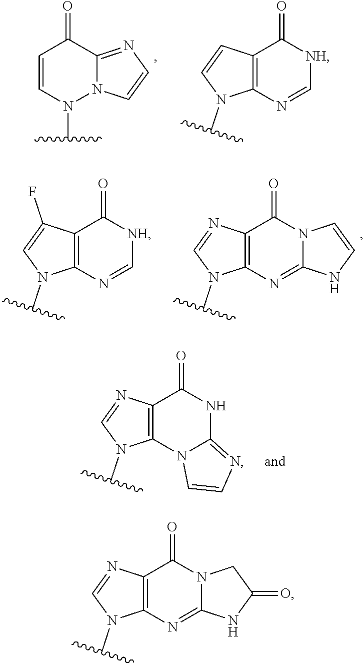
and Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O— and —S—;
X^{a} and X^{a1} are each —O—;
X^{b} and X^{b1} are each —O—;
X^{c} and X^{c1} are each independently selected from the group consisting of —SH and —OH;
X^{d} and X^{d1} are each independently selected from the group consisting of O and S;
R¹ and R^{1a} are each H;
R^{2a} is selected from the group consisting of H, F, and OH;
R³ and R^{3a} are each independently selected from the group consisting of H, F, OH, —CH₃, —CH₂OH, —CH₂CH₃, —OCH₃, —OCH₂, and —OCH₂CH₃;
R⁴ is selected from the group consisting of H, F, and OH;
R^{4a} is H;
R⁵ is selected from the group consisting of H, F, and OH;
R⁶ and R^{6a} are each H;
R⁷ and R^{7a} are each H; and
R⁸ and R^{8a} are each H.

9. The compound according to claim 1, or a pharmaceutically acceptable salt thereof, wherein
Base¹ is selected from the group consisting of 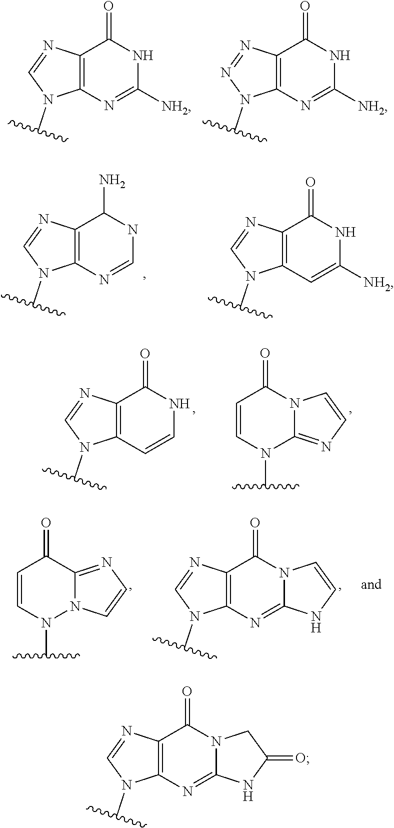
Base² is selected from the group consisting of 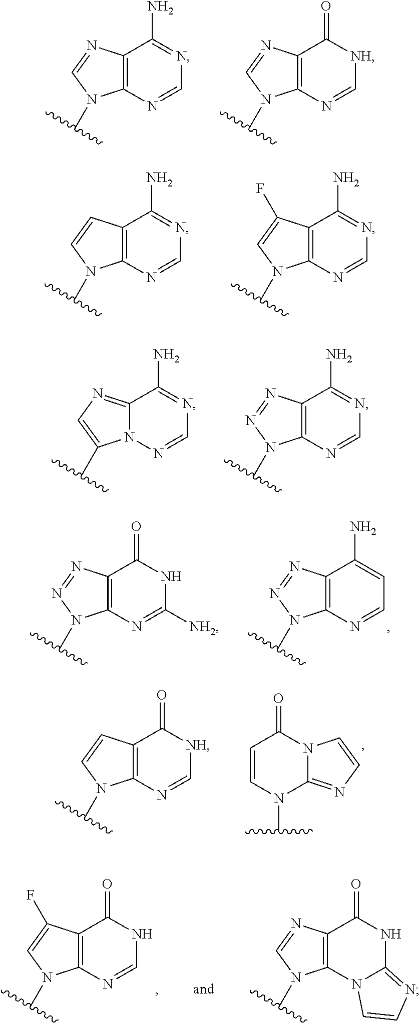
and
Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O— and —S—;
X^{a} and X^{a1} are each —O—;
X^{b} and X^{b1} are each —O—;
X^{c} and X^{c1} are each independently selected from the group consisting of —SH and —OH;
X^{d} and X^{d1} are each independently selected from the group consisting of O and S;
R¹ and R^{1a} are each H;
R^{2a} is selected from the group consisting of H, F, and OH;
R³ and R^{3a} are each independently selected from the group consisting of H, F, OH, —CH₃, —CH₂OH, —CH₂CH₃, —OCH₃, —OCH₂, and —OCH₂CH₃;
R⁴ is selected from the group consisting of H, F, and OH;
R^{4a} is H;
R⁵ is selected from the group consisting of H, F, and OH;
R⁶ and R^{6a} are each H;
R⁷ and R^{7a} are each H;
R⁸ and R^{8a} are each H; and
R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position.

10. The compound according to claim 1, or a pharmaceutically acceptable salt thereof, wherein
Base¹ is selected from the group consisting of 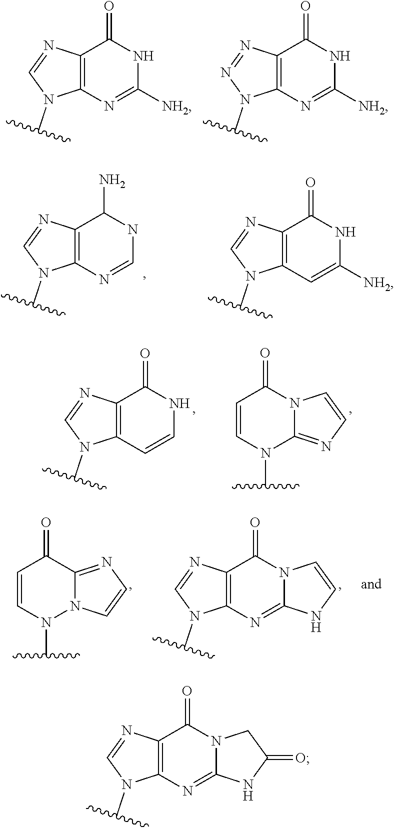
Base² is selected from the group consisting of 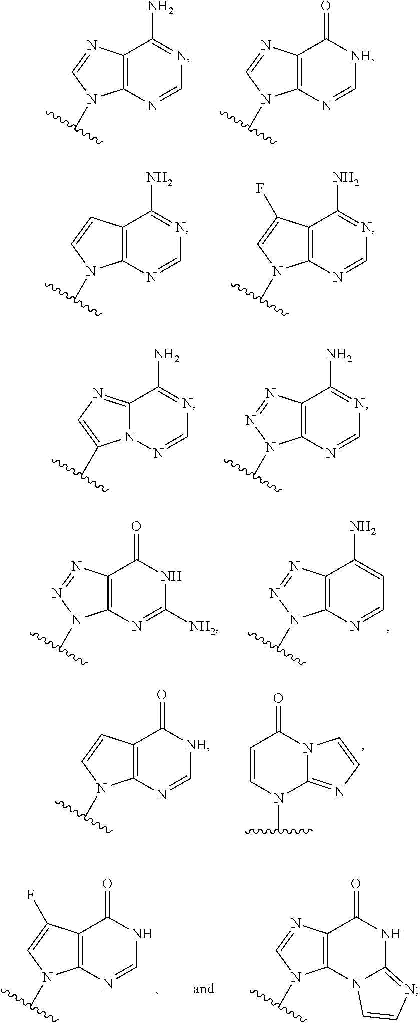
and
Base¹ and Base² each may be independently substituted by 0-3 substituents R¹⁰, where each R¹⁰ is independently selected from the group consisting of F, Cl, I, Br, OH, SH, NH₂, C₁₋₃ alkyl, C₃₋₆ cycloalkyl, O(C₁₋₃ alkyl), O(C₃₋₆ cycloalkyl), S(C₁₋₃ alkyl), S(C₃₋₆ cycloalkyl), NH(C₁₋₃ alkyl), NH(C₃₋₆ cycloalkyl), N(C₁₋₃ alkyl)₂, and N(C₃₋₆ cycloalkyl)₂;
Y and Y^{a} are each independently selected from the group consisting of —O— and —S—;
X^{a} and X^{a1} are each —O—;
X^{b} and X^{b1} are each —O—;
X^{c} and X^{c1} are each independently selected from the group consisting of —SH and —OH;
X^{d} and X^{d1} are each independently selected from the group consisting of O and S;
R¹ and R^{1a} are each H;
R^{2a} is selected from the group consisting of H, F, and OH;
R³ and R^{3a} are each independently selected from the group consisting of H, F, OH, —CH₃, —CH₂OH, —CH₂CH₃, —OCH₃, —OCH₂, and —OCH₂CH₃;
R⁴ is selected from the group consisting of H, F, and OH;
R^{4a} is H;
R⁵ is selected from the group consisting of H, F, and OH;
R⁶ and R^{6a} are each H;
R⁷ and R^{7a} are each H; and
R⁸ and R^{8a} are each H.

11. The compound according to claim 1, or a pharmaceutically acceptable salt thereof, wherein
Base¹ is selected from the group consisting of 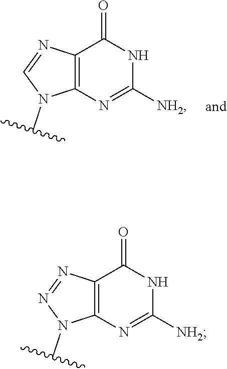 [SMILES: *n1cnc2c(=O)[nH]c(N)nc21 ; *n1nnc2c(=O)[nH]c(N)nc21]
Base² is selected from the group consisting of 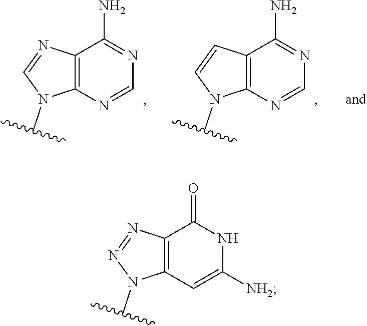 [SMILES: *n1cnc2c(N)ncnc21 ; *n1ccc2c(N)ncnc21 ; *n1nnc2c(=O)[nH]c(N)cc21]
and
Y and Y^{a} are each —O—;
X^{a} and X^{a1} are each —O—;
X^{b} and X^{b1} are each —O—;
X^{c} and X^{c1} are each independently selected from the group consisting of —SH and —OH;
X^{d} and X^{d1} are each independently selected from the group consisting of O and S;
R¹ and R^{1a} are each H;
R^{2a} is selected from the group consisting of H and F;
R³ and R^{3a} are each independently selected from the group consisting of H, OH, —CH₃, —CH₂OH, —CH₂CH₃, —OCH₃, —OCH₂, and —OCH₂CH₃;
R⁴ and R^{4a} are each H;
R⁵ is selected from the group consisting of H, F, and OH;
R⁶ and R^{6a} are each H;
R⁷ and R^{7a} are each H;
R⁸ and R^{8a} are each H; and
R^{3a} and R^{6a} are connected to C₂-C₆ alkylene, C₂-C₆ alkenylene, —O—C₁-C₆ alkylene, or —O—C₂-C₆ alkenylene, such that where R^{3a} and R^{6a} are connected to form —O—C₁-C₆ alkylene or —O—C₂-C₆ alkenylene, said O is bound at the R^{3a} position.

12. A compound selected from the group consisting of: 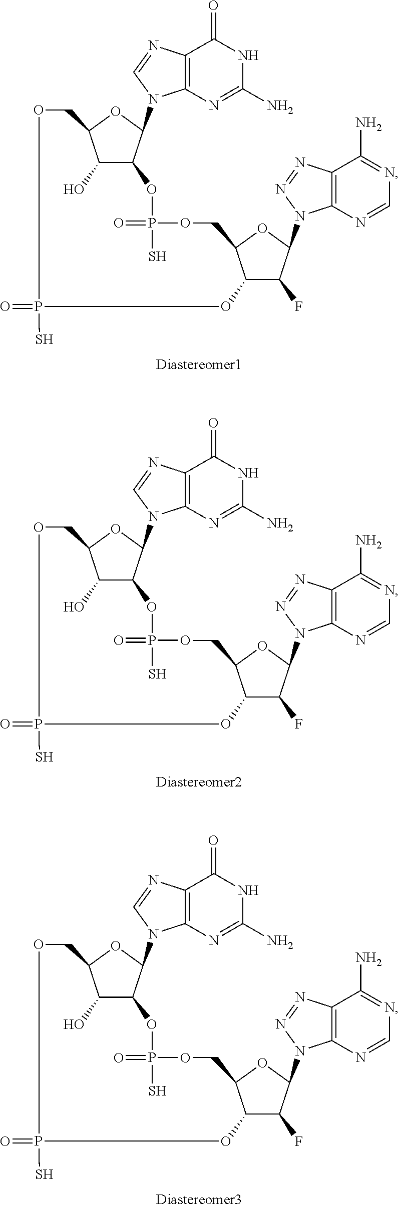 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3O)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3O)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3O)c(=O)[nH]1] 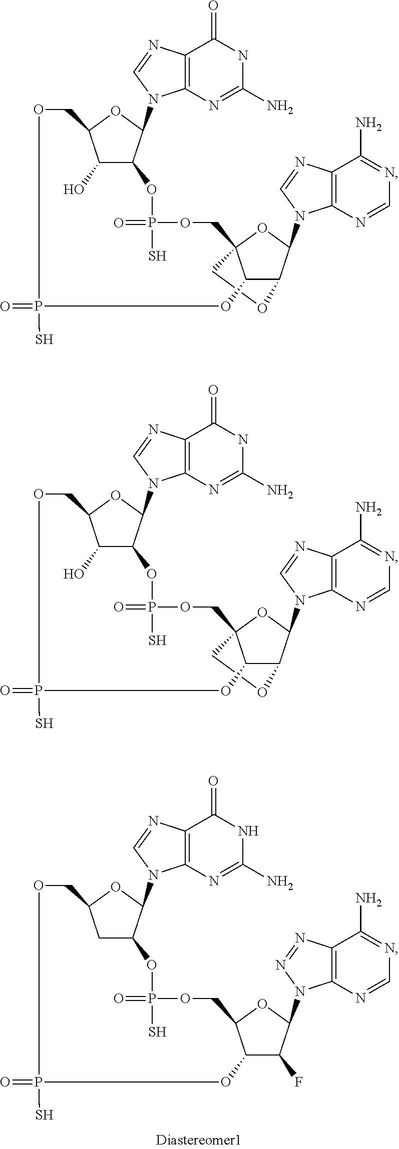 [SMILES: Nc1nc2c(ncn2[C@@H]2O[C@@H]3COP(=O)(S)O[C@H]4[C@H]5OC[C@]4(COP(=O)(S)O[C@H]2[C@@H]3O)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@H]2[C@@H]3O)O[C@H]5n2cnc3c(N)ncnc32)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1] 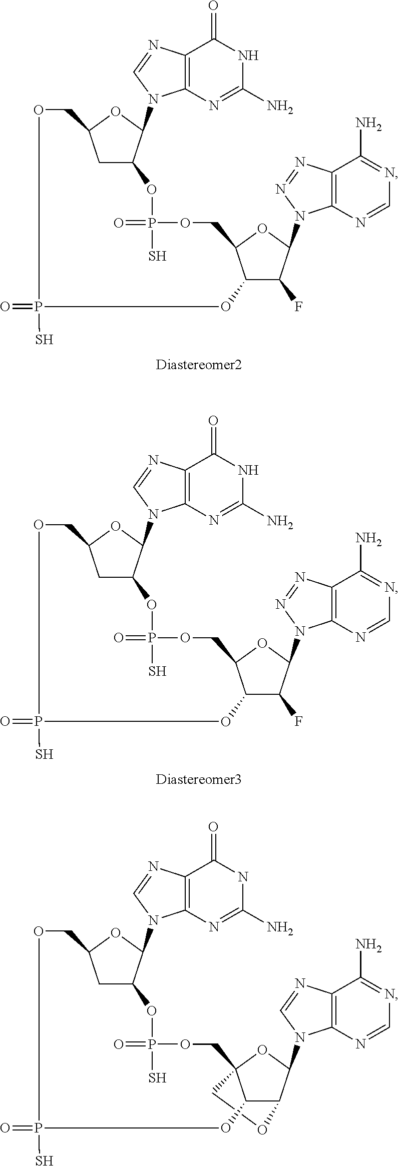 [SMILES: Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1 ; Nc1nc2c(ncn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1 ; Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1] 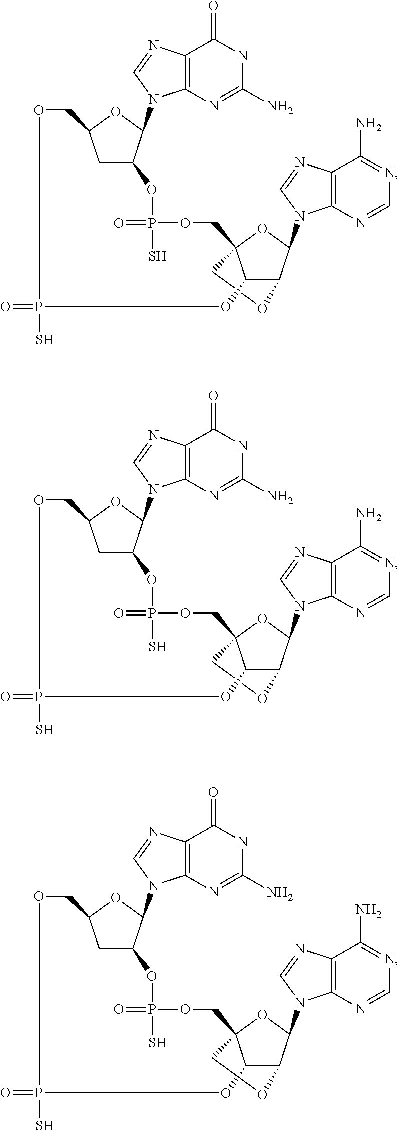 [SMILES: Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1 ; Nc1nc2c(ncn2[C@@H]2O[C@@H]3COP(=O)(S)O[C@H]4[C@H]5OC[C@]4(COP(=O)(S)O[C@H]2C3)O[C@H]5n2cnc3c(N)ncnc32)c(=O)[nH]1] 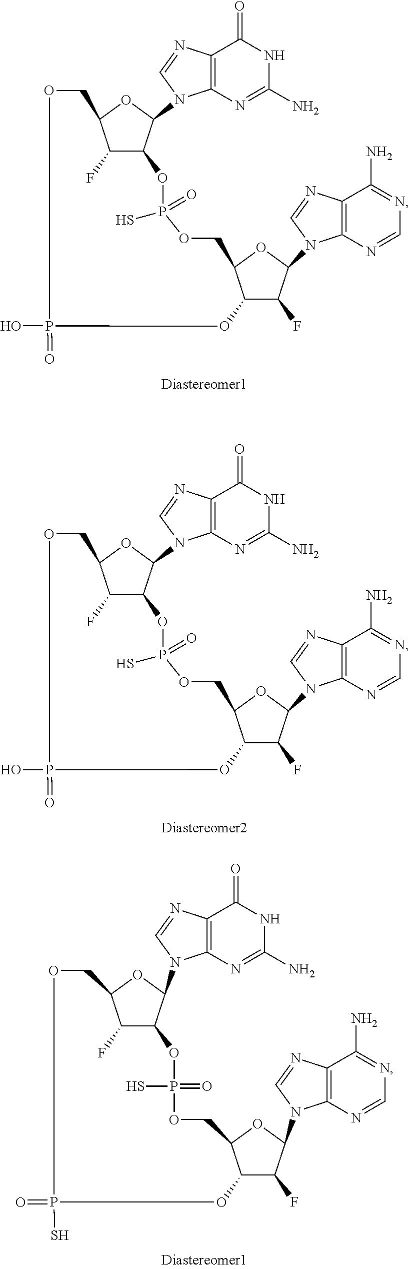 [SMILES: Nc1nc2c(ncn2[C@@H]2O[C@@H]3COP(=O)(O)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(O)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1] 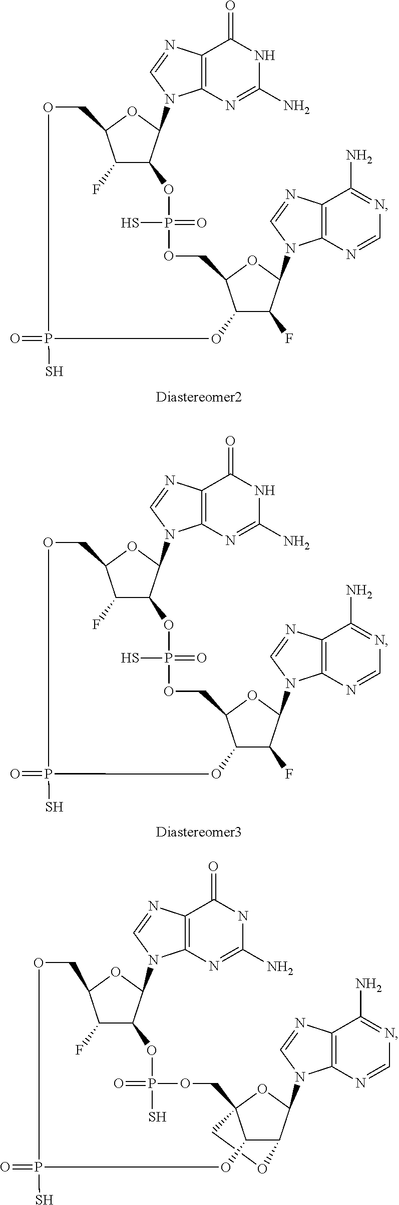 [SMILES: Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(S)O[C@H]4[C@H](F)[C@H](n5cnc6c(N)ncnc65)O[C@@H]4COP(=O)(S)O[C@H]2[C@@H]3F)c(=O)[nH]1 ; Nc1nc2c(ncn2[C@@H]2O[C@@H]3COP(=O)(S)O[C@H]4[C@H]5OC[C@]4(COP(=O)(S)O[C@H]2[C@@H]3F)O[C@H]5n2cnc3c(N)ncnc32)c(=O)[nH]1] 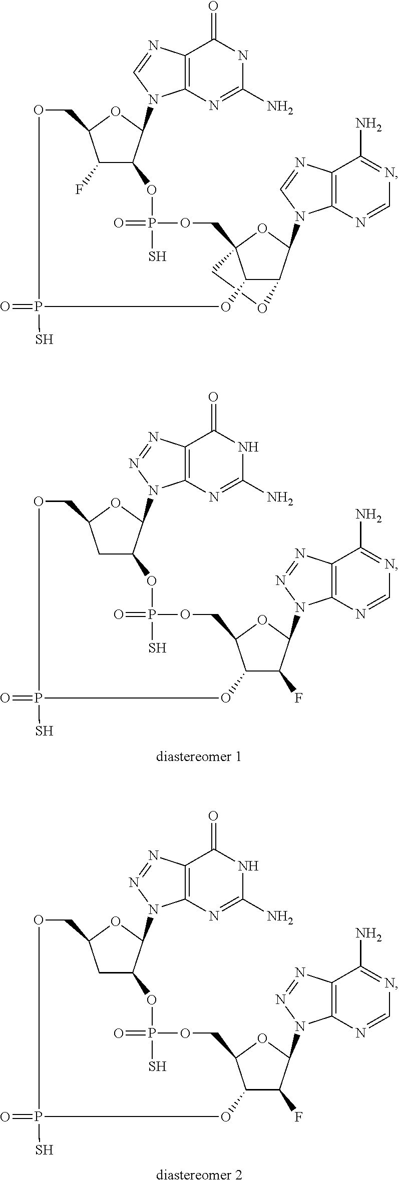 [SMILES: Nc1nc2c(ncn2[C@@H]2O[C@@H]3COP(=O)(S)O[C@H]4[C@H]5OC[C@]4(COP(=O)(S)O[C@H]2[C@@H]3F)O[C@H]5n2cnc3c(N)ncnc32)c(=O)[nH]1 ; Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1 ; Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@H]2C3)c(=O)[nH]1] 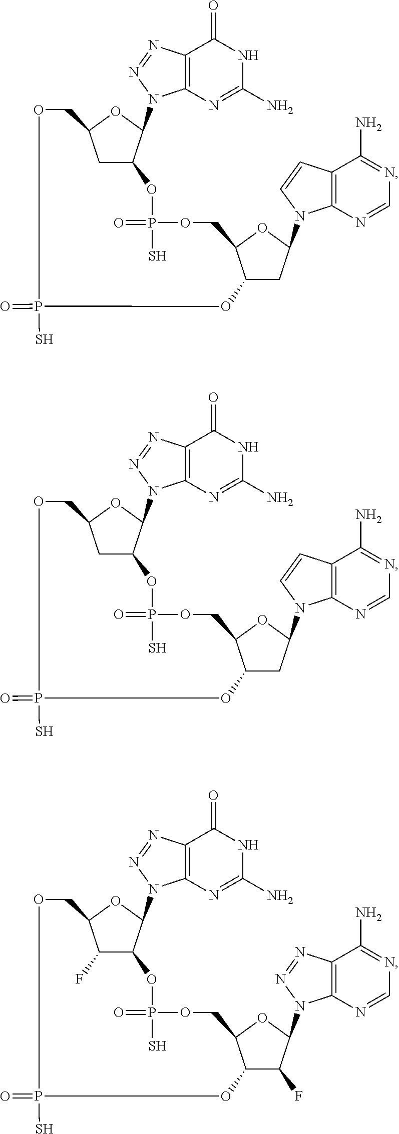 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2C3)c(=O)[nH]1 ; Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2C3)c(=O)[nH]1 ; Nc1nc2c(nnn2[C@@H]2O[C@@H]3COP(=O)(S)O[C@H]4[C@H](F)[C@H](n5nnc6c(N)ncnc65)O[C@@H]4COP(=O)(S)O[C@H]2[C@@H]3F)c(=O)[nH]1] 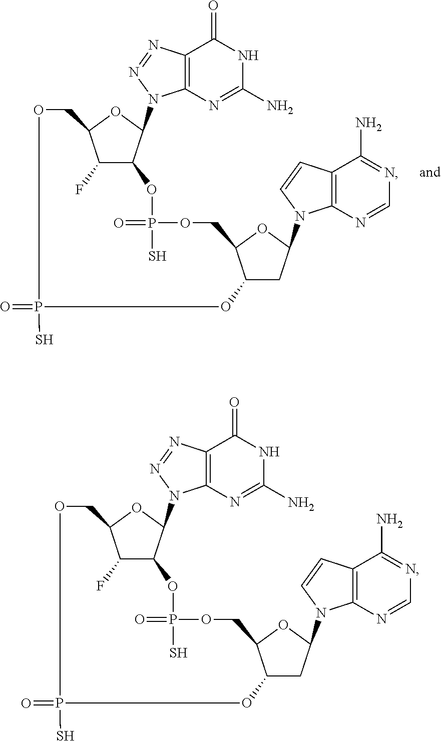 [SMILES: Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2[C@@H]3F)c(=O)[nH]1 ; Nc1nc2c(nnn2[C@@H]2O[C@@H]3COP(=O)(S)O[C@H]4C[C@H](n5ccc6c(N)ncnc65)O[C@@H]4COP(=O)(S)O[C@H]2[C@@H]3F)c(=O)[nH]1]
and pharmaceutically acceptable salts thereof.

13. A pharmaceutical composition, said pharmaceutical composition comprising:
(a) a compound according to claim 1 or a pharmaceutically acceptable salt, hydrate, solvate, or prodrug thereof; and
(b) a pharmaceutically acceptable carrier.

14. A method of inducing an immune response in a subject, said method comprising administering a therapeutically effective amount of a compound according to claim 1 to the subject.

15. A method of inducing an immune response in a subject, said method comprising administering a therapeutically effective amount of a pharmaceutical composition according to claim 13 to the subject.

16. A method of inducing a STING-dependent type I interferon production in a subject, said method comprising administering a therapeutically effective amount of a compound according to claim 1 to the subject.

17. A method of inducing a STING-dependent type I interferon production in a subject, said method comprising administering a therapeutically effective amount of a pharmaceutical composition according to claim 13 to the subject.

18. A method of treating a cell proliferation disorder in a subject, said method comprising administering a therapeutically effective amount of a compound according to claim 1 to the subject.

19. The method of claim 18, wherein the cell proliferation disorder is cancer.

20. A method of treating a cell proliferation disorder in a subject, said method comprising administering a therapeutically effective amount of a pharmaceutical composition according to claim 13 to the subject.

21. The method of claim 20, wherein the cell proliferation disorder is cancer.


SEQUENCE LISTING (4 sequences)

SEQ ID NO: 1 - protein, 414 aa, Artificial Sequence
  Full-Length HAQ STING [STING(1-379)R71H,G230A,H232R,R293Q-GG-AviTag-GS-HRV3C-HIS8]Amino Acid Sequence
MPHSSLHPSIPCPRGHGAQKAALVLLSACLVTLWGLGEPPEHTLRYLVLHLASLQLGLLLNGVCSLAEELHHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTADRAGIKDRVYSNSIYELLENGQRAGTCVLEYATPLQTLFAMSQYSQAGFSREDRLEQAKLFCQTLEDILADAPESQNNCRLIAYQEPADDSSFSLSQEVLRHLRQEEKEEVTVGSLKTSAVPSTSTMSQEPELLISGMEKPLPLRTDFSGGGLNDIFEAQKIEWHEGSLEVLFQGPHHHHHHHH

SEQ ID NO: 2 - DNA, 6482 bp, Artificial Sequence
  Full-length HAQ [STING(1-379)R71H,G230A,H232R,R293Q-GG-AviTag-GS-HRV3C-HIS8/pBAC1 ] Plasmid DNA Sequence
ggaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatcagatctgatcatggagataattaaaatgataaccatctcgcaaataaataagtattttactgttttcgtaacagttttgtaataaaaaaacctataaatataggatccatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcaccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccgctgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccagacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctctggcggtggcctgaacgacatcttcgaagcccagaaaatcgaatggcatgaaggcagcctggaagtgctgttccagggcccacaccaccatcatcaccatcaccattaatgagcggccgcactcgagcaccaccaccaccaccactaacctaggtagctgagcgcatgcaagctgatccgggttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcgatcccgcacgccgataacaagccttttcatttttactacagcattgtagtggcgagacacttcgctgtcgtcgaggtttaaacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgcca

SEQ ID NO: 3 - protein, 414 aa, Artificial Sequence
  Full-Length STING WT [STING(1-379)H232R-gg-AviTag-gs-HRV3C-HIS8] Amino Acid Sequence
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFSGGGLNDIFEAQKIEWHEGSLEVLFQGPHHHHHHHH

SEQ ID NO: 4 - DNA, 6482 bp, Artificial Sequence
  Full-length WT STING [STING(1-379)H232R-gg-AviTag-gs-HRV3C-HIS8/pBAC1] plasmid sequence
ggaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgtcaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatcagatctgatcatggagataattaaaatgataaccatctcgcaaataaataagtattttactgttttcgtaacagttttgtaataaaaaaacctataaatataggatccatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaagggcctggccccagctgagatctctgcagtgtgtgaaaaagggaatttcaacgtggcccatgggctggcatggtcatattacatcggatatctgcggctgatcctgccagagctccaggcccggattcgaacttacaatcagcattacaacaacctgctacggggtgcagtgagccagcggctgtatattctcctcccattggactgtggggtgcctgataacctgagtatggctgaccccaacattcgcttcctggataaactgccccagcagaccggtgaccgtgctggcatcaaggatcgggtttacagcaacagcatctatgagcttctggagaacgggcagcgggcgggcacctgtgtcctggagtacgccacccccttgcagactttgtttgccatgtcacaatacagtcaagctggctttagccgggaggataggcttgagcaggccaaactcttctgccggacacttgaggacatcctggcagatgcccctgagtctcagaacaactgccgcctcattgcctaccaggaacctgcagatgacagcagcttctcgctgtcccaggaggttctccggcacctgcggcaggaggaaaaggaagaggttactgtgggcagcttgaagacctcagcggtgcccagtacctccacgatgtcccaagagcctgagctcctcatcagtggaatggaaaagcccctccctctccgcacggatttctctggcggtggcctgaacgacatcttcgaagcccagaaaatcgaatggcatgaaggcagcctggaagtgctgttccagggcccacaccaccatcatcaccatcaccattaatgagcggccgcactcgagcaccaccaccaccaccactaacctaggtagctgagcgcatgcaagctgatccgggttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcgatcccgcacgccgataacaagccttttcatttttactacagcattgtagtggcgagacacttcgctgtcgtcgaggtttaaacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgcca